(19) United States
(12) Patent Application Publication
(10) Pub. No.: US 2024/0301038 A1
(43) Pub. Date: Sep. 12, 2024

(54) ANTIBODIES TO SARS-COV-2

(71) Applicants: Atreca, Inc., San Carlos, CA (US); University of Vermont and State Agricultural College, Brulington, VT (US)

(72) Inventors: Katherine L. Williams, San Carlos, CA (US); Daniel Eric Emerling, San Carlos, CA (US); Shaun M. Lippow, San Carlos, CA (US); Ngan Nguyen Atkins, San Carlos, CA (US); Jason Botten, Burlington, VT (US); Annalis Whitaker, Burlington, VT (US)

(73) Assignees: Atreca, Inc., San Carlos, CA (US); University of Vermont and State Agricultural College, Burlington, VT (US)

(21) Appl. No.: 18/552,107

(22) PCT Filed: Mar. 24, 2022
(86) PCT No.: PCT/US2022/071333

§ 371 Date: Sep. 22, 2023

Related U.S. Application Data
(60) Provisional application No. 63/166,521, filed on Mar. 26, 2021.
(60) Provisional application No. 63/271,087, filed on Oct. 22, 2021.

(51) Int. Cl. C07K 16/10; A61K 39/00; A61P 31/14; G01N 33/569; G01N 33/68

(52) U.S. Cl. CPC C07K 16/1003; A61P 31/14; G01N 33/56983; G01N 33/6854; A61K 2039/505; C07K 2317/565; C07K 2317/567; C07K 2317/76; G01N 2333/165

(57) ABSTRACT

Described herein are antibodies or variants thereof that specifically bind to coronavirus antigens, such as SARS-CoV-2 antigens. The antibodies can be neutralizing antibodies. Also provided are methods of using the antibodies, including methods of treating a subject infected with SARS-CoV-2, and methods of diagnosing a subject infected with SARS-CoV-2.

### RELATED APPLICATIONS

This application claims priority to U.S. Provisional Application No. 63/166,521, filed on Mar. 26, 2021, and U.S. Provisional Application No. 63/271,087, filed on Oct. 22, 2021. The entire content of said provisional applications is incorporated herein for all purposes.

### BACKGROUND

Coronavirus disease 19 (Covid-19) is the name of the disease caused by the virus known as Severe Acute Respiratory Syndrome Coronavirus 2 (SARS-CoV-2). The most well-defined mechanism by which SARS-CoV-2 infects cells is by binding to the ACE2 receptor on the surface of human cells. Following fusion of the viral and host cell membranes, the virus enters the cell and releases the viral RNA. The viral RNA is translated into proteins that are used to assemble new viral particles, that are then released into the body by the infected cell.

SARS-CoV-2 belongs to a class of genetically diverse viruses found in a wide range of host species, including birds and mammals (see Sun et al., “COVID-19: Epidemiology, Evolution, and Cross-Disciplinary Perspectives,” Volume 26, Issue 5, Pages e1-e2, 435-528 (May 2020)). Coronaviruses (CoVs) cause intestinal and respiratory infections in animals and in humans. SARS-CoV-2 is the seventh member of the Coronaviridae known to infect humans, and coronaviruses (CoVs). Bats are thought to be natural carriers for many SARS-like CoVs (species of Alphacoronavirus and Betacoronavirusare).

The symptoms of Covid-19 include fever, cough, shortness of breath (dyspnea), muscular soreness, chills, sore throat, and a new loss of taste or smell. Less common symptoms include gastrointestinal symptoms like nausea, vomiting, or diarrhea. In addition, older adults and people who have severe underlying medical conditions like heart or lung disease, or diabetes seem to be at higher risk for developing more serious complications from COVID-19 illness. For example, 8 out of 10 deaths reported in the U.S. have been in adults aged 65 years old and older. See the Centers for Disease Control website at https://www.cdc.gov/coronavirus/2019-ncov/symptoms.

The NIH classifies patients with COVID-19 into the following illness categories:
Asymptomatic or Presymptomatic Infection: Individuals who test positive for SARS-CoV-2 but have no symptoms
Mild Illness: Individuals who have any of various signs and symptoms (e.g., fever, cough, sore throat, malaise, headache, muscle pain) without shortness of breath, dyspnea, or abnormal imaging
Moderate Illness: Individuals who have evidence of lower respiratory disease by clinical assessment or imaging and a saturation of oxygen (SpO2) >93% on room air at sea level
Severe Illness: Individuals who have respiratory frequency >30 breaths per minute, SpO2≤93% on room air at sea level, ratio of arterial partial pressure of oxygen to fraction of inspired oxygen (PaO2/FiO2)<300, or lung infiltrates >50%
Critical Illness: Individuals who have respiratory failure, septic shock, and/or multiple organ dysfunction.

Current treatment options for COVID-19 include antivirals, steroids, immune-based therapies, neutralizing antibodies, and mechanical ventilators for patients with respiratory failure. Despite advances in treatment approaches over the past year, there is no identified cure for COVID-19 infection. MRNA-based vaccines from Moderna and BioNTech/Pfizer have both been issued Emergency Use Authorization by the FDA. However, these vaccines may have limited efficacy in vulnerable populations like those suffering from cancer or autoimmune disease. Accordingly, a need exists to develop safe and effective prophylactic and treatment options for COVID-19 infection.

### BRIEF SUMMARY

Described herein are antibodies that inhibit the binding of a coronavirus to a cell or reduce infection of a cell by a coronavirus, and methods of using the antibodies. In some embodiments, the coronavirus is a member of the betacoronavirus genus. In some embodiments, the coronavirus is a Severe Acute Respiratory Syndrome Coronavirus 2 (SARS-CoV-2). In some embodiments, the coronavirus is a variant of SARS-CoV-2 selected from the group consisting of the Alpha, Beta, Gamma, Delta, Epsilon, Mu and Omicron variants.

In some embodiments, the antibody is selected from the list of antibodies in Table 4 or 5. In some embodiments, the antibody comprises all six CDRs of an antibody selected from the group consisting of AB-009614, AB-010019, AB-009270, AB-009346, AB-009681, AB-009761, AB-009620, AB-009734, AB-009760, and AB-009281, or wherein the antibody comprises both the VH and VL of an antibody selected from the group consisting of AB-009614, AB-010019, AB-009270, AB-009346, AB-009681, AB-009761, AB-009620, AB-009734, AB-009760, and AB-009281.

In some embodiments, wherein the antibody inhibits binding of Alpha, Beta, Gamma, Delta, Epsilon, Mu and Omicron variants of SARS-CoV-2 and/or reduces infection of a cell by Alpha, Beta, Gamma, Delta, Epsilon, Mu and Omicron variants of SARS-CoV-2. In some embodiments, the antibody comprises all six CDRs of an antibody selected from the group consisting of AB-009270 and AB-009620, or wherein the antibody comprises both VH and VL of an antibody selected from the group consisting of AB-009270 and AB-009620.

In some embodiments, the antibody comprises the LCDRs and the HCDRs of an antibody listed in Table 4. In some embodiments, the antibody comprises a VH amino acid sequence and a VL amino acid sequence listed in Table 5, or ii) a VH amino acid sequence with at least 70% identity to the VH amino acid sequence in Table 5 and a VL amino acid sequence with at least 70% identity to the VL amino acid sequence in Table 5, wherein variations as compared to the VH amino acid sequence or the VL amino acid sequence in Table 5 are in the framework regions only.

In some embodiments, the antibody comprises HCDR1, HCDR2, and/or HCDR3 of an antibody listed in Table 4, or variants of the HCDR1, HCDR2, and/or HCDR3 in which 1 or more amino acids are substituted. In some embodiments, the antibody comprises the LCDR1, LCDR2, and/or LCDR3 of an antibody listed in Table 4, or variants of the LCDR1, LCDR2, and/or LCDR3 in which 1 or more amino acids are substituted.

In some embodiments, the antibody comprises the HCDR1, HCDR2, HCDR3, LCDR1, LCDR2, and LCDR3 of an antibody listed in Table 4, or variants of the HCDR1, HCDR2, HCDR3, LCDR1, LCDR2, and/or LCDR3 in which 1 or more amino acids are substituted. In some embodiments, the antibody comprises the HCDR1, HCDR2, HCDR3, LCDR1, LCDR2, and LCDR3 of an antibody listed in Table 4.

In some embodiments, the antibody comprises a heavy chain variable region sequence listed in Table 5, or a variant thereof. In some embodiments, the antibody comprises a light chain variable region sequence listed in Table 5, or a variant thereof. In some embodiments, the antibody comprises a heavy chain variable region sequence listed in Table 5 and a light chain variable region sequence listed in Table 5, or a variant thereof.

In some embodiments, the antibody comprises the LCDR1, LDCR2, LCDR3, and HCDR1, HCDR2, HCDR3 of antibody AB-009614 of Table 4, or variants of the LCDR1, LCDR2, LCDR3, HCDR1, HCDR2, and/or HCDR3 of antibody AB-009614 in which 1 or more amino acids are substituted. In some embodiments, the comprises the heavy chain variable region sequence of AB-009614 of Table 5, or a variant thereof. In some embodiments, the antibody comprises the light chain variable region sequence of AB-009614 of Table 5, or a variant thereof.

In some embodiments, the antibody binds to a spike glycoprotein (S-protein) encoded by the coronavirus. In some embodiments, the antibody binds to a membrane (M) protein, an envelope (E) protein, or a nucleocapsid (N) protein encoded by the coronavirus.

In some embodiments, the antibody binds to the S trimer encoded by the coronavirus. In some embodiments, the antibody binds to RBD, S1 monomer and S trimer. In some embodiments, the antibody binds to RBD, S1 monomer and S trimer and does not bind to the S2 protein. In some embodiments, the antibody binds to S2 and S trimer.

In some embodiments, the antibody inhibits binding of the coronavirus to a receptor on the surface of the cell. In some embodiments, the cell surface receptor is ACE2.

In some embodiments, the antibody is an isolated antibody. In some embodiments, the antibody is one that competes with any antibody disclosed herein.

In one aspect, a pharmaceutical composition is provided. In some embodiments, the pharmaceutical composition comprises an antibody described herein.

In another aspect, an expression vector is provided. In some embodiments, the expression vector comprises a heterologous polynucleotide encoding a heavy chain variable region listed in Table 5. In some embodiments, the expression vector comprises a heterologous polynucleotide encoding a light chain variable region listed in Table 5. In some embodiments, the expression vector comprises a heterologous polynucleotide encoding a cognate pair of heavy and light chain variable regions listed in Table 5. In another aspect, provided herein is a recombinant nucleic acid encoding an antibody or an antibody fragment of any one of the antibody disclosed herein.

In another aspect, a host cell is provided. In some embodiments, the host cell comprises an expression vector described herein.

In another aspect, a method of producing an antibody that inhibits binding of a coronavirus to a cell is described, the method comprising culturing a host cell comprising an expression vector described herein under conditions in which the polynucleotide encoding the heavy chain and the polynucleotide encoding the light chain are expressed.

In another aspect, a method of producing an antibody that inhibits binding of a coronavirus to a cell is described, the method comprising synthesizing the amino acid sequence of the heavy and/or light chains of an antibody described herein.

In another aspect, a method of inducing an immune response is described, the method comprising administering an antibody described herein to a subject. In some embodiments, the immune response comprises antibody-dependent cellular cytotoxicity (ADCC), antibody dependent cellular phagocytosis (ADCP), and/or complement-dependent cytotoxicity (CDC). In some embodiments, the antibody is administered intravenously.

In another aspect, a method of treating a patient infected with a coronavirus is described, the method comprising administering a therapeutically effective amount of an antibody or pharmaceutical composition described herein to the patient. In some embodiments, the antibody or pharmaceutical composition is administered intravenously. In some embodiments, the method further comprises administering a second treatment to the patient, wherein the second treatment is selected from an anti-viral agent or an anti-inflammatory agent. In some embodiments, the method further comprises administering a second treatment to the patient, wherein the second treatment comprises an antibody that binds SARS-CoV-2. In some embodiments, the second treatment comprises an antibody selected from the group consisting of casirivimab, imdevimab, etesevima, bamlanivimab, CT-P59, BRII-196, BRII-198, VIR-7831, AZD7442, AZD8895, AZD1061, TY027, SCTA01, MW33, JS016, DXP593, DXP604, STI-2020, BI 767551/DZIF-10c, COR-101, HLX70, ADM03820, HFB30132A, ABBV-47D11, C144-LS, C-135-LS, LY-CovMab, JMB2002, and ADG20. In some embodiments, the second treatment comprises an antibody selected from the group consisting of casirivimab, imdevimab, etesevima, and bamlanivimab.

In another aspect, a method of identifying a patient that is infected with a coronavirus is described, the method comprising detecting binding of an antibody described herein to a sample obtained from the patient, wherein binding greater than a negative control value indicates the patient is infected with the coronavirus. In some embodiments, the method is an in vitro method that is not practiced on an animal or human subject. In some embodiments, the method further comprises contacting a sample obtained from the patient with an antibody described herein. In some embodiments, the sample is a blood or serum sample. In some embodiments, the method further comprises treating the patient with an antibody or pharmaceutical composition described herein.

In another aspect, a method of identifying an antibody having anti-viral activity is described, the method comprising mutagenizing a polynucleotide encoding a heavy chain variable region, or a light chain variable region of an antibody described herein, expressing the antibody comprising the mutagenized heavy chain or light chain variable region; and selecting an antibody that inhibits binding of the virus to a cell.

In another aspect, an in vitro method for detecting an immune response is described, the method comprising identifying a cell infected with a coronavirus by contacting the cell with an antibody described herein, and detecting binding of the antibody to the cell.

In another aspect, a method of preventing infection of a subject with a coronavirus is described, the method comprising administering an antibody or pharmaceutical composition described herein, to the subject, wherein the antibody or pharmaceutical composition is administered at a dose sufficient to prevent or reduce infection of one or more host cells in the subject by the coronavirus. In some embodiments, the coronavirus is SARS-CoV-2.

In another aspect, a method of diagnosing a subject that is infected with a coronavirus is described, the method comprising detecting binding of an antibody described herein to a sample obtained from the subject, wherein binding greater than a negative control value indicates the subject is infected with the coronavirus. In some embodiments, the coronavirus is SARSCoV-2.

In another aspect, use of an antibody described herein in a method of inducing an immune response in vivo is described.

In another aspect, use of an antibody described herein in a method of treating a coronavirus infection is described. In some embodiments, the coronavirus is SARS-CoV-2.

### BRIEF DESCRIPTION OF THE DRAWINGS

FIGS. 1A-C shows binding of control antibodies to the RBD, S1 and S2 proteins of SARS-CoV-2.

FIG. 2 shows neutralization activity and binding of antibody AB-009614 to the RBD, S1, S2, and S trimer antigens of SARS-CoV-2.

FIG. 3 shows neutralization activity and binding of antibody AB-009270 to the RBD, S1, S2, and S trimer antigens of SARS-CoV-2.

FIG. 4 shows neutralization activity and binding of antibody AB-009281 to the RBD, S1, S2, and S trimer antigens of SARS-CoV-2.

FIG. 5 shows neutralization activity and binding of antibody AB-009346 to the RBD, S1, S2, and S trimer antigens of SARS-CoV-2.

FIG. 6 shows neutralization activity and binding of antibody AB-009615 to the RBD, S1, S2, and S trimer antigens of SARS-CoV-2.

FIG. 7 shows neutralization activity and binding of antibody AB-009760 to the RBD, S1, S2, and S trimer antigens of SARS-CoV-2.

FIG. 8 shows neutralization activity and binding of antibody AB-009620 to the RBD, S1, S2, and S trimer antigens of SARS-CoV-2.

FIG. 9 shows neutralization activity and binding of antibody AB-009661 to the RBD, S1, S2, and S trimer antigens of SARS-CoV-2.

FIG. 10 shows neutralization activity and binding of antibody AB-009761 to the RBD, S1, S2, and S trimer antigens of SARS-CoV-2.

FIG. 11 shows neutralization activity and binding of antibody AB-009769 to the RBD, S1, S2, and S trimer antigens of SARS-CoV-2.

FIG. 12 shows neutralization activity and binding of antibody AB-009681 to the RBD, S1, S2, and S trimer antigens of SARS-CoV-2.

FIG. 13 shows neutralization activity and binding of antibody AB-009734 to the RBD, S1, S2, S trimer and N antigens of SARS-CoV-2.

FIGS. 14A-B show alignment of certain antibodies VH and VL sequences with CDRs designated by Kabat and IMGT.

### DETAILED DESCRIPTION

As used in herein, the singular forms “a”, “an”, and “the” include plural referents unless the content clearly dictates otherwise. Thus, for example, reference to “an antibody” optionally includes a combination of two or more such molecules and the like.

The term “about” as used herein refers to the usual error range for the respective value readily known to the skilled person in this technical field, for example, ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of antibody or fragment thereof that exhibits the desired biological activity, e.g., binding the specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab)₂, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab)₂ fragment that has two antigen combining sites and is still capable of cross-linking antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

As used herein, “V-region” refers to an antibody variable region domain comprising the segments of Framework 1, CDR1, Framework 2, CDR2, Framework 3, CDR3, and Framework 4. The heavy chain V-region, VH, is a consequence of rearrangement of a V-gene (HV), a D-gene (HD), and a J-gene (HJ), in what is known as V(D)J recombination during B-cell differentiation. The light chain V-region, VL, is a consequence of rearrangement of a V-gene (LV) and a J-gene (LJ).

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the CDR is located. Thus, a V_{H} CDR3 (HCDR3) is in the variable domain of the heavy chain of the antibody in which it is found, whereas a V_{L} CDR3 (LCDR3) is the CDR3 from the variable domain of the light chain of the antibody in which it is found. The term “CDR” is used interchangeably with “HVR” when referring to CDR sequences.

The amino acid sequences of the CDRs and framework regions can be determined using various well known definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J.Mol.Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al., Proc. Natl Acad. Sci. USA, 86, 9268-9272 (1989); Martin, et al., Methods Enzymol., 203, 121-153, (1991); Pedersen et al., Immunomethods, 1, 126, (1992); and Rees et al., In Sternberg M. J. E. (ed.), Protein Structure Prediction. Oxford University Press, Oxford, 141-172 1996). Reference to CDRs as determined by Kabat numbering is based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

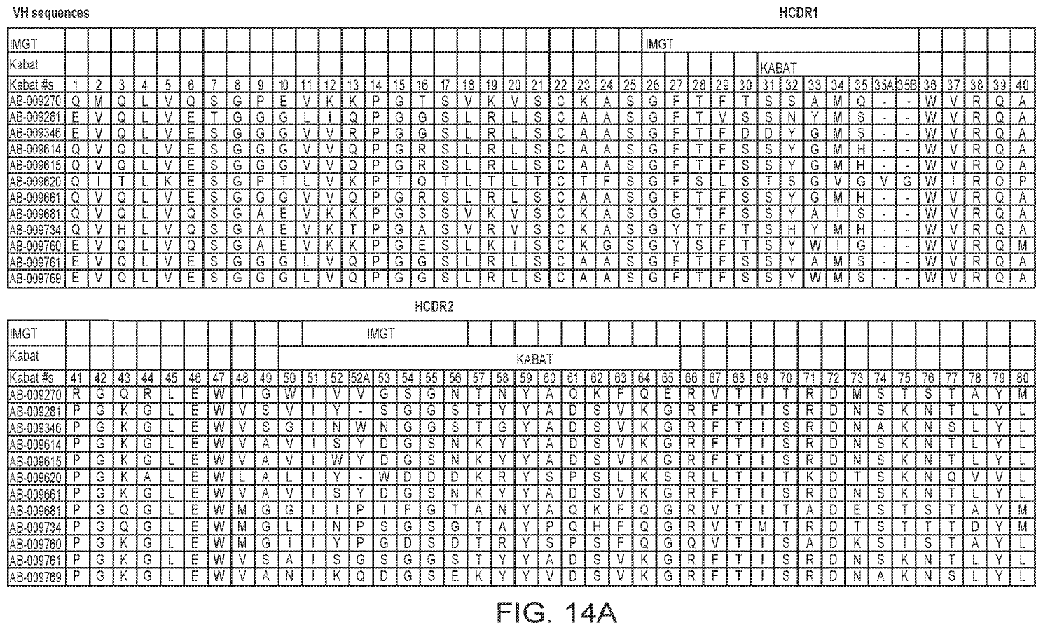

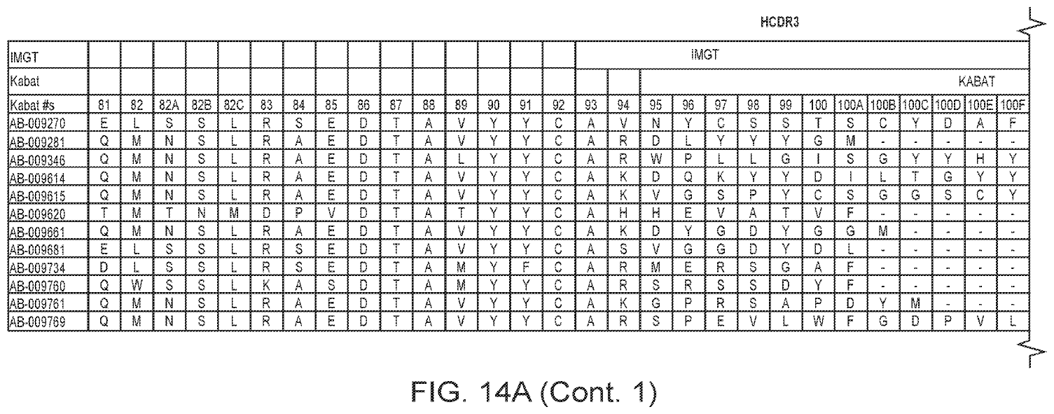

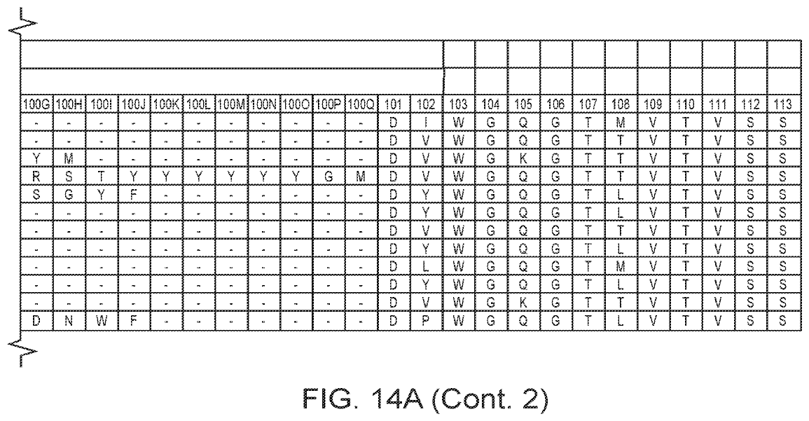

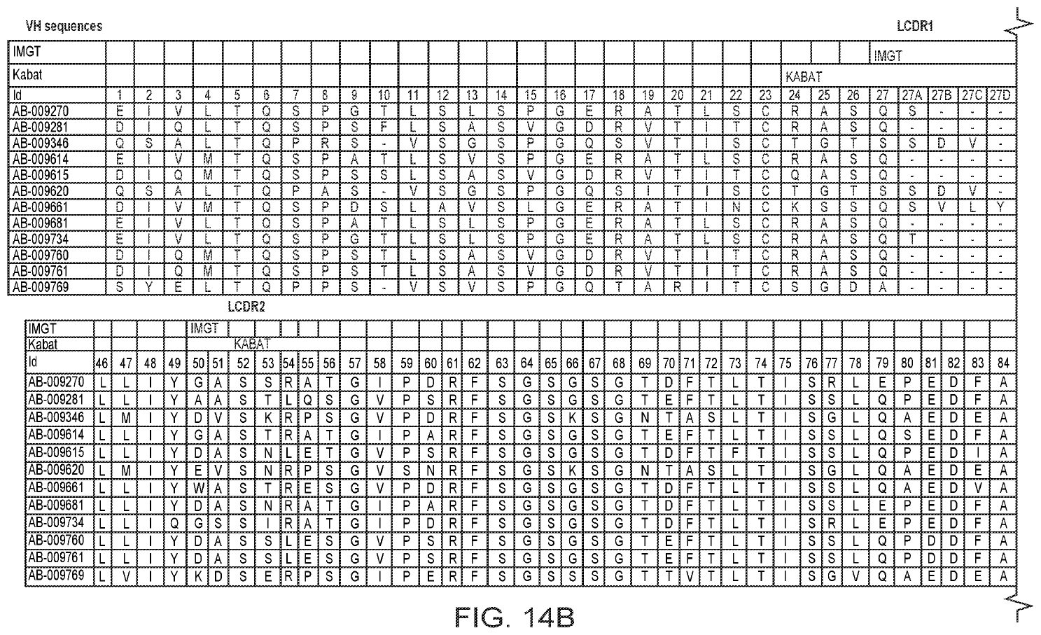

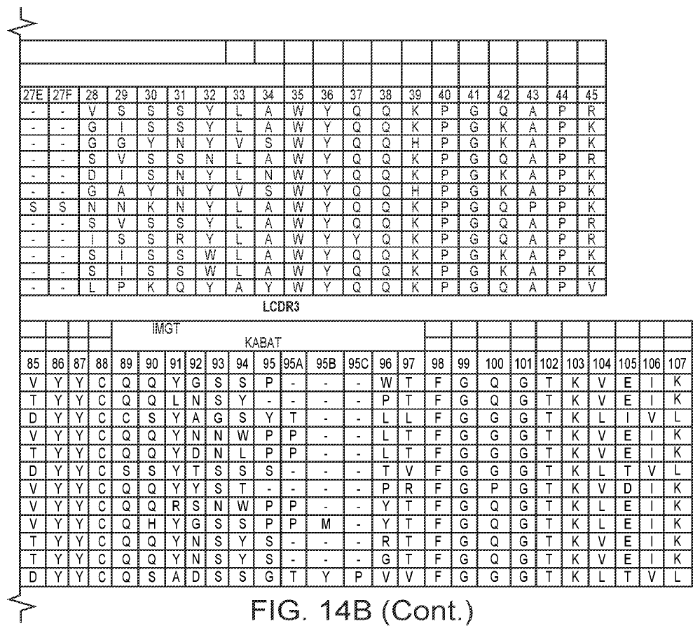

CDRs as shown in Table 4 are defined by IMGT and Kabat. The V_{H} CDRs as listed in Table 4 are defined as follows: HCDR1 is defined by combining Kabat and IMGT; HCDR2 is defined by Kabat; and the HCDR3 is defined by IMGT. The V_{L} CDRs as listed in Table 4 are defined by Kabat. FIGS. 14 A-B show alignment of VH and VL sequences of certain antibodies in Table 5 with CDRs designated by Kabat and IMGT. As known in the art, numbering and placement of the CDRs can differ depending on the numbering system employed. It is understood that disclosure of a variable heavy and/or variable light sequence includes the disclosure of the associated CDRs, regardless of the numbering system employed.

An “Fc region” refers to the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ2. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, VA). The term “Fc region” may refer to this region in isolation or this region in the context of an antibody or antibody fragment. “Fc region” includes naturally occurring allelic variants of the Fc region as well as modifications that modulate effector function. Fc regions also include variants that do not result in alterations to biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibodies (see, e.g., Angal, et al., Mol Immunol 30:105-108, 1993).

An “EC₅₀” as used herein in the context of an Fc receptor engagement assay, refers to the half-maximal effective concentration, which is the concentration of an antibody that induces a response (signal generated in engagement assay) halfway between the baseline and maximum after a specified exposure time. In some embodiments, the “fold over EC50” is determined by dividing the EC50 of a reference antibody by the EC₅₀ of the test antibody.

The 50% inhibitory concentration (“IC₅₀”) is the concentration of antibody at which either pseudovirus or full-length virus infectivity is reduced by at least 50% as compared to viral infection in the absence of anti-SARS-CoV-2 antibody, or in the presence of a negative control antibody not expected neutralize SARS-CoV-2. The IC₅₀ may also reference the reciprocal dilution of plasma or serum at which 50% inhibition of viral infection is calculated. In some embodiments, neutralizing activity can also be measured as a function of the area under the positive portion of the neutralization curve.

The term “endpoint titer” refers to the lowest concentration of antibody or the highest dilution of plasma or serum where binding to an antigen is significantly higher than the negative control.

The term “equilibrium dissociation constant” abbreviated (K_{D}), refers to the dissociation rate constant (k_{d}, time⁻¹) divided by the association rate constant (kₐ, time⁻¹ M⁻¹). Equilibrium dissociation constants can be measured using any method. Thus, in some embodiments antibodies of the present disclosure have a K_{D} of less than about 50 nM, typically less than about 25 nM, or less than 10 nM, e.g., less than about 5 nM or than about 1 nM and often less than about 10 nM as determined by bio-layer interferometry analysis using a biosensor system such as an Octet® system performed at 25° C. In some embodiments, an antibody of the present disclosure has a K_{D} of less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a bivalent antibody. In the context of the present invention, an “improved” K_{D} refers to a lower K_{D}. In some embodiments, an antibody of the present disclosure has a K_{D} of less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a monovalent antibody, such as a monovalent Fab. In some embodiments, an anti-SARS-CoV-2 antibody of the present disclosure has K_{D} less than 100 pM, e.g., or less than 75 pM, e.g., in the range of 1 to 100 pM, when measured by biolayer interferometry using a biosensor system such as an Octet® system performed at 25° C. In some embodiments, an anti-SARS-CoV-2 antibody of the present disclosure has K_{D} of greater than 100 pM, e.g., in the range of 100-1000 pM or 500-1000 pM when measured by biolayer interferometry using a biosensor system such as a Octet® system performed at 25° C.

The term “monovalent molecule” as used herein refers to a molecule that has one antigen-binding site, e.g., a Fab or scFv.

The term “bivalent molecule” as used herein refers to a molecule that has two antigen-binding sites. In some embodiments, a bivalent molecule of the present invention is a bivalent antibody or a bivalent fragment thereof. In some embodiments, a bivalent molecule of the present invention is a bivalent antibody. In some embodiments, a bivalent molecule of the present invention is an IgG. In general. monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” as used herein refer to the binding of both antigen-binding sites of a bivalent molecule to its antigen. Preferably both antigen-binding sites of a bivalent molecule share the same antigen specificity.

The term “valency” as used herein refers to the number of different binding sites of an antibody for an antigen. A monovalent antibody comprises one binding site for an antigen. A bivalent antibody (e.g., a bivalent IgG antibody) comprises two binding sites for the same antigen.

The term “avidity” as used herein in the context of antibody binding to an antigen refers to the combined binding strength of multiple binding sites of the antibody. Thus, “bivalent avidity” refers to the combined strength of two binding sites

The term “affinity” as used herein refers to either the single or combined strength of one or both arms of an antibody (e.g., an IgG antibody) binding to either a simple or complex antigen expressing one or more epitopes. As defined here, the term “affinity” does not imply a specific number of valencies between the two binding partners.

The phrase “specifically (or selectively) binds” to an antigen or target or “specifically (or selectively) immunoreactive with,” when referring to a protein or peptide, refers to a binding reaction whereby the antibody binds to the antigen or target of interest. In the context of this invention, the antibody selectively binds to a SARS-CoV-2 antigen.

The terms “identical” or percent “identity,” in the context of two or more polynucleotide or polypeptide sequences, refer to two or more sequences or subsequences that are the same or have a specified percentage of amino acid residues that are the same (e.g., 100% identity) or have a specified percentage of nucleotides or amino acid residues are the same (e.g., at least 70%, at least 75%, at least 80%, 85%, 90%, 91%, 92%, 93%, 94%, 95%, 96%, 97%, 98%, 99%, or higher sequence identity; or 70%, 75%, 80%, 85%, 90%, 91%, 92%, 93%, 94%, 95%, 96%, 97%, 98%, or 99% sequence identity)) identity over a specified region, e.g., the length of the two sequences, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity include the BLAST 2.0 algorithms, which are described in Altschul et al., Nuc. Acids Res. 25:3389-3402 (1977) and Altschul et al., J. Mol. Biol. 215:403-410 (1990). Thus, for purposes of this disclosure, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a V_{H} region polypeptide “corresponds to” an amino acid in the V_{H} region of SEQ ID NO:1 when the residue aligns with the amino acid in SEQ ID NO:1 when optimally aligned to SEQ ID NO:1. The polypeptide that is aligned to the reference sequence need not be the same length as the reference sequence.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gin; and (xi) beta-branched amino acids Thr, Val, and Ile. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, the substitution of one or more of the naturally occurring nucleotides with an analog, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular, and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors can direct the expression of nucleic acids to which they are operatively linked. Such vectors are referred to herein as “expression vectors”.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cell and includes the primary transformed cell and progeny derived therefrom without regard to the number of passages.

A polypeptide “variant,” as the term is used herein, is a polypeptide that typically differs from a polypeptide specifically disclosed herein in one or more substitutions, deletions, additions and/or insertions. As used herein, a “variant” refers to an engineered sequence, rather than a naturally occurring sequence.

The term “comparable,” in the context of describing the strength of binding of two antibodies to the same target, refers to two dissociation constant (K_{D}) values calculated from two binding reactions that are within three (3) fold from each other. In other words, the ratio between the first K_{D} (the K_{D} of the binding reaction between the first antibody and the target) and the second K_{D} (the K_{D} of the binding reaction between the second antibody and the target) is within the range of 1:3 or 3:1, endpoints exclusive. A lower K_{D} value denotes stronger binding. For example, an antibody variant that has stronger binding as compared to a reference antibody binds to the target with a K_{D} that is at least ⅓ of the K_{D} measured against the same target for the reference antibody.

As used herein, “therapeutic agent” refers to an agent that when administered to a patient suffering from a disease, in a therapeutically effective dose, will cure, or at least partially arrest the symptoms of the disease and complications associated with the disease.

As used herein, a “neutralizing antibody” is an antibody that acts by preventing a virus or other infectious pathogen from infecting a host target cell.

As used herein a “B cell” refers to any cell that has at least one rearranged immunoglobulin gene locus.

As used herein, “variable region” refers to a variable nucleotide sequence that arises from a recombination event, for example, it can include a V, J, and/or D region of an immunoglobulin or T cell receptor sequence isolated from a T cell or B cell of interest, such as an activated T cell or an activated B cell.

As used herein “B cell variable immunoglobulin region” refers to a variable immunoglobulin nucleotide sequence isolated from a B cell. For example, a variable immunoglobulin sequence can include a V, J, and/or D region of an immunoglobulin sequence isolated from a B cell of interest such as a memory B cell, an activated B cell, or plasmablast.

As used herein “immunoglobulin region” refers to a contiguous portion of nucleotide sequence from one or both chains (heavy and light) of an antibody.

As used herein “identification region” refers to a nucleotide sequence label (e.g., a unique barcode sequence) that can be coupled to at least one nucleotide sequence for, e.g., later identification of at least one nucleotide sequence.

As used herein, “barcode” or “barcode sequence” refers to any unique sequence label that can be coupled to at least one nucleotide sequence for, e.g., later identification of at least one nucleotide sequence.

The term “paired” heavy and light chains, or “paired” heavy and light chain variable regions, or “cognate pair” refers to native pairs of immunoglobulin heavy and light variable regions that are expressed by a single B cell.

### Compositions

Described herein are antibodies or variants thereof that inhibit the binding of SARS-CoV-2 to a cell or reduce or prevent infection of a cell by SARS-CoV-2. In some embodiments, the antigen-binding protein is an antibody or antigen-binding fragment thereof.

Antibodies that Bind to SARS-CoV-2 Antigens

Described herein are antibodies or variants thereof that specifically bind to coronavirus antigens. In some embodiments, the antibodies are neutralizing antibodies. In some embodiments, the antibodies specifically bind to SARS-CoV-2 antigens. In some embodiments, the antibodies specifically bind to a protein antigen that is translated from a viral RNA, such as a SARS-CoV-2 viral RNA. In some embodiments, the antibodies specifically bind to a viral spike protein. In some embodiments, the antibodies specifically bind to the SARS-CoV-2 spike protein. In some embodiments, the antibodies specifically bind to either the S1 or S2 subunits of the SARS-CoV-2 spike protein. In some embodiments, the antibodies specifically bind to a SARS-CoV-2 S1 or S2 glycoprotein. In some embodiments, the antibodies specifically bind to the ectodomain of S1 and S2. In some embodiments, the antibodies specifically bind to the receptor-binding domain (RBD) of the S1 subunit. In some embodiments, the antibodies specifically bind to the S2 subunit. In some embodiments, the antibodies specifically bind to a SARS-CoV-2 membrane (M) protein. In some embodiments, the antibodies specifically bind to a SARS-CoV-2 envelope (E) protein. In some embodiments, the antibodies specifically bind to a SARS-CoV-2 nucleocapsid (N) protein.

In some embodiments, the antibody is a neutralizing antibody against SARS-CoV-2. Non-limiting examples of such antibody include AB-009614, AB-010019, AB-009270, AB-009346, AB-009681, AB-009761, AB-009620, AB-009734, AB-009760, and AB-009281. The CDRs and the VH and VL sequences of each of the antibodies are disclosed in Table 4. The VH and VL sequences of these antibodies are disclosed in Table 5. In some embodiments, the antibody is a pan-neutralizing antibody, i.e., the antibody is capable of neutralizing Alpha, Beta, Gamma, Delta, Epsilon, Mu, and Omicron variants of SARS-CoV-2. Such pan-neutralizing antibodies include, for example, AB-009620.

It is thought that the SARS-CoV-2 virus utilizes components of the renin-angiotensin system (RAS) (ACE2 and TMPRSS2) to enter cells. Current research indicates that SARS-CoV-2 binds to the cell surface receptor Angiotensin-converting enzyme 2 (ACE2). See, e.g., Walls et al., 2020, Cell 180, 1-12, Mar. 19, 2020: doi.org/10.1016/j.cell.2020.02.058. Thus, in some embodiments, the antibodies specifically bind to the ACE2 protein (www.uniprot.org/uniprot/Q9BYF1), or an antigenic fragment thereof. In some embodiments, the antibodies specifically bind to TMPRSS2 (serine protease, uniprot O15393), or an antigenic fragment thereof.

In some embodiments, the antibodies inhibit the binding of the coronavirus to a cell more efficiently as compared to a control, non-specific antibody. In some embodiments, the antibodies reduce infection of a cell by a coronavirus. The term “reduce infection” refers to the experimental antibody decreasing the number of viruses that enter a cell as compared to a control antibody.

In some embodiments, the antibodies inhibit the binding of the coronavirus to the ACE2 receptors on the surface of human cells. In some embodiments, the antibodies are neutralizing antibodies.

The antibodies described herein can be IgG, IgM, or IgA antibodies.

In some embodiments, the antibodies described herein are selected from the antibodies listed in Table 4 or Table 5. In some embodiments, the antibodies described herein comprise a heavy chain variable region sequence listed in Table 5, or a non-naturally occurring variant thereof. In some embodiments, the antibodies described herein comprise a light chain variable region sequence listed in Table 5, or a non-naturally occurring variant thereof. In some embodiments, the antibodies described herein comprise a nucleic acid sequence encoding an amino acid sequence listed in Table 5, or a non-naturally occurring variant thereof. In some embodiments, the antibodies described herein comprise a nucleic acid sequence encoding a heavy chain variable region sequence listed in Table 5, or a non-naturally occurring variant thereof. In some embodiments, the antibodies described herein comprise a nucleic acid sequence encoding a light chain variable region sequence listed in Table 5, or a non-naturally occurring variant thereof.

The antibodies described herein can be monoclonal antibodies. In some embodiments, the antibody is an antibody fragment, e.g., a Fv, Fab, Fab′, scFv, diabody, or F(ab)₂ fragment. In another embodiment, the antibody is a full-length antibody, e.g., an IgG antibody or other antibody class or isotype as defined herein. For a review of certain antibody fragments, see Hudson et al. Nat. Med. 9: 129-134 (2003). Antibody fragments can be made by various techniques, including but not limited to proteolytic digestion of an intact antibody as well as production by recombinant host cells (e.g., E. coli or phage), as described herein.

In some embodiments an antibody described herein is in a monovalent format. In some embodiments, the antibody is in a fragment format, e.g., an Fv, Fab, Fab′, scFv, diabody, or F(ab)₂ fragment.

In some embodiments, an antibody of the present disclosure is employed in a bispecific or multi-specific format. For example, in some embodiments, the antibody may be incorporated into a bispecific or multi-specific antibody that comprises a further binding domain that binds to the same or a different antigen.

In some embodiments, an antibody of the present disclosure comprises an Fc region that has effector function, e.g., exhibits antibody-dependent cellular cytotoxicity ADCC. In some embodiments, the Fc region may be an Fc region engineered to alter one or more functional properties of the antibody, such as serum half-life, complement fixation, Fc receptor binding, and/or ADCC. Furthermore, an antibody of the disclosure may be chemically modified (e.g., one or more chemical moieties can be attached to the antibody) to alter its glycosylation or to alter other functional properties of the antibody. Additional modifications may also be introduced. For example, the antibody can be linked to one of a variety of polymers, for example, polyethylene glycol.

### Variants

Also provided are variants of the antibodies described herein. For example, the antibodies of the disclosure can be modified to include one or more amino acid substitutions in a heavy and/or light chain variable region CDR sequence in Table 4. In some embodiments, the heavy and/or light chain variable region CDR sequences can be modified to include 1, 2, 3, 4, or 5 amino acid substitutions relative to the sequence in Table 4. The heavy and/or light chain variable regions can also include variants in the framework region. In some embodiments, one or more of the four framework regions (Framework 1, Framework 2, Framework 3 and/or Framework 4) can be modified to include one or more amino acid substitutions (e.g., 1, 2, 3, 4, 5, or more amino acid substitutions) relative to the natural or wild-type frameword sequence. In some embodiments, the modifications to the framework regions do not decrease binding affinity of the antibody to target SARS-CoV-2 antigens or epitopes. In some embodiments, the heavy and/or light chain variable regions can be modified to include variants in one or more of the framework regions only. In some embodiments, the heavy and/or light chain variable regions can be modified to include variants in one or more of the framework regions and not variants in the CDR sequences. Thus, in some embodiments, the antibody can comprise a modified VH region having at least 70% sequence identity (e.g., at least 70%, 75%, 80%, 85%, 90%, 91%, 92%, 93%, 94%, 95%, 96%, 97%, 98%, 99% sequence identity) to a VH amino acid sequence in Table 5, wherein the sequence variations relative to the VH amino acid sequence in Table 5 are in the framework region only. In some embodiments, the antibody can comprise a modified VL region having at least 70% sequence identity (e.g., at least 70%, 75%, 80%, 85%, 90%, 91%, 92%, 93%, 94%, 95%, 96%, 97%, 98%, 99% sequence identity) to a VH amino acid sequence in Table 5, wherein the sequence variations relative to the VL amino acid sequence in Table 5 are in the framework region only. In some embodiments, an antibody disclosed herein comprises all six CDRs of an antibody in Table 4, and the antibody comprises heavy chain framework regions (all four framework regions together) having at least 70% sequence identity to the heavy chain framework regions in the same antibody in Table 4, and/or light chain framework regions (all four framework regions together) having at least 70% sequence identity to the light chain framework regions of the same antibody in Table 4.

### Pharmaceutical Compositions

In a further aspect, provided herein are pharmaceutical compositions for administration of an anti-SARS-CoV-2 antibody described herein to a mammalian subject, such as a human or companion animal, who is either at risk of infection with SARS-CoV-2, who has been exposed to a known SARS-CoV-2 case or who is infected with SARS-CoV-2 or has symptoms of coronavirus disease 2019 (COVID-19). The pharmaceutical composition can be administered in an amount and according to a schedule sufficient to prevent infection by SARS-CoV-2, to prevent development of disease following exposure to SARS-CoV-2, to prevent development of disease following exposure to SARS-CoV-2, or to reduce a symptom of COVID-19 disease. Such compositions may comprise an antibody described herein, or a polynucleotide encoding the antibody, and a pharmaceutically acceptable diluent or carrier. In some embodiments, a polynucleotide encoding the antibody may be contained in a plasmid vector for delivery, or a viral vector. In some embodiments, the pharmaceutical composition comprises a therapeutically effective amount of the antibody. As used herein, a “therapeutically effective dose” or a “therapeutically effective amount” refers to an amount sufficient to prevent, cure, or treat one or more symptoms of COVID-19 disease. A therapeutically effective dose can be determined by monitoring a patient's response to therapy. Typical benchmarks indicative of a prophylactically effective dose includes prevention of SARS-CoV-2 infection, or if infected, reduced severity of COVID-19 disease symptoms. Typical benchmarks indicative of a therapeutically effective dose includes amelioration or prevention of symptoms of COVID-19 disease in the patient, including, for example, reduction in lung inflammation. Amounts effective for either prophylactic or therapeutic use will depend upon the severity of the disease and the general state of the patient's health, including other factors such as age, weight, gender, administration route, and the like. Single or multiple administrations of the antibody will be dependent on the dosage and frequency as required and tolerated by the patient.

As used herein, a “prophylactically effective dose” or a “prophylactically effective amount” refers to an amount sufficient to prevent infection by SARS-CoV-2 or onset of one or more symptoms of COVID-19 disease.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in Remington: The Science and Practice of Pharmacy 20th Ed. (Lippincott, Williams & Wilkins 2012). In particular embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials that can serve as pharmaceutically-acceptable carriers, diluents or excipients include water; buffers, e.g., phosphate-buffered saline; sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

The pharmaceutical composition can be formulated for any suitable route of administration, including for example, systemic, parenteral, intrapulmonary, intranasal, or local administration. Parenteral administration can include intramuscular, intravenous (e.g., as a bolus or by continuous infusion over a period of time), intraarterial, intraperitoneal, intracerobrospinal, intrasynovial, inhalation, oral or subcutaneous administration. In certain embodiments, the pharmaceutical composition is formulated for intravenous administration and has a concentration of antibody of 10-100 mg/mL, 10-50 mg/mL, 20 to 40 mg/mL, or about 30 mg/mL. In certain embodiments, the pharmaceutical composition is formulated for subcutaneous injection and has a concentration of antibody of 50-500 mg/mL, 50-250 mg/mL, or 100 to 150 mg/mL, and a viscosity less than 50 cP, less than 30 cP, less than 20 cP, or about 10 cP. In some embodiments, the pharmaceutical compositions are liquids or solids. In particular embodiments, the pharmaceutical compositions are formulated for parenteral, e.g., intravenous, subcutaneous, intraperiotoneal, or intramuscular administration. A subject may be administered an antibody or pharmaceutical composition comprising an antibody of the present disclosure one or more times; and may be administered before, after, or concurrently with another therapeutic agent as further described below.

Formulations include those in which the antibody is encapsulated in micelles, liposomes or drug-release capsules (active agents incorporated within a biocompatible coating designed for slow-release); ingestible formulations; formulations for topical use, such as creams, ointments, and gels; and other formulations such as inhalants, aerosols, and sprays.

In some embodiments, e.g., for parenteral administration, the antibodies or antigen-binding fragments thereof are formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate may also be used.

The dose and dosage regimen depends upon a variety of factors readily determined by a physician, such as the nature of the viral infection, the characteristics of the subject, and the subject's history. In particular embodiments, the amount of antibody or antigen-binding fragment thereof administered or provided to the subject is in the range of about 0.1 mg/kg to about 50 mg/kg of the subject's body weight. Depending on the type and severity of the infection, in certain embodiments, about 0.1 mg/kg to about 50 mg/kg body weight (e.g., about 0.1-15 mg/kg/dose) of antibody or antigen-binding fragment thereof may be provided as an initial candidate dosage to the patient, whether, for example, by one or more separate administrations, or by continuous infusion. The progress of the therapy is readily monitored by conventional methods and assays and based on criteria known to the physician or other persons of skill in the art.

### Vectors

In some aspects, a composition described herein includes a vector. In some embodiments, the vector comprises one or more polynucleotide sequences that encode an antibody described herein or encode a heavy and light chain described herein. In some embodiments, the vector comprises one or more polynucleotide sequences that encode a cognate pair of heavy and light chains described herein. Vectors can be used in the transformation of a host cell with a nucleic acid sequence. In some aspects, a vector can include one or more polynucleotides encoding an antibody described herein. In one embodiment, a library of nucleic acid sequences encoding an antibody described herein may be introduced into a population of cells, thereby allowing screening of a library. The term “vector” is used to refer to a carrier nucleic acid molecule into which a nucleic acid sequence can be inserted for introduction into a cell where it can be replicated. A nucleic acid sequence can be “exogenous” or “heterologous” which means that it is foreign to the cell into which the vector is being introduced or that the sequence is homologous to a sequence in the cell but in a position within the host cell nucleic acid in which the sequence is ordinarily not found. Vectors include plasmids, cosmids, and viruses (e.g., bacteriophage). One of skill in the art may construct a vector through standard recombinant techniques, which are described in Maniatis et al., 1988 and Ausubel et al., 1994, both of which references are incorporated herein by reference. In some aspects, a vector can be a vector with the constant regions of an antibody pre-engineered in. In this way, one of skill can clone just the VDJ regions of an antibody of interest and clone those regions into the pre-engineered vector.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences for the transcription and possibly translation of an operably linked coding sequence in a particular host organism. In addition, to control sequences that govern transcription and translation, vectors and expression vectors may contain nucleic acid sequences that serve other functions as well.

In some aspects, a vector can include a promoter. In some aspects, a vector can include an enhancer. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic cell, promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (see U.S. Pat. Nos. 4,683,202, 5,928,906, each incorporated herein by reference).

In some aspects, a promoter and/or enhancer that effectively directs the expression of the DNA segment in the cell type chosen for expression. One example of such promoter that may be used is the E. coli arabinose or T7 promoter. Those of skill in the art of molecular biology generally are familiar with the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

In some aspects, vectors can include initiation signals and/or internal ribosome binding sites. A specific initiation signal also may be included for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In some aspects, a vector can include sequences that increase or optimize the expression level of the DNA segment encoding the gene of interest. An example of such sequences includes the addition of introns in the expressed mRNA (Brinster, R. L. et al. (1988) Introns increase transcriptional efficiency in transgenic mice. Proc. Natl. Acad. Sci. USA 85, 836-40; Choi, T. et al. (1991) A generic intron increases gene expression in transgenic mice. Mol. Cell. Biol. 11, 3070-4). Another example of a method for optimizing the expression of the DNA segment is “codon optimization”. Codon optimization involves the insertion of silent mutations in the DNA segment to reduce the use of rare codons to optimize protein translation (Codon engineering for improved antibody expression in mammalian cells. Carton J M, Sauerwald T, Hawley-Nelson P, Morse B, Peffer N, Beck H, Lu J, Cotty A, Amegadzie B, Sweet R. Protein Expr Purif. 2007 October; 55(2):279-86. Epub 2007 Jun. 16.).

In some aspects, a vector can include multiple cloning sites. Vectors can include a multiple cloning site (MCS), which is a nucleic acid region that contains multiple restriction enzyme sites, any of which can be used in conjunction with standard recombinant technology to digest the vector (see Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. The use of such enzymes is understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

In some aspects, a vector can include a termination signal. The vectors or constructs will generally comprise at least one termination signal. A “termination signal” or “terminator” is comprised of the DNA sequences involved in the specific termination of an RNA transcript by an RNA polymerase. Thus, in certain embodiments, a termination signal that ends the production of an RNA transcript is contemplated. A terminator may be necessary in vivo to achieve desirable message levels.

Terminators contemplated for use include any known terminator of transcription described herein or known to one of ordinary skill in the art, including but not limited to, for example, rho dependent or rho independent terminators. In certain embodiments, the termination signal may be a lack of transcribable or translatable sequence, such as due to a sequence truncation.

In some aspects, a vector can include an origin of replication.

In order to propagate a vector in a host cell, it may contain one or more origins of replication sites (often termed “ori”), which is a specific nucleic acid sequence at which replication is initiated.

In some aspects, a vector can include one or more selectable and/or screenable markers. In certain embodiments, cells containing a nucleic acid construct may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually, the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

In one aspect, the vector can express DNA segments encoding multiple polypeptides of interest. For example, DNA segments encoding both the immunoglobulin heavy chain and light chain can be encoded and expressed by a single vector. In one aspect, both DNA segments can be included on the same expressed RNA and internal ribosome binding site (IRES) sequences used to enable expression of the DNA segments as separate polypeptides (Pinkstaff J K, Chappell S A, Mauro V P, Edelman G M, Krushel L A., Internal initiation of translation of five dendritically localized neuronal mRNAs., Proc Natl Acad Sci USA. 2001 Feb. 27; 98(5):2770-5. Epub 2001 Feb. 20.). In another aspect, each DNA segment has its own promoter region resulting in the expression of separate mRNAs (Andersen C R, Nielsen L S, Baer A, Tolstrup A B, Weilguny D. Efficient Expression from One CMV Enhancer Controlling Two Core Promoters. Mol Biotechnol. 2010 Nov. 27. [Epub ahead of print]).

### Host Cells and Expression Systems

In some aspects, a composition can include a host cell. In some aspects, a host cell can include a polynucleotide or vector described herein. In some aspects, a host cell can include a eukaryotic cell (e.g., insect, yeast, or mammalian) or a prokaryotic cell (e.g., bacteria). In the context of expressing a heterologous nucleic acid sequence, “host cell” can refer to a prokaryotic cell, and it includes any transformable organism that is capable of replicating a vector and/or expressing a heterologous gene encoded by a vector. A host cell can and has been, used as a recipient for vectors. A host cell may be “transfected” or “transformed,” which refers to a process by which exogenous nucleic acid is transferred or introduced into the host cell. A transformed cell includes the primary subject cell and its progeny.

In particular embodiments, a host cell is a Gram-negative bacterial cell. These bacteria are suited for use in that they possess a periplasmic space between the inner and outer membrane and, particularly, the aforementioned inner membrane between the periplasm and cytoplasm, which is also known as the cytoplasmic membrane. As such, any other cell with such a periplasmic space could be used. Examples of Gram-negative bacteria include, but are not limited to, E. coli, Pseudomonas aeruginosa, Vibrio cholera, Salmonella typhimurium, Shigella flexneri, Haemophilus influenza, Bordetella pertussi, Erwinia amylovora, Rhizobium sp. The Gram-negative bacterial cell may be still further defined as a bacterial cell which has been transformed with the coding sequence of a fusion polypeptide comprising a candidate binding polypeptide capable of binding a selected ligand. The polypeptide is anchored to the outer face of the cytoplasmic membrane, facing the periplasmic space, and may comprise an antibody coding sequence or another sequence. One means for expression of the polypeptide is by attaching a leader sequence to the polypeptide capable of causing such directing.

Numerous prokaryotic cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for the replication of many vectors. Bacterial cells used as host cells for vector replication and/or expression include DH5-alpha, JM109, and KC8, as well as a number of commercially available bacterial hosts such as SURE™ Competent Cells and SOLOPACK™ Gold Cells (STRATAGENE™, La Jolla). In some aspects, other bacterial cells such as E. coli LE392 are contemplated for use as host cells.

Many host cells from various cell types and organisms are available and would be known to one of skill in the art. Similarly, a viral vector may be used in conjunction with a prokaryotic host cell, particularly one that is permissive for replication or expression of the vector. Some vectors may employ control sequences that allow it to be replicated and/or expressed in both prokaryotic and eukaryotic cells. One of skill in the art would further understand the conditions under which to incubate all of the above-described host cells to maintain them and to permit replication of a vector. Also understood and known are techniques and conditions that would allow large-scale production of vectors, as well as production of the nucleic acids encoded by vectors and their cognate polypeptides, proteins, or peptides.

In some aspects, a host cell is mammalian. Examples include CHO cells, CHO-K1 cells, or CHO-S cells. Other mammalian host cells include NS0 cells and CHO cells that are dhfr-, e.g., CHO-dhfr-, DUKX-B11 CHO cells, and DG44 CHO cells.

Numerous expression systems exist can that comprise at least a part or all of the compositions disclosed herein. Expression systems can include eukaryotic expression systems and prokaryotic expression systems. Such systems could be used, for example, to produce a polypeptide product identified as capable of binding a particular ligand. Prokaryote-based systems can be employed to produce nucleic acid sequences, or their cognate polypeptides, proteins, and peptides. Many such systems are commercially and widely available. Other examples of expression systems comprise of vectors containing a strong prokaryotic promoter such as T7, Tac, Trc, BAD, lambda pL, Tetracycline or Lac promoters, the pET Expression System and an E. coli expression system.

In some embodiments, vertebrate host cells are used for producing the antibodies of the present disclosure. For example, mammalian cell lines such as a monkey kidney CV1 line transformed by SV40 (COS-7); human embryonic kidney line (293 or 293 cells as described, e.g., in Graham et al., J. Gen Virol. 36:59, 1977; baby hamster kidney cells (BHK); mouse Sertoli cells (TM4 cells as described, e.g., in Mather, Biol. Reprod. 23:243-251, 1980 monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68, 1982; MRC 5 cells; and FS4 cells may be used to express anti-coronavirus antibodies. Other useful mammalian host cell lines include Chinese hamster ovary (CHO) cells, including DHFR-CHO cells (Urlaub et al., Proc. Natl. Acad. Sci. USA 77:4216, 1980); and myeloma cell lines such as Y0, NS0, and Sp2/0. Host cells of the present disclosure also include, without limitation, isolated cells, in vitro cultured cells, and ex vivo cultured cells. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268, 2003.

A host cell transfected with an expression vector encoding an anti-SARS-CoV-2 antibody of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

### Methods

The methods described herein can include, unless otherwise indicated, conventional methods of protein chemistry, biochemistry, recombinant DNA techniques, and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Green & Sambrook, et al., Molecular Cloning: A Laboratory Manual (4th Edition, 2012); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992); Current Protocols in Molecular Biology (2002-; Wiley; Online ISBN: 9780471142720; DOI: 10.1002/04711142727); Current Protocols in Immunology (2001-; Wiley; Online ISBN: 9780471142737; DOI: 10.1002/0471142735).

### Methods of Generating Variants

Methods for producing variants include identifying the heavy and light chain sequences (nucleic acid or amino acid) of naturally occurring antibodies, and introducing mutations therein that result in increased binding to SARS-CoV-2 antigens, enhanced neutralizing activity, improved developability, and/or reduced risk of clinical immunogenicity, as described below.

### Binding Activity

The activity of antibodies or variants thereof as described herein can be assessed for binding to SARS-CoV-2 antigens, such as the S protein or receptor-binding domain (RBD). Binding can be determined using any assay that measures binding to SARS-CoV-2 antigens, e.g., surface plasmon resonance (SPR) analysis using a biosensor system or bio-layer interferometry (BLI) or enzyme-linked immunosorbent assay (ELISA). Systems suitable for use in SPR are commercially available, for example, LSA™ (Carterra, Dublin, CA), Biacore™ (General Electric, Boston, MA), and OpenSPR (Nicoya, East Kitchener, ON, Canada). Systems suitable for use in BLI include, but are not limited to, Octet™ (ForteBio, Fremont, CA) and Gator™ (Probelife, Palo Alto, CA). In an exemplary SPR assay, each antibody or variant thereof can be either directly immobilized to a Carterra CMD200M Chip or captured to the CMD200M Carterra Chip with a goat anti-human IgG Fc antibody. The uncoupled antibodies can be washed off and various concentration gradients of the targets can be flowed over the antibodies. In some cases, the highest concentration of each target can be in the range of 0.5-8 μg/mL. For better accuracy, each antibody can be immobilized in different locations (e.g., at least 2) on the chip, and the affinity for each antibody-target combination can be determined using multiple (e.g., 4-5) target concentrations according to standard methods. In some cases, if the variation between the two duplicates is >3-fold, the antibody-target measurement is repeated. For BLI, each of the antigens can be immobilized on sensors according to the manufacturer's instructions. In one illustrative example, the antigen can be biotinylated and immobilized to streptavidin sensors. For better accuracy, each antibody can be evaluated in replicates at a suitable concentration (e.g., 5 μg/mL). In some cases, if the variation between the two duplicates is >3-fold, the antibody-target measurement is repeated. The assays are typically performed under conditions according to the manufacturer's instructions. In some cases, the assays are performed under a temperature in the range of 20° C. to 37° C., for example, 20° C.-25° C. In one embodiment, the assay is performed at 25° C. In one embodiment, the assay is performed at 37° C.

In some embodiments, binding to SARS-CoV-2 antigens is assessed in a competitive assay format with a reference antibody or a reference antibody having the same variable regions. In some embodiments, an antibody or variant thereof may block binding of the reference antibody in a competition assay by about 50% or more.

### Functional Assays

Antibodies of the present disclosure may also be evaluated in various assays for their ability to mediate FcR-dependent activity. In some embodiments, either plasma or serum obtained from patients potentially infected, or with known infections, of SARS-CoV-2, or an antibody of the present disclosure has enhanced antibody dependent cellular cytotoxicity (ADCC), antibody dependent cellular phagocytosis (ADCP), or complement-dependent cytotoxicity (CDC), or neutralization activity as and/or serum stability compared to a control or reference antibody. when the antibodies are assayed in a human IgG1 isotype format.

Examples of neutralization assays that employ fully infections SARS-CoV-2 virus include focus-forming assays and plaque-reduction assays. Alternatively, neutralization assays may employ a pseudotyped virus system where a chimeric virus particle is generated by combining plasmids encoding the genetic material for a backbone viral system, such as Vesicular stomatitis virus, with a second plasmid encoding the spike protein that sits on the surface of the virus. Such a “pseudotyped” virus can infect the same cells susceptible to fully infectious SARS-CoV-2 virus but cannot form viral progeny. Breadth and potency are two typical measures that may be employed to characterize an antibody's neutralizing activity. Breadth is the proportion of tested viruses with IC₅₀ scores that fall below an IC₅₀ cutoff value for neutralizing activity. Potency can be calculated using the geometric mean IC₅₀ (see, e.g., Hraber et al., J Virol. 88:12623-43, 2014; Rademeyer).

In some embodiments, activity of an antibody or variant thereof is evaluated in vivo in an animal model, e.g., as described in the Examples section. In one embodiment, the assay comprises passive transfer/challenge experiments in a Syrian hamster animal model.

A variant as described herein has at least 50/a, or at least 60%, or 70%, or greater, of neutralizing activity of a reference antibody (e.g., an unmodified antibody or a modified parent antibody) when evaluated under the same assay conditions. In some embodiments, an antibody exhibits increased activity, i.e., greater than 100%, activity compared to a reference antibody. In some embodiments, the antibody or variants disclosed herein have similar activity against SARS-CoV-2 infection as compared to a reference antibody. The term “similar activity,” when used to compare in vivo activity of antibodies, refers to two measurements of the activity which are no more than 30%, no more than 25%, no more than 20%, no more than 15% different, no more than 10%, no more than 8%, or no more than 5% different from each other.

In some embodiments, the antibody or variant thereof is modified to have improved developability (i.e., reduced development liabilities), including but not limited to, decreased heterogeneity, increased yield, increased stability, improved net charges to improve pharmacokinetics, and or/reduced immunogenicity. In some embodiments, antibodies having improved developability can be obtained by introducing mutations to reduce or eliminate potential development liabilities, as described in Table 6 or Table 7. In some embodiments, antibodies having improved developability possess modifications as compared to a reference or control antibody in their amino acid sequence.

In some embodiments, the antibodies or variants thereof disclosed herein have improved developability while maintaining comparable or improved binding affinity to the target antigen as compared to a reference or control (unmodified) antibody. In some embodiments, the antibodies or variants thereof disclosed herein have improved developability while maintaining activities that are similar to a reference or control (unmodified) antibody.

In some embodiments, the antibodies or variants thereof have improved developability, e.g., as identified through various in vitro assays, such as aggregation assessment by HPLC or UPLC, hydrophobic interaction chromatography (HIC), polyspecificity assays (e.g., baculovirus particle binding), self-interaction nanoparticle spectroscopy (SINS), or mass spec analysis after incubation in an accelerated degradation condition such as high temperature, low pH, high pH, or oxidative H₂O₂. Mutations are successful if the activity is maintained (or enhanced) while removing or reducing the severity of the liability.

Improved properties of antibodies or variants thereof as described herein include: (1) fits a standard platform (expression, purification, formulation); (2) high yield; (3) low heterogeneity (glycosylation, chemical modification, and the like); (4) consistent manufacturability (batch-to-batch, and small-to-large scale); (5) high stability (years in liquid formulation), e.g., minimal chemical degradation, fragmentation, and aggregation; and (6) long PK (in vivo half-life), e.g., no off-target binding, no impairment of Fern recycling, and stable. Antibody liabilities are further described in Table 7.

TABLE 6
Description of potential development liabilities

| | | sequence | |
|---|---|---|---|
| | | comprises an odd | |
| | Yield, heterogeneity, stability, | number of | |
| Free cysteine⁶ | activity | cysteines | High |
| N-linked glycosylation | Yield, heterogeneity, activity | N(~P)(S, T)¹ | High |
| Abnormal net charge | Platform fit, PK | Sharma 2014² | High |
| Patches of hydrophobicity | Stability, PK | Sharma 2014 | High |
| Patches of same charge | Stability, PK | N/A (based on | Medium |
| | | structure) | |
| Proteolysis | Stability, PK | (K, R)(K, R)³ | Medium |
| Proteolysis | Stability, PK | DP | Medium |
| Asparagine deamidation | Heterogeneity, stability, activity | NG; N(A, N, S, T)⁴ | Medium; |
| | | | Low |
| Aspartate isomerization | Heterogeneity, stability, activity | DG; D(A, D, S, T)⁵ | Medium; |
| | | | Low |
| Lysine glycation | Heterogeneity, stability, activity | K | Low |
| Methionine oxidation | Heterogeneity, stability, activity | M | Low |
| Tryptophan oxidation | Heterogeneity, stability, activity | W | Low |

Note:
¹The N-linked glycosylation site is N-X-S/T, where X is any residue other than proline.
²Sharma et al., Proc. Natl. Acad. Sci. USA 111: 18601-18606, 2014
³This motif consists of a K or R, followed by a K or R. Stated differently, the motif can be KK, KR, RK, or RR.

⁴The dipeptide NG poses a medium risk of development liability. The dipeptides NA, NN, NS, and NT pose a low risk of development liability. N may also exhibit a low risk of liability for other successor residues, e.g., D, H, or P. Stated differently, dipeptide ND, NH, or NP poses a low risk of development liability.

⁵Similarly to the above, the dipeptide DG poses a medium risk of development liability. The dipeptides DA, DD, DS, and DT pose a low risk of development liability. D may also exhibit a low risk of development liability for other successor residues, e.g., N, H, or P.

⁶“Free cysteine” refers to a cysteine that does not forma disulfide bond with another cysteine and thus is left “free” as thiols. The presence of free cysteines in the antibody can be a potential development liability. Typically, an odd net number of cysteines in the protein shows a likelihood there is a free cysteine.

Another goal for engineering variants is to reduce the risk of clinical immunogenicity: the generation of anti-drug antibodies against the therapeutic antibody. To reduce risk, the antibody sequences are evaluated to identify residues that can be engineered to increase similarity to the intended population's native immunoglobulin variable region sequences.

The factors that drive clinical immunogenicity can be classified into two groups. First are factors that are intrinsic to the drug, such as sequence; post-translational modifications; aggregates; degradation products; and contaminants. Second are factors related to how the drug is used, such as dose level; dose frequency; route of administration; patient immune status; and patient HLA type.

One approach to engineering a variant to be as much like self as possible is to identify a close germline sequence and mutate as many mismatched positions (also known as “germline deviations”) to the germline residue type as possible. This approach applies for germline genes IGHV, IGHJ, IGKV, IGKJ, IGLV, and IGLJ, and accounts for all the variable heavy (VH) and variable light (VL) regions except for part of H-CDR3. Germline gene IGHD codes for part of the H-CDR3 region but typically exhibits too much variation in how it is recombined with IGHV and IGHJ (e.g., forward, or reverse orientation, any of three translation frames, and 5′ and 3′ modifications and non-templated additions) to present a “self” sequence template from a population perspective.

Each germline gene can present as different alleles in the population. The least immunogenic drug candidate, in terms of minimizing the percent of patients with an immunogenic response, would likely be one that matches an allele commonly found in the patient population. Single nucleotide polymorphism (SNP) data from the human genome can be used to approximate the frequency of alleles in the population.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for the prediction of HLA binding and in vitro assessment to further identify potential epitopes.

Antibody variants can be designed to enhance the efficacy of the antibody. In some embodiments, design parameters can focus on CDRs, e.g., CDR3. Positions to be mutated can be identified based on structural analysis of antibody-antigen co-crystals (Oyen et al., Proc. Natl. Acad Sci. USA 114:E10438-E10445, 2017; Epub Nov. 14 2017) and based on sequence information of other antibodies from the same lineage.

### Approaches to Mutation Design

Development liabilities can be removed or reduced by one or more mutations. Mutations are designed to preserve antibody structure and function while removing or reducing development liabilities and to improve function. In some embodiments, mutations to chemically similar residues can be identified that maintain size, shape, charge, and/or polarity. Illustrative mutations are described in Table 7.

TABLE 7
preferred mutations to remove development liabilities

| | | | → |
|---|---|---|---|
| Free cysteine | Odd # C | High | C→(A, S) |
| N-linked glycosylation | N(~P)(S, T) | High | N→(Q, D, S, A); (S, T)→(A, N) |
| Proteolytic cleavage | (K, R)(K, R) | Medium | K, R→(Q, S, A) |
| Proteolytic cleavage | DP | Medium | D→(E, S, A) |
| Asparagine deamidation | NG; N(A, N, S, T)* | Medium; Low | N→(Q, S, A); G→(A, S) |
| Aspartate isomerization | DG; D(A, D, S, T)* | Medium; Low | D→(E, S, A); G→(A, S) |
| Lysine glycation | K | Low | K→(R, Q, S, A) |
| Methionine oxidation | M | Low | M→(Q, L, S, A) |
| Tryptophan oxidation | W | Low | W→(Y, F) |

Note:
the last column of Table 7 shows preferred mutations. For example, C→(A, S) refers to that C can be mutated to either A or S in order to remove development liabilities.

### In Vitro Assays

### Neutralizing Assays

In some embodiments, in vitro assays can be used to determine if the antibodies described herein produce a neutralizing response to SARS-CoV-2. In some embodiments, an assay using live, or replication-competent virus can be used. In some embodiments, a pseudovirus (PSV) neutralization assay can be used. In some embodiments, the target cells used in the neutralization assays are HeLa cells that express the cell surface receptor ACE2. In some embodiments, the target cells used in the neutralization assays are cells of the Vero E6 cell line.

In some embodiments, a neutralization assay can be used to determine inhibition of virus infectivity in cell culture in the presence of a single antibody or a combination of antibodies as described herein. In some embodiments, a neutralization assay can also be used to determine inhibition of virus infectivity in cell culture in the presence of serum or plasma from a potentially infected or confirmed infected animal or human.

### Binding Affinity Assays

In some embodiments, the assay is a Bio-layer interferometry (BLI) assay. An exemplary BLI assay is described in the Examples. In some embodiments, the assay is a Surface Plasmon Resonance (SPR) assay. An exemplary SPR assay is described in the Examples.

### Other In Vitro Assays

In some embodiments, the assay is an enzyme-linked immunosorbent assay (ELISA) that detects binding of the antibody to a coronavirus antigen, such as a SARS-CoV-2 antigen. One example of a serological ELISA is described in Amanat, F. et al., A serological assay to detect SARS-CoV-2 seroconversion in humans (doi.org/10.1101/2020.03.17.20037713). Another example is described in Krammer, F. and Simon, V., Serology assays to manage COVID-19, Science, Published Online, 15 May 2020 (DOI: 10.1126/science.abc1227).

In some embodiments, a lateral flow assay can be used to detect antibodies described herein in bodily fluids derived from a subject, such as blood serum or plasma. In some embodiments, the assay is a Western-blot assay.

In some embodiments, the assay is a transcytosis assay to determine IgG trafficking in vitro. Examples of suitable transcytosis assays are described in Claudia A. Castro Jaramillo, et al. (2017) Toward in vitro-to-in vivo translation of monoclonal antibody pharmacokinetics: Application of a neonatal Fc receptor-mediated transcytosis assay to understand the interplaying clearance mechanisms, mAbs, 9:5, 781-791, DOI: 10.1080/19420862.2017.1320008; and Chung S, Nguyen V, Lin Y. L., et al., An in vitro FcRn-dependent transcytosis assay as a screening tool for predictive assessment of nonspecific clearance of antibody therapeutics in humans. MAbs. 2019; 11(5):942-955. doi:10.1080/19420862.2019.1605270.

### T Cell Assays

Other assays include determining T cell responses to infection by SARS-CoV-2. Such assays include determining CD4⁺ and CD8⁺ T cell responses. One assay for determining CD4⁺ T cell responses is a T cell receptor- (TCR) dependent Activation Induced Marker (AIM) assay, which allow for allows quantification of SARS-CoV-2-specific CD4⁺ T cells in subjects who were exposed to SARS-CoV-2 or who recovered from COVID-19. Markers for CD4⁺ T cells include OX40 and CD137. Assays for determining CD8⁺ T cell responses include AIM assays and intracellular cytokine staining (ICS). Markers for CD8⁺ T cells include CD69 and CD137. The expression of cytokines such as IFNγ, granzyme B, and TNF can be used for ICS.

### In Vivo Assays

In some embodiments, in vivo assays can be used to determine if the antibodies described herein produce a neutralizing response to SARS-CoV-2. In one embodiment, the in vivo assay comprises passive transfer/challenge experiments in a Syrian hamster animal model, as described in the Examples. In other embodiments, transgenic mice expressing the human ACE2 receptor, or non-human primates, can be used. Assays describing all three models are described in the Examples.

Methods of Producing or Generating Antibodies that Bind SARS-CoV-2

The antibodies described herein can be produced using multiple different technologies, such as 1) isolation of antibodies of interest from B cells of a subject that mounted an immune response to the virus; and 2) isolation of antibodies derived from expression libraries of immunoglobulin molecules, or derivatives thereof, expressed heterologously and screened using one or more display technologies (reviewed in Hoogenboom H R, Trends Biotechnol., 1997, 15:62-70; Hammond P W, MAbs, 2010, 2:157-64; Nissim A, Chemajovsky Y, Handb Exp Pharmacol., 2008, (181):3-18; Steinitz M, Hum Antibodies, 2009; 18:1-10; Bradbury A R, Sidhu S, Dübel S, and McCafferty, Nat Biotechnol., 2011, 29:245-54; Antibody Engineering (Kontermann R E and Dübel S eds., Springer, 2nd edition)).

In some embodiments, peripheral blood mononuclear cells (PBMC) can be isolated from human subjects that are acutely infected with SARS-CoV-2 or have symptoms consistent with COVID-19, or are asymptomatic, but have had known contact with a SARS-CoV-2 infected individual. An appropriate test, such as a PCR test using a sample from a nasopharyngeal swab, can be used to confirm the subject is infected with SARS-CoV-2. To determine if a donor was infected with SARS-CoV-2, donor plasma or serum can be tested for IgM, IgG or IgA antibodies that bind to specific SARS-CoV-2 antigens. The antigens included in this assay could be any of the structural and non-structural proteins expressed by any of the SARS-CoV-2 open reading frames (ORFs). Alternatively, donor plasma or sera can be tested in neutralization assays using to pseudovirus expressing SARS-CoV-2 spike protein or full-length, infectious SARS-CoV-2 Binding to antigens from closely related viruses, such as SARS-CoV-1, may also be tested.

Peripheral blood mononuclear cells (PBMC) isolated from either acutely infected or previously infected subjects can be sorted for B cells using B cell markers by FACS and/or SARS-CoV-2 antigens as bait. In some embodiments, PBMCs isolated from either acutely infected or previously infected subjects will be stained with propidium iodide as a live/dead marker in addition to a panel of antibodies including CD3, CD14, IgM, IgA and IgD, CD19, CD20, CD27 and CD38. Cells that are CD19-, CD20⁺, CD27, CD38⁺ are initially selected. The sub-population of cells that are negative for CD3, CD14, IgM, IgA and IgD are considered plasmablasts.

### Methods for Inducing a Prophylactic Immune Response

In one aspect, methods for inducing an in vivo immune response in a subject at risk of SARS-CoV-2 infection, or a subject potentially exposed to another person infected with SARS-Cov-2, following administration of a prophylactic antibody are provided. In some embodiments, the method comprises administering an antibody described herein to the subject and detecting an immune response. In some embodiments of the method, the antibody is administered intravenously. In some embodiments, the successful administration of antibody can be evaluated by testing if the serum or plasma from the donor binds to SARS-CoV-2 antigens in an ELISA or other binding assay. In some embodiments, the plasma or serum from the subject could also be tested for functional activity, including antibody-dependent cellular cytotoxicity (ADCC), antibody-dependent cellular phagocytosis (ADCP), and/or complement-dependent cytotoxicity (CDC) or antibody-mediated virus neutralization. In some embodiments, the immune response results in protection from infection for a specific period of time following administration of the antibody described herein.

### Methods for Inducing a Therapeutic Immune Response

In another aspect, methods for inducing an in vivo immune response in a subject infected with SARS-CoV-2 following administration of a therapeutic antibody are provided. In some embodiments, the method comprises administering an antibody described herein to the subject and detecting an immune response. In some embodiments of the method, the antibody is administered intravenously. In some embodiments, the successful administration of antibodies is evaluated by testing if the serum or plasma from the donor binds to SARS-CoV-2 antigens in an ELISA or other binding assay. In some embodiments, the plasma or serum from the subject is also tested for functional activity, including antibody-dependent cellular cytotoxicity (ADCC), antibody-dependent cellular phagocytosis (ADCP), and/or complement-dependent cytotoxicity (CDC) or antibody-mediated virus neutralization. In some embodiments, the immune response is measured by the amount of time to clinical improvement as defined by discharge from hospital, or reduction in 2 points on a 6-point scale, where 1 is sufficiently healthy for discharge, and 6 is death. In some embodiments, the immune response is measured by seroconversion from positive SARS-CoV-2 RT-qPCR test to negative SARS-CoV-2 RT-qPCR test within a specific number of hours. In some embodiments, the total number of hours by which to quantify seroconversion is approximately 72 hours (see Li L, Zhang W, Hu Y, et al., Effect of Convalescent Plasma Therapy on Time to Clinical Improvement in Patients with Severe and Life-threatening COVID-19: A Randomized Clinical Trial. JAMA Published online Jun. 3, 2020, doi: 10.1001/jama.2020.10044). In some embodiments, the immune response is measured by the absolute reduction in viral load or the percent reduction in viral load from treatment initiation to treatment cessation. In some embodiments, the immune is quantified by whether the patient required supplemental oxygen therapy, including mechanical ventilation or the duration of mechanical ventilation or supplemental oxygen therapy (see Wang, Y., et al., Remdesivir in adults with severe COVID-19: a randomised, double-blind, placebo-controlled, multicentre trial. The Lancet, Vol. 395; pages 1569-1578, May 16, 2020).

### Methods of Treatment

In another aspect, a method for treating or preventing one or more symptoms of COVID-19 in a subject is provided, the method comprising administering to the subject a therapeutically effective amount of an antibody as disclosed herein, or a pharmaceutical composition comprising the antibody. In certain embodiments, an antibody described herein is administered to the subject in combination with one or more additional therapeutic agents used to treat a viral infection or the side effects or associated symptoms thereof. In certain embodiments, the method comprises administering to a subject a therapeutically effective amount of an antibody as disclosed herein, or a pharmaceutical composition comprising the antibody, in combination with a therapeutically effective amount of one or more additional therapeutic agents. In one embodiment, a method for treating COVID-19 in a human having or at risk of having an infection by SARS-CoV-2 is described, the method comprising administering to the human a therapeutically effective amount of an antibody as disclosed herein, or a pharmaceutical composition comprising the antibody, in combination with a therapeutically effective amount of one or more additional therapeutic agents.

Symptoms of Covid-19 include fever or chills, cough, shortness of breath (dyspnea), fatigue, muscle or body aches, headache, sore throat, a new loss of taste or smell, congestion or runny nose, nausea, vomiting, diarrhea, inflammation of the skin, confusion, eye symptoms such as enlarged blood vessels, swollen eyelids, excessive watering and increased discharge, light sensitivity and irritation, and neurological complications such as delirium, brain inflammation, stroke, and nerve damage. Symptoms may appear two to 14 days after exposure to SARS-CoV-2.

In some embodiments, one or more additional therapeutic agents comprise an antibody that binds to SARS-CoV-2. In some embodiments, the antibody that binds to SARS-CoV-2 is casirivimab (Regeneron Pharmaceuticals), imdevimab (Regeneron Pharmaceuticals), etesevima (Eli Lilly and Company), bamlanivimab (Eli Lilly and Company), CT-P59 (Celltrion Healthcare), BRII-196 (Brii Biosciences), BRII-198 (Brii Biosciences), VIR-7831 (Vir Biotechnology), AZD7442 (AstraZeneca), AZD8895 (AstraZeneca), AZD1061 (AstraZeneca), TY027 (Tychan Pte. Ltd.), SCTA01 (Sinocelltech Ltd.), MW33 (Mabwell Bioscience Co., Ltd.), JS016 (Junshi Biosciences), DXP593 (Singlomics/Beigene), DXP604 (Singlomics/Beigene), STI-2020 (Sorrento Therapeutics), BI 767551/DZIF-10c (U. Cologne/Boehringer Ingelheim), COR-101 (CORAT Therapeutics), HLX70 (Hengenix Biotech), ADM03820 (Ology Bioservices), HFB30132A (HiFiBiO Therapeutics), ABBV-47D11 (AbbVie), C144-LS (Bristol-Myers Squibb, Rockefeller University), C-135-LS (Bristol-Myers Squibb, Rockefeller University), LY-CovMab (Luye Pharma), JMB2002 (Jemincare), or ADG20 (Adagio Therapeutics), or combinations thereof. In some embodiments, an antibody of the present disclosure as described herein is combined with at least one of casirivimab (Regeneron Pharmaceuticals), imdevimab (Regeneron Pharmaceuticals), etesevima (Eli Lilly and Company), bamlanivimab (Eli Lilly and Company), CT-P59 (Celltrion Healthcare), BRII-196 (Brii Biosciences), BRII-198 (Brii Biosciences), VIR-7831 (Vir Biotechnology), AZD7442 (AstraZeneca), AZD8895 (AstraZeneca), AZD1061 (AstraZeneca), TY027 (Tychan Pte. Ltd.), SCTA01 (Sinocelltech Ltd.), MW33 (Mabwell Bioscience Co., Ltd.), JS016 (Junshi Biosciences), DXP593 (Singlomics/Beigene), DXP604 (Singlomics/Beigene), STI-2020 (Sorrento Therapeutics), BI 767551/DZIF-10c (U. Cologne/Boehringer Ingelheim), COR-101 (CORAT Therapeutics), HLX70 (Hengenix Biotech), ADM03820 (Ology Bioservices), HFB30132A (HiFiBiO Therapeutics), ABBV-47D11 (AbbVie), C144-LS (Bristol-Myers Squibb, Rockefeller University), C-135-LS (Bristol-Myers Squibb, Rockefeller University), LY-CovMab (Luye Pharma), JMB2002 (Jemincare), or ADG20 (Adagio Therapeutics). In some embodiments, an antibody of the present disclosure as described herein is combined with at least one of casirivimab (Regeneron Pharmaceuticals), imdevimab (Regeneron Pharmaceuticals), etesevima (Eli Lilly and Company), or bamlanivimab (Eli Lilly and Company), or combinations thereof.

In some embodiments, one or more additional therapeutic agents comprise antiviral drugs. In some embodiments, the one or more additional therapeutic agents comprise the antiviral drug molnupiravir (MK-4482/EIDD-2801). In some embodiments, one or more additional therapeutic agents comprise the antiviral drug remdesivir (GS-5734™). Molnupiravir is an investigational, orally administered form of a potent ribonucleoside analog that inhibits the replication of SARS-CoV-2. Remdesivir has demonstrated in vitro and in vivo activity in animal models against the viral pathogens that cause MERS and SARS, which are coronaviruses structurally similar to SARS-CoV-2.

Examples of additional therapeutic agents that may be useful for treating COVID-19 are shown below (see the internet at www.drugs.com/condition/covid-19.html)

Baricitinib: a Janus kinase (JAK) inhibitor (marketed under the brand name Olumiant for the treatment of rheumatoid arthritis).

Bemcentinib: An AXL kinase inhibitor. Bemcentinib has been reported to exhibit potent antiviral activity in preclinical models against several enveloped viruses, including Ebola and Zika virus, and recent data have expanded this to include SARS-CoV-2.

Bevacizumab. A VEGF inhibitor (marketed under the brand name Avastin for certain types of cancer) is being studied as a treatment for acute lung injury (ALI) and acute respiratory distress syndrome (ARDS) in critically ill patients with COVID-19 pneumonia.

Chloroquine phosphate. An anti-malaria drug that has been shown to have a wide range of antiviral effects, including anti-coronavirus. Studies in Guangdong Province in China suggest that chloroquine may help improve patient outcomes in people with novel coronavirus pneumonia.

Colchicine. An anti-inflammatory drug being studied to prevent complications of COVID-19 in high risk patients. Colchicine has long been used in the treatment of gout.

EIDD-2801. A broad-spectrum oral antiviral that could be used as a potential prophylactic or treatment for COVID-19 and other coronaviruses.

Favipiravir. An antiviral drug approved in some countries for the treatment of influenza, was also approved for use in clinical trials as a treatment for novel coronavirus pneumonia.

Fingolimod. An approved drug called fingolimod (marketed under the brand name Gilenya for the treatment of relapsing forms of multiple sclerosis) is being studied as a treatment for COVID-19.

Hydroxychloroquine and azithromycin. the anti-malaria drug hydroxychloroquine and the macrolide antibacterial drug azithromycin (Zithromax) are currently in clinical trials in the U.S.

Hydroxychloroquine sulfate. a malaria drug was shown to be effective in killing the coronavirus in laboratory experiments. Hydroxychloroquine was first approved by the FDA in 1995 under the brand name Plaquenil, and it is also used in the treatment of patients with lupus and arthritis. In March 2020, the US FDA issued an emergency use authorization (EUA) to allow the emergency use of hydroxychloroquine sulfate supplied from the Strategic National Stockpile (SNS) for the treatment of COVID-19 in certain hospitalized patients.

Ivermectin. An anti-parasitic drug was shown to be effective against the SARS-CoV-2 virus in an in-vitro laboratory study. Further clinical trials need to be completed to confirm the effectiveness of the drug in humans with COVID-19.

Leronlimab. A CCR5 antagonist has shown promise in reducing the “cytokine storm” in a small number of critically ill COVID-19 patients hospitalized in the New York area.

Lopinavir and ritonavir. A drug combination called lopinavir/ritonavir is approved to treat HIV under the brand name Kaletra. Currently being studied in combination with the flu drug oseltamivir (Tamiflu) in Thailand.

Methylprednisolone. A widely used glucocorticoid is being studied for safety and effectiveness in the treatment of novel coronavirus pneumonia in several hospitals in the Hubei province of China.

Sarilumab. An interleukin-6 (IL-6) receptor antagonist (marketed under the brand name Kevzara for the treatment of rheumatoid arthritis) is being studied as a potential treatment for acute respiratory distress syndrome (ARDS) in patients critically ill from COVID-19.

Tocilizumab. An interleukin-6 receptor antagonist (marketed under the brand name Actemra for the treatment of rheumatoid arthritis and other inflammatory conditions) is being studied in a number of locations worldwide for the treatment of patients with COVID-19.

Umifenovir. An antiviral drug (marketed in Russia under the brand name Arbidol, and also available in China for the treatment of influenza) is being studied in China and other countries as a treatment for COVID-19.

In certain embodiments, when an antibody of the present disclosure as described herein is combined with one or more additional therapeutic agents as described above, the components of the composition are administered as a simultaneous or sequential regimen. When administered sequentially, the combination may be administered in two or more administrations.

In some embodiments, an antibody as disclosed herein is combined with one or more additional therapeutic agents in a unitary dosage form for simultaneous administration to a patient.

A “patient” refers to any subject receiving the antibody regardless of whether they have COVID-19. In some embodiments, a “patient” is a non-human subject, e.g., an animal that is used as a model of evaluating the effects of antibody administration.

“Co-administration” of an antibody as disclosed herein with one or more additional therapeutic agents generally refers to simultaneous or sequential administration of an antibody or fragment thereof disclosed herein and one or more additional therapeutic agents, such that therapeutically effective amounts of the antibody or fragment thereof disclosed herein and one or more additional therapeutic agents are both present in the body of the patient.

Co-administration includes administration of unit dosages of the antibody disclosed herein before or after administration of unit dosages of one or more additional therapeutic agents, for example, administration of the antibody within seconds, minutes, or hours of the administration of one or more additional therapeutic agents. For example, in some embodiments, a unit dose of an antibody disclosed herein is administered first, followed within seconds or minutes by administration of a unit dose of one or more additional therapeutic agents. Alternatively, in other embodiments, a unit dose of one or more additional therapeutic agents is administered first, followed by the administration of a unit dose of an antibody within seconds or minutes. In some embodiments, a unit dose of an antibody disclosed herein is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of the antibody.

The combined administration may be co-administration, using separate pharmaceutical compositions or a single pharmaceutical composition, or consecutive administration in either order, wherein there is optionally a time period while both (or all) therapeutic agents simultaneously exert their biological activities. Such combined therapy may result in a synergistic therapeutic effect. In certain embodiments, it is desirable to combine administration of an antibody of the invention with another antibody directed against another Plasmodium falciparum: antigen, or against a different SARS-CoV-2 target epitope.

As described herein, the antibody may also be administered by gene therapy via a nucleic acid comprising one or more polynucleotides encoding the antibody. In certain embodiments, the polynucleotide encodes an scFv. In particular embodiments, the polynucleotide comprises DNA, cDNA or RNA. In certain embodiments, the polynucleotide is present in a vector, e.g., a viral vector. In one embodiment, the antibody is administered via in vitro-transcribed (IVT) mRNA to express the antibody. See US20190309067, at Examples 12-14, hereby incorporated by reference.

### Methods of Preventing Infection

In another aspect, a method for preventing infection by SARS-CoV-2 in a subject is provided, the method comprising administering to the subject an antibody described herein, or a pharmaceutical composition comprising the antibody, wherein the antibody or pharmaceutical composition is administered at a dose sufficient to prevent or reduce infection of one or more host cells in the subject by SARS-CoV-2.

In some embodiments, the antibody or pharmaceutical composition is administered to the subject in combination with one or more additional therapeutic agents that are effective at preventing infection of host cells by SARS-CoV-2. In some embodiments, the one or more additional therapeutic agents that are effective at preventing infection of host cells by SARS-CoV-2 comprise an antibody that binds to SARS-CoV-2. In some embodiments, the antibody that binds to SARS-CoV-2 is casirivimab (Regeneron Pharmaceuticals), imdevimab (Regeneron Pharmaceuticals), etesevima (Eli Lilly and Company), bamlanivimab (Eli Lilly and Company), CT-P59 (Celltrion Healthcare), BRII-196 (Brii Biosciences), BRII-198 (Brii Biosciences), VIR-7831 (Vir Biotechnology), AZD7442 (AstraZeneca), AZD8895 (AstraZeneca), AZD1061 (AstraZeneca), TY027 (Tychan Pte. Ltd.), SCTA01 (Sinocelltech Ltd.), MW33 (Mabwell Bioscience Co., Ltd.), JS016 (Junshi Biosciences), DXP593 (Singlomics/Beigene), DXP604 (Singlomics/Beigene), STI-2020 (Sorrento Therapeutics), BI 767551/DZIF-10c (U. Cologne/Boehringer Ingelheim), COR-101 (CORAT Therapeutics), HLX70 (Hengenix Biotech), ADM03820 (Ology Bioservices), HFB30132A (HiFiBiO Therapeutics), ABBV-47D11 (AbbVie), C144-LS (Bristol-Myers Squibb, Rockefeller University), C-135-LS (Bristol-Myers Squibb, Rockefeller University), LY-CovMab (Luye Pharma), JMB2002 (Jemincare), or ADG20 (Adagio Therapeutics). In some embodiments, an antibody of the present disclosure as described herein is combined with at least one of casirivimab (Regeneron Pharmaceuticals), imdevimab (Regeneron Pharmaceuticals), etesevima (Eli Lilly and Company), bamlanivimab (Eli Lilly and Company), CT-P59 (Celltrion Healthcare), BRII-196 (Brii Biosciences), BRII-198 (Brii Biosciences), VIR-7831 (Vir Biotechnology), AZD7442 (AstraZeneca), AZD8895 (AstraZeneca), AZD1061 (AstraZeneca), TY027 (Tychan Pte. Ltd.), SCTA01 (Sinocelltech Ltd.), MW33 (Mabwell Bioscience Co., Ltd.), JS016 (Junshi Biosciences), DXP593 (Singlomics/Beigene), DXP604 (Singlomics/Beigene), STI-2020 (Sorrento Therapeutics), BI 767551/DZIF-10c (U. Cologne/Boehringer Ingelheim), COR-101 (CORAT Therapeutics), HLX70 (Hengenix Biotech), ADM03820 (Ology Bioservices), HFB30132A (HiFiBiO Therapeutics), ABBV-47D11 (AbbVie), C144-LS (Bristol-Myers Squibb, Rockefeller University), C-135-LS (Bristol-Myers Squibb, Rockefeller University), LY-CovMab (Luye Pharma), JMB2002 (Jemincare), or ADG20 (Adagio Therapeutics). In some embodiments, an antibody of the present disclosure as described herein is combined with at least one of casirivimab (Regeneron Pharmaceuticals), imdevimab (Regeneron Pharmaceuticals), etesevima (Eli Lilly and Company), or bamlanivimab (Eli Lilly and Company).

In some embodiments, the one or more additional therapeutic agents that are effective at preventing infection of host cells by SARS-CoV-2 comprise antiviral drugs, such as remdesivir (GS-5734™). In some embodiments, the one or more additional therapeutic agents that are effective at preventing infection of host cells by SARS-CoV-2 comprise baricitinib, bemcentinib, bevacizumab, chloroquine phosphate, colchicine, EIDD-2801, favipiravir, fingolimod, hydroxychloroquine and azithromycin, hydroxychloroquine sulfate, ivermectin, leronlimab, lopinavir and ritonavir, methylprednisolone, sarilumab, tocilizumab, or umifenovir, or combinations thereof.

In one embodiment, the subject has not responded to treatment with a SARS-CoV-2 vaccination regimen. In one embodiment, the subject is not eligible for treatment with a SARS-CoV-2 vaccination regimen. In one embodiment, the subject is not likely to respond to treatment with a SARS-CoV-2 vaccination regimen, such as an elderly subject (a subject who is 65 years of age or older) or a subject with altered immunocompetence.

### Diagnostics

The antibodies described herein can also be used to detect viral antigens in a biological sample isolated from a subject, and therefore can be useful for diagnosing infection by SARS-CoV-2 in a subject. The sample can be, for example, blood, plasma, or serum. The antibodies described herein can also be used as biomarkers for monitoring the response to treatment of COVID-19 or for determining whether a patient will respond to a particular therapy. Thus, in another embodiment, a method of diagnosing a patient that is or was infected with a coronavirus is described, the method comprising detecting binding of an antibody described herein to a sample obtained from the patient, wherein binding greater than a negative control value indicates the patient is infected with the coronavirus. In some embodiments, the method is an in vitro method, such that detecting binding of an antibody described herein to a sample obtained from the patient is performed in vitro. It will be understood that in vitro methods are not the same as methods performed on an animal or human subject.

In another aspect, a method of identifying a patient that is infected with a coronavirus is described, the method comprising detecting binding of an antibody described herein to a sample obtained from the patient, wherein binding greater than a negative control value indicates the patient is infected with the coronavirus. In some embodiments, the method comprises testing a patient sample for binding to an antibody described herein and detecting binding of the antibody to components of the sample. Detecting binding of the antibody can be performed, for example, by a serological assay, including enzyme-linked immunosorbent assays (ELISAs), lateral flow assays, or Western blot-based assays. In some embodiments, the sample is a blood, plasma, or serum sample.

### B Cells

In some embodiments, samples described herein comprise immune cells. The immune cells can be B cells. B-cells include, for example, activated B cells, blasting B cells, plasma cells, plasmablasts, memory B cells, B1 cells, B2 cells, marginal zone B cells, and follicular B cells.

In general, a “B cell” refers to any cell that has at least one rearranged immunoglobulin gene locus. A B cell can include at least one rearranged immunoglobulin heavy chain locus or at least one rearranged immunoglobulin light chain locus. A B cell can include at least one rearranged immunoglobulin heavy chain locus and at least one rearranged immunoglobulin light chain locus. B cells are lymphocytes that are part of the adaptive immune system. B cells can include any cells that express antibodies either in the membrane-bound form as the B-cell receptor (BCR) on the cell surface or as secreted antibodies. B cells can express immunoglobulins (antibodies, B cell receptor). Antibodies can include heterodimers formed from the heavy and light immunoglobulin chains. The heavy chain is formed from gene rearrangements of the variable, diversity, and junctional (VDJ) genes to form the variable region, which is joined to the constant region. The light chain is formed from gene rearrangements of the variable and junctional (VJ) genes to form the variable region, which is then joined to the constant region. Owing to a large possible number of junctional combinations, the variable regions of the antibody gene (which is also the BCR) have huge diversity, enabling B cells to recognize any foreign antigen and mount a response against it.

### B-Cell Activation and Differentiation

B cells are activated and differentiate when they recognize an antigen in the context of an inflammatory immune response. They usually include 2 signals to become activated, one signal delivered through BCR (a membrane-bound form of the rearranged immunoglobulin), and another delivered through CD40 or another co-stimulatory molecule. This second signal can be provided through interaction with helper T cells, which express the ligand for CD40 (CD40L) on their surface. B cells then proliferate and may undergo somatic hypermutation, where random changes in the nucleotide sequences of the antibody genes are made, and B cells whose antibodies have higher affinity B cells are selected. They may also undergo “class-switching”, in which the constant region of the heavy chain encoding the IgM isotype is switched to the constant region encoding the IgG, IgA, or IgE isotype. Differentiating B cells may end up as memory B cells, which are usually of higher affinity and class-switched, though some memory B cells are still of the IgM isotype. Memory B cells can also become activated and differentiate into plasmablasts and ultimately, into plasma cells. Differentiating B cells may also first become plasmablasts, which then differentiate to become plasma cells.

### Affinity Maturation and Clonal Families

A clonal family is generally defined using related immunoglobulin heavy chain and/or light chain V(D)J sequences by 2 or more antibodies or B-cell receptors. Related immunoglobulin heavy chain V(D)J sequences can be identified by their shared usage of V(D)J gene segments encoded in the genome. Within a clonal family there are generally subfamilies that vary based on shared mutations within their V(D)J segments, that can arise during B cell gene recombination and somatic hypermutation.

Activated B cells migrate and form germinal centers within lymphoid or other tissues, where they undergo affinity maturation. B cells may also undergo affinity maturation outside of germinal centers. During affinity maturation, B cells undergo random mutations in their antibody genes, concentrated in the complementary determining regions (CDRs) of the genes, which encode the parts of the antibody that directly bind to and recognize the target antigen against which the B cell was activated. This creates sub-clones from the original proliferating B cell that express immunoglobulins that are slightly different from the original clone and from each other. Clones compete for antigen and the higher-affinity clones are selected, while the lower-affinity clones die by apoptosis. This process results in the “affinity maturation” of B cells and consequently in the generation of B cells expressing immunoglobulins that bind to the antigen with higher affinity. All of the B cells that originate from the same ‘parent’ B cell form clonal families, and these clonal families include B cells that recognize the same or similar antigenic epitopes. In some aspects, we expect that clones present at higher frequencies represent clones that bind to antigen with higher affinity because the highest-affinity clones are selected during affinity maturation. In some aspects, clones with different V(D)J segment usage exhibit different binding characteristics. In some aspects, clones with the same V(D)J segment usage but different mutations exhibit different binding characteristics.

### Memory B Cells

Memory B cells are usually affinity-matured B cells and may be class-switched. These are cells that can respond more rapidly to a subsequent antigenic challenge, significantly reducing the time included for affinity-matured antibody secretion against the antigen from ˜14 days in a naive organism to ˜7 days.

### Plasmablasts and Plasma Cells

Plasma cells can be either long-lived or short-lived. Long-lived plasma cells may survive for the lifetime of the organism, whereas short-lived plasma cells can last for 3-4 days. Long-lived plasma cells reside either in areas of inflammation, in the mucosal areas (in the case of IgA-secreting plasma cells), in secondary lymphoid tissues (such as the spleen or lymph nodes), or in the bone marrow. To reach these divergent areas, plasmablasts fated to become long-lived plasma cells may first travel through the bloodstream before utilizing various chemokine gradients to traffic to the appropriate areas. Plasmablasts are cells that are affinity matured, are typically classed-switched, and usually secrete antibodies, though generally in lower quantities than the quantity of antibody produced by plasma cells. Plasma cells are dedicated antibody secretors.

Following either vaccination or infection, naïve or memory B cells enter germinal centers and are exposed to antigen. Following appropriate antigen presentation, a specific B cell subset, plasmablasts, are released into the blood and are responsible for making large amounts of antigen-specific antibodies. As an infection progresses, memory B cells re-enter germinal centers and undergo somatic hypermutation, a process which increases the affinity of a given B cell for the antigen of interest. Thus, the plasmablast population reflects not only the naïve B cell response to a given pathogen, but also maturation of the B cell response to that pathogen in real time. Other B cell sorting techniques rely on bait-based selection methods to identify the extremely rare pathogen-specific, circulating memory B cell populations. Such bait-based selection methods bias the selected B cells for those that bind most strongly to the bait and exclude antibodies that either target different antigens/epitopes altogether or bind to conformationally-specific epitopes not formed when the bait is recombinantly expressed. During an acute infection or following vaccination, the plasmablast population can comprise up to 47% of the peripheral B cell population (Wrammert et al, 2012), making them ideal candidates to interrogate using an agnostic sorting strategy. Thus, plasmablasts represent a good source of antibodies that bind SARS-CoV-2 antigens.

### EXAMPLES

### Example 1

This example describes isolating plasmablast cell populations from peripheral blood mononuclear cells (PBMCs) from human subjects actively infected with SARS-CoV-2.

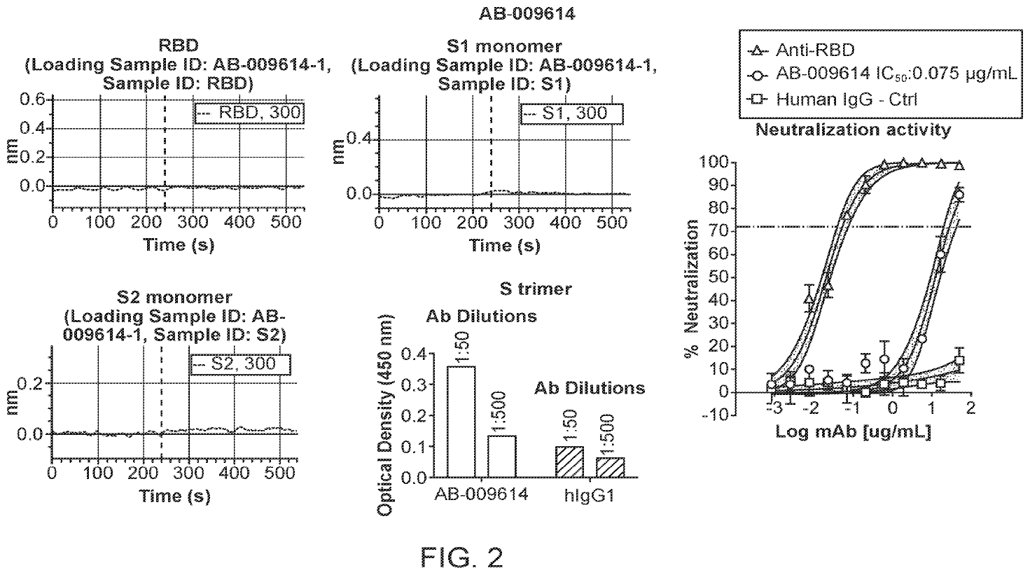

PBMCs will be stained with propidium iodide as a live/dead marker in addition to a panel of antibodies including CD3, CD14, IgM, IgA and IgD, CD19, CD20, CD27, and CD38. Cells that are CD19⁺, CD20-, CD27⁺, CD38⁺ are initially selected. The sub-population of cells that are negative for CD3, CD14, IgM, IgA, and IgD are considered “IgG⁺ plasmablasts” and will be single-cell sorted into 384 well plates containing lysis buffer (FIG. 2). To ensure broad coverage of the pathogen-specific antibody repertoire, sequences will be generated from ˜500 PBs per patient sample. mRNA from sorted plasmablasts will be reverse transcribed into cDNA using the protocols below.

### Example 2

This example describes methods for generating high-quality, paired heavy and light chain sequences.

Following production of cDNA, high-quality, paired heavy and light chain sequences were generated. In brief, cDNA samples were thawed, pooled, and amplified using a nested PCR reaction. PCR products were sequenced using Illumina® next-generation sequencing technology and processed with the IRC™ bioinformatics pipeline, resulting in the generation of variable heavy and light chain sequences. Barcodes employed early in the amplification process facilitated the pairing of native variable heavy and light chain sequences and allowed for the generation of error-corrected sequences with 99.998% accuracy. See PCT/US2012/000221 (corresponding to US 2015/0133317) and PCT/US2014/072898 (corresponding to US 2015/0329891), which are incorporated by reference herein. Thus, the end product was a highly accurate, natively paired variable heavy and light chain sequences that included the signal sequence peptide on the 5′ end and extended through part of the constant region at the 3′ end, allowing for accurate identification of each antibody's subclass (See DeFalco et al., 2018).

Each paired set of sequences was analyzed using an Atreca-generated informatics platform and assigned to a specific lineage. Two sequences that originated from the same donor, used the same putative germline heavy and light variable (V) and joining (J) genes, have H-CDR3 and L-CDR3 sequences of the same length and that had at least 75% nucleotide sequence identity in the H-CDR3 and L-CDR3 regions were grouped together into lineages. Phylogenetic trees incorporating these sequence and lineage-specific data were constructed for each donor using rapidNJ software.

### Example 3

This example describes methods for sequence characterization and selection.

Using the methods described in Example 2, 17,704 paired heavy and light chain sequences were generated from 4 donors who were infected with SARS-CoV-2. The selection procedures defined below identified 235 of the 17,704 paired sequences for expression as human IgG1 antibodies.
1) Lineage abundance—Lineages containing the largest number of plasmablast-derived sequences were selected from each donor, and one representative sequence was selected per lineage.
2) Lineage persistence—Lineages that were observed in multiple samples obtained at different timepoints were selected, one representative sequence per lineage.
3) Convergence—Antibodies were determined to be convergent in this disclosure if their H-CDR3 and L-CDR3 are of comparable length and have a similar sequence in this application. Convergent antibodies identified herein include those having two or more sequences that originated from different donors or one or more sequences from the Atreca set (i.e., a set of antibodies including any of the antibodies disclosed in this application) and a sequence reported in the literature that originated from the same or similar germline genes. A representative sequence was selected from pairs or groups of convergent sequences.
4) Unique sequence features: Sequences that exhibited long H-CDR3 lengths and/or multiple cysteine residues in H-CDR3 were selected in this category.

Using these methods, the 235 paired sequences were selected and expressed as hIgG1 as described in Example 4. Of the 235 Abs expressed, 227 generated sufficient material for testing in binding and neutralization assays as described in Example 5 and as specified in Tables 1-3.

### Example 4

This example describes methods for producing antibodies.

After sequences were selected, gene fragments were synthesized and cloned into human variable heavy (VH) or variable light (VL) chain expression vectors. Fully human, IgG1 antibodies were expressed using a 293F transfection system and purified using high-throughput Protein A columns in sufficient quantities (≥250 μg) to be tested in downstream binding and neutralization assays.

### Example 5

This example describes methods for identifying antibodies that bind to SARS-CoV-2 antigens. Antibodies were screened for binding to 6 antigens: Envelope (E), Nucleocapsid (N), the native Spike (S) trimer and subunits of the monomeric form of the Spike protein, including the S1 and S2 subunits and the Receptor Binding Domain (RBD) and tested for neutralization activity using fully infectious SARS-CoV-2 virus.

Spike Trimer and Envelope Proteins: ELISA assays were conducted to measure binding to the spike trimer (Acro Biosystems) and the envelope protein (Abcam). Briefly, antibodies were diluted to two concentrations, 1:50 and 1:500, irrespective of initial antibody concentration, and tested for binding against the two proteins in singlet (i.e., individually). An antibody was positive for binding to the antigen if the optical density value for the antibody diluted 1:50 measured at least two-fold greater than the human isotype negative control.

S1, S2, RBD, and Nucleocapsid Proteins: Bio-layer interferometry (BLI) was used to quantify binding avidity for all antibodies to the S1, S2, RBD (all obtained from Sino Biological) & N (Acro Biosystems) proteins. In these experiments, antigens were diluted to 5 μg/mL and loaded onto anti-human IgG Fc Capture sensors (AHC). Loaded sensors were dipped into antigen at a concentration of 300 nM. Data was captured using an Octet instrument and kinetic constants were calculated using a monovalent (1:1) binding model. On- and off-rates and estimated KD values were reported for those antibody-antigen pairs with a binding response (nm) ≥0.05. Antibody-antigen pairs where the binding response (nm) was <0.05 was labeled as less than the lower limit of detection (<LLOD).

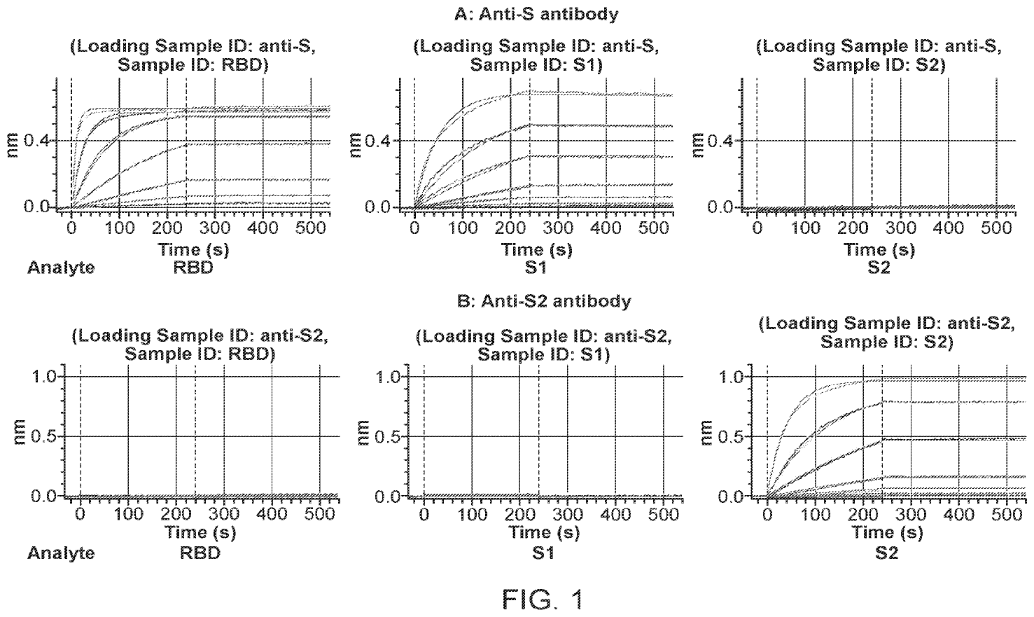

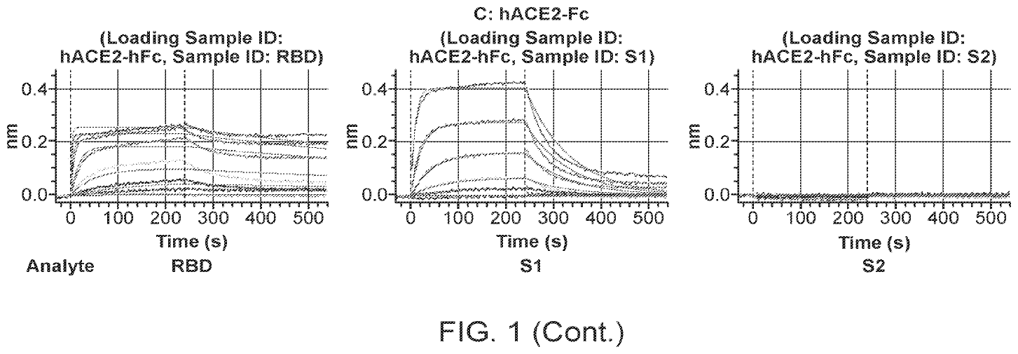

To determine the appropriate concentration of antigen to add as the analyte, the RBD, S1 and S2 antigens were tested at two concentrations (300 nM and 1,000 nM) for binding to both positive control antibodies expected to bind to the test antigen, and negative control antibodies not expected to bind to the test antigen. FIG. 1 demonstrates control binding of three antibodies to the three proteins. The control anti-spike (anti-S) antibody (Sino Biological) binds to RBD and S1 proteins with KD values of 5.70E-12M and 2.69E-9M as expected. In contrast, this antibody demonstrates no measurable binding against S2 protein. Reciprocally, the anti-S2 control antibody (Sino Biological) binds to the S2 protein with a KD value of 2.06E-11M but does not bind to either the RBD or S1 proteins. ACE2-hFc (LakePharma), a fusion protein consisting of human angiotensin I-converting enzyme-2 fused to a human IgG1 Fc, is a third control that binds to both RBD and S1 with KD values of 1.75E-9M and 1.03E-7M respectively, but not to the S2 protein (Table 1). Data from this experiment suggested that all three proteins could be diluted to 300 nM for estimating antibody KD values. The N protein (Acro Biosystems) was similarly evaluated using an anti-N antibody (Acro Biosystems) as a positive control. The data indicated that the N protein could similarly be diluted to 300 nM for evaluation of experimental antibodies.

Neutralization Assay: Irrespective of the binding results, antibodies were also tested for functional activity against fully infectious SARS-CoV-2 in an in vitro neutralization assay. Antibodies were first screened against the virus at two concentrations: 50 μg/mL and 5 μg/mL. Antibodies that demonstrated a reduction in plaque formation (indicating potential neutralizing activity) at 50 μg/mL were tested in a dilution series using 8, 3-fold dilutions beginning at 50 μg/mL. The antibody concentration required to inhibit 50% of the viral infection (IC₅₀) was calculated in Prism software using a dose-response model with a variable slope.

### Results

In total, 235 antibodies were expressed and 227 generated sufficient material to be tested in binding and neutralization assays. Ninety-seven antibodies demonstrated binding to at least 1 of the proteins tested (Table 2 and Tables 3A-C). Most antibodies bound to either the N or E protein or to at least one form of the Spike protein. A few antibodies demonstrated measurable binding to two or more distinct proteins (e.g., Spike and Envelope). All combinations of antibody binding are reported in Table 2, where the numbers represent the number of antibodies from the donor that recognizes the viral protein. Fifty-eight antibodies demonstrated spike-specific binding to at least one of the spike antigens tested, and 34 antibodies demonstrated binding to at least two of the spike antigens tested. Additionally, 9 antibodies demonstrated binding to E, and 17 antibodies to N. An additional 13 antibodies bound to some combination of the three proteins. The binding activity for these thirteen antibodies may indicate poly-specific activity that is not limited to SARS-CoV-2.

All antibodies, irrespective of binding specificity, were tested for functional activity in a neutralization assay using fully infectious SARS-CoV-2. We chose to take this epitope-agnostic screening approach so that antibodies with unique binding attributes not observed with traditional ELISA and BLI methods but with neutralizing activity were identified. Seventeen antibodies met the initial criteria in the preliminary screen by demonstrating a reduction in virus infectivity at 50 μg/mL. These antibodies were subsequently tested in a dilution series using 8, 3-fold dilutions beginning at 50 μg/mL. Of the 17 antibodies, 12 demonstrated measurable neutralization activity ≤50 μg/mL. ICs values ranged from 0.075 μg/mL to 33.7 μg/mL (Table 3A-C).

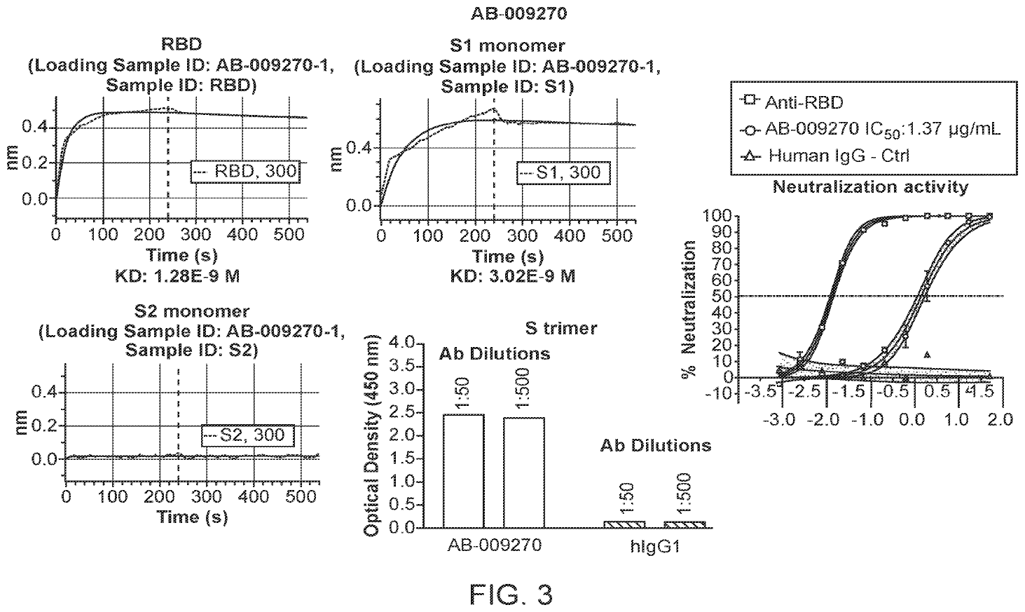

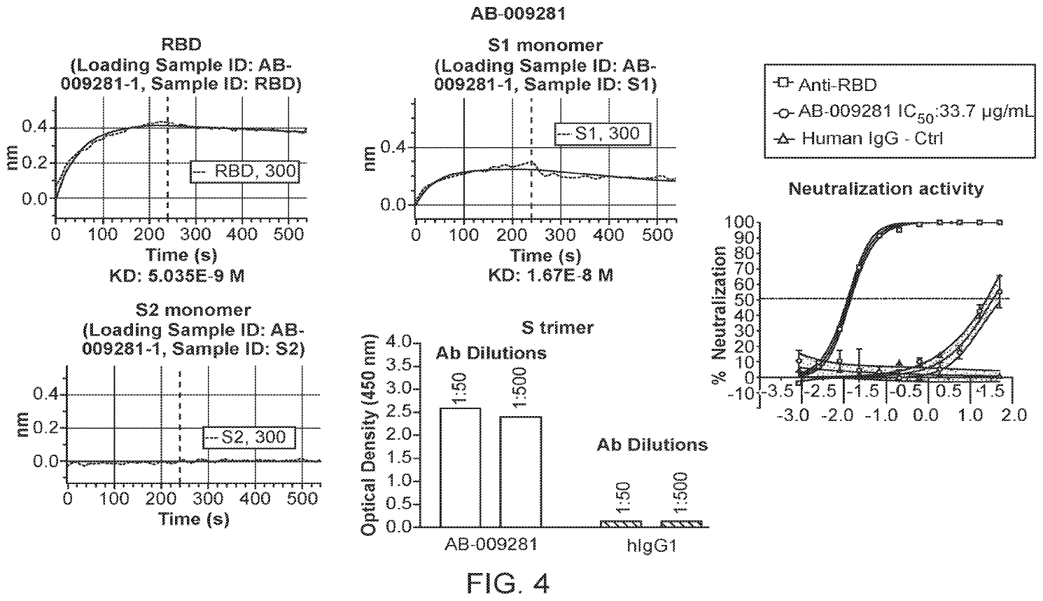

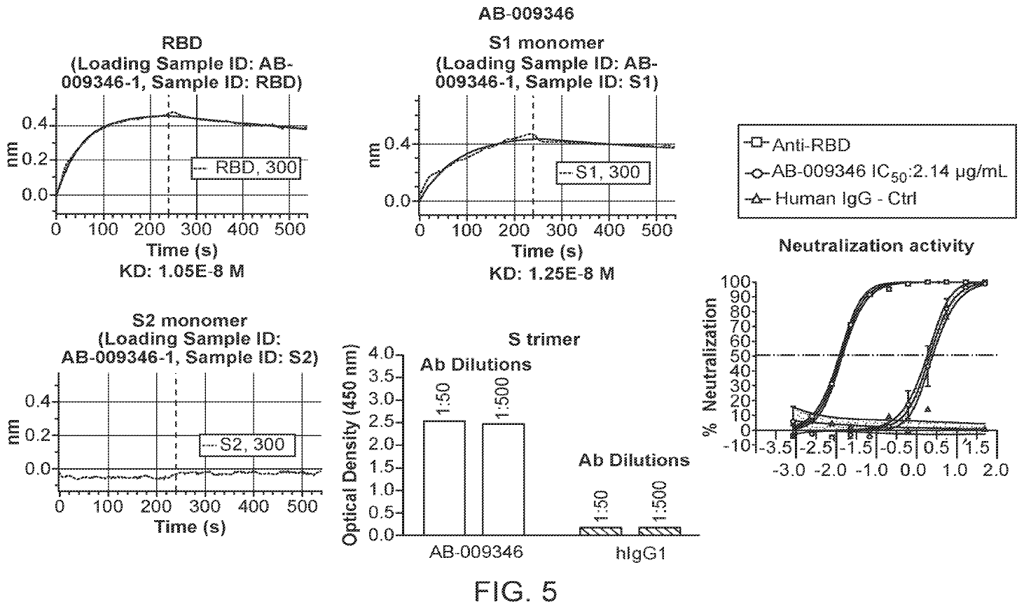

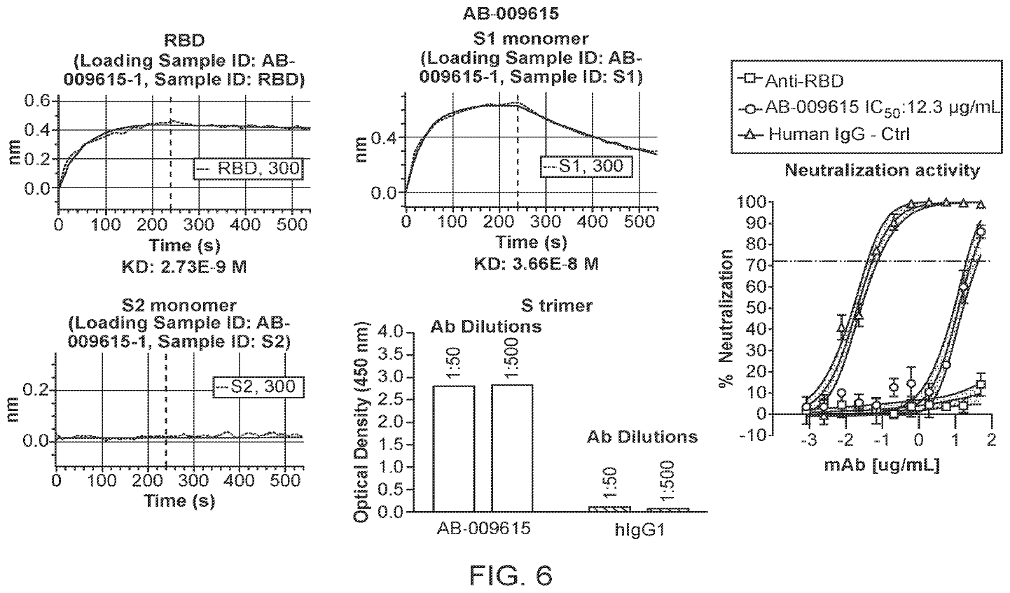

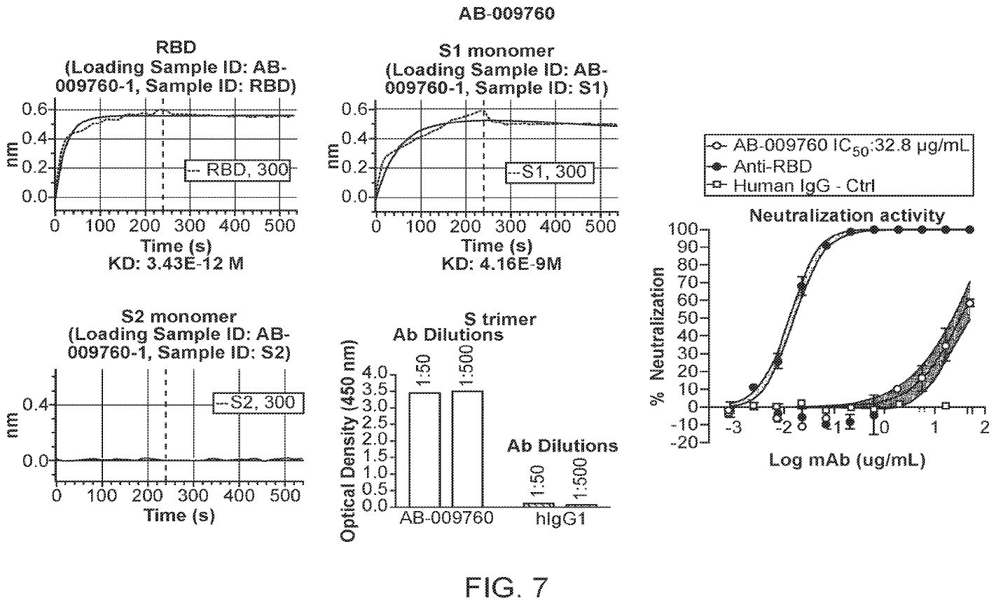

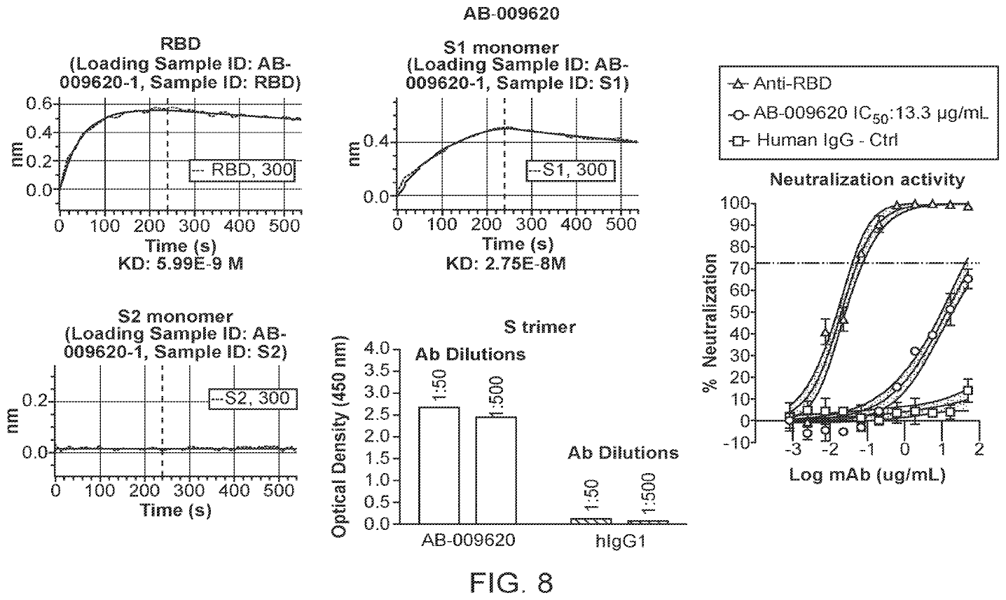

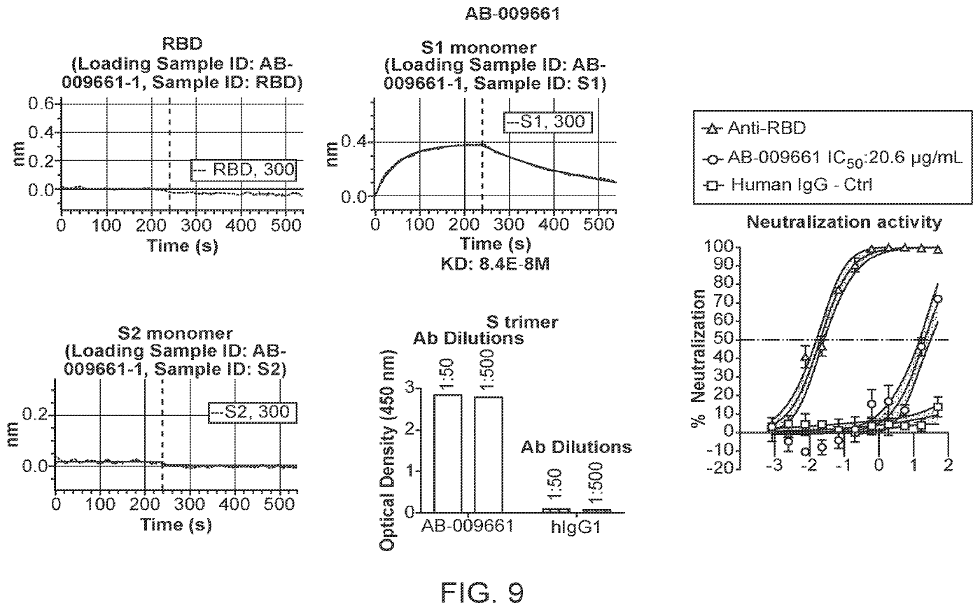

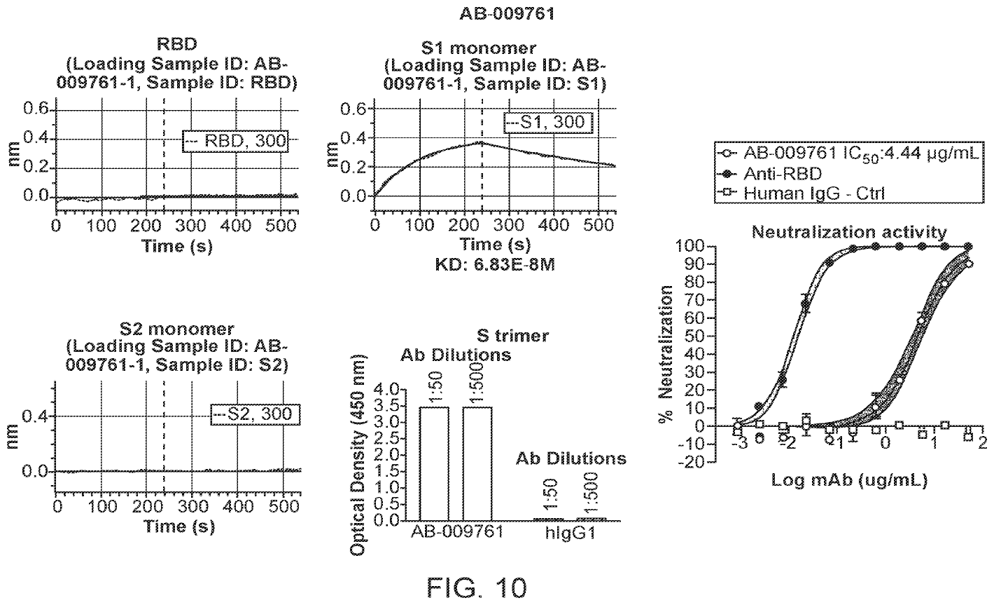

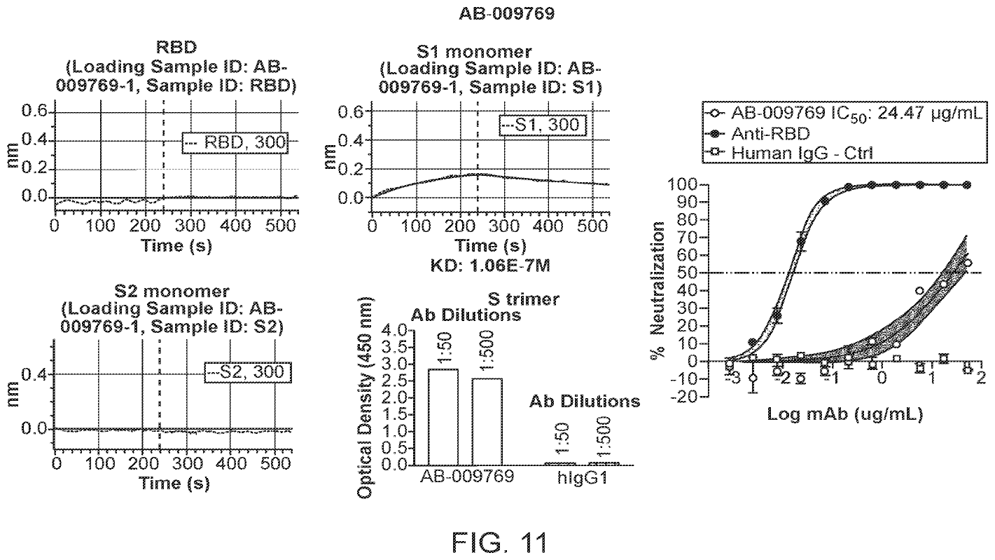

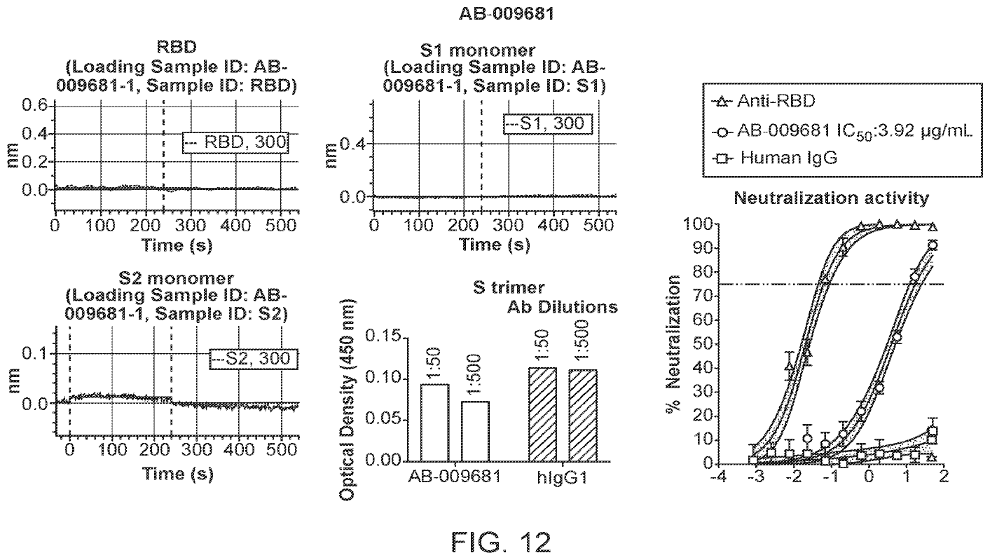

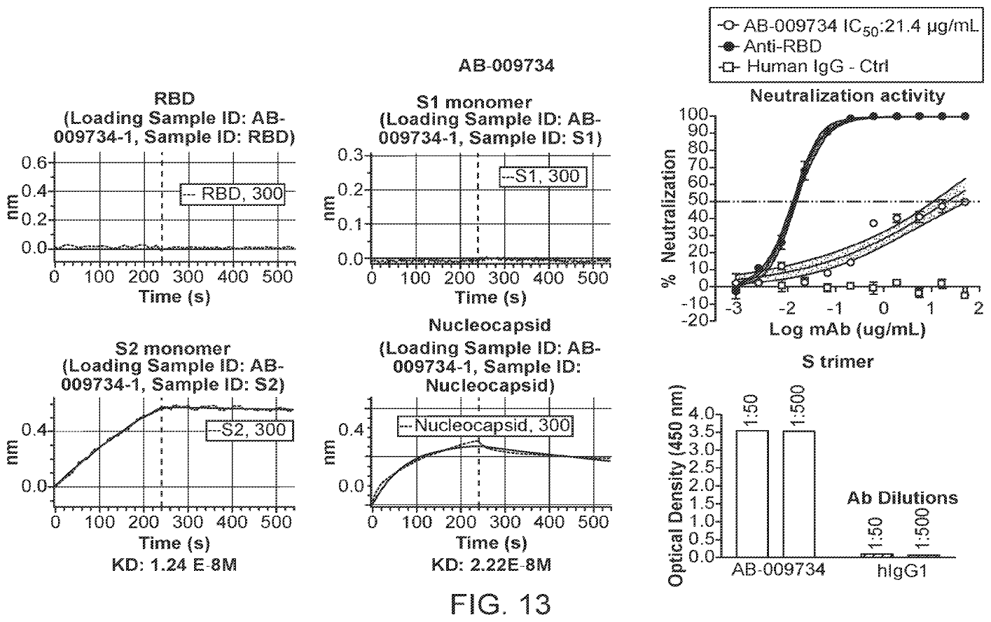

The majority of the 12 neutralizing antibodies demonstrated IC₅₀ values ≥1 μg/mL except the one antibody, AB-009614 (IC₅₀: 0.075 μg/mL; FIG. 2). This antibody demonstrated weak binding to the Spike trimer, but none of the monomeric forms of Spike despite demonstrating the most potent neutralizing activity. These data suggest that the antibody targets a conformationally sensitive epitope only exposed in the context of the complete trimer. AB-009614 bound to neither the E nor N proteins (FIG. 2). Among the remaining 11 antibodies with neutralizing activity ≥1 μg/mL, 6 bound to the RBD and S1 monomers as measured by BLI and to the Spike trimer by ELISA (FIGS. 3-8). Three other antibodies bound to the S1 monomer and Spike trimer, but did not bind to the RBD monomer, suggesting they target a different epitope than the first 6 Abs (FIGS. 9-11). One antibody, AB-009681, surprisingly did not demonstrate binding to any of the SARS-CoV-2 proteins despite an IC₅₀ of 3.9 μg/mL (FIG. 12). The last antibody, AB-009734, demonstrated binding to the S2 monomer and to the Spike trimer, as well as the Nucleocapsid protein and neutralized SARS-CoV-2 with an IC₅₀ of 21.4 μg/mL (FIG. 13). The binding data for this antibody suggests that this antibody may bind to a unique target found on both the S2 and Nucleocapsid proteins.

### Example 6

The antibodies identified to potentially have neutralizing activity in Example 5 were also assayed for neutralizing activity against the SARS CoV-2 Wuhan virus and variants using the Phenosense® Anti-SARS-CoV-2 Neutralizing Antibody Assay (CoV nAb Assay) (Monogram Biosciences/Labcorp). (Huang, Y., et al., medRxiv 2021.09.09.21263049, incorporated by reference herein in its entirety.) Neutralizing antibody activity was measured in a formally validated assay that utilized lentiviral particles pseudotyped with full-length SARS-CoV-2 Spike protein and containing a firefly luciferase (Luc) reporter gene for quantitative measurements of infection by relative luminescence units (RLU). The backbone vector used in pseudovirus creation, F-lucP.CNDOΔU3 encodes the HIV genome with firefly luciferase replacing the HIV env gene. A codon-optimized version of the full-length spike gene of the Wuhan-1 SARS-CoV-2 strain (MN908947.3) (GenScript) was cloned into the Monogram proprietary env expression vector, pCXAS-PXMX, for use in the assay. All the spike mutations described in FIG. 2 including the D614G spike mutation were introduced into the original Wuhan sequence by site-directed mutagenesis. Sequences of the spike gene and expression vector were confirmed by full-length sequencing using Illumina MiSeq NGS.

Pseudovirus stock was produced in HEK 293 cells via a calcium phosphate transfection using a combination of spike plasmid (pCXAS-SARS-CoV-2-D614G) and lentiviral backbone plasmid (F-lucP.CNDOΔU3). Transfected 10 cm² plates were re-fed the next day and harvested on Day 2 post transfection. The pseudovirus stock (supernatant) was collected, filtered and frozen at 570° C. in single-use aliquots. Pseudovirus infectivity was screened at multiple dilutions using HEK293 cells transiently transfected with ACE2 and TMPRSS2 expression vectors. RLUs were adjusted to ˜50,000 for use in the neutralization assay. Neutralization was performed in white 96-well plates by incubating pseudovirus with 8, serial 4-fold dilutions of antibody starting at a concentration of 50 μg/mL for one hour at 37° C.

HEK293 target cells, which had been transfected the previous day with ACE2 and TMPRSS2 expression plasmids, were detached from 10 cm2 plates using trypsin/EDTA and re-suspended in culture medium to a final concentration that accommodated the addition of 10,000 cells per well. Cell suspension was added to the antibody-virus mixtures and assay plates were incubated at 37° C. in 7% CO2 for 3 days. On the day of assay read, Steady Glo (Promega) was added to each well. Reactions were incubated briefly and luciferase signal (RLU) was measured using a luminometer. Neutralization titers represent the inhibitory dilution (ID) of serum samples at which RLUs were reduced by either 50% (ID50) or 80% (ID80) compared to virus control wells (no serum wells).

The Monogram assay employs a specificity control which is created using the same HIV backbone/Luc sequence used in the SARS-CoV-2 pseudovirus. The envelope is 1949 Influenza A H10N3. It is unlikely for antibodies to have been discovered against this rare avian influenza virus in humans. The specificity control is designed to detect non-antibody factors (e.g., ART therapy) that could inhibit SARS-CoV-2 pseudovirus and result in false positive measurements of antibody neutralization. Positive anti-SARS-CoV-2 nAb activity was defined as an anti-SARS-CoV-2 nAb titer >3 times greater than the titer of the same serum tested with the specificity control.

Using the Monogram assay, the antibodies were assayed for neutralizing activity against the following SARS-CoV-2 variants: Alpha (B.1.1.7), Beta (B.1.351), Gamma (P.1.B.1.1.28), Delta (B.1.617.2), Delta-plus (B.1.617.2.1), Epsilon (B.1.427/9), Mu (B.1.621) and Omicron (B.1.1.529). Table 9). Known neutralizing antibodies COVA1-21; CA1; REGN10933 (Casirivimab); REGN10987 (Imdevimab); LY-CoV016 (CB-6, JS-016, LY3832479, etesevimab) and LY-CoV555 (bamlanivimab) were included as positive controls. CR3022 and an anti-nucleocapsid antibody were included as negative controls.

Of the antibodies tested, AB-009614, AB-010019, AB-009270, AB-009346, AB-009681, AB-009761, AB-009615, AB-009620, AB-009734, AB-009760, AB-009281 showed neutralizing activity against the SARS-CoV2 Wuhan virus and at least one of the viral variants. AB-009270 neutralized all but the Omicron variant, and AB-009620 neutralized all of the variants tested (Table 8).

### Example 7

This example describes methods for generating antibody variants.

Antibodies produced as described herein will be modified to generate variant antibodies having improved developability and/or to reduce the risk of clinical immunogenicity, as described above, using methods described in PCT/US2020/018745. Briefly, to reduce risk of clinical immunogenicity, the antibody sequences are evaluated to identify residues that can be engineered to increase similarity to the intended population's native immunoglobulin variable region sequences. One approach to engineering a variant to be as much like self as possible is to identify a close germline sequence and mutate as many mismatched positions (also known as “germline deviations”) to the germline residue type as possible. Each germline gene can present as different alleles in the population. The least immunogenic drug candidate, in terms of minimizing the percent of patients with an immunogenic response, would likely be one that matches an allele commonly found in the patient population. Single nucleotide polymorphism (SNP) data from the human genome can be used to approximate the frequency of alleles in the population.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for the prediction of HLA binding and in vitro assessment to further identify potential epitopes.

Antibody variants can also be designed to enhance the efficacy of the antibody. For example, design parameters can focus on CDRs, e.g., CDR3. Positions to be mutated can be identified based on structural analysis of antibody-antigen co-crystals (Oyen et al., Proc. Natl. Acad Sci. USA 114:E10438-E10445, 2017; Epub Nov. 14 2017) and based on sequence information of other antibodies from the same lineage as the parent or reference antibody.

### Approaches to Mutation Design

Development liabilities can be removed or reduced by one or more mutations. Mutations are designed to preserve antibody structure and function while removing or reducing development liabilities and to improve function. In one aspect, mutations to chemically similar residues can be identified that maintain size, shape, charge, and/or polarity. Illustrative mutations are described in Table 6 or Table 7.

The antibody sequences described herein can be aligned to the putative, D and germline genes. CDRs, germline deviations, and potential liabilities can be identified. For example, non-canonical cysteines and N-glycosylation sites can be identified across the full VH and VL sequences, whereas other potential liability motifs can be identified within the CDRs.

Potential PK risk can also be estimated (Sharma et al., Proc. Natl. Acad. Sci. USA 111:18601-18606, 2014). High hydrophobicity index (HI) correlates with faster clearance, where HI<5 is preferred to reduce risk, and HI<4 is most preferred to reduce risk. However, some antibodies with HI >4, or HI >5, will not exhibit fast clearance. Secondly, too high or too low Fv charge as calculated at pH 5.5 correlates with faster clearance, where a charge between (−2, +8) is preferred to reduce risk, and a charge between (0, +6.2) is most preferred to reduce the risk of fast clearance. Table 6 and Table 7 summarize the types and number of potential liabilities.

### Design of Variants to Germline Antibodies

Framework and complementary-determining region (CDR) germline deviations in antibody sequences can be analyzed for their potential to be mutated, individually or in combination, to germline sequence, without negatively impacting binding to target SARS-CoV-2 antigens/epitopes. For each of the candidate mutations from antibody sequence to germline sequence, the risk of making the mutation can be assessed based on: (1) the change in charge if any since the change in charge is intrinsically risky; (2) conservation of the native residue in the antibody lineage versus the presence of the germline residue or other mutations at that position in the lineage and (3) the structural location of the position with respect to the target antigen epitope. Some mutations may be coupled to at least one other mutation, meaning that the risk prediction is based on making the mutation in conjunction with the other mutation(s).

Design of Variants to Remove Liabilities from Antibody Sequences

Various sequence-based liabilities in antibody sequences described herein can be analyzed for their potential to be mutated to reduce or remove the risk of liability without negatively impacting binding to SARS-CoV-2 antigens or potency of the variant antibody. Residues that contributed to the hydrophobicity index, or to reducing the Fv charge can also be assessed. Similar to the germline design, risk can be assessed based on the change in charge, shape, polarity, backbone conformation preference, and maintenance or enhancement of side-chain interactions.

### Example 8

This example describes a representative in vivo assay to test antibodies for protection from SARS-CoV-2 infection.

### Passive Transfer of Neutralizing Antibodies and SARS-CoV-2 Challenge in Syrian Hamsters

Antibodies described herein can be tested in vivo for the ability to prevent infection by SARS-CoV-2. Antibodies are selected for passive transfer/challenge experiments in a Syrian hamster animal model. See Rogers et al., Id. Candidate neutralizing antibodies are injected intraperitoneally into Syrian hamsters at a starting dose of 2 mg/animal (on average 16.5 mg/kg) and 8 μg/animal at the lowest dose. Control animals can receive 2 mg of a control IgG1. Each group of 6 animals are then is challenged intranasally 12 h post-infusion with 1×10⁶ PFU of SARS-CoV-2. The serum is collected at the time of challenge (Day 0) and Day 5, and the weight of the animals is monitored as an indicator of disease progression. On day 5, lung tissue is collected for viral burden assessment.

Syrian hamsters typically clear the virus within one week after SARS-CoV-2 infection. Hamsters were weighed as a measure of disease due to infection. Lung tissues were collected to measure the viral load on day 5. Lung viral loads are measured by real-time PCR. It is expected that animals that receive an effective dose of neutralizing antibody will show less weight loss than animals that receive a control antibody.

Antibody serum concentrations can also be measured to determine the amount of circulating antibody required for protection against SARS-CoV-2 in vivo. Antibody serum concentrations can be measured prior to the intranasal virus challenge. It is expected that antibody serum concentrations of approximately 22 μg/mL of neutralizing antibody will provide complete protection and a serum concentration of about 12 μg/mL is adequate for 50% reduced disease as measured by weight loss. Sterilizing immunity at serum concentrations that represent a large multiplier of the in vitro neutralizing IC₅₀ is observed for many viruses.

TABLE 1

| | Anti-S (Sino) | | | | Anti-S2 | | | | ACE2-hFc | | | |
|---|---|---|---|---|---|---|---|---|---|---|---|---|
| | Response | Kₐ (1/Ms) | K_{d} (1/s) | K_{D} (M) | Response | Kₐ (1/Ms) | K_{d} (1/s) | K_{D} (M) | Response | Kₐ (1/Ms) | K_{d} (1/s) | K_{D} (M) |
| RBD | 0.59 | 1.21E+05 | 6.89E−07 | 5.70E−12 | N/A | N/A | N/A | N/A | 0.26 | 5.38E+05 | 9.42E−04 | 1.75E−09 |
| S1 | 0.7 | 1.94E+04 | 5.23E−05 | 2.69E−09 | N/A | N/A | N/A | N/A | 0.43 | 1.04E+05 | 1.07E−02 | 1.03E−07 |
| S2 | N/A | N/A | N/A | N/A | 0.99 | 2.37E+04 | 4.88E−07 | 2.06E−11 | N/A | N/A | N/A | N/A |

TABLE 2

| Donor | 117-001-001 | 117-001-002 | 117-001-003 | 117-001-004 |
|---|---|---|---|---|
| Envelope | 2 | 2 | 1 | 4 |
| Nucleocapsid | 7 | 2 | 5 | 3 |
| At least one form of Spike protein | 12 | 4 | 24 | 18 |
| S Trimer | 6 | 1 | 4 | 10 |
| S2 monomer | | | 2 | 1 |
| RBD, S1 & S Trimer | 1 | 1 | 6 | 1 |
| S1, S Trimer | 5 | 1 | 7 | 3 |
| S2, S Trimer | | 1 | 5 | 3 |
| Other combinations | | | | |
| Nucleocapsid, Envelope, S Trimer | 2 | | | 1 |
| Nucleocapsid, S Trimer | 1 | | | |
| RBD, S1, S Trimer, Envelope | | | 1 | |
| S Trimer, Envelope | 3 | | | 2 |
| S2, S Trimer, Envelope | 1 | | | 1 |
| S2, S Trimer, Nucleocapsid | 1 | | | |
| No binding | 52 | 13 | 19 | 46 |

TABLE 3A

| | | Number of nucleotide | | | |
|---|---|---|---|---|---|
| | | mutations from | | | |
| | Lineage | putative germline in | | | Neutralization Activity: |
| Antibody | Size | VH and VL | Donor | Epitope | IC₅₀ (μg/mL) |
| AB-009614 | 1 | 5 | 117-001-001 | S Trimer | 0.075 |
| AB-009270 | 1 | 1 | 117-001-003 | RBD, S1, S Trimer | 1.37 |
| AB-009346 | 17 | 5 | 117-001-003 | RBD, S1, S Trimer | 2.14 |
| AB-009681 | 7 | 2 | 117-001-001 | No binding observed | 3.92 |
| AB-009761 | 9 | 7 | 117-001-001 | S1, S Trimer | 4.44 |
| AB-009615 | 1 | 2 | 117-001-003 | RBD, S1, S Trimer | 12.3 |
| AB-009620 | 1 | 3 | 117-001-002 | RBD, S1, S Trimer | 13.3 |
| AB-009661 | 2 | 1 | 117-001-001 | S1, S Trimer | 20.6 |
| AB-009734 | 11 | 34 | 117-001-001 | S2, S Trimer, Nucleocapsid | 21.43 |
| AB-009769 | 8 | 1 | 117-001-003 | S1, S Trimer | 24.47 |
| AB-009760 | 6 | 5 | 117-001-001 | RBD, S1, S Trimer | 32.76 |
| AB-009281 | 1 | 5 | 117-001-003 | RBD, S1, S Trimer | 33.7 |
| AB-009619 | 6 | 5 | 117-001-004 | S1, S Trimer | >50 |
| AB-009649 | 1 | 1 | 117-001-003 | No binding observed | >50 |
| AB-009655 | 2 | 1 | 117-001-001 | No binding observed | >50 |
| AB-009358 | 13 | 93 | 117-001-003 | S2, S Trimer | >50 |
| AB-009609 | 1 | 8 | 117-001-004 | S1, S Trimer | >50 |

TABLE 3B

| | RBD: BLI | | | | S1: BLI | | | |
|---|---|---|---|---|---|---|---|---|
| Antibody | Response | KD (M) | Kₐ (1/Ms) | K_{d} (1/s) | Response | KD (M) | Kₐ (1/Ms) | K_{d} (1/s) |
| AB-009614 | <LLOD | <LLOD | <LLOD | <LLOD | <LLOD | <LLOD | <LLOD | <LLOD |
| AB-009270 | 0.515 | 1.28E−09 | 1.57E+05 | 2.02E−04 | 0.6616 | 3.02E−09 | 6.90E+04 | 2.08E−04 |
| AB-009346 | 0.465 | 1.05E−08 | 6.06E+04 | 6.33E−04 | 0.4734 | 1.25E−08 | 4.17E+04 | 5.23E−04 |
| AB-009681 | <LLOD | <LLOD | <LLOD | <LLOD | <LLOD | <LLOD | <LLOD | <LLOD |
| AB-009761 | <LLOD | <LLOD | <LLOD | <LLOD | 0.3714 | 6.83E−08 | 2.75E+04 | 1.88E−03 |
| AB-009615 | 0.453 | 2.73E−09 | 6.51E+04 | 1.78E−04 | 0.6535 | 3.66E−08 | 7.54E+04 | 2.76E−03 |
| AB-009620 | 0.571 | 5.99E−09 | 7.09E+04 | 4.25E−04 | 0.5122 | 2.75E−08 | 2.84E+04 | 7.80E−04 |
| AB-009661 | <LLOD | <LLOD | <LLOD | <LLOD | 0.3861 | 8.40E−08 | 5.10E+04 | 4.28E−03 |
| AB-009734 | <LLOD | <LLOD | <LLOD | <LLOD | <LLOD | <LLOD | <LLOD | <LLOD |
| AB-009769 | <LLOD | <LLOD | <LLOD | <LLOD | 0.1617 | 1.06E−07 | 1.84E+04 | 1.94E−03 |
| AB-009760 | 0.597 | 3.43E−12 | 1.43E+05 | 4.88E−07 | 0.5929 | 4.16E−09 | 6.41E+04 | 2.67E−04 |
| AB-009281 | 0.439 | 5.03E−09 | 6.39E+04 | 3.21E−04 | 0.2913 | 1.67E−08 | 8.12E+04 | 1.36E−03 |
| AB-009619 | <LLOD | <LLOD | <LLOD | <LLOD | 0.1436 | 2.58E−08 | 2.81E+04 | 7.26E−04 |
| AB-009649 | <LLOD | <LLOD | <LLOD | <LLOD | <LLOD | <LLOD | <LLOD | <LLOD |
| AB-009655 | <LLOD | <LLOD | <LLOD | <LLOD | <LLOD | <LLOD | <LLOD | <LLOD |
| AB-009358 | <LLOD | <LLOD | <LLOD | <LLOD | <LLOD | <LLOD | <LLOD | <LLOD |
| AB-009609 | <LLOD | <LLOD | <LLOD | <LLOD | 0.521 | 4.94E−08 | 4.64E+04 | 2.29E−03 |

TABLE 3C

| | | | | | S | | | | | |
|---|---|---|---|---|---|---|---|---|---|---|
| | | | | | Trimer: | | | | | Envelope |
| | | | | | ELISA | | | | | ELISA |
| | S2: BLI | | | | OD (450 | Nucleocapsid | | | | OD (450 |
| Antibody | Response | KD (M) | Kₐ (1/Ms) | K_{d} (1/s) | nm)* | Response | KD (M) | Kₐ (1/Ms) | K_{d} (1/s) | nm)* |
| AB-009614 | <LLOD | <LLOD | <LLOD | <LLOD | 0.361 | <LLOD | <LLOD | <LLOD | <LLOD | <LLOD |
| AB-009270 | <LLOD | <LLOD | <LLOD | <LLOD | 2.481 | <LLOD | <LLOD | <LLOD | <LLOD | <LLOD |
| AB-009346 | <LLOD | <LLOD | <LLOD | <LLOD | 2.55 | <LLOD | <LLOD | <LLOD | <LLOD | <LLOD |
| AB-009681 | <LLOD | <LLOD | <LLOD | <LLOD | 0.095 | <LLOD | <LLOD | <LLOD | <LLOD | <LLOD |
| AB-009761 | <LLOD | <LLOD | <LLOD | <LLOD | 3.475 | <LLOD | <LLOD | <LLOD | <LLOD | <LLOD |
| AB-009615 | <LLOD | <LLOD | <LLOD | <LLOD | 2.801 | <LLOD | <LLOD | <LLOD | <LLOD | <LLOD |
| AB-009620 | <LLOD | <LLOD | <LLOD | <LLOD | 2.697 | <LLOD | <LLOD | <LLOD | <LLOD | <LLOD |
| AB-009661 | <LLOD | <LLOD | <LLOD | <LLOD | 2.863 | <LLOD | <LLOD | <LLOD | <LLOD | <LLOD |
| AB-009734 | 0.555 | 1.24E−08 | 8.28E+03 | 1.03E−04 | 3.568 | 0.661 | 2.22E−08 | 4.40E+04 | 9.79E−04 | <LLOD |
| AB-009769 | <LLOD | <LLOD | <LLOD | <LLOD | 2.868 | <LLOD | <LLOD | <LLOD | <LLOD | <LLOD |
| AB-009760 | <LLOD | <LLOD | <LLOD | <LLOD | 3.475 | <LLOD | <LLOD | <LLOD | <LLOD | <LLOD |
| AB-009281 | <LLOD | <LLOD | <LLOD | <LLOD | 2.599 | <LLOD | <LLOD | <LLOD | <LLOD | <LLOD |
| AB-009619 | <LLOD | <LLOD | <LLOD | <LLOD | 1.264 | <LLOD | <LLOD | <LLOD | <LLOD | <LLOD |
| AB-009649 | <LLOD | <LLOD | <LLOD | <LLOD | 0.155 | <LLOD | <LLOD | <LLOD | <LLOD | <LLOD |
| AB-009655 | <LLOD | <LLOD | <LLOD | <LLOD | 0.071 | <LLOD | <LLOD | <LLOD | <LLOD | <LLOD |
| AB-009358 | 0.341 | 4.78E−07 | 3.69E+03 | 1.76E−03 | 2.762 | <LLOD | <LLOD | <LLOD | <LLOD | <LLOD |
| AB-009609 | <LLOD | <LLOD | <LLOD | <LLOD | 2.226 | <LLOD | <LLOD | <LLOD | <LLOD | <LLOD |

TABLE 4
CDR sequences

| Anti- | | | | | | |
|---|---|---|---|---|---|---|
| body | HCDR1 | HCDR2 | HCDR3 | LCDR1 | LCDR2 | LCDR3 |
| AB- | GYTFTSYGI | WISAYNGNTNYAQ | AREGDCSGGSCYSGYYYYY | RASQSVSSYLA | DASNRAT | QQRSNWGT |
| 009269 | S (SEQ ID | KLQG (SEQ ID | GMDV (SEQ ID NO: | (SEQ ID NO: | (SEQ ID | (SEQ ID |
| | NO: 1) | NO: 236) | 471) | 706) | NO: 941) | NO: 1176) |
| AB- | GFTFTSSA | WIVVGSGNTNYAQ | AVNYCSSTSCYDAFDI | RASQSVSSSYLA | GASSRAT | QQYGSSPWT |
| 009270 | MQ (SEQ | KFQE 9 (SEQ | (SEQ ID NO: 472) | (SEQ ID NO: | (SEQ ID | (SEQ ID |
| | ID NO: 2) | ID NO: 237) | | 707) | NO: 942) | NO: 1177) |
| AB- | GYTLTELS | GFDPEDGETIYAQ | ATAPPYCSSTSCPINWFDP | SGSSSNIGNNYV | DNNKRPS | GTWDSSLSEW |
| 009272 | MH (SEQ | KFQG (SEQ ID | (SEQ ID NO: 473) | S (SEQ ID | (SEQ ID | V (SEQ ID |
| | ID NO: 3) | NO: 238) | | NO: 708) | NO: 943) | NO: 1178) |
| AB- | GGTFSSYAI | GIIPIFGTANYAQK | ARVGVYDSSGYYFDY | RASQSVSSYLA | DASNRAT | QQRSNWPPRL |
| 009273 | S (SEQ ID | FQG (SEQ ID | (SEQ ID NO: 474) | (SEQ ID NO: | (SEQ ID | T (SEQ ID |
| | NO: 4) | NO: 239) | | 709) | NO: 944) | NO: 1179) |
| AB- | GFSLSTSG | RIDWDDDKFYSTS | ARSYSSSLPFDY (SEQ | KSSQSVLYSSNN | WASTRES | QQYYSTPFT |
| 009274 | MRVS (SEQ | LKT (SEQ ID | ID NO: 475) | KNYLA (SEQ | (SEQ ID | (SEQ ID |
| | ID NO: 5) | NO: 240) | | ID NO: 710) | NO: 945) | NO: 1180) |
| AB- | GFSLSTSG | RIDWDDDKFYSTS | ARSNYDILTGRLDAFDI | SGSSSNIGSNYV | RNNQRPS | AAWDDSLSG |
| 009275 | MRVS (SEQ | LKT (SEQ ID | (SEQ ID NO: 476) | Y (SEQ ID | (SEQ ID | WV (SEQ ID |
| | ID NO: 6) | NO: 241) | | NO: 711) | NO: 946) | NO: 1181) |
| AB- | GFTFSSYA | AISGSGGSTYYADS | AKDRNYDFWSGYYFDY | RASQGISSYLA | AASTLQS | QQLNSYPLT |
| 009276 | MS (SEQ | VKG (SEQ ID | (SEQ ID NO: 477) | (SEQ ID NO: | (SEQ ID | (SEQ ID |
| | ID NO: 7) | NO: 242) | | 712) | NO: 947) | NO: 1182) |
| AB- | GFTFSSYA | VISYDGSNKYYAD | ARDGGGLLLN (SEQ ID | SGDKLGDKYAC | QDSKRPS | QAWDSSTVV |
| 009277 | MH (SEQ | SVKG (SEQ ID | NO: 478) | (SEQ ID NO: | (SEQ ID | (SEQ ID |
| | ID NO: 8) | NO: 243) | | 713) | NO: 948) | NO: 1183) |
| AB- | GFTFSSYA | VISYDGSNKYYAD | ARDGSSWYIDY (SEQ | GGNNIGSKSVH | DDSDRPS | QVWDSSSDHY |
| 009278 | MH (SEQ | SVKG (SEQ ID | ID NO: 479) | (SEQ ID NO: | (SEQ ID | V (SEQ ID |
| | ID NO: 9) | NO: 244) | | 714) | NO: 949) | NO: 1184) |
| AB- | GFTFSSYA | VISYDGSNKYYAD | ARPRGGSYYSAFDI | RASQSVSSSYLA | GASSRAT | QQYGSSPWT |
| 009279 | MH (SEQ | SVKG (SEQ ID | (SEQ ID NO: 480) | (SEQ ID NO: | (SEQ ID | (SEQ ID |
| | ID NO: | NO: 245) | | 715) | NO: 950) | NO: 1185) |
| | 10) | | | | | |
| AB- | GFTFSSYG | VISYDGSNKYYAD | AKSRGGGYYYYYMDV | RASQGISNYLA | AASSLQS | QQYNSYPPT |
| 009280 | MH (SEQ | SVKG (SEQ ID | (SEQ ID NO: 481) | (SEQ ID NO: | (SEQ ID | (SEQ ID |
| | ID NO: | NO: 246) | | 716) | NO: 951) | NO: 1186) |
| | 11) | | | | | |
| AB- | GFTVSSNY | VIYSGGSTYYADS | ARDLYYYGMDV (SEQ | RASQGISSYLA | AASTLQS | QQLNSYPT |
| 009281 | MS (SEQ | VKG (SEQ ID | ID NO: 482) | (SEQ ID NO: | (SEQ ID | (SEQ ID |
| | ID NO: | NO: 247) | | 717) | NO: 952) | NO: 1187) |
| | 12) | | | | | |
| AB- | GFTVSSNY | VIYSGGSTYYADS | ARILPLNWYYFDY (SEQ | TGSSSNIGAGYD | GNSNRPS | QSYDSSLSGS |
| 009282 | MS (SEQ | VKG (SEQ ID | ID NO: 483) | VH (SEQ ID | (SEQ ID | GV (SEQ ID |
| | ID NO: | NO: 248) | | NO: 718) | NO: 953) | NO: 1188) |
| | 13) | | | | | |
| AB- | GFTVSSNY | VIYSGGSTYYADS | ATSSFDYYYYFMDV | SGDKLGDKYAC | QDTKRPS | QAWDSSIVV |
| 009283 | MS (SEQ | VKG (SEQ ID | (SEQ ID NO: 484) | (SEQ ID NO: | (SEQ ID | (SEQ ID |
| | ID NO: | NO: 249) | | 719) | NO: 954) | NO: 1189) |
| | 14) | | | | | |
| AB- | GGSISSGG | YIYYSGSTYYNPSL | ARAPTIFGVVIGWFDP | RASQSVSSSYLA | GASSRAT | QQYGSSPLT |
| 009284 | YYWS | KS (SEQ ID | (SEQ ID NO: 485) | (SEQ ID NO: | (SEQ ID | (SEQ ID |
| | (SEQ ID | NO: 250) | | 720) | NO: 955) | NO: 1190) |
| | NO: 15) | | | | | |
| AB- | GGSISSGG | YIYYSGSTYYNPSL | ARAPRITIFGVVSPFDY | RASQSVSSSYLA | GASSRAT | QQYGSSPLT |
| 009285 | YYWS | KS (SEQ ID | (SEQ ID NO: 486) | (SEQ ID NO: | (SEQ ID | (SEQ ID |
| | (SEQ ID | NO: 251) | | 721) | NO: 956) | NO: 1191) |
| | NO: 16) | | | | | |
| AB- | GGSISSGD | YIYYSGSTYYNPSL | AREGRFLEWLLSVNYYYYM | QGDSLRSYYAS | GKNNRPS | NSRDSSGNHG |
| 009286 | YYWS | KS (SEQ ID | DV (SEQ ID NO: 487) | (SEQ ID NO: | (SEQ ID | V (SEQ ID |
| | (SEQ ID | NO: 252) | | 722) | NO: 957) | NO: 1192) |
| | NO: 17) | | | | | |
| AB- | GGSISSSSY | SIYYSGSTYYNPSL | ARTRITIFGGERYFDL | RASQGISSYLA | AASTLQS | QQLNSYPLT |
| 009287 | YWG (SEQ | KS (SEQ ID | (SEQ ID NO: 488) | (SEQ ID NO: | (SEQ ID | (SEQ ID |
| | ID NO: | NO: 253) | | 723) | NO: 958) | NO: 1193) |
| | 18) | | | | | |
| AB- | GGSISSYY | YIYYSGSTNYNPSL | ARGFDY (SEQ ID NO: | RASQSVSSSYLA | GASSRAT | QQYGSSPWT |
| 009288 | WS (SEQ | KS (SEQ ID | 489) | (SEQ ID NO: | (SEQ ID | (SEQ ID |
| | ID NO: | NO: 254) | | 724) | NO: 959) | NO: 1194) |
| | 19) | | | | | |
| AB- | GYSFTSYW | IIYPGDSDTRYSPSF | ARRGFGLTYYGMDV | RSSQSLLHSNGY | LGSNRAS | MQALQTPQT |
| 009289 | IG (SEQ | QG (SEQ ID NO: | (SEQ ID NO: 490) | NYLD (SEQ ID | (SEQ ID | (SEQ ID |
| | ID NO: | 255) | | NO: 725) | NO: 960) | NO: 1195) |
| | 20) | | | | | |
| AB- | GFAFSDHH | RIRDKADSYTTEY | ASSHSRGYYAFDY | RASQSVNKFVLA | GASTRAT | QLYDRSPPP |
| 009290 | MD (SEQ | AASVRG (SEQ | (SEQ ID NO: 491) | (SEQ ID NO: | (SEQ ID | (SEQ ID |
| | ID NO: | ID NO: 256) | | 726) | NO: 961) | NO: 1196) |
| | 21) | | | | | |
| AB- | GFTFTTYA | AINAGGAKTHYAD | AKVPSYIYDSRDDYLDF | RASQSVSSYLA | DASSRAT | QQRSDWPLLT |
| 009291 | MF (SEQ | SVRG (SEQ ID | (SEQ ID NO: 492) | (SEQ ID NO: | (SEQ ID | (SEQ ID |
| | ID NO: | NO: 257) | | 727) | NO: 962) | NO: 1197) |
| | 22) | | | | | |
| AB- | GFTVRYNY | IIYRGDRGGSTYYI | ASSHSADYLGPFDY | RASQSITNNYLA | HASKRAS | QQFGTFGT |
| 009292 | MS (SEQ | DSVKG (SEQ ID | (SEQ ID NO: 493) | (SEQ ID NO: | (SEQ ID | (SEQ ID |
| | ID NO: | NO: 258) | | 728) | NO: 963) | NO: 1198) |
| | 23) | | | | | |
| AB- | GFNFNNYG | FISYEGSITHYADS | AKRREVLHLWRDEMDV | RASQDITNSLA | AASTLHS | QQYKTYPFT |
| 009293 | MH (SEQ | VKG (SEQ ID | (SEQ ID NO: 494) | (SEQ ID NO: | (SEQ ID | (SEQ ID |
| | ID NO: | NO: 259) | | 729) | NO: 964) | NO: 1199) |
| | 24) | | | | | |
| AB- | GFTLSTYS | VFSATGGTTDYAD | VKYLQSGDEGY (SEQ | RASQSVSAYFA | DATNRAT | QQRSFWPLT |
| 009294 | VT (SEQ | SVRG (SEQ ID | ID NO: 495) | (SEQ ID NO: | (SEQ ID | (SEQ ID |
| | ID NO: | NO: 260) | | 730) | NO: 965) | NO: 1200) |
| | 25) | | | | | |
| AB- | GFTFSSYA | ILWPDGGKKYFAD | ARDPASGWGLDP (SEQ | TGHNSNIGAGSD | GNRNRPS | QSFDSSLNVY |
| 009295 | MH (SEQ | SVKG (SEQ ID | ID NO: 496) | (SEQ ID NO: | (SEQ ID | V (SEQ ID |
| | ID NO: | NO: 261) | | 731)VH | NO: 966) | NO: 1201) |
| | 26) | | | | | |
| AB- | GYTFTSYDI | WMNPNSGNTGYA | ARAAKRITIFGTRGYYFDY | SGDKLGDKYAC | QDSKRPS | QAWDSSTAWE |
| 009296 | N (SEQ ID | QKFQG (SEQ | (SEQ ID NO: 497) | (SEQ ID NO: | (SEQ ID | V (SEQ ID |
| | NO: 27) | ID NO: 262) | | 732) | NO: 967) | NO: 1202) |
| AB- | GYTFTSYGI | WISAYSGNTNYAQ | ARDSSSWPYVSLGY | RSSQSLLHSNGY | LGSNRAS | MQALQTPRT |
| 009297 | S (SEQ ID | KLQG (SEQ ID | (SEQ ID NO: 498) | (SEQ ID NO: | (SEQ ID | (SEQ ID |
| | NO: 28) | NO: 263) | | 733)NYLD | NO: 968) | NO: 1203) |
| AB- | GGSISSHY | YIYYSGSTNYNPSL | ARIPRGWLQSPSYYFDY | RASQSISSYLN | AASSLQS | QQSYSTPQT |
| 009298 | WS (SEQ | KS (SEQ ID | (SEQ ID NO: 499) | (SEQ ID NO: | (SEQ ID | (SEQ ID |
| | ID NO: | NO: 264) | | 734) | NO: 969) | NO: 1204) |
| | 29) | | | | | |
| AB | GYTFSNYY | IIKPSAGRTIYAQK | ARGGGPTDRDY (SEQ | TGNTNNIGSHAV | RNSLPS | STWDNSLSAV |
| 009299 | MH (SEQ | FVG (SEQ ID | ID NO: 500) | (SEQ ID NO: | (SEQ ID | V (SEQ ID |
| | ID NO: | NO: 265) | | 735)G | NO: 970) | NO: 1205) |
| | 30) | | | | | |
| AB- | GFTFSSYR | SISSSSSYIYYANSV | ARVVWQLVENYYYYMDV | RASQSVSSNLA | GASTRAT | QQYNNWPPIT |
| 009300 | MN (SEQ | KG (SEQ ID NO: | (SEQ ID NO: 501) | (SEQ ID NO: | (SEQ ID | (SEQ ID |
| | ID NO: | 266) | | 736) | NO: 971) | NO: 1206) |
| | 31) | | | | | |
| AB- | GGSFTNYY | EINHSGSTNYKPSL | ARRLGYDNGWGTYFYGLD | QGDSLRKYYPT | GKNTRHS | NSRDSSDSRN |
| 009301 | WN (SEQ | KS (SEQ ID | V (SEQ ID NO: 502) | (SEQ ID NO: | (SEQ ID | WV (SEQ ID |
| | ID NO: | NO: 267) | | 737) | NO: 972) | NO: 1207) |
| | 32) | | | | | |
| AB- | GGSIISTSH | TIYYSGNTYYNPSL | ARRSRAVAGKYYWYYDL | SGDALPKKYAY | EDTKRPS | YSTDSSGYHE |
| 009302 | YWG (SEQ | TS (SEQ ID | (SEQ ID NO: 503) | (SEQ ID NO: | (SEQ ID | V (SEQ ID |
| | ID NO: | NO: 268) | | 738) | NO: 973) | NO: 1208) |
| | 33) | | | | | |
| AB- | GFTFSTYW | NIKPDESEIHYVDS | ARLRAVGGDDTFDV | RASQSINTYLN | LASSLQS | QQSYRSWT |
| 009303 | MT (SEQ | VKG (SEQ ID | (SEQ ID NO: 504) | (SEQ ID NO: | (SEQ ID | (SEQ ID |
| | ID NO: | NO: 269) | | 739) | NO: 974) | NO: 1209) |
| | 34) | | | | | |
| AB- | GFTFSDYA | AITENGFTTYYADS | AKYWSLSGPTELLDS | RASQAITNYLA | SASTLQS | QQYYSYPLT |
| 009304 | MT (SEQ | MRG (SEQ ID | (SEQ ID NO: 505) | (SEQ ID NO: | (SEQ ID | (SEQ ID |
| | ID NO: | NO: 270) | | 740) | NO: 975) | NO: 1210) |
| | 35) | | | | | |
| AB- | GFTFSSYS | VIDNRGGNIHYSDS | AQYVQEGMDV (SEQ | RASQSVRNFFA | GASNRAT | QQRFDWPLT |
| 009305 | MS (SEQ | VRV (SEQ ID | ID NO: 506) | (SEQ ID NO: | (SEQ ID | (SEQ ID |
| | ID NO: | NO: 271) | | 741) | NO: 976) | NO: 1211) |
| | 36) | | | | | |
| AB- | GYTFTSYQ | LIDPDTGGTRYTQ | ARGGRVGATYPYNMDV | SGDALPKKYAY | EDSKRPS | FSTDSSGNHG |
| 009306 | MH (SEQ | NFQG (SEQ ID | (SEQ ID NO: 507) | (SEQ ID NO: | (SEQ ID | V (SEQ ID |
| | ID NO: | NO: 272) | | 742) | NO: 977) | NO: 1212) |
| | 37) | | | | | |
| AB- | GGSISGRG | DISSSGSTNYNPFL | ARDRRLYGDFYYYGMDV | AGSSSNIGTGYD | GNTNRPS | QSYDSSLAGS |
| 009307 | YFWT | KS (SEQ ID | (SEQ ID NO: 508) | (SEQ ID NO: | (SEQ ID | RV (SEQ ID |
| | (SEQ ID | NO: 273) | | 743)VH | NO: 978) | NO: 1213) |
| | NO: 38) | | | | | |
| AB- | GFSFNSYW | SIKQDGSETYYVD | ARGVDGISTIVVLVFWNWY | QGDSLRNYYAS | GKNNRPS | NSRDANTNHV |
| 009308 | MS (SEQ | SVKG (SEQ ID | FDL (SEQ ID NO: | (SEQ ID NO: | (SEQ ID | L (SEQ ID |
| | ID NO: | NO: 274) | 509) | 744) | NO: 979) | NO: 1214) |
| | 39) | | | | | |
| AB- | VGSISSHY | YIYYSGSTNYNPSL | ARSGRGWLQLNPSYYFDY | RASQSVSSSYLA | GASSRAT | QQYGSSPPGT |
| 009309 | WS (SEQ | KS (SEQ ID | (SEQ ID NO: 510) | (SEQ ID NO: | (SEQ ID | (SEQ ID |
| | ID NO: | NO: 275) | | 745) | NO: 980) | NO: 1215) |
| | 40) | | | | | |
| AB- | GFTFSSYA | AISGSGTSTYYADS | AKGYPRYYYGMDV (SEQ | RASQSLSSSNLA | GASSRAT | QQYGSSPFT |
| 009310 | MS (SEQ | VKG (SEQ ID | ID NO: 511) | (SEQ ID NO: | (SEQ ID | (SEQ ID |
| | ID NO: | NO: 276) | | 746) | NO: 981) | NO: 1216) |
| | 41) | | | | | |
| AB- | GYTFTSYDI | WMNPNSGNTGYA | ARRRAATWSIYYYYYYMD | TGTSSDVGGYN | DVSNRPS | TSYTSSSTLE |
| 009311 | N (SEQ | QKFQG (SEQ | V (SEQ ID NO: 512) | (SEQ ID NO: | (SEQ ID | GWV (SEQ |
| | ID NO: | ID NO: 277) | | 747)YVS | NO: 982) | ID NO: |
| | 42) | | | | | 1217) |
| AB- | GFIFSNYW | NIKQDGGEQYYVD | ARSTYLPAAGTMNF | RASHGISTWLA | AASNLQS | HQYNSYPFT |
| 009312 | MS (SEQ | SVKG (SEQ ID | (SEQ ID NO: 513) | (SEQ ID NO: | (SEQ ID | (SEQ ID |
| | ID NO: | NO: 278) | | 748) | NO: 983) | NO: 1218) |
| | 43) | | | | | |
| AB- | GGSFSGYY | EINHSGSTNYNPSL | ARGNIVVVPAAMGVFLYYY | QGDSLRSYYAS | GKNNRPS | NSRDSSGNHL |
| 009313 | WS (SEQ | KS (SEQ ID | YSMDV (SEQ ID NO: | (SEQ ID NO: | (SEQ ID | VV (SEQ ID |
| | ID NO: | NO: 279) | 514) | 749) | NO: 984) | NO: 1219) |
| | 44) | | | | | |
| AB- | GGSISSHY | YIYYSGSTNYNPSL | ARGERQWLQLYSYYGMDV | RASQSVSSSYLA | GASSRAT | QQYGSSPLT |
| 009314 | WS (SEQ | KS (SEQ ID | (SEQ ID NO: 515) | (SEQ ID NO: | (SEQ ID | (SEQ ID |
| | ID NO: | NO: 280) | | 750) | NO: 985) | NO: 1220) |
| | 45) | | | | | |
| AB- | GYTFTSYGI | WISAYNGNTNYAQ | ARDVRMITFGGVIVYFDY | QGDSLRSYYAS | GKNNRPS | NSRDSSGNHR |
| 009315 | S (SEQ | KLQG (SEQ ID | (SEQ ID NO: 516) | (SEQ ID NO: | (SEQ ID | V (SEQ ID |
| | ID NO: | NO: 281) | | 751) | NO: 986) | NO: 1221) |
| | 46) | | | | | |
| AB- | GFTFSNYW | SINNDGSNTRYAD | ARPWGY (SEQ ID | KSSQSLLQSDGK | EVSKRFS | MQSIQVPIT |
| 009316 | MH (SEQ | SVKG (SEQ ID | NO: 517) | TYLY (SEQ ID | (SEQ ID | (SEQ ID |
| | ID NO: | NO: 282) | | NO: 752) | NO: 987) | NO: 1222) |
| | 47) | | | | | |
| AB- | GYTFTSYDI | WMNPNSGNTGYA | ARGMKVVTIFGRREYYFDY | SGDKLGDKYAC | QDSKRPS | QAWDSSTVV |
| 009317 | N (SEQ ID | QKFQG (SEQ | (SEQ ID NO: 518) | (SEQ ID NO: | (SEQ ID | (SEQ ID |
| | NO: 48) | ID NO: 283) | | 753) | NO: 988) | NO: 1223) |
| AB- | GAFISTSNS | SENYGGSSYFNPSL | ARRPSLGFGSSWNFDL | TGTSSDIGGYNY | DVNKRPS | SSYAGSYTYV |
| 009318 | FWG (SEQ | KR (SEQ ID NO: | (SEQ ID NO: 519) | (SEQ ID NO: | (SEQ ID | V (SEQ ID |
| | ID NO: | 284) | | 754) VS | NO: 989) | NO: 1224) |
| | 49) | | | | | |
| AB- | GFSFSNDW | NINQDGSEKYSVD | VRDAGG (SEQ ID | RSSQSLLYSNGN | KVSIRDS | VQGTHWPFT |
| 009319 | LS (SEQ | SVKG (SEQ ID | NO: 520) | TYLS (SEQ ID | (SEQ ID | (SEQ ID |
| | ID NO: | NO: 285) | | NO: 755) | NO: 990) | NO: 1225) |
| | 50) | | | | | |
| AB- | GGTLSRCV | RIIPIFGTVNYAQK | ANTDIGDVRFYGMDV | RSSQSLLDSEDG | TLSYRAS | MQSMEFPWT |
| 009320 | IT (SEQ | FQG (SEQ ID | (SEQ ID NO: 521) | NTYLD (SEQ | (SEQ ID | (SEQ ID |
| | ID NO: | NO: 286) | | ID NO: 756) | NO: 991) | NO: 1226) |
| | 51) | | | | | |
| AB- | GGSISDGD | YISYSGSTYYNPSL | ARIRGERITIFAVLVTGAF | RASQSVTSSYLA | GASSRAT | QQYGSSPLT |
| 009321 | YYWS (SEQ | KS (SEQ ID | DV (SEQ ID NO: 522) | (SEQ ID NO: | (SEQ ID | (SEQ ID |
| | ID NO: | NO: 287) | | 757) | NO: 992) | NO: 1227) |
| | 52) | | | | | |
| AB- | GDSISGTK | SFYYNWGSTSYNA | ARRLEPRHYFDT (SEQ | TRSSGSIASNYV | EDNQRPS | QSYDKGWV |
| 009322 | NYWA (SEQ | SLKS (SEQ ID | ID NO: 523) | Q (SEQ ID | (SEQ ID | (SEQ ID |
| | ID NO: | NO: 288) | | NO: 758) | NO: 993) | NO: 1228) |
| | 53) | | | | | |
| AB- | GYSLSLLT | SFDPQDGETMYAQ | ATDDVSLVVIYALAI | RASQSVSSNYLA | GASSRAT | HQYGTSPHT |
| 009323 | MY (SEQ | KFQG (SEQ ID | (SEQ ID NO: 524) | (SEQ ID NO: | (SEQ ID | (SEQ ID |
| | ID NO: | NO: 289) | | 759) | NO: 994) | NO: 1229) |
| | 54) | | | | | |
| AB- | GFTFSDYDI | HITLTGRTYYVDS | AKHLIRAYDY (SEQ ID | RASQSVTTFLG | DASNRAP | QQRYNWPLT |
| 009324 | S (SEQ ID | VQG (SEQ ID | NO: 525) | (SEQ ID NO: | (SEQ ID | (SEQ ID |
| | NO: 55) | NO: 290) | | 760) | NO: 995) | NO: 1230) |
| AB- | GFTFNNYG | LISYEGSLKYYGDS | AKAGGIFAIHYYGMNV | SGSSSNIGKNYV | DNNKRPS | VTWDSSLSAG |
| 009325 | MH (SEQ | VRG (SEQ ID | (SEQ ID NO: 526) | S (SEQ ID | (SEQ ID | V (SEQ ID |
| | ID NO: | NO: 291) | | NO: 761) | NO: 996) | NO: 1231) |
| | 56) | | | | | |
| AB- | GGSISSSSY | SIYYSGSTYYNPSL | ARDGAPLYDILTHDAFDI | RASQSISSWLA | KASSLES | QQYNSYPYT |
| 009326 | YWG (SEQ | KS (SEQ ID | (SEQ ID NO: 527) | (SEQ ID NO: | SEQ ID | (SEQ ID |
| | ID NO: | NO: 292) | | 762) | NO: 997) | NO: 1232) |
| | 57) | | | | | |
| AB- | GLPFSNPR | RIRSKADGGTSDY | ATSHSFYYDGPFDY | RASQTVSRNYLA | SASIRAT | QHYGSSPPYT |
| 009327 | MS (SEQ | AAPVKG (SEQ | (SEQ ID NO: 528) | (SEQ ID NO: | (SEQ ID | (SEQ ID |
| | ID NO: | ID NO: 293) | | 763) | NO: 998) | NO: 1233) |
| | 58) | | | | | |
| AB- | GTTFSDQF | RSRNKARSYTTEY | TRDGGEGGYTAFDI | RASQVITNDLG | ATSRLQS | LQDSNFPLT |
| 009328 | MD (SEQ | AASVKG (SEQ | (SEQ ID NO: 529) | (SEQ ID NO: | (SEQ ID | (SEQ ID |
| | ID NO: | ID NO: 294) | | 764) | NO: 999) | NO: 1234) |
| | 59) | | | | | |
| AB- | GGSISSSSY | SIYYSGSTYYNPSL | ARHKMGWLQFAGWFDP | QGDSLRSYYAS | GKNNRPS | NSRDSSGNHW |
| 009329 | YWG (SEQ | KS (SEQ ID | (SEQ ID NO: 530) | (SEQ ID NO: | (SEQ ID | V (SEQ ID |
| | ID NO: | NO: 295) | | 765) | NO: 1000) | NO: 1235) |
| | 60) | | | | | |
| AB- | GGSISSSSY | SIYYSGSTYYNPSL | ARHAMSGITIFGVVTNNNW | RASQSVSSNLA | GASTRAT | QQYNNWPPYT |
| 009330 | YWG (SEQ | KS (SEQ ID | FDP (SEQ ID NO: | (SEQ ID NO: | (SEQ ID | (SEQ ID |
| | ID NO: | NO: 296) | 531) | 766) | NO: 1001) | NO: 1236) |
| | 61) | | | | | |
| AB- | GFSLNTAG | HIDWEDEKFYSTS | ARNEWDFDAFDI (SEQ | TATSSDIGNYNY | EVSSRPS | SSYTTSRTYV |
| 009331 | MRVN (SEQ | LKT (SEQ ID | ID NO: 532) | VS (SEQ ID | (SEQ ID | (SEQ ID |
| | ID NO: | NO: 297) | | NO: 767) | NO: 1002) | NO: 1237) |
| | 62) | | | | | |
| AB- | TFIFNNYA | VISGSGSATYYADS | AKVRAGSGSHKAGYYFEK | RASESVSTWLA | KASNLES | QQYSTYST |
| 009332 | MS (SEQ | VKG (SEQ ID | (SEQ ID NO: 533) | (SEQ ID NO: | (SEQ ID | (SEQ ID |
| | ID NO: | NO: 298) | | 768) | NO: 1003) | NO: 1238) |
| | 63) | | | | | |
| AB- | GGSFAGYY | EINHSGGTNYNPFL | VRDSDMTLAAYYKGFQYG | RTSQIVGVNLA | GASTRAT | QQYDNWPRT |
| 009333 | WS (SEQ | KS (SEQ ID | MDV (SEQ ID NO: | (SEQ ID NO: | (SEQ ID | (SEQ ID |
| | ID NO: | NO: 299) | 534) | 769) | NO: 1004) | NO: 1239) |
| | 64) | | | | | |
| AB- | GGSITSRFD | SISHGGNTYYTPSL | ARAYCSGGSCYVYYYFYVD | SGSSSNIGSNFVH | RNNERPS | AAWDDTLSSP |
| 009334 | YWV (SEQ | KS (SEQ ID | V (SEQ ID NO: 535) | (SEQ ID NO: | (SEQ ID | NWV (SEQ |
| | ID NO: | NO: 300) | | 770) | NO: 1005) | ID NO: |
| | 65) | | | | | 1240) |
| AB- | GGSISSHY | YIYYSGSTNYNPSL | ARVRREWLAAYFYYGMDV | RASQGISNYLA | AASTLQS | QKYNSAPLT |
| 009335 | WS (SEQ | KS (SEQ ID | (SEQ ID NO: 536) | (SEQ ID NO: | (SEQ ID | (SEQ ID |
| | ID NO: | NO: 301) | | 771) | NO: 1006) | NO: 1241) |
| | 66) | | | | | |
| AB- | GFSFSSYA | VISFDQTTKYYAD | ARQEDYDYVWGIFRPRGWY | RASQSLGSSYLA | GASTRAT | QQYDSPPPYT |
| 009336 | MH (SEQ | SVKG (SEQ ID | IDL (SEQ ID NO: | (SEQ ID NO: | (SEQ ID | (SEQ ID |
| | ID NO: | NO: 302) | 537) | 772) | NO: 1007) | NO: 1242) |
| | 67) | | | | | |
| AB- | GFTFSSYA | AISGSGGSTYYADS | AKTTGVTMIVVVIEGGYFD | RASQSVSSNLA | GASTRAT | QQYNNWLSYT |
| 009337 | MS (SEQ | VKG (SEQ ID | Y (SEQ ID NO: 538) | (SEQ ID NO: | (SEQ ID | (SEQ ID |
| | ID NO: | NO: 303) | | 773) | NO: 1008) | NO: 1243) |
| | 68) | | | | | |
| AB- | GFTFSSYG | VIWYDGSNKYCA | AREAKPAGGRSFTIFGVAT | TGTSSDVGGYN | DVSNRPS | SSYTSSSTLI |
| 009338 | MH (SEQ | DSVKG (SEQ | YYYYMDV (SEQ ID NO: | YVS (SEQ ID | (SEQ ID | (SEQ ID |
| | ID NO: | ID NO: | 539) | NO: 774) | NO: 1009) | NO: 1244) |
| | 69) | 304) | | | | |
| AB- | GGSISSHY | YIYYSGSTNYNPSL | ARAPRGWLQLSPYYFDL | RASQSVSSSYLA | GASSRAT | HQYGSSPT |
| 009339 | WS (SEQ | KS (SEQ ID | (SEQ ID NO: 540) | (SEQ ID NO: | (SEQ ID | (SEQ ID |
| | ID NO: | NO: 305) | | 775) | NO: 1010) | NO: 1245) |
| | 70) | | | | | |
| AB- | GYTLTELS | GFDPEDGETIYAQ | ATAFAYCGGDCYYPSY | TGTSSDVGGYN | DVSKRPS | CSYAGSSSPW |
| 009340 | MH (SEQ | KFQG (SEQ ID | (SEQ ID NO: 541) | YVS (SEQ ID | (SEQ ID | V (SEQ ID |
| | ID NO: | NO: 306) | | NO: 776) | NO: 1011) | NO: 1246) |
| | 71) | | | | | |
| AB- | GGSISSSSY | SIYYSGSTYYNPSL | ARQMPNRPMIVVVTIRGWF | RASQSVSSNLA | GASTRAT | QQYNNWPRT |
| 009341 | YWG (SEQ | KS (SEQ ID | DP (SEQ ID NO: 542) | (SEQ ID NO: | (SEQ ID | (SEQ ID |
| | ID NO: | NO: 307) | | 777) | NO: 1012) | NO: 1247) |
| | 72) | | | | | |
| AB- | GFTFSTYA | AISGGGHFTYYAD | AKDPRLVTTGWYYDL | RASQSVSSNLA | GASTRAT | QQYHNWPPA |
| 009342 | MS (SEQ | SVEG (SEQ ID | (SEQ ID NO: 543) | (SEQ ID NO: | (SEQ ID | (SEQ ID |
| | ID NO: | NO: 308) | | 778) | NO: 1013) | NO: 1248) |
| | 73) | | | | | |
| AB- | GFTFNNHG | VISYEGSKKDYAD | AKDAAIFMIYPSRYFDL | SGYILGDVSAS | QDTKRPS | QAWDSRTHVV |
| 009343 | MH (SEQ | SVKG (SEQ ID | (SEQ ID NO: 544) | (SEQ ID NO: | (SEQ ID | (SEQ ID |
| | ID NO: | NO: 309) | | 779) | NO: 1014) | NO: 1249) |
| | 74) | | | | | |
| AB- | GFTFSSYS | SISSSSSYIYYADSV | ARGWYQLLLPYYYMDV | RASQSVSSSYLA | GASSRAT | QQYGSSPPKY |
| 009344 | MN (SEQ | KG (SEQ ID NO: | (SEQ ID NO: 545) | (SEQ ID NO: | (SEQ ID | T (SEQ ID |
| | ID NO: | 310) | | 780) | NO: 1015) | NO: 1250) |
| | 75) | | | | | |
| AB- | GFSFSTYA | FMSYDGRNEYYA | ARVPGGVLGTFDI (SEQ | RASQSISTYLN | AASSLQS | QQSYSAPPT |
| 009345 | MH (SEQ | DSVKG (SEQ | ID NO: 546) | (SEQ ID NO: | (SEQ ID | (SEQ ID |
| | ID NO: | ID NO: 311) | | 781) | NO: 1016) | NO: 1251) |
| | 76) | | | | | |
| AB- | GFTFDDYG | GINWNGGSTGYAD | ARWPLLGISGYYHYYMDV | TGTSSDVGGYN | DVSKRPS | CSYAGSYTLL |
| 009346 | MS (SEQ | SVKG (SEQ ID | (SEQ ID NO: 547) | YVS (SEQ ID | (SEQ ID | (SEQ ID |
| | ID NO: | NO: 312) | | NO: 782) | NO: 1017) | NO: 1252) |
| | 77) | | | | | |
| AB- | SFIFSRYAM | TISSNGSNTYYAKS | ARSPGLAIHSSTSLDV | TLSSGYYSYKVD | VGTGGIV | GADHGSGGNF |
| 009347 | H (SEQ ID | VKD (SEQ ID | (SEQ ID NO: 548) | (SEQ ID NO: | KAKGD | VWV (SEQ |
| | NO: 78) | NO: 313) | | 783) | (SEQ ID | ID NO: |
| | | | | | NO: 1018) | 1253) |
| AB- | GFSLSNAR | HIFSNDEKSYSTSL | ARIRAQWLTFYYYMDV | RASQSVSSSYLA | GASSRAT | QQYGSSPPYT |
| 009348 | MGVS | KS (SEQ ID | (SEQ ID NO: 549) | (SEQ ID NO: | (SEQ ID | (SEQ ID |
| | (SEQ ID | NO: 314) | | 784) | NO: 1019) | NO: 1254) |
| | NO: 79) | | | | | |
| AB- | GFSFSTHG | AIWYDGSNENYAD | ARNHIPGSSLGFDP | RASQGISSRLA | ASSTLQS | QQASSLPYT |
| 009349 | MH (SEQ | SVRG (SEQ ID | (SEQ ID NO: 550) | (SEQ ID NO: | (SEQ ID | (SEQ ID |
| | ID NO: | NO: 315) | | 785) | NO: 1020) | NO: 1255) |
| | 80) | | | | | |
| AB- | GFIIDDYA | FIRSKTYGGTTEYA | TKDGRYDWNFILDSYYYYY | QGDSLRRYYAS | GKNKRPS | NSRDTSGDHR |
| 009350 | MS (SEQ | ASVKG (SEQ ID | MDV (SEQ ID NO: | (SEQ ID NO: | (SEQ ID | V (SEQ ID |
| | ID NO: | NO: 316) | 551) | 786) | NO: 1021) | NO: 1256) |
| | 81) | | | | | |
| AB- | GGTLSTYA | RITPIFGTTNYAQK | ARGASGWFGMDV (SEQ | GKNNIESESVH | DDSDRPS | QVWDSTGVH |
| 009351 | IS (SEQ | FQG (SEQ ID | ID NO: 552) | (SEQ ID NO: | (SEQ ID | VV (SEQ ID |
| | ID NO: | NO: 317) | | 787) | NO: 1022) | NO: 1257) |
| | 82) | | | | | |
| AB- | GYMFSKFG | WITPYNSDTVYAQ | ARDPWRDCSSC (SEQ | RASQNVGENFA | GKSIRAT | QQYDLFPWT |
| 009352 | IS (SEQ | NFQG (SEQ ID | ID NO: 553) | (SEQ ID NO: | (SEQ ID | (SEQ ID |
| | ID NO: | NO: 318) | | 788) | NO: 1023) | NO: 1258) |
| | 83) | | | | | |
| AB- | GYAFGSSD | WMHPDSGNTVYA | ARGPRLVRFLQWSLNYMDV | RASQSVSNSYLA | GASSRAT | QQYGSSRGIT |
| 009353 | IN (SEQ | QNFQD (SEQ | (SEQ ID NO: 554) | (SEQ ID NO: | (SEQ ID | (SEQ ID |
| | ID NO: | ID NO: 319) | | 789) | NO: 1024) | NO: 1259) |
| | 84) | | | | | |
| AB- | GFNFSNYG | AISGGGDGASYAD | AKIAD (SEQ ID | KSSQSLLHSDGK | EVSNRFS | MQSIQLPLT |
| 009354 | MS (SEQ | SVKG (SEQ ID | NO: 555) | TFLY (SEQ ID | (SEQ ID | (SEQ ID |
| | ID NO: | NO: 320) | | NO: 790) | NO: 1025) | NO: 1260) |
| | 85) | | | | | |
| AB- | SGSIRSSNY | SVYDSGRTEYNPS | ARHAGPHMVRGVMRNNWF | RASQNIDTYLN | AASSLRS | QQSYSIPWT |
| 009355 | YWG (SEQ | LKS (SEQ ID | DP (SEQ ID NO: | (SEQ ID NO: | (SEQ ID | (SEQ ID |
| | ID NO: | NO: 321) | 556) | 791) | NO: 1026) | NO: 1261) |
| | 86) | | | | | |
| AB- | GYTFVDYA | WIHAGNGNTKDSE | AIAAAGTQVV (SEQ ID | RASQSVANNLA | TASTRAP | QQYYDWPPVT |
| 009356 | IH (SEQ | KFQG (SEQ ID | NO: 557) | (SEQ ID NO: | (SEQ ID | (SEQ ID |
| | ID NO: | NO: 322) | | 792) | NO: 1027) | NO: 1262) |
| | 87) | | | | | |
| AB- | GGSISSSSY | SIYYSGSTYYNPSL | ARHPLSSITIFGVVKREYY | RASQGISSWLA | AASSLQS | QQANSFPLL |
| 009357 | YWG (SEQ | KS (SEQ ID | FDY (SEQ ID NO: | (SEQ ID NO: | (SEQ ID | (SEQ ID |
| | ID NO: | NO: 323) | 558) | 793) | NO: 1028) | NO: 1263) |
| | 88) | | | | | |
| AB- | GYTFTSYY | IIKPSGAGTIYAQK | ARGGGVTDRDY (SEQ | TGNTNNIGGYA | GNSLPS | STWDSSLGAV |
| 009358 | VH (SEQ | FQG (SEQ ID | ID NO: 559) | VG (SEQ ID | (SEQ ID | L (SEQ ID |
| | ID NO: | NO: 324) | | NO: 794) | NO: 1029) | NO: 1264) |
| | 89) | | | | | |
| AB- | GYRYTNY | IIYPADSDTRYSPSF | ARSFEYCTGTTCQSWFDF | TGTSSNIGAGYG | DNTNRPS | HSYDTTLSGQ |
| 009359 | WVA (SEQ | QG (SEQ ID NO: | (SEQ ID NO: 560) | VH (SEQ ID | (SEQ ID | GV (SEQ ID |
| | ID NO: | 325) | | NO: 795) | NO: 1030) | NO: 1265) |
| | 90) | | | | | |
| AB- | GYTFTSYDI | WMNPNSGNTGYA | ARGRAYQLLFNYYYYMDV | SGDKLGDKYAC | QDSKRPS | QAWDSSTVV |
| 009360 | N (SEQ ID | QKFQG (SEQ ID | (SEQ ID NO: 561) | (SEQ ID NO: | (SEQ ID | (SEQ ID |
| | NO: 91) | NO: 326) | | 796) | NO: 1031) | NO: 1266) |
| AB- | GFIFNNYG | VLSGSSYIKYEADS | ARKGVGSQDYGLDV | SGSTSNIGSNFV | RNTQRPS | AAWDDSLSGG |
| 009361 | LT (SEQ | VRG (SEQ ID | (SEQ ID NO: 562) | Y (SEQ ID | (SEQ ID | V (SEQ ID |
| | ID NO: | NO: 327) | | NO: 797) | NO: 1032) | NO: 1267) |
| | 92) | | | | | |
| AB- | GYTFTSYY | IINPSGGSTSYAQK | ARDPTTVTTYDYYYGMDV | SGDALPKQYAY | KDSERPS | QSADSSGTFV |
| 009362 | MH (SEQ | FQG (SEQ ID | (SEQ ID NO: 563) | (SEQ ID NO: | (SEQ ID | V (SEQ ID |
| | ID NO: | NO: 328) | | 798) | NO: 1033) | NO: 1268) |
| | 93) | | | | | |
| AB- | GFTFSSFA | AIGGDGGATYYVD | ATPSPTRYS (SEQ ID | KSSQSVLYSSNN | WASTRQS | QQYHKTPHT |
| 009363 | MT (SEQ | SIKG (SEQ ID | NO: 564) | KNYLA (SEQ | (SEQ ID | (SEQ ID |
| | ID NO: | NO: 329) | | ID NO: 799) | NO: 1034) | NO: 1269) |
| | 94) | | | | | |
| AB- | GFTFSSYA | VISYDGSNKYYAD | ARDPDYYDSSSDAFDI | RSSQSLVHSDGN | KVSNRDS | MQGTHWPPW |
| 009364 | MH (SEQ | SVKG (SEQ ID | (SEQ ID NO: 565) | TYLN (SEQ ID | (SEQ ID | T (SEQ ID |
| | ID NO: | NO: 330) | | NO: 800) | NO: 1035) | NO: 1270) |
| | 95) | | | | | |
| AB- | GYTLTELS | GFDPEDAETIYAQ | ATALAVTGTSDSYYYYYGM | RSSQSLVHSDGN | KISNRFS | MQATQFPWT |
| 009609 | MH (SEQ | KFQG (SEQ ID | DV (SEQ ID NO: 566) | TYLS (SEQ ID | (SEQ ID | (SEQ ID |
| | ID NO: | NO: 331) | | NO: 801) | NO: 1036) | NO: 1271) |
| | 96) | | | | | |
| AB- | GFSLSTSG | RIDWDDDKYYSTS | AREGVATISLDY (SEQ | RASQSISSYLN | AASSLQS | QQSYSTPRT |
| 009611 | MCVS | LKT (SEQ ID | ID NO: 567) | (SEQ ID NO: | (SEQ ID | (SEQ ID |
| | (SEQ ID | NO: 332) | | 802) | NO: 1037) | NO: 1272) |
| | NO: 97) | | | | | |
| AB- | GFSLSTSG | RIDWDDDKYYSTS | ARTQANRVAFDI (SEQ | RASQSISSYLN | AASSLQS | QQSYSTPRT |
| 009612 | MCVS | LKT (SEQ ID | ID NO: 568) | (SEQ ID NO: | (SEQ ID | (SEQ ID |
| | (SEQ ID | NO: 333) | | 803) | NO: 1038) | NO: 1273) |
| | NO: 98) | | | | | |
| AB- | GFTFSSYG | VISYDGSNKYYAD | AKDQKYYDILTGYYRSTYY | RASQSVSSNLA | GASTRAT | QQYNNWPPLT |
| 009614 | MH (SEQ | SVKG (SEQ ID | YYYYGMDV (SEQ ID | (SEQ ID NO: | (SEQ ID | (SEQ ID |
| | ID NO: | NO: 334) | NO: 569) | 804) | NO: 1039) | NO: 1274) |
| | 99) | | | | | |
| AB- | GFTFSSYG | VIWYDGSNKYYA | AKVGSPYCSGGSCYSGYFD | QASQDISNYLN | DASNLET | QQYDNLPPLT |
| 009615 | MH (SEQ | DSVKG (SEQ | Y (SEQ ID NO: 570) | (SEQ ID NO: | (SEQ ID | (SEQ ID |
| | ID NO: | ID NO: 335) | | 805) | NO: 1040) | NO: 1275) |
| | 100) | | | | | |
| AB- | GFTFSSYG | VISYDGSNKYYAD | AKSPEGAAAGTGYFDY | GGNNIGSKSVH | DDSDRPS | QVWDSSSDPW |
| 009616 | MH (SEQ | SVKG (SEQ ID | (SEQ ID NO: 571) | (SEQ ID NO: | (SEQ ID | V (SEQ ID |
| | ID NO: | NO: 336) | | 806) | NO: 1041) | NO: 1276) |
| | 101) | | | | | |
| AB- | GYTLIELS | GFDPEDGETIYAQ | ATGPAAATSGWFDP | TGTSSDVGGYN | DVSNRPS | SSYTSSSTRV |
| 009619 | MH (SEQ | KFQG (SEQ ID | (SEQ ID NO: 572) | YVS (SEQ ID | (SEQ ID | (SEQ ID |
| | ID NO: | NO: 337) | | NO: 807) | NO: 1042) | NO: 1277) |
| | 102) | | | | | |
| AB- | GFSLSTSG | LIYWDDDKRYSPS | AHHEVATVFDY (SEQ | TGTSSDVGAYN | EVSNRPS | SSYTSSSTV |
| 009620 | VGVG | LKS (SEQ ID | ID NO: 573) | YVS (SEQ ID | (SEQ ID | (SEQ ID |
| | (SEQ ID | NO: 338) | | NO: 808) | NO: 1043) | NO: 1278) |
| | NO: 103) | | | | | |
| AB- | GGSFSGYY | EINHSGSTNYNPSL | ARGTGIAVAGVLVNFYYCY | QGDSLRSYYAS | GKNNRPS | NSRDSSGNHL |
| 009629 | WS (SEQ | KS (SEQ ID | YMDV (SEQ ID NO: | (SEQ ID NO: | (SEQ ID | V (SEQ ID |
| | ID NO: | NO: 339) | 574) | 809) | NO: 1044) | NO: 1279) |
| | 104) | | | | | |
| AB- | GFNFSFYW | NIKQDGSEKYYVD | ARLSGGSWYFDY (SEQ | SGDKLGDKYAC | QDSKRPS | QAWDSSTGV |
| 009631 | MS (SEQ | SVKG (SEQ ID | ID NO: 575) | (SEQ ID NO: | (SEQ ID | (SEQ ID |
| | ID NO: | NO: 340) | | 810) | NO: 1045) | NO: 1280) |
| | 105) | | | | | |
| AB- | GGSISSYY | YIYYSGSTNYNPSL | ARGFDY (SEQ ID | RASQSVGSIYLA | GASSRAT | QQYGSSLTWT |
| 009633 | WS (SEQ | KS (SEQ ID | NO: 576) | (SEQ ID NO: | (SEQ ID | (SEQ ID |
| | ID NO: | NO: 341) | | 811) | NO: 1046) | NO: 1281) |
| | 106) | | | | | |
| AB- | GFSLSTSG | RIDWDDDKFYSTS | ARYYYYGMDV (SEQ | KSSQSVLYSSNN | WASTRES | QQYYSTLWT |
| 009634 | MRVS | LKT (SEQ ID | ID NO: 577) | KNYLA (SEQ | (SEQ ID | (SEQ ID |
| | (SEQ ID | NO: 342) | | ID NO: 812) | NO: 1047) | NO: 1282) |
| | NO: 107) | | | | | |
| AB- | GFTFSNAW | RIKSKTDGGTTDY | TTGRYYYDSSGYANDAFDI | SGDALPKKYAY | EDSKRPS | YSTDSSGNHR |
| 009635 | MS (SEQ | AAPVKG (SEQ | (SEQ ID NO: 578) | (SEQ ID NO: | (SEQ ID | V (SEQ ID |
| | ID NO: | ID NO: 343) | | 813) | NO: 1048) | NO: 1283) |
| | 108) | | | | | |
| AB- | GGTFSSYAI | GIIPILGTANYAQK | AREGGFYGSGRNFDY | RSSQSLLHSNGY | LGSNRAS | MQALQTPYT |
| 009637 | S (SEQ ID | FQG (SEQ ID | (SEQ ID NO: 579) | NYLD (SEQ ID | (SEQ ID | (SEQ ID |
| | NO: 109) | NO: 344) | | NO: 814) | NO: 1049) | NO: 1284) |
| AB- | GFTFSDYY | YISSSGSTIYYADS | ARDSTRYCSSTSCSSYYYG | SGDALPKQYAY | KDSERPS | QSADSSGTWV |
| 009638 | MS (SEQ | VKG (SEQ ID | MDV (SEQ ID NO: | (SEQ ID NO: | (SEQ ID | (SEQ ID |
| | ID NO: | NO: 345) | 580) | 815) | NO: 1050) | NO: 1285) |
| | 110) | | | | | |
| AB- | GFTFSSYA | VISYDGSNKYYAD | ARDSDDYGDYYFDY | RSSQSLVHSDGN | KVSNRDS | MQGTHWPPW |
| 009640 | MH (SEQ | SVKG (SEQ ID | (SEQ ID NO: 581) | TYLN (SEQ ID | (SEQ ID | T (SEQ ID |
| | ID NO: | NO: 346) | | NO: 816) | NO: 1051) | NO: 1286) |
| | 111) | | | | | |
| AB- | GGSFSGYS | EINHSGSTNYNPSL | AREGRFLEWLLGVNYYYY | QGDSLRSYYAS | GKNNRPS | HSRDSSGNHL |
| 009642 | WS (SEQ | KS (SEQ ID | MDV (SEQ ID NO: | (SEQ ID NO: | (SEQ ID | V (SEQ ID |
| | ID NO: | NO: 347) | 582) | 817) | NO: 1052) | NO: 1287) |
| | 112) | | | | | |
| AB- | GFTFGDYA | FIRSKAYGGTTEY | TREDFWSGYSDY (SEQ | RASQSISSWLA | KASSLES | QQYNSYPWT |
| 009643 | MS (SEQ | AASVKG (SEQ | ID NO: 583) | (SEQ ID NO: | (SEQ ID | (SEQ ID |
| | ID NO: | ID NO: 348) | | 818) | NO: 1053) | NO: 1288) |
| | 113) | | | | | |
| AB- | GFTFSSYG | VISYDGSNKYYAD | AKDPGITIFGVVYYYYMDV | RASQSISSYLN | AASSLQS | QQSYSTPHT |
| 009644 | MH (SEQ | SVKG (SEQ ID | (SEQ ID NO: 584) | (SEQ ID NO: | (SEQ ID | (SEQ ID |
| | ID NO: | NO: 349) | | 819) | NO: 1054) | NO: 1289) |
| | 114) | | | | | |
| AB- | GFTFSSYA | VISYDGSNKYYAD | ARACSSTSCYKGDAFDI | SGDALPKQYAY | KDSERPS | QSADSSGTYS |
| 009646 | MH (SEQ | SVKG (SEQ ID | (SEQ ID NO: 585) | (SEQ ID NO: | (SEQ ID | WV (SEQ ID |
| | ID NO: | NO: 350) | | 820) | NO: 1055) | NO: 1290) |
| | 115) | | | | | |
| AB- | GYTFAGYD | WASTKNDNTGYA | ARGVDQGVDY (SEQ ID | TLRSGINVDSYR | YKSAADK | MIWHNSAYF |
| 009647 | IN (SEQ | QKFQG (SEQ | NO: 586) | IY (SEQ ID | EQGS | (SEQ ID |
| | ID NO: | ID NO: 351) | | NO: 821) | (SEQ ID | NO: 1291) |
| | 116) | | | | NO: 1056) | |
| AB- | GFTFSSYW | NIKQDGSEKYYVD | ARDFNDFWSGYYGPSFDY | SGDKLGDKYAC | QDSKRPS | QAWDSSTGV |
| 009649 | MS (SEQ | SVKG (SEQ ID | (SEQ ID NO: 587) | (SEQ ID NO: | (SEQ ID | (SEQ ID |
| | ID NO: | NO: 352) | | 822) | NO: 1057) | NO: 1292) |
| | 117) | | | | | |
| AB- | GFTFSSYA | VISYDGSNKYYAD | ARVWVPKVYYYGMDV | QASQDISNYLN | DASNLET | QQYDNLPIT |
| 009650 | MH (SEQ | SVKG (SEQ ID | (SEQ ID NO: 588) | (SEQ ID NO: | (SEQ ID | (SEQ ID |
| | ID NO: | NO: 353) | | 823) | NO: 1058) | NO: 1293) |
| | 118) | | | | | |
| AB- | GGSFSGYY | EINHSGSTNYNPSL | ARSWSSWSYAFDI (SEQ | TGTSSDVGSYNL | EGSKRPS | CSYAGSSTWV |
| 009654 | WS (SEQ | KS (SEQ ID | ID NO: 589) | VS (SEQ ID | (SEQ ID | (SEQ ID |
| | ID NO: | NO: 354) | | NO: 824) | NO: 1059) | NO: 1294) |
| | 119) | | | | | |
| AB- | GFTFSSYW | NIKQDGSEKYYVD | ARDSSDPIGAFDI (SEQ | SGDKLGDKYAC | QDSKRPS | QAWDSSTVV |
| 009655 | MS (SEQ | SVKG (SEQ ID | ID NO: 590) | (SEQ ID NO: | (SEQ ID | (SEQ ID |
| | ID NO: | NO: 355) | | 825) | NO: 1060) | NO: 1295) |
| | 120) | | | | | |
| AB- | GFTFSSYS | SISSSSSYIYYADSV | ATIPAAMESAYYYYGMDV | SGDKLGDKYAC | QDSKRPS | QAWDSSTVV |
| 009657 | MN (SEQ | KG (SEQ ID NO: | (SEQ ID NO: 591) | (SEQ ID NO: | (SEQ ID | (SEQ ID |
| | ID NO: | 356) | | 826) | NO: 1061) | NO: 1296) |
| | 121) | | | | | |
| AB- | GFTFSSYA | AISGSGGSTYYADS | AKRLDY (SEQ ID | RSSQSLVHSDGN | KISNRFS | TQATQFPWT |
| 009659 | MS (SEQ | VKG (SEQ ID | NO: 592) | TYLS (SEQ ID | (SEQ ID | (SEQ ID |
| | ID NO: | NO: 357) | | NO: 827) | NO: 1062) | NO: 1297) |
| | 122) | | | | | |
| AB- | GFTFSSYG | VISYDGSNKYYAD | AKDYGDYGGMDV (SEQ | KSSQSVLYSSNN | WASTRES | QQYYSTPR |
| 009661 | MH (SEQ | SVKG (SEQ ID | ID NO: 593) | KNYLA (SEQ | (SEQ ID | (SEQ ID |
| | ID NO: | NO: 358) | | ID NO: 828) | NO: 1063) | NO: 1298) |
| | 123)) | | | | | |
| AB- | GFSFRSFG | LITYDATEKHFGDS | VKDLGYKDN (SEQ ID | QGDSLRNYYAS | AQNNRPS | NSRDSSGDRL |
| 009664 | MH (SEQ | VKG (SEQ ID | NO: 594) | (SEQ ID NO: | (SEQ ID | V (SEQ ID |
| | ID NO: | NO: 359) | | 829) | NO: 1064) | NO: 1299) |
| | 124) | | | | | |
| AB- | GFTFSSYA | AISGSGGSTYYADS | AKHARRHDYGDYFDY | SGDKLGDKYAC | QDTKRPS | QAWDSSTAW |
| 009667 | MS (SEQ | VKG (SEQ ID | (SEQ ID NO: 595) | (SEQ ID NO: | (SEQ ID | V (SEQ ID |
| | ID NO: | NO: 360) | | 830) | NO: 1065) | NO: 1300) |
| | 125) | | | | | |
| AB- | GGSISSYY | YIYYSGSTYYNPSL | ARALRGIFGVVNWFDP | RASQSVSSNYLA | GASSRAT | QQYGSSPLT |
| 009671 | WS (SEQ | KS (SEQ ID | (SEQ ID NO: 596) | (SEQ ID NO: | (SEQ ID | (SEQ ID |
| | ID NO: | NO: 361) | | 831) | NO: 1066) | NO: 1301) |
| | 126) | | | | | |
| AB- | GGSFSGYY | EINHSGSTNYNPSL | ARLSRQWLVRPHIYYFDY | RASQSVSSYLN | AASSLQS | QQSYSTPRT |
| 009676 | WS (SEQ | KS (SEQ ID | (SEQ ID NO: 597) | (SEQ ID NO: | (SEQ ID | (SEQ ID |
| | ID NO: | NO: 362) | | 832) | NO: 1067) | NO: 1302) |
| | 127) | | | | | |
| AB- | GFSLSTSG | RIDWDDDKYYSTS | ARSPIAARAGGFDY | RASQSISSYLN | AASSLQS | QQSYSTPRT |
| 009677 | MCVS | LKT (SEQ ID | (SEQ ID NO: 598) | (SEQ ID NO: | (SEQ ID | (SEQ ID |
| | (SEQ ID | NO: 363) | | 833) | NO: 1068) | NO: 1303) |
| | NO: 128) | | | | | |
| AB- | GFTFSTYG | AIWYDGSNKYYA | ARRGVTISYYYMDV | GLSSGSVSTTYY | NTKTRSS | VLYMGSGIWV |
| 009680 | MH (SEQ | DSVKG (SEQ | (SEQ ID NO: 599) | (SEQ ID NO: | (SEQ ID | (SEQ ID |
| | ID NO: | ID NO: 364) | | 834)PS | NO: 1069) | NO: 1304) |
| | 129) | | | | | |
| AB- | GGTFSSYAI | GIIPIFGTANYAQK | ASVGGDYDLDY (SEQ | RASQSVSSYLA | DASNRAT | QQRSNWPPYT |
| 009681 | S (SEQ ID | FQG (SEQ ID | ID NO: 600) | (SEQ ID NO: | (SEQ ID | (SEQ ID |
| | NO: 130) | NO: 365) | | 835) | NO: 1070) | NO: 1305) |
| AB- | GGSISSYY | YIYYSGSTNYNPSL | ARGATIFGVVIRPFDY | RASQSVSSSYLA | GASSRAT | QQYGSSPLT |
| 009682 | WS (SEQ | KS (SEQ ID | (SEQ ID NO: 601) | (SEQ ID NO: | (SEQ ID | (SEQ ID |
| | ID NO: | NO: 366) | | 836) | NO: 1071) | NO: 1306) |
| | 131) | | | | | |
| AB- | GGSISSSSY | SIYYSGSTYYNPSL | ARHTFGITIFGVVTRTGFD | RASQSISSYLN | AASSLQS | QQSYSTPYT |
| 009687 | YWG (SEQ | KS (SEQ ID | P (SEQ ID NO: 602) | (SEQ ID NO: | (SEQ ID | (SEQ ID |
| | ID NO: | NO: 367) | | 837) | NO: 1072) | NO: 1307) |
| | 132) | | | | | |
| AB- | GFTFSSYA | VISYDGSNKYYAD | AREMGSGMDV (SEQ ID | SGDKLGDKYAC | QDSKRPS | QAWDSSTGV |
| 009689 | MH (SEQ | SVKG (SEQ ID | NO: 603) | (SEQ ID NO: | (SEQ ID | (SEQ ID |
| | ID NO: | NO: 368) | | 838) | NO: 1073) | NO: 1308) |
| | 133) | | | | | |
| AB- | RFSTSENY | RSKNRASSFATEY | AGSIAVAGAI (SEQ ID | RSSQSLLHSNGY | FGSNRAS | MQSLQTPVT |
| 009691 | VD (SEQ | AASVKG (SEQ | NO: 604) | NYLV (SEQ ID | (SEQ ID | (SEQ ID |
| | ID NO: | ID NO: 369) | | NO: 839) | NO: 1074) | NO: 1309) |
| | 134) | | | | | |
| AB- | GFTFSSYA | VISYDGSNKYYAD | ARARGGNYYYGMDV | RASQSISSWLA | KASSLES | QQYNSYSWT |
| 009695 | MH (SEQ | SVKG (SEQ ID | (SEQ ID NO: 605) | (SEQ ID NO: | (SEQ ID | (SEQ ID |
| | ID NO: | NO: 370) | | 840) | NO: 1075) | NO: 1310) |
| | 135) | | | | | |
| AB- | GGSISSSSY | SIYYSGSTYYNPSL | ASREVVLRYFDWSSPMGGF | TGTSSDVGGYN | DVSNRPS | SSYTSSSTVV |
| 009697 | YWG (SEQ | KS (SEQ ID | DP (SEQ ID NO: 606) | YVS (SEQ ID | (SEQ ID | (SEQ ID |
| | ID NO: | NO: 371) | | NO: 841) | NO: 1076) | NO: 1311) |
| | 136) | | | | | |
| AB- | GFTLNTYA | SSSGTGATTNYAD | VRDGYNMIPFDM (SEQ | GASRSLGSNYLA | GAFHRAN | QQYNSSPYT |
| 009703 | MS (SEQ | SVNG (SEQ ID | ID NO: 607) | (SEQ ID NO: | (SEQ ID | (SEQ ID |
| | ID NO: | NO: 372) | | 842) | NO: 1077) | NO: 1312) |
| | 137) | | | | | |
| AB- | GGSFSGYY | EINHAGSLSYNPSL | VRWGPVGNTWAFDY | TLSSEHSTYTID | IKSDGSHI | GQSHTNNGQA |
| 009706 | WS (SEQ | KS (SEQ ID | (SEQ ID NO: 608) | (SEQ ID NO: | KGG (SEQ | GVV (SEQ |
| | ID NO: | NO: 373) | | 843) | ID NO: | ID NO: |
| | 138) | | | | 1078) | 1313) |
| AB- | GDSIYSDY | EVYRSGTRQYSPS | AAMRDGYGSGWL (SEQ | GLSSGSVSAHYF | RTNTRSS | ALSMNSGSFW |
| 009707 | WWT (SEQ | LES (SEQ ID | ID NO: 609) | PT (SEQ ID | (SEQ ID | V (SEQ ID |
| | ID NO: | NO: 374) | | NO: 844) | NO: 1079) | NO: 1314) |
| | 139) | | | | | |
| AB- | GGSLGGYY | ESDESGKTDYNPS | ARGHTSIYGVLIMLFHASW | KSSQSVLYNSNN | WASTRTS | QQYYTSPRT |
| 009708 | WT (SEQ | LKS (SEQ ID | LDP (SEQ ID NO: | KDYLA (SEQ | (SEQ ID | (SEQ ID |
| | ID NO: | NO: 375) | 610) | ID NO: 845) | NO: 1080) | NO: 1315) |
| | 140) | | | | | |
| AB- | GFVFRRYW | RTNEDGTITNYAD | ARDVGGADGH (SEQ ID | RSSESLVHPNGK | SASIRAS | MQGLQTPYT |
| 009709 | MH (SEQ | SVKG (SEQ ID | NO: 611) | TWLD (SEQ ID | (SEQ ID | (SEQ ID |
| | ID NO: | NO: 376) | | NO: 846) | NO: 1081) | NO: 1316) |
| | 141) | | | | | |
| AB- | GGSYSSYT | RISPVLNTTYYAQ | ARLASMNGVDI (SEQ | RASQSVYGSYLA | GGSSRAT | HQYGTSRYS |
| 009710 | LS (SEQ | KLEG (SEQ ID | ID NO: 612) | (SEQ ID NO: | (SEQ ID | (SEQ ID |
| | ID NO: | NO: 377) | | 847) | NO: 1082) | NO: 1317) |
| | 142) | | | | | |
| AB- | GYTFTHYY | RINPNSGGTHYAQ | ARDSETDTSLAVAYYYYYM | RASQSVAGSSFG | GASTRAT | QQYDKSPIT |
| 009711 | IH (SEQ | KFQG (SEQ ID | DV (SEQ ID NO: 613) | GSYLA (SEQ | (SEQ ID | (SEQ ID |
| | ID NO: | NO: 378) | | ID NO: 848) | NO: 1083) | NO: 1318) |
| | 143) | | | | | |
| AB- | QFIFSSYA | MIYSDSSEKHYAD | AREGREYSDNSGKRGYFDS | RASQSIRNWLA | DASNLKS | QQYILYWT |
| 009712 | MH (SEQ | SVKG (SEQ ID | (SEQ ID NO: 614) | (SEQ ID NO: | (SEQ ID | (SEQ ID |
| | ID NO: | NO: 379) | | 849) | NO: 1084) | NO: 1319) |
| | 144) | | | | | |
| AB- | GFTLRTYA | IISYSGDTEYDAGS | AREGEDYSSSYFDH | TLSSGYSTYAIA | LNSDGSH | QTWGSGIQV |
| 009713 | MH (SEQ | VRG (SEQ ID | (SEQ ID NO: 615) | (SEQ ID NO: | TKGD | (SEQ ID |
| | ID NO: | NO: 380) | | 850) | (SEQ ID | NO: 1320) |
| | 145) | | | | NO: 1085) | |
| AB- | GGSFSYYS | EIIHRGRTNSNPSL | ARHRIVEATFSRYDYFDS | QGDSLRTYYAT | AKNSRPS | NSRDSSGNYV |
| 009714 | WSWS | KS (SEQ ID | (SEQ ID NO: 616) | (SEQ ID NO: | (SEQ ID | (SEQ ID |
| | (SEQ ID | NO: 381) | | 851) | NO: 1086) | NO: 1321)V |
| | NO: 146) | | | | | |
| AB- | GGSFNNHA | EVIPHGSTKCDPSL | ARRSGESNGWGTYYHYGM | ASSTGAVTSGYY | NTNYRHS | LLYYGGAQPW |
| 009715 | WT (SEQ | KS (SEQ ID | DV (SEQ ID NO: | PI (SEQ ID | (SEQ ID | V (SEQ ID |
| | ID NO: | NO: 382) | 617) | NO: 852) | NO: 1087) | NO: 1322) |
| | 147) | | | | | |
| AB- | GGSFTNYW | EIDRSGTTNYSPSL | ARGRTPTGTFDYYSYLDV | QGDSLRSYYAS | GKNSRPS | NSRDSSGNSV |
| 009716 | WRWT | KS (SEQ ID | (SEQ ID NO: 618) | (SEQ ID NO: | (SEQ ID | V (SEQ ID |
| | (SEQ ID | NO: 383) | | 853) | NO: 1088) | NO: 1323) |
| | NO: 148) | | | | | |
| AB- | GGSFSGYY | EITHSGRTNYNPSL | ARWDCTDGTCYAVY | SGDELGRKYVS | RDNRRPS | QAWDSTTVI |
| 009717 | WS (SEQ | TR (SEQ ID | (SEQ ID NO: 619) | (SEQ ID NO: | (SEQ ID | (SEQ ID |
| | ID NO: | NO: 384) | | 854) | NO: 1089) | NO: 1324) |
| | 149) | | | | | |
| AB- | GGSFSNYY | EINYRGNTKYNPS | ARGLGPPVGSLAITFGGLR | RASQSISSYLN | AASTLQS | QQSYSAPFT |
| 009718 | WN (SEQ | LRS (SEQ ID | RSFDI (SEQ ID | (SEQ ID NO: | (SEQ ID | (SEQ ID |
| | ID NO: | NO: 385) | NO: 620) | 855) | NO: 1090) | NO: 1325) |
| | 150) | | | | | |
| AB- | GYTLSKLF | GSVPEDDNTIYAE | ATFRRQRGGYYYYGLDL | QASQDISNYLN | DASNLET | QQADNLPYT |
| 009719 | MH (SEQ | KFRG (SEQ ID | (SEQ ID NO: 621) | (SEQ ID NO: | (SEQ ID | (SEQ ID |
| | ID NO: | NO: 386) | | 856) | NO: 1091) | NO: 1326) |
| | 151) | | | | | |
| AB- | GFKFDDYA | GISWNSGSIDYADS | AKVKLTVFGVEQSPLDS | RSSQSLLHNDGY | LGFHRAS | VQALQTPLT |
| 009720 | MH (SEQ | VKG (SEQ ID | (SEQ ID NO: 622) | NYLD (SEQ ID | (SEQ ID | (SEQ ID |
| | ID NO: | NO: 387) | | NO: 857) | NO: 1092) | NO: 1327) |
| | 152) | | | | | |
| AB- | GFTFSRYA | TISDTDGSTIYYTD | TIQARVPSAMKVYFQN | TGSSSNIGAGYD | ANNNRPS | QSYDNSLSGS |
| 009721 | MA (SEQ | SVKG (SEQ ID | (SEQ ID NO: 623) | VH (SEQ ID | (SEQ ID | RV (SEQ ID |
| | ID NO: | NO: 388) | | NO: 858) | NO: 1093) | NO: 1328) |
| | 153) | | | | | |
| AB- | GGIFNSYKI | GIIAIFGTADYAQR | TRERFSNYFDP (SEQ | RASQSVSSFLG | DASNRAS | QQRSRGPSGI |
| 009722 | T (SEQ | FQG (SEQ ID | ID NO: 624) | (SEQ ID NO: | (SEQ ID | T (SEQ ID |
| | ID NO: | NO: 389) | | 859) | NO: 1094) | NO: 1329) |
| | 154) | | | | | |
| AB- | GYIFTNYD | WMNPYSGNTGFS | ARGRDVGQWLVGEGFDY | RASQDISSALA | DASSLEN | QQFNTYPLT |
| 009723 | LS (SEQ | QKFQG (SEQ | (SEQ ID NO: 625) | (SEQ ID NO: | (SEQ ID | (SEQ ID |
| | ID NO: | ID NO: 390) | | 860) | NO: 1095) | NO: 1330) |
| | 155) | | | | | |
| AB- | GGTFSNNA | RIIPMVGVADYAQ | ARGKPFDFWSGLFDS | KSSQTLLHSDGK | EVSNRFS | MQSIELPPLT |
| 009724 | IS (SEQ | KLQG (SEQ ID | (SEQ ID NO: 626) | TYLY (SEQ ID | (SEQ ID | (SEQ ID |
| | ID NO: | NO: 391) | | NO: 861) | NO: 1096) | NO: 1331) |
| | 156) | | | | | |
| AB- | HGSIRSQY | YIYDSGSSKYNPSL | ARRKVSFPRPSDDSSFDWF | QASQDISNFLN | DASILEA | QQYDNLAPLT |
| 009725 | WS (SEQ | KS (SEQ ID | FDL (SEQ ID NO: | (SEQ ID NO: | (SEQ ID | (SEQ ID |
| | ID NO: | NO: 392) | 627) | 862) | NO: 1097) | NO: 1332) |
| | 157) | | | | | |
| AB- | GYTFINYW | IIYPGDSDTRYSPSF | AREDIIENERGGFDI | SGDELPKQYAS | KDTERPS | QSPDSSDTYR |
| 009726 | IG (SEQ | QG (SEQ ID NO: | (SEQ ID NO: 628) | (SEQ ID NO: | (SEQ ID | V (SEQ ID |
| | ID NO: | 393) | | 863) | NO: 1098) | NO: 1333) |
| | 158) | | | | | |
| AB- | GFTFDNFG | VISHDGTNEYYGD | GRDRIALAEIDY (SEQ | RASQSVGNNVA | GASLRAT | QQYNNWPPSI |
| 009727 | FH (SEQ | SVKG (SEQ ID | ID NO: 629) | (SEQ ID NO: | (SEQ ID | T (SEQ ID |
| | ID NO: | NO: 394) | | 864) | NO: 1099) | NO: 1334) |
| | 159) | | | | | |
| AB- | GFNFNYYG | SISSSSSHIYYADSV | VRDRSFEYGDYGGDN | SGDALPNQYVY | KDKERPS | QSADSSGTYP |
| 009728 | MN (SEQ | KG (SEQ ID NO: | (SEQ ID NO: 630) | (SEQ ID NO: | (SEQ ID | NWV (SEQ |
| | ID NO: | 395) | | 865) | NO: 1100) | ID NO: |
| | 160) | | | | | 1335) |
| AB- | GYTFTDYY | WINPDTSATNYAQ | TRVPHYYGSDTYYKPWYYY | TGTSSDVGGYN | EVTNRPS | SSYTSVSTLM |
| 009729 | IH (SEQ | NFQG (SEQ ID | YAMDV (SEQ ID | YVS (SEQ ID | (SEQ ID | L (SEQ ID |
| | ID NO: | NO: 396) | NO: 631) | NO: 866) | NO: 1101) | NO: 1336) |
| | 161) | | | | | |
| AB- | GGTFSSYAI | EIIPVFGAPKFAQK | AVGYCSSSSCEPQDAFDI | KSSQSLLHSAGK | EVSNRFS | MQTIQLPIS |
| 009730 | S (SEQ ID | FRG (SEQ ID | (SEQ ID NO: 632) | TYLY (SEQ ID | (SEQ ID | (SEQ ID |
| | NO: 162) | NO: 397) | | NO: 867) | NO: 1102) | NO: 1337) |
| AB- | GYSLTSYA | WLNGGNGNTKYS | ARADHSTYWYGWFDP | SGSTSNIGSNYV | RNDQRPS | TSWDDSLNGP |
| 009731 | MH (SEQ | QKFQA (SEQ | (SEQ ID NO: 633) | F (SEQ ID | (SEQ ID | L (SEQ ID |
| | ID NO: | ID NO: 398) | | NO: 868) | NO: 1103) | NO: 1338) |
| | 163) | | | | | |
| AB- | GFTFDDYT | LISWDGGSKYYAD | VKDISSSPANYGFAI | RASQSISTFVN | DASSLQS | QQSFSTLLT |
| 009732 | MH (SEQ | SVMG (SEQ ID | (SEQ ID NO: 634) | (SEQ ID NO: | (SEQ ID | (SEQ ID |
| | ID NO: | NO: 399) | | 869) | NO: 1104) | 1NO: 339) |
| | 164) | | | | | |
| AB- | GGSISSTAN | CISYSGRTYDNPSL | ARHAGWFGEHLDY | RASQSVSSGYLA | GASSRAT | QQYGSSLTWT |
| 009733 | YWG (SEQ | KS (SEQ ID | (SEQ ID NO: 635) | (SEQ ID NO: | (SEQ ID | (SEQ ID |
| | ID NO: | NO: 400) | | 870) | NO: 1105) | NO: 1340) |
| | 165) | | | | | |
| AB- | GYTFTSHY | LINPSGSGTAYPQH | ARMERSGAFDL (SEQ | RASQTISSRYLA | GSSIRAT | QHYGSSPPMY |
| 009734 | MH (SEQ | FQG (SEQ ID | ID NO: 636) | (SEQ ID NO: | (SEQ ID | T (SEQ ID |
| | ID NO: | NO: 401) | | 871) | NO: 1106) | NO: 1341) |
| | 166) | | | | | |
| AB- | GGTFNSYA | TVLPIFDTTHSAQK | VGRYGSGNYEGY | TRSSGSIASNYV | EDNQRPS | QSYDSRNPQW |
| 009735 | FT (SEQ | FQG (SEQ ID | (SEQ ID NO: 637) | Q (SEQ ID | (SEQ ID | V (SEQ ID |
| | ID NO: | NO: 402) | | NO: 872) | NO: 1107) | NO: 1342) |
| | 167) | | | | | |
| AB- | GGTFVNFA | GIIPVFGATNYAQK | AKDLLSDRQDPS (SEQ | RSSESLVHTDGN | KVSNRDS | MQGTHWPWT |
| 009736 | IC (SEQ | FRG (SEQ ID | ID NO: 638) | TYLN (SEQ ID | (SEQ ID | (SEQ ID |
| | ID NO: | NO: 403) | | NO: 873) | NO: 1108) | NO: 1343) |
| | 168) | | | | | |
| AB- | GLALSNGR | HIFSNDAKSYSTSL | ASIDERLGILYWYFHL | RASQTFSSNLA | GASTRAT | QQYNDWPRT |
| 009737 | MGVS | KG (SEQ ID NO: | (SEQ ID NO: 639) | (SEQ ID NO: | (SEQ ID | (SEQ ID |
| | (SEQ ID | 404 | | 874) | NO: 1109) | NO: 1344) |
| | NO: 169) | | | | | |
| AB- | GGTFTNYA | GIIPVFGAPNYAQK | ARVGYCSTTGCDMGGLDY | RASQSISSTYLA | GASSRAT | QQYGVSPNT |
| 009738 | IN (SEQ | FQG (SEQ ID | (SEQ ID NO: 640) | (SEQ ID NO: | (SEQ ID | (SEQ ID |
| | ID NO: | NO: 405) | | 875) | NO: 1110) | NO: 1345) |
| | 170) | | | | | |
| AB- | GYTFTSYW | IIYPGDSDTRYSLA | ARLSAQRGNSGPAVY | GGNNIGRKSVH | DDTDRPS | QVWDSSSDHV |
| 009739 | IV (SEQ | FQG (SEQ ID | (SEQ ID NO: 641) | (SEQ ID NO: | (SEQ ID | V (SEQ ID |
| | ID NO: | NO: 406) | | 876) | NO: 1111) | NO: 1346) |
| | 171) | | | | | |
| AB- | GFTFSSYG | SISGSGGSPYYADS | AKSEETMTTVTTFFDY | TLSSGHSSYPIA | LNSDGSH | QTWGTATPW |
| 009740 | MN (SEQ | VKG (SEQ ID | (SEQ ID NO: 642) | (SEQ ID NO: | SKGD | V (SEQ ID |
| | ID NO: | NO: 407) | | 877) | (SEQ ID | NO: 1347) |
| | 172) | | | | NO: 1112) | |
| AB- | GFTFSSYA | LINGGGGRTYYTD | ERSVGY (SEQ ID | RTSQSIATYLN | AASSLHS | QQTYSAPYT |
| 009741 | MS (SEQ | SVKG (SEQ ID | NO: 643) | (SEQ ID NO: | (SEQ ID | (SEQ ID |
| | ID NO: | NO: 408) | | 878) | NO: 1113) | NO: 1348) |
| | 173) | | | | | |
| AB- | GITFEDYA | GISWNGANIGYAD | AKSTVTTLRRTYFDS | RASQGISNLLA | AASRLQS | QQANTFPPAL |
| 009742 | LH (SEQ | SVKG (SEQ ID | (SEQ ID NO: 644) | (SEQ ID NO: | (SEQ ID | L (SEQ ID |
| | ID NO: | NO: 409) | | 879) | NO: 1114) | NO: 1349) |
| | 174) | | | | | |
| AB- | GNTLSELYI | GFDPEDGETIYAQ | ATHDFGDSEGEY | GSSTGPVTSGRY | DTNTKYS | LLSYSGARV |
| 009743 | Y (SEQ ID | NFQG (SEQ ID | (SEQ ID NO: 645) | PY (SEQ ID | (SEQ ID | (SEQ ID |
| | NO: 175) | NO: 410) | | NO: 880) | NO: 1115) | NO: 1350) |
| AB- | GYTFTNYA | WINAGNGNTKYSQ | AKDQWADYHTTGDFEY | TGSSSDVGGYN | DVGKRPS | CSYAGTHTYI |
| 009744 | VH (SEQ | KFQG (SEQ ID | (SEQ ID NO: 646) | YVS (SEQ ID | (SEQ ID | L (SEQ ID |
| | ID NO: | NO: 411) | | NO: 881) | NO: 1116) | NO: 1351) |
| | 176) | | | | | |
| AB- | GFTFGDYA | FIRSKVYGETTEYA | TRDGRLLWFEESDWGDAFG | RSSQSLVYSDGN | RVSNWDS | MQGTHWPYT |
| 009745 | MN (SEQ | ASVKG (SEQ ID | I (SEQ ID NO: 647) | TYLN (SEQ ID | (SEQ ID | (SEQ ID |
| | ID NO: | NO: 412) | | NO: 882) | NO: 1117) | NO: 1352) |
| | 177) | | | | | |
| AB- | GASLISNSY | SIFYSGTTHYNPSL | ARLTVDRRLQGYYYYYYM | TGSSSNIGAGYD | DNTNRPS | QSYDNSLSGS |
| 009746 | YWG (SEQ | KS (SEQ ID | DV (SEQ ID NO: | VH (SEQ ID | (SEQ ID | L (SEQ ID |
| | ID NO: | NO: 413) | 648) | NO: 883) | NO: 1118) | NO: 1353) |
| | 178) | | | | | |
| AB- | GFTFSNYG | FIRYDGSNEYYAD | AKQGLLSNYYYGMDV | KSSQSVLYSSNN | WASTRES | QQYYSSVT |
| 009747 | MH (SEQ | SVKG (SEQ ID | (SEQ ID NO: 649) | KNYLT (SEQ | (SEQ ID | (SEQ ID |
| | ID NO: | NO: 414) | | ID NO: 884) | NO: 1119) | NO: 1354) |
| | 179) | | | | | |
| AB- | GFTFSSHG | FIRYDGSNKYYAD | AKDNYYDILTGYYRKYYYF | RASQGISNYLA | AASTLQS | QKYNSAPFT |
| 009748 | MH (SEQ | SVKG (SEQ ID | GMDV (SEQ ID NO: | (SEQ ID NO: | (SEQ ID | (SEQ ID |
| | ID NO: | NO: 415) | 650) | 885) | NO: 1120) | NO: 1355) |
| | 180) | | | | | |
| AB- | GGSISSYY | YIHYSGSTIYNPSL | ARTAGSDDSGYLPFDY | SGDALPKKYAS | EDTKRPS | YSTDSSGNHR |
| 009749 | WS (SEQ | KS (SEQ ID | (SEQ ID NO: 651) | (SEQ ID NO: | (SEQ ID | V (SEQ ID |
| | ID NO: | NO: 416) | | 886) | NO: 1121) | NO: 1356) |
| | 181) | | | | | |
| AB- | GGSISSSSY | SIYYSGSTYYNPSL | SRSPFSISIFGVVIRYGMD | RASQGISSYLA | AASTLQS | QQLNSYPRT |
| 009750 | YWG (SEQ | KS (SEQ ID | V (SEQ ID NO: 652) | (SEQ ID NO: | (SEQ ID | (SEQ ID |
| | ID NO: | NO: 417) | | 887) | NO: 1122) | NO: 1357) |
| | 182) | | | | | |
| AB- | GGSISSGD | YIYYSGSTYYNPSL | ARAITKITIFGVVIENFDY | RASQSVSSSYLA | GASSRAT | QQYGSSPLT |
| 009751 | YYWS (SEQ | KS (SEQ ID | (SEQ ID NO: 653) | (SEQ ID NO: | (SEQ ID | (SEQ ID |
| | ID NO: | NO: 418) | | 888) | NO: 1123) | NO: 1358) |
| | 183) | | | | | |
| AB- | GYTFTSYA | WINAGNGNTKYSQ | AREVFEAAAGMLLPYYYYY | QGDSLRSYYAS | GKNNRPS | NSRDSSGNHL |
| 009752 | MH (SEQ | KFQG (SEQ ID | MDV (SEQ ID NO: | (SEQ ID NO: | (SEQ ID | V (SEQ ID |
| | ID NO: | NO: 419) | 654) | 889) | NO: 1124) | NO: 1359) |
| | 184) | | | | | |
| AB- | GGSISSHC | YIYYSGSTNYNPSL | ARQRREWLGSYYYYGMDV | RASQSISSYLN | AASSLQS | QQSYSTPLT |
| 009753 | WN (SEQ | KS (SEQ ID | (SEQ ID NO: 655) | (SEQ ID NO: | (SEQ ID | (SEQ ID |
| | ID NO: | NO: 420) | | 890) | NO: 1125) | NO: 1360) |
| | 185) | | | | | |
| AB- | GGSISSSSY | IIYYSGSTYYNPSL | ARVYAGFLEWLSYFDY | QGDSLRSYYAS | GKNNRPS | NSRDSSGNHW |
| 009754 | YWG (SEQ | KS (SEQ ID | (SEQ ID NO: 656) | (SEQ ID NO: | (SEQ ID | V (SEQ ID |
| | ID NO: | NO: 421) | | 891) | NO: 1126) | NO: 1361) |
| | 186) | | | | | |
| AB- | GDSISSGA | YIYYSGSTYYNPSL | ARARRITIFGVVGDFGV | RASQSVSSSYLA | GASSRAT | QQYGSSPLT |
| 009755 | YYWS | KS (SEQ ID | (SEQ ID NO: 657) | (SEQ ID NO: | (SEQ ID | (SEQ ID |
| | (SEQ ID | NO: 422) | | 892) | NO: 1127) | NO: 1362) |
| | NO: 187) | | | | | |
| AB- | GFTFSSFA | AISGSGGSTYYADS | AKEERVYDILTGYDY | SGDALPKQYAY | KDTERPS | QSADSSGSSV |
| 009756 | MS (SEQ | VKG (SEQ ID | (SEQ ID NO: 658) | (SEQ ID NO: | (SEQ ID | V (SEQ ID |
| | ID NO: | NO: 423) | | 893) | NO: 1128) | NO: 1363) |
| | 188) | | | | | |
| AB- | GGSISSSSY | SIYYSGSTYYNPSL | ARLKNWNTIFGVVIMAPYY | RASQSVSSYLA | DASNRAT | QQRSKGIT |
| 009757 | YWG (SEQ | KS (SEQ ID | MDV (SEQ ID NO: | (SEQ ID NO: | (SEQ ID | (SEQ ID |
| | ID NO: | NO: 424) | 659) | 894) | NO: 1129) | NO: 1364) |
| | 189) | | | | | |
| AB- | GYTLTELS | GFDPEDGETIYAQ | ATGYYGSRFDP (SEQ | GGNNIGSKSVH | DDSDRPS | QVWDSSTDHP |
| 009758 | MH (SEQ | KFQA (SEQ ID | ID NO: 660) | (SEQ ID NO: | (SEQ ID | GVV (SEQ |
| | ID NO: | NO: 425) | | 895) | NO: 1130) | ID NO: |
| | 190) | | | | | 1365) |
| AB- | GGTFSSYAI | GIIPIFGTANYAQK | ARALVQGVYYYYMDV | RASQSVSSKLA | GASTRAT | QQYNNWPPYT |
| 009759 | S (SEQ ID | FQG (SEQ ID | (SEQ ID NO: 661) | (SEQ ID NO: | (SEQ ID | (SEQ ID |
| | NO: 191) | NO: 426) | | 896) | NO: 1131) | NO: 1366) |
| AB- | GYSFTSYW | IIYPGDSDTRYSPSF | ARSRSSDYFDY (SEQ | RASQSISSWLA | DASSLES | QQYNSYSRT |
| 009760 | IG (SEQ | QG (SEQ ID NO: | ID NO: 662) | (SEQ ID NO: | (SEQ ID | (SEQ ID |
| | ID NO: | 427) | | 897) | NO: 1132) | NO: 1367) |
| | 192) | | | | | |
| AB- | GFTFSSYA | AISGSGGSTYYADS | AKGPRSAPDYMDV | RASQSISSWLA | DASSLES | QQYNSYSGT |
| 009761 | MS (SEQ | VKG (SEQ ID | (SEQ ID NO: 663) | (SEQ ID NO: | (SEQ ID | (SEQ ID |
| | ID NO: | NO: 428) | | 898) | NO: 1133) | NO: 1368) |
| | 193) | | | | | |
| AB- | GFTFSDYY | YISRSGSTIYYVDS | ARDQRLWSTLGVYYYYYM | RASQSISSYLN | AASSLQS | QQSYSHPLT |
| 009762 | MS (SEQ | VKG (SEQ ID | DV (SEQ ID NO: | (SEQ ID NO: | (SEQ ID | (SEQ ID |
| | ID NO: | NO: 429) | 664) | 899) | NO: 1134) | NO: 1369) |
| | 194) | | | | | |
| AB- | GGSFSGYY | EINHSGSTNYNPSL | ARRAAPAWTWFGELSPFDY | RASQSINNWLA | KASSLES | QQYNSYSQYT |
| 009763 | WS (SEQ | KS (SEQ ID | (SEQ ID NO: 665) | (SEQ ID NO: | (SEQ ID | (SEQ ID |
| | ID NO: | NO: 430) | | 900) | NO: 1135) | NO: 1370) |
| | 195) | | | | | |
| AB- | GGSISSHY | YIYYSGSTNYNPSL | ARSARRWLQYGPDWYFDL | TGTSSDVGGYN | DVSKRPS | SSYAGSNNWV |
| 009764 | WS (SEQ | KS (SEQ ID | (SEQ ID NO: 666) | YVS (SEQ ID | (SEQ ID | (SEQ ID |
| | ID NO: | NO: 431) | | NO: 901) | NO: 1136) | NO: 1371) |
| | 196) | | | | | |
| AB- | GFTFSDYY | YISSSGNTIYYADS | ARDPPPRITIFGVGTRLGF | RASQSVSSNLA | GASTRAT | QQYNNWPPW |
| 009765 | MS (SEQ | VKG (SEQ ID | DY (SEQ ID NO: | (SEQ ID NO: | (SEQ ID | T (SEQ ID |
| | ID NO: | NO: 432) | 667) | 902) | NO: 1137) | NO: 1372) |
| | 197) | | | | | |
| AB- | GGSISRSSY | SIYYSGSTYYNPSL | ARQLLGIWGSYRYIDY | RASQGISSYLA | AASTLQS | QQLNSYPIT |
| 009766 | YWG (SEQ | KS (SEQ ID | (SEQ ID NO: 668) | (SEQ ID NO: | (SEQ ID | (SEQ ID |
| | ID NO: | NO: 433) | | 903) | NO: 1138) | NO: 1373) |
| | 198) | | | | | |
| AB- | GYSFTSYW | IIYPGDSDTRYSPSF | ASGRLHLGELLDY | TGTSSDVGGYN | DVSNRPS | SSYTSSSTLV |
| 009767 | IG (SEQ | QG (SEQ ID NO: | (SEQ ID NO: 669) | YVS (SEQ ID | (SEQ ID | V (SEQ ID |
| | ID NO: | 434) | | NO: 904) | NO: 1139) | NO: 1374) |
| | 199) | | | | | |
| AB- | GGSISSSSY | SIYYSGSTYYNPSL | ARQPRRITIFGVVIMPTNN | TGSSSNIGAGYD | GNSNRPS | QSYDSSLSDV |
| 009768 | YWG (SEQ | KS (SEQ ID | WFDP (SEQ ID NO: | VH (SEQ ID | (SEQ ID | V (SEQ ID |
| | ID NO: | NO: 435) | 670) | NO: 905) | NO: 1140) | NO: 1375) |
| | 200) | | | | | |
| AB- | GFTFSSYW | NIKQDGSEKYYVD | ARSPEVLWFGDPVLDNWFD | SGDALPKQYAY | KDSERPS | QSADSSGTYP |
| 009769 | MS (SEQ | SVKG (SEQ ID | P (SEQ ID NO: 671) | (SEQ ID NO: | (SEQ ID | VV (SEQ ID |
| | ID NO: | NO: 436) | | 906) | NO: 1141) | NO: 1376) |
| | 201) | | | | | |
| AB- | GFTFSSYS | SISSSSSYIYYADSV | ARDPYYDILTGYYLY | RASQSISSYLN | AASSLQS | QQSYSTPLIT |
| 009770 | MN (SEQ | KG (SEQ ID NO: | (SEQ ID NO: 672) | (SEQ ID NO: | (SEQ ID | (SEQ ID |
| | ID NO: | 437) | | 907) | NO: 1142) | NO: 1377) |
| | 202) | | | | | |
| AB- | GFTFSSYW | RINSDGSSTSYADS | ARDGRNIAVAGLDY | RASQSISRWLA | KASSLES | QQYNSYQGT |
| 009771 | MH (SEQ | VKG (SEQ ID | (SEQ ID NO: 673) | (SEQ ID NO: | (SEQ ID | (SEQ ID |
| | ID NO: | NO: 438) | | 908) | NO: 1143) | NO: 1378) |
| | 203) | | | | | |
| AB- | GGSISSSSY | SIYYSGSTYYNPSL | ARHLQPSYGGNAFDI | TGSSSNIGAGYD | GNSNRPS | QSYDSSLSGY |
| 009772 | YWG (SEQ | KS (SEQ ID | (SEQ ID NO: 674) | VH (SEQ ID | (SEQ ID | V (SEQ ID |
| | ID NO: | NO: 439) | | NO: 909) | NO: 1144) | NO: 1379) |
| | 204) | | | | | |
| AB- | GYTFTGYY | RINPNSGGTNYAQ | ARDGCSGGSCYSWWFDP | RASQSISSYLN | AASSLQS | QQSYSTPLT |
| 009773 | MH (SEQ | KFQG (SEQ ID | (SEQ ID NO: 675) | (SEQ ID NO: | (SEQ ID | (SEQ ID |
| | ID NO: | NO: 440) | | 910) | NO: 1145) | NO: 1380) |
| | 205) | | | | | |
| AB- | GGSISSSSY | SIYYSGSTYYNPSL | ARQKTDIVVVVATEVAFDY | KSSQSVLYSSNN | WASTRES | QQYYSTPLT |
| 009774 | YWG (SEQ | KS (SEQ ID | (SEQ ID NO: 676) | KNYLA (SEQ | (SEQ ID | (SEQ ID |
| | ID NO: | NO: 441) | | ID NO: 911) | NO: 1146) | NO: 1381) |
| | 206) | | | | | |
| AB- | GGSISSSSY | SIYYSGSTYYNPSL | ARQVRSGIVVVTPYYFDY | RASQSVSSSYLA | GASSRAT | QQYGSSPRT |
| 009775 | YWG (SEQ | KS (SEQ ID | (SEQ ID NO: 677) | (SEQ ID NO: | (SEQ ID | (SEQ ID |
| | ID NO: | NO: 442) | | 912) | NO: 1147) | NO: 1382) |
| | 207) | | | | | |
| AB- | GGSISSGG | YIYYSGSTYYNPSL | ARDQYDSSGYAFGAFDY | SGDKLGDKYAC | QDSKRPS | QAWDSSTAW |
| 009776 | YYWS (SEQ | KS (SEQ ID | (SEQ ID NO: 678) | (SEQ ID NO: | (SEQ ID | V (SEQ ID |
| | ID NO: | NO: 443) | | 913) | NO: 1148) | NO: 1383) |
| | 208) | | | | | |
| AB- | GFTFSSYS | SISSSSSYIYYADSV | ARDESVAHYYGSGSVDV | QGDSLRSYYAS | GKNNRPS | NSRDSSGNRW |
| 009777 | MN (SEQ | KG (SEQ ID NO: | (SEQ ID NO: 679) | (SEQ ID NO: | (SEQ ID | V (SEQ ID |
| | ID NO: | 444) | | 914) | NO: 1149) | NO: 1384) |
| | 209) | | | | | |
| AB- | GGSISSSSY | SIYYSGSTYYNPSL | ARQFRRWLQLGPSYFDY | RASQSISSWLA | KASSLES | QQYNSYSRAW |
| 009778 | YWG (SEQ | KS (SEQ ID | (SEQ ID NO: 680) | (SEQ ID NO: | (SEQ ID | T (SEQ ID |
| | ID NO: | NO: 445) | | 915) | NO: 1150) | NO: 1385) |
| | 210) | | | | | |
| AB- | GFTFSNAW | RIKTKSDGGTTDY | SRNPFLDY | RASQGIKYYLA | AASSLLS | QQYNSYPYT |
| 009779 | MS (SEQ | AAPVKG (SEQ | (SEQ ID NO: 681) | (SEQ ID NO: | (SEQ ID | (SEQ ID |
| | ID NO: | ID NO: 446) | | 916) | NO: 1151) | NO: 1386) |
| | 211) | | | | | |
| AB- | GGSISSGD | YIYYSGSTYYNPSL | ARDRWGITIFGVVNRDGMD | RASQSISSYLN | AASSLQS | QQSYSFPYT |
| 009780 | YYWS (SEQ | KS (SEQ ID | V (SEQ ID NO: 682) | (SEQ ID NO: | (SEQ ID | (SEQ ID |
| | ID NO: | NO: 447) | | 917) | NO: 1152) | NO: 1387) |
| | 212) | | | | | |
| AB- | GFTFSNAW | RIKSKTDGGTTDY | TTLGHQYYDSSGYDYYYG | RASQSISSWLA | DASSLES | QQYNSYTWT |
| 009781 | MS (SEQ | AAPVKG (SEQ | MDV (SEQ ID NO: | (SEQ ID NO: | (SEQ ID | (SEQ ID |
| | ID NO: | ID NO: 448) | 683) | 918) | NO: 1153) | NO: 1388) |
| | 213) | | | | | |
| AB- | GGSISSYY | RIYTSGSTNYNPSL | ARGRLFYDSSGYYFDY | SGDALPKKYAY | EDSKRPS | YSTDSSGNHY |
| 009782 | WS (SEQ | KS (SEQ ID | (SEQ ID NO: 684) | (SEQ ID NO: | (SEQ ID | V (SEQ ID |
| | ID NO: | NO: 449) | | 919) | NO: 1154) | NO: 1389) |
| | 214) | | | | | |
| AB- | AGSISSHY | YIYYSGSTNYNPSL | ARTLRQWLARGYYYFDY | RASQSVSSSYLA | GASSRAT | QQYGSSPYT |
| 009783 | WS (SEQ | RS (SEQ ID | (SEQ ID NO: 685) | (SEQ ID NO: | (SEQ ID | (SEQ ID |
| | ID NO: | NO: 450) | | 920) | NO: 1155) | NO: 1390) |
| | 215) | | | | | |
| AB- | GGSISSSSY | SIYYSGSTYYNPSL | VRHRGWIQLWFFDY | TGTSSDVGGYN | DVINRPS | SSYTSSSTVV |
| 009784 | YWG (SEQ | KS (SEQ ID | (SEQ ID NO: 686) | (SEQ ID NO: | (SEQ ID | (SEQ ID |
| | ID NO: | NO: 451) | | 921)YVS | NO: 1156) | NO: 1391) |
| | 216) | | | | | |
| AB- | GGSISSYY | YIYYSGSTNYNPSL | ARLRAHTIFGVPIADAFDI | RASQSVSSSYLA | GASSRAT | QQYGSSPLT |
| 009785 | WS (SEQ | KS (SEQ ID | (SEQ ID NO: 687) | (SEQ ID NO: | (SEQ ID | (SEQ ID |
| | ID NO: | NO: 452) | | 922) | NO: 1157) | NO: 1392) |
| | 217) | | | | | |
| AB- | GGSFSGYY | EINHSGSTNYNPSL | ARHLSLITVIVVVTYQLDA | QGDSLRSYYAS | GKNNRPS | NSRDSSGNHR |
| 009786 | WS (SEQ | KS (SEQ ID | FDI (SEQ ID NO: | (SEQ ID NO: | (SEQ ID | V (SEQ ID |
| | ID NO: | NO: 453) | 688) | 923) | NO: 1158) | NO: 1393) |
| | 218) | | | | | |
| AB- | GGSISSSSY | SIYYSGSTYYNPSL | ARHYSSITIFGVVSGRYMD | TGTSSDVGSYNL | EGSKRPS | CSYAGSAV |
| 009787 | YWG (SEQ | KS (SEQ ID | V (SEQ ID NO: 689) | VS (SEQ ID | (SEQ ID | (SEQ ID |
| | ID NO: | NO: 454) | | NO: 924) | NO: 1159) | NO: 1394) |
| | 219) | | | | | |
| AB- | GGSISSHY | YIYYSGSTNYNPSL | ARDLRGWLQSPNWYFDL | RASQSVSSSYLA | GASSRAT | QQYGSSPAYT |
| 009788 | WS (SEQ | KS (SEQ ID | (SEQ ID NO: 690) | (SEQ ID NO: | (SEQ ID | (SEQ ID |
| | ID NO: | NO: 455) | | 925) | NO: 1160) | NO: 1395) |
| | 220) | | | | | |
| AB- | GFTFSSYA | AISGSGGSTYYADS | AKESGSGYYN (SEQ | SGDKLGDKYAC | QDSKRPS | QAWDSSSVV |
| 009789 | MS (SEQ | VKG (SEQ ID | ID NO: 691) | (SEQ ID NO: | (SEQ ID | (SEQ ID |
| | ID NO: | NO: 456) | | 926) | NO: 1161) | NO: 1396) |
| | 221) | | | | | |
| AB- | GFTFSSYA | AISGSGGSTYYADS | AKTSGTGTLAY (SEQ | SGDKLGDKYAC | QDSKRPS | QAWDSSTLV |
| 009790 | MS (SEQ | VKG (SEQ ID | ID NO: 692) | (SEQ ID NO: | (SEQ ID | (SEQ ID |
| | ID NO: | NO: 457) | | 927) | NO: 1162) | NO: 1397) |
| | 222) | | | | | |
| AB- | GGSISSSSY | SIYYSGSTYYNPSL | AREPRRVKQWLVVPYYYM | SGDKLGDKYAC | QDSKRPS | QAWDSSIVV |
| 009791 | YWG (SEQ | KS (SEQ ID | DV (SEQ ID NO: 693) | (SEQ ID NO: | (SEQ ID | (SEQ ID |
| | ID NO: | NO: 458) | | 928) | NO: 1163) | NO: 1398) |
| | 223) | | | | | |
| AB- | GGSISSYY | YIYYSGSTNYNPSL | ARTLHSTIFGVVIMPSSFD | RASQSISSWLA | KASSLES | QQYNSYAT |
| 009792 | WS (SEQ | KS (SEQ ID | Y (SEQ ID NO: 694) | (SEQ ID NO: | (SEQ ID | (SEQ ID |
| | ID NO: | NO: 459) | | 929) | NO: 1164) | NO: 1399) |
| | 224) | | | | | |
| AB- | GYTFTSYGI | WISAYNGNTNYAQ | ARLHRMITFGGVIAQLDV | RASQSVSSSYLA | GASSRAT | QQYGSSPWT |
| 009793 | S (SEQ ID | KLQG (SEQ ID | (SEQ ID NO: 695) | (SEQ ID NO: | (SEQ ID | (SEQ ID |
| | NO: 225) | NO: 460) | | 930) | NO: 1165) | NO: 1400) |
| AB- | GYTLTELS | GFDPEDGETIYAQ | ATAGEQWLDRYYFDY | TGSSSNIGAGYD | GNSNRPS | QSYDSSLSAR |
| 009794 | MH (SEQ | KFQG (SEQ ID | (SEQ ID NO: 696) | VH (SEQ ID | (SEQ ID | YV (SEQ ID |
| | ID NO: | NO: 461) | | NO: 931) | NO: 1166) | NO: 1401) |
| | 226) | | | | | |
| AB- | GYTFTSYDI | WMNPNSGNTGYA | ARARGYQLLFNYYYYMDV | SGDKLGDKYAC | QDSKRPS | QAWDSSTAV |
| 009795 | N (SEQ ID | QKFQG (SEQ ID | (SEQ ID NO: 697) | (SEQ ID NO: | (SEQ ID | (SEQ ID |
| | NO: 227) | NO: 462) | | 932) | NO: 1167) | NO: 1402) |
| AB- | GGSISSSSY | SIYYSGSTYYNPSL | ARHKGQWLVRANWFDP | QGDSLRSYYAS | GKNNRPS | NSRDSSGNHL |
| 009796 | YWG (SEQ | KS (SEQ ID | (SEQ ID NO: 698) | (SEQ ID NO: | (SEQ ID | (SEQ ID |
| | ID NO: | NO: 463) | | 933) | NO: 1168) | NO: 1403) |
| | 228) | | | | | |
| AB- | GGSISSSSY | SIYYSGSTYYNPSL | ARHLRGGVIVATIYGVDY | RASQSVSSSYLA | GASSRAT | QQYGSSQT |
| 009797 | YWG (SEQ | KS (SEQ ID | (SEQ ID NO: 699) | (SEQ ID NO: | (SEQ ID | (SEQ ID |
| | ID NO: | NO: 464) | | 934) | NO: 1169) | NO: 1404) |
| | 229) | | | | | |
| AB- | GYTFTSYY | IINPSGGSTSYAQK | AREVPTFTIFWPLGRGGGW | TGTSSDVGSYNL | EGSKRPS | CSYAGSVV |
| 009798 | MH (SEQ | FQG (SEQ ID | FDP (SEQ ID NO: | VS (SEQ ID | (SEQ ID | (SEQ ID |
| | ID NO: | NO: 465) | 700) | NO: 935) | NO: 1170) | NO: 1405) |
| | 230) | | | | | |
| AB- | GYTFTSYDI | WMNPNSGNTGYA | ARGRLRITIFNRVYYFDY | SGDKLGDKYAC | QDSKRPS | QAWDSSTAW |
| 009799 | N (SEQ ID | QKFQG (SEQ ID | (SEQ ID NO: 701) | (SEQ ID NO: | (SEQ ID | V (SEQ ID |
| | NO: 231) | NO: 466) | | 936) | NO: 1171) | NO: 1406) |
| AB- | GFTFSSYS | SISSSSSYIYYADSV | ARGHFDWLLYETYYFDY | TGTSSDVGGYN | EVSKRPS | SSYAGSNNVV |
| 009800 | MN (SEQ | KG (SEQ ID NO: | (SEQ ID NO: 702) | YVS (SEQ ID | (SEQ ID | (SEQ ID |
| | ID NO: | 467) | | NO: 937) | NO: 1172) | NO: 1407) |
| | 232) | | | | | |
| AB- | GFTFSSYW | NIKQDGSEKYYVD | ARDTVRREQWLVLDYYYM | SGDKLGDKYAC | QDSKRPS | QAWDSSIHVV |
| 009801 | MS (SEQ | SVKG (SEQ ID | DV (SEQ ID NO: | (SEQ ID NO: | (SEQ ID | (SEQ ID |
| | ID NO: | NO: 468) | 703) | 938) | NO: 1173) | NO: 1408) |
| | 233) | | | | | |
| AB- | GGSISSSSY | SIYYSGSTYYNPSL | ARQPRRITIFGVVITPTNN | TGSSSNIGAGYD | GNSNRPS | QSYDSSLSDV |
| 009802 | YWG (SEQ | KS (SEQ ID | WFDP (SEQ ID | VH (SEQ ID | (SEQ ID | V (SEQ ID |
| | ID NO: | NO: 469) | NO: 704) | NO: 939) | NO: 1174) | NO: 1409) |
| | 234) | | | | | |
| AB- | GFTVSSNY | VIYSGGSTYYADS | ARDYGDFYFDY | RASQSVSSNLA | GASTRAT | QQYNNWPRT |
| 010019 | MS (SEQ | VKG (SEQ ID | (SEQ ID NO: 705) | (SEQ ID NO: | (SEQ ID | (SEQ ID |
| | ID NO: | NO: 470) | | 940) | NO: 1175) | NO: 1410) |
| | 235) | | | | | |
| AB- | GFTFSSYG | VISYDGSNKYYAD | AKDYGDYGAFDI (SEQ | KSSQSVLYSSNN | WASTRES | QQYYSTPH |
| 4497999 | MH (SEQ | SVKG (SEQ ID | ID NO: 1897) | KNYLA (SEQ | (SEQ ID | (SEQ ID |
| 20 | ID NO: | NO: 1889) | | ID NO: 1905) | NO: 1913) | NO: 1921) |
| | 1881) | | | | | |
| AB- | GGTFSSYAI | GIIPIFGTANYAQK | ASVGGDYDLDY (SEQ | RASQSVSSYLA | DASNRAT | QQRSNWPPYT |
| 4496014 | S (SEQ ID | FQG (SEQ ID | ID NO: 1898) | (SEQ ID NO: | (SEQ ID | (SEQ ID |
| 61 | NO: 1882) | NO: 1890) | | 1906) | NO: 1914) | NO: 1922) |
| AB- | GGTFSSY | GIIPIFGTANYAQK | ASVGGDYDLDY (SEQ | RASQSVSSYLA | WPP | QQRSNWPPYT |
| 4496352 | AIS (SEQ | [symbol] | ID NO: 1899) | (SEQ ID NO: | DASNRAT | (SEQ ID |
| 24 | ID NO: | | ASVGGDYDLD | [symbol] | (SEQ ID | NO: 1923) |
| | 1883) | | RASQSVSS | DASNRAT YTF | NO: 1915) | |
| | | | YW YLA | | | |
| AB- | GGTFSSYAI | GIIPIFGTANYAQK | ASVGGDYDLDY (SEQ | RASQSVSSYLA | DASNRAT | QQRSNWPPYT |
| 4497472 | S (SEQ ID | FQG (SEQ ID | ID NO: 1900) | (SEQ ID NO: | (SEQ ID | (SEQ ID |
| 65 | NO: 1884) | NO: 1892) | | 1908) | NO: 1916) | NO: 1924) |
| AB- | GGTFSSYAI | GIIPIFGTANYAQK | ASVGGDYDLDY (SEQ | RASQSVSSYLA | DASNRAT | QQRSNWPPYT |
| 4497813 | S (SEQ ID | FQG (SEQ ID | ID NO: 1901) | (SEQ ID NO: | (SEQ ID | (SEQ ID |
| 53 | NO: 1885) | NO: 1893) | | 1909) | NO: 1917) | NO: 1925) |
| AB- | GGTFSSYAI | GIIPIFGTANYAQK | ASVGGDYDLDY (SEQ | RASQSVSSYLA | DASNRAT | QQRSNWPPYT |
| 4498417 | S (SEQ ID | FQG (SEQ ID | ID NO: 1902) | (SEQ ID NO: | (SEQ ID | (SEQ ID |
| 46 | NO: 1886) | NO: 1894) | | 1910) | NO: 1918) | NO: 1926) |
| AB- | GFSLSTSG | LIYWDDDKRYSPS | AHLTTLTIFDY (SEQ | TGASSDVGAYN | DVSNRPS | SSYTSSSVV |
| 4496458 | VGVG (SEQ | LKS (SEQ ID | ID NO: 1903) | YVS (SEQ ID | (SEQ ID | (SEQ ID |
| 91 | ID NO: | NO: 1895) | | NO: 1911) | NO: 1919) | NO: 1927) |
| | 1887) | | | | | |
| AB- | GFSPSTSGV | LIYWDDDKRYSPS | AHHSVATIWDY (SEQ | TGTRSDVGGYN | EVSNRPS | SSYTSSSTLV |
| 4495981 | GVG (SEQ | LKS (SEQ ID | ID NO: 1904) | YVS (SEQ ID | (SEQ ID | (SEQ ID |
| 46 | ID NO: | NO: 1896) | | NO: 1912) | NO: 1920) | NO: 1928) |
| | 1888) | | | | | |

[symbol]

indicates data missing or illegible when filed

TABLE 5
VH and VL sequences

| Anti- | | |
|---|---|---|
| body | VH | VL |
| AB- | QVQLVQSGAEVKKPGASVKVSCKASGYTFTSYGISWVRQA | EIVLTQSPATLSLSPGERATLSCRASQSVSSYLAWYQQ |
| 009269 | PGQGLEWMGWISAYNGNTNYAQKLQGRVTMTTDTSTSTA | KPGQAPRLLIYDASNRATGIPARFSGSGSGTDFTLTIS |
| | YMELRSLRSDDTAVYYCAREGDCSGGSCYSGYYYYYGMD | SLEPEDFAVYYCQQRSNWGTFGPGTKVDIK |
| | VWGQGTTVTVSS (SEQ ID NO: 1411) | (SEQ ID NO: 1646) |
| AB- | QMQLVQSGPEVKKPGTSVKVSCKASGFTFTSSAMQWVRQ | EIVLTQSPGTLSLSPGERATLSCRASQSVSSSYLAWYQ |
| 009270 | ARGQRLEWIGWIVVGSGNTNYAQKFQERVTITRDMSTSTA | QKPGQAPRLLIYGASSRATGIPDRFSGSGSGTDFTLTI |
| | YMELSSLRSEDTAVYYCAVNYCSSTSCYDAFDIWGQGTMV | SRLEPEDFAVYYCQQYGSSPWTFGQGTKVEIK |
| | TVSS (SEQ ID NO: 1412) | (SEQ ID NO: 1647) |
| AB- | QVQLVQSGAEVKKPGASVKVSCKVSGYTLTELSMHWVRQ | QSVLTQPPSVSAAPGQKVTISCSGSSSNIGNNYVSWYQ |
| 009272 | APGKGLEWMGGFDPEDGETIYAQKFQGRVTMTEDTSTDTA | QLPGTAPKLLIYDNNKRPSGIPDRFSGSKSGTSATLGI |
| | YMELSSLRSEDTAVYYCATAPPYCSSTSCPINWFDPWGQG | TGLQTGDEADYYCGTWDSSLSEWVFGGGTKLTVL |
| | TLVTVSS (SEQ ID NO: 1413) | (SEQ ID NO: 1648) |
| AB- | QVQLVQSGAEVKKPGSSVKVSCKASGGTFSSYAISWVRQA | EIVLTQSPATLSLSPGERATLSCRASQSVSSYLAWYQQ |
| 009273 | PGQGLEWMGGIIPIFGTANYAQKFQGRVTITTDESTSTAY | KPGQAPRLLIYDASNRATGIPARFSGSGSGTDFTLTIS |
| | MELSSLRSEDTAVYYCARVGVYDSSGYYFDYWGQGTLVTV | SLEPEDFAVYYCQQRSNWPPRLTFGGGTKVEIK |
| | SS (SEQ ID NO: 1414) | (SEQ ID NO: 1649) |
| AB- | QVTLRESGPALVKPTQTLTLTCTFSGFSLSTSGMRVSWIR | DIVMTQSPDSLAVSLGERATINCKSSQSVLYSSNNKNY |
| 009274 | QPPGKALEWLARIDWDDDKFYSTSLKTRLTISKDTSKNQV | LAWYQQKPGQPPKLLIYWASTRESGVPDRFSGSGSGT |
| | VLTMTNMDPVDTATYYCARSYSSSLPFDYWGQGTLVTVSS | DFTLTISSLQAEDVAVYYCQQYYSTPFTFGPGTKVDIK |
| | (SEQ ID NO: 1415) | (SEQ ID NO: 1650) |
| AB- | QVTLRESGPALVKPTQTLTLTCTFSGFSLSTSGMRVSWIR | QSVLTQPPSASGTPGQRVTISCSGSSSNIGSNYVYWYQ |
| 009275 | QPPGKALEWLARIDWDDDKFYSTSLKTRLTISKDTSKNQV | QLPGTAPKLLIYRNNQRPSGVPDRFSGSKSGTSASLAI |
| | VLTMTNMDPVDTATYYCARSNYDILTGRLDAFDIWGQGTM | SGLRSEDEADYYCAAWDDSLSGWVFGGGTKLTVL |
| | VTVSS (SEQ ID NO: 1416) | (SEQ ID NO: 1651) |
| AB- | EVQLLESGGGLVQPGGSLRLSCAASGFTFSSYAMSWVRQA | DIQLTQSPSFLSASVGDRVTITCRASQGISSYLAWYQQ |
| 009276 | PGKGLEWVSAISGSGGSTYYADSVKGRFTISRDNSKNTLY | KPGKAPKLLIYAASTLQSGVPSRFSGSGSGTEFTLTIS |
| | LQMNSLRAEDTAVYYCAKDRNYDFWSGYYFDYWGQGTLV | SLQPEDFATYYCQQLNSYPLTFGGGTKVEIK |
| | TVSS (SEQ ID NO: 1417) | (SEQ ID NO: 1652) |
| AB- | QVQLVESGGGVVQPGRSLRLSCAASGFTFSSYAMHWVRQA | SYELTQPPSVSVSPGQTASITCSGDKLGDKYACWYQQ |
| 009277 | PGKGLEWVAVISYDGSNKYYADSVKGRFTISRDNSKNTLY | KPGQSPVLVIYQDSKRPSGIPERFSGSNSGNTATLTIS |
| | LQMNSLRAEDTAVYYCARDGGGLLLNWGQGTLVTVSS | GTQAMDEADYYCQAWDSSTVVFGGGTKLTVL |
| | (SEQ ID NO: 1418) | (SEQ ID NO: 1653) |
| AB- | QVQLVESGGGVVQPGRSLRLSCAASGFTFSSYAMHWVRQA | SYVLTQPPSVSVAPGKTARITCGGNNIGSKSVHWYQQ |
| 009278 | PGKGLEWVAVISYDGSNKYYADSVKGRFTISRDNSKNTLY | KPGQAPVLVVYDDSDRPSGIPERFSGSNSGNTATLTIS |
| | LQMNSLRAEDTAVYYCARDGSSWYIDYWGQGTLVTVSS | RVEAGDEADYYCQVWDSSSDHYVFGTGTKVTVL |
| | (SEQ ID NO: 1419) | (SEQ ID NO: 1654) |
| AB- | QVQLVESGGGVVQPGRSLRLSCAASGFTFSSYAMHWVRQA | EIVLTQSPGTLSLSPGERATLSCRASQSVSSSYLAWYQ |
| 009279 | PGKGLEWVAVISYDGSNKYYADSVKGRFTISRDNSKNTLY | QKPGQAPRLLIYGASSRATGIPDRFSGSGSGTDFTLTI |
| | LQMNSLRAEDTAVYYCARPRGGSYYSAFDIWGQGTMVTV | SRLEPEDFAVYYCQQYGSSPWTFGQGTKVEIK |
| | SS (SEQ ID NO: 1420) | (SEQ ID NO: 1655) |
| AB- | QVQLVESGGGVVQPGRSLRLSCAASGFTFSSYGMHWVRQA | DIQMTQSPSSLSASVGDRVTITCRASQGISNYLAWFQQ |
| 009280 | PGKGLEWVAVISYDGSNKYYADSVKGRFTISRDNSKNTLY | KPGKAPKSLIYAASSLQSGVPSKFSGSGSGTDFTLTIS |
| | LQMNSLRAEDTAVYYCAKSRGGGYYYYYMDVWGKGTTV | SLQPEDFATYYCQQYNSYPPTFGQGTKLEIK |
| | TVSS (SEQ ID NO: 1421) | (SEQ ID NO: 1656) |
| AB- | EVQLVETGGGLIQPGGSLRLSCAASGFTVSSNYMSWVRQA | DIQLTQSPSFLSASVGDRVTITCRASQGISSYLAWYQQ |
| 009281 | PGKGLEWVSVIYSGGSTYYADSVKGRFTISRDNSKNTLYL | KPGKAPKLLIYAASTLQSGVPSRFSGSGSGTEFTLTIS |
| | QMNSLRAEDTAVYYCARDLYYYGMDVWGQGTTVTVSS | SLQPEDFATYYCQQLNSYPTFGQGTKVEIK |
| | (SEQ ID NO: 1422) | (SEQ ID NO: 1657) |
| AB- | EVQLVESGGGLIQPGGSLRLSCAASGFTVSSNYMSWVRQA | QSVLTQPPSVSGAPGQRVTISCTGSSSNIGAGYDVHWY |
| 009282 | PGKGLEWVSVIYSGGSTYYADSVKGRFTISRDNSKNTLYL | QQLPGTAPKLLIYGNSNRPSGVPDRFSGSKSGTSASLA |
| | QMNSLRAEDTA VYYCARILPLNWYYFDYWGQGTLVTVSS | ITGLQAEDEADYYCQSYDSSLSGSGVFGTGTKVTVL |
| | (SEQ ID NO: 1423) | (SEQ ID NO: 1658) |
| AB- | EVQLVESGGGLIQPGGSLRLSCAASGFTVSSNYMSWVRQA | SYELTQPPSVSVSPGQTASITCSGDKLGDKYACWYHQ |
| 009283 | PGKGLEWVSVIYSGGSTYYADSVKGRFTISRDNSKNTLYL | KPGQSPVLVIYQDTKRPSGIPERFSASNSGNTATLTIS |
| | QMNSLRAEDTAVYYCATSSFDYYYYFMDVWGKGTTVTVSS | GTQALDEADYYCQAWDSSIVVFGGGTKLTVL |
| | (SEQ ID NO: 1424) | (SEQ ID NO: 1659) |
| AB- | QVQLQESGPGLVKPSQTLSLTCTVSGGSISSGGYYWSWIR | EIVLTQSPGTLSLSPGERATLSCRASQSVSSSYLAWYQ |
| 009284 | QHPGKGLEWIGYIYYSGSTYYNPSLKSRVTISVDTSKNQF | QKPGQAPRLLIYGASSRATGIPDRFSGSGSGTDFTLTI |
| | SLKLSSVTAADTAVYYCARAPTIFGVVIGWFDPWGQGTLV | SRLEPEDFAVYYCQQYGSSPLTFGQGTRLEIK |
| | TVSS (SEQ ID NO: 1425) | (SEQ ID NO: 1660) |
| AB- | QVQLQESGPGLVKPSQTLSLTCTVSGGSISSGGYYWSWIR | EIVLTQSPGTLSLSPGERATLSCRASQSVSSSYLAWYQ |
| 009285 | QHPGKGLEWIGYIYYSGSTYYNPSLKSRVTISVDTSKNQF | QKPGQAPRLLIYGASSRATGIPDRFSGSGSGTDFTLTI |
| | SLKLSSVTAADTAVYYCARAPRITIFGVVSPFDYWGQGTL | SRLEPEDFAVYYCQQYGSSPLTFGGGTKVEIK |
| | VTVSS (SEQ ID NO: 1426) | (SEQ ID NO: 1661) |
| AB- | QVQLQESGPGLVKPSQTLSLTCTVSGGSISSGDYYWSWIC | SSELTQDPAVSVALGQTVRITCQGDSLRSYYASWYQQ |
| 009286 | QAEAKGLEWIGYIYYSGSTYYNPSLKSRVTISVDTSKNQF | KPGQAPVLVIYGKNNRPSGIPDRFSGSSSGNTASLTIT |
| | SLKLSSVTAADTAVYYCAREGRFLEWLLSVNYYYYMDVWG | GTQAEDEADYYCNSRDSSGNHGVFGTGTKVTVL |
| | KGTTVTVSS (SEQ ID NO: 1427) | (SEQ ID NO: 1662) |
| AB- | QLQLQESGPGLVKPSETLSLTCTVSGGSISSSSYYWGWIR | DIQLTQSPSFLSASVGDRVTITCRASQGISSYLAWYQQ |
| 009287 | QPPGKGLEWIGSIYYSGSTYYNPSLKSRVTISVDTSKNQF | KPGKAPKLLIYAASTLQSGVPSRFSGSGSGTEFTLTIS |
| | SLKLSSVTAADTAVYYCARTRITIFGGERYFDLWGRGTLV | SLQPEDFATYYCQQLNSYPLTFGGGTKVEIK |
| | TVSS (SEQ ID NO: 1428) | (SEQ ID NO: 1663) |
| AB- | QVQLQESGPGLVKPSETLSLTCTVSGGSISSYYWSWIRQP | EIVLTQSPGTLSLSPGERATLSCRASQSVSSSYLAWYQ |
| 009288 | PGKGLEWIGYIYYSGSTNYNPSLKSRVTISVDTSKNQFSL | QKPGQAPRLLIYGASSRATGIPDRFSGSGSGTDFTLTI |
| | KLSSVTAADTAVYYCARGFDYWGQGTLVTVSS | SRLEPEDFAVYYCQQYGSSPWTFGQGTKVEIK |
| | (SEQ ID NO: 1429) | (SEQ ID NO: 1664) |
| AB- | EVQLVQSGAEVKKPGESLKISCKGSGYSFTSYWIGWVRQL | EIVMTQSPLFLPVTPGEPASISCRSSQSLLHSNGYNYL |
| 009289 | PGKGLEWMGIIYPGDSDTRYSPSFQGQVTISADKSISTAY | DWYLQKPGQSPQLLIYLGSNRASGVPDRFSGSGSGTDF |
| | LQWSSLKASDTAMYYCARRGFGLTYYGMDVWGQGTTVTVS | TLKISRVEAEDVGVYYCMQALQTPQTFGQGTKVEIK |
| | S (SEQ ID NO: 1430) | (SEQ ID NO: 1665) |
| AB- | DVQLVESGGGLVQPGGSLRLSCIASGFAFSDHHMDWVRQA | EIVLTQSPGTLSLPPGERATLSCRASQSVNKFVLAWYQ |
| 009290 | PGKGLEWVGRIRDKADSYTTEYAASVRGRFTISRDDLKNS | HKPGQAPRLLIYGASTRATGIPDRFSGSGSGTDFTLTI |
| | QYLQMNSLKTEDTAMYYCASSHSRGYYAFDYWGQGALVTV | NRLEPEDLAVYYCQLYDRSPPPIGQGTKVEIK |
| | SS (SEQ ID NO: 1431) | (SEQ ID NO: 1666) |
| AB- | EVHLLESGGGLVQPGGSLRLSCAASGFTFTTYAMFWVRQA | EIVLTQSPATLSLSPGERATLSCRASQSVSSYLAWYQQ |
| 009291 | PGKGLEWVSAINAGGAKTHYADSVRGRFTISRDNSKSTLY | RPGQAPRLLIYDASSRATGIPVRFSGSGSGTDFTLTIS |
| | LHMDRLRVGDTAIYYCAKVPSYIYDSRDDYLDFWGQGTLV | SLEPEDFAVYYCQQRSDWPLLTFGGGTKVEIK |
| | TVSS (SEQ ID NO: 1432) | (SEQ ID NO: 1667) |
| AB- | EVQLVDSGGGLVQPGGALRLSCVVSGFTVRYNYMSWVRQ | ESVLTQSPGTLSLSPGETVTLSCRASQSITNNYLAWYQ |
| 009292 | APGKGLEWVSIIYRGDRGGSTYYIDSVKGRFIISRDDSRN | QKPGQAPRLVVYHASKRASGIPDRFSGSGSGTDFALTI |
| | TVYLQMNSLSAEDTAVYYCASSHSADYLGPFDYWGRGTLV | SRLEPEDFAVYYCQQFGTFGTFGGGTKLEIK |
| | TVSS (SEQ ID NO: 1433) | (SEQ ID NO: 1668) |
| AB- | QAQLVESGGGVVQPGRSLRLSCAASGFNFNNYGMHWVRL | DIQMTQSPPSLSASVGDRVTFTCRASQDITNSLAWFQQ |
| 009293 | AAGKGLEWVAFISYEGSITHYADSVKGRFTVSRDFSKNTL | RPGKAPKSLIYAASTLHSGVPSKFSGSGSGTDFTLTIT |
| | YLQMNRLTTGDTAVYYCAKRREVLHLWRDEMDVWGQGTT | SLQPEDVATYYCQQYKTYPFTFGQGTRLEIK |
| | VTVSS (SEQ ID NO: 1434) | (SEQ ID NO: 1669) |
| AB- | EVRLLDSGGGLVQPGGSLRLSCVASGFTLSTYSVTWYRQA | EIVLTQSPATLSLSPGERATLSCRASQSVSAYFAWYQQ |
| 009294 | PGKGPEWVAVFSATGGTTDYADSVRGRFTFSRDNSENTLY | KPGQAPRLLIYDATNRATGIPARFSGSGFGTDFTLTIS |
| | VQMTSLRADDTAFYYCVKYLQSGDEGYWGPGTLVTVSS | NLEPDDFAVYYCQQRSFWPLTFGGGTKVEIK |
| | (SEQ ID NO: 1435) | (SEQ ID NO: 1670) |
| AB- | QVQLVESGGGVVQPGRSLRLSCAASGFTFSSYAMHWVRQA | QSVLTQPPSVSGAPGQRVTISCTGHNSNIGAGSDVHWY |
| 009295 | PGKGLEWVAILWPDGGKKYFADSVKGRFTISRDNSKNTLF | QQLPGTAPKLLIYGNRNRPSGVPDRFSGSKSGTSGSLA |
| | LQMSSLRAEDTATYYCARDPASGWGLDPWGQGTLVTVSS | ITGLQTEDEADYYCQSFDSSLNVYVFGTGTKVTVL |
| | (SEQ ID NO: 1436) | (SEQ ID NO: 1671) |
| AB- | QVQLVQSGAEVKKPGASVKVSCKASGYTFTSYDINWVRQA | SYELTQPPSVSVSPGQTASITCSGDKLGDKYACWYQQ |
| 009296 | TGQGLEWMGWMNPNSGNTGYAQKFQGRVTMTRNTSISTA | KPGQSPVLVIYQDSKRPSGIPERFSGSNSGNTATLTIS |
| | YMELSSLRSEDTAVYYCARAAKRITIFGTRGYYFDYWGQG | GTQAMDEADYYCQAWDSSTAWEVFGGGTKLTVL |
| | TLVTVSS (SEQ ID NO: 1437) | (SEQ ID NO: 1672) |
| AB- | QVQLVQSGAEVKKPGASVKVSCKASGYTFTSYGISWVRQA | DIVMTQSPLSLPVTPGEPASISCRSSQSLLHSNGYNYL |
| 009297 | PGQGLEWMGWISAYSGNTNYAQKLQGRVTMTTDTSTSTT | DWYLQKPGQSPQLLIYLGSNRASGVPDRFSGSGSGTDF |
| | YMELRSLRSDDTAVYYCARDSSSWPYVSLGYWGQGTLVT | TLKISRVEAEDVGVYYCMQALQTPRTFGQGTKLEIK |
| | VSS (SEQ ID NO: 1438) | (SEQ ID NO: 1673) |
| AB- | QVQLQESGPGLVKPSETLSLTCTVSGGSISSHYWSWIRQP | DIQMTQSPSSLSASVGDRVTITCRASQSISSYLNWYQQ |
| 009298 | PGKGLEWIGYIYYSGSTNYNPSLKSRVTISVDTSKNQFSL | KPGKAPKLLIYAASSLQSGVPSRFSGSGSGTDFTLTIS |
| | KLSSVTAADTAVYYCARIPRGWLQSPSYYFDYWGQGTLVT | SL QPEDFATYYCQQSYSTPQTFGQGTKVEIK |
| | VSS (SEQ ID NO: 1439) | (SEQ ID NO: 1674) |
| AB- | QVQLVQSGAEVKKPGASVKVSCKASGYTFSNYYMHWVRQ | QSALTQEASVSGTVGQKVTLSCTGNTNNIGSHAVGWY |
| 009299 | APGQGLEWMGIIKPSAGRTIYAQKFVGRVTMTRDTSTSTV | QQISHGAPKTVMFRNSLPSGIPGRFSGSQSGTTASLTI |
| | YMELSSLRSDDTAVYYCARGGGPTDRDYWGPGTLVAVSS | SGLQPEDEADYYCSTWDNSLSAVVFGGGTRLTVL |
| | (SEQ ID NO: 1440) | (SEQ ID NO: 1675) |
| AB- | EVQLVESGGGLVKPGGSLRLSCAASGFTFSSYRMNWVRQA | EIVMTQSPATLSVSPGERATLSCRASQSVSSNLAWYQQ |
| 009300 | PGKGLEWVSSISSSSSYIYYANSVKGRFTISRDNAKNSLY | KPGQAPRLLIYGASTRATGIPARFSGSGSGTEFTLTIS |
| | LQMNSLRAEDTAVYYCARVVWQLVENYYYYMDVWGKGTT | SLQSEDFAVYYCQQYNNWPPITFGQGTRLEIK |
| | VTVSS (SEQ ID NO: 1441) | (SEQ ID NO: 1676) |
| AB- | QVQLQQWGAGLLKPSETLSLTCAVYGGSFTNYYWNWIRQP | SSELTQDPAVSVALGQTVRITCQGDSLRKYYPTWYQQ |
| 009301 | PGRGLEWIGEINHSGSTNYKPSLKSRVTISIDTSKNQFSL | KPGQAPVLVIYGKNTRHSGIPDRFSGSSSGNTASLTIT |
| | KLSSVTAADTAVYFCARRLGYDNGWGTYFYGLDVWGQGTT | GAQAEDEADYYCNSRDSSDSRNWVFGGGTKLTIL |
| | VTVTS (SEQ ID NO: 1442) | (SEQ ID NO: 1677) |
| AB- | QLQLQESGPGLVKPSETLSLICNVSGGSIISTSHYWGWIR | SYELTQPPSVSVSPGQTARITCSGDALPKKYAYWYQQ |
| 009302 | QPPGKGLEWIGTIYYSGNTYYNPSLTSRATMSVDTSKNHF | RSGQAPVLVIYEDTKRPSGIPERFSGSSSGTVASLTIS |
| | SLNLSSVTAADTAVYFCARRSRAVAGKYYWYYDLWGRGAL | GAQVEDEADYYCYSTDSSGYHEVFGGGTKLTVL |
| | VSVSS (SEQ ID NO: 1443) | (SEQ ID NO: 1678) |
| AB- | EMELVESGGGLVQPGGSLRLSCAASGFTFSTYWMTWLRQT | DIQMTQSPSSLSAFVGDRVTITCRASQSINTYLNWYQQ |
| 009303 | PGKGLDWVANIKPDESEIHYVDSVKGRFTVSRDNAKNSVY | RPGKAPKLLIFLASSLQSGVPSRFRGGGSGTDFTLTIS |
| | LHMNSLRAEDTAVYYCARLRAVGGDDTFDVWGQGTMVT | SLQPEDFATYYCQQSYRSWTFGQGTKVEIK |
| | VSS (SEQ ID NO: 1444) | (SEQ ID NO: 1679) |
| AB- | EVQLVESGGGLVQPGGSLRLSCAASGFTFSDYAMTWVRQT | AIRMTQSPSSLSASTGDRVTITCRASQAITNYLAWYQQ |
| 009304 | PGKGLEWISAITENGFTTYYADSMRGRFTISRDNSKNTLY | KPGKAPKLLIASASTLQSGVPSRFSGSGSGTDFTLTIS |
| | LQMSSLRAEDTAVYYCAKYWSLSGPTELLDSWGQGTLVTV | SLQSEDFASYYCQQYYSYPLTFGGGTKVEIK |
| | SS (SEQ ID NO: 1445) | (SEQ ID NO: 1680) |
| AB- | EVQLLESGGNLVQPGGSLRLSCAASGFTFSSYSMSWFRQA | EIVLTQSPATLSLSPGERATLSCRASQSVRNFFAWYQQ |
| 009305 | PGKGLEWVSVIDNRGGNIHYSDSVRVRFTISRDNSKNTLY | KPGQAPRLLIYGASNRATGIPARFSGSGSGTDFTLTIS |
| | LQMSSLRAEDTAIYYCAQYVQEGMDVWGPGTTVTVSS | SLEPEDFAVYFCQQRFDWPLTFGAGTKVEIK |
| | (SEQ ID NO: 1446) | (SEQ ID NO: 1681) |
| AB- | QVQLVQSGAEVKKPGASVKVPCKASGYTFTSYQMHWVRR | SYELTQPPSVSVSPGQTARITCSGDALPKKYAYWYQQ |
| 009306 | APGQGLEWMGLIDPDTGGTRYTQNFQGRVTMTRDTSTTTV | KSGQAPVLVIYEDSKRPSGIPERFSGSSSGTMATLTIS |
| | YMELNSLTSGDTAVYYCARGGRVGATYPYNMDVWGKGT | GAQVEDDSDYYCFSTDSSGNHGVFGGGTRLTVL |
| | TVTVSS (SEQ ID NO: 1447) | (SEQ ID NO: 1682) |
| AB- | QVQLQESCPGLAKPSQTLSLTCTVSGGSISGRGYFWTWIR | QSVLTQPPSVSGAPGQRVTISCAGSSSNIGTGYDVHWY |
| 009307 | QPAGKGLEWIGDISSSGSTNYNPFLKSRVSVSLDASKNTF | QQLPGGAPKLLIYGNTNRPSGVPDRFSGSRSGTSASLA |
| | SLNLRSVTAADTAVYFCARDRRLYGDFYYYGMDVWGQGTT | ITGLQAEDEADYYCQSYDSSLAGSRVFGGGTKLTVL |
| | VTVSS (SEQ ID NO: 1448) | (SEQ ID NO: 1683) |
| AB- | EVQLVESGGGTVQPGGSLRLSCAGSGFSFNSYWMSWVRQA | SSELTQDPAVSVALGQTIRITCQGDSLRNYYASWYQQ |
| 009308 | PGKGLEWVASIKQDGSETYYVDSVKGRVSISRDNTKRSLF | KPGQAPLLVIYGKNNRPSGIPDRFSGSTSINSASLTIT |
| | LQMNSLRADDTAVYYCARGVDGISTIVVLVFWNWYFDLWG | GAQAEDEADYYCNSRDANTNHVLFGGGTKLTVV |
| | RGTLVTVSS (SEQ ID NO: 1449) | (SEQ ID NO: 1684) |
| AB- | QVQLQESGPGLVKPSGTLSLTCTVSVGSISSHYWSWIRQP | EIVLTQSPGTLSLSPGERATLSCRASQSVSSSYLAWYQ |
| 009309 | PGKGLEWIGYIYYSGSTNYNPSLKSRVTISVDTSKNQFSL | QKPGQAPRLLIYGASSRATGIPDRFSGSGSGTDFTLTI |
| | KLSSVTAADTAVYYCARSGRGWLQLNPSYYFDYWGQGTLV | SRLEPEDFAVYYCQQYGSSPPGTFGQGTRLEIK |
| | TVSS (SEQ ID NO: 1450) | (SEQ ID NO: 1685) |
| AB- | EVQLLESGGGLVQPGGSLRLSCAASGFTFSSYAMSWVRQA | EIVLTQSPGTLSLSPGERATLSCRASQSLSSSNLAWYQ |
| 009310 | PGKGLEWVSAISGSGTSTYYADSVKGRFTISRDNSKNTLY | QKPGQAPRLLIYGASSRATGIPDRFSGSGSGTDFTLTI |
| | LQMNSLRAEDTAVYYCAKGYPRYYYGMDVWGQGTTVTVS | SRLEPEDFAVYYCQQYGSSPFTFGPGTKVDIK |
| | S (SEQ ID NO: 1451) | (SEQ ID NO: 1686) |
| AB- | QVQLVQSGAEVKKPGASVKVSCKASGYTFTSYDINWVRQA | QSALTQPASVSGSPGQSITISCTGTSSDVGGYNYVSWY |
| 009311 | TGQGLEWMGWMNPNSGNTGYAQKFQGRVTMTRNTSISTA | QQHPGKAPKLMIYDVSNRPSGVSNRFSGSKSGNTASLT |
| | YMELSSLRSEDTAVYYCARRRAATWSIYYYYYYMDVWGK | ISGLQAEDEADYYCTSYTSSSTLEGWVFGGGTKLTVL |
| | GTTVTVSS (SEQ ID NO: 1452) | (SEQ ID NO: 1687) |
| AB- | EVQLVESGGGLVQPGGSLRLSCAASGFIFSNYWMSWVRQA | DIQMTQSPSSLSASVGDRVTITCRASHGISTWLAWYQL |
| 009312 | PGKGLEWVANIKQDGGEQYYVDSVKGRFTISRDNGKNSLY | KPGKAPKSLIYAASNLQSGVPSRFSGSGSGTDFTLTIS |
| | LQLNSLRADDTAVYYCARSTYLPAAGTMNFWGRGTLVTV | SLQPEDFATYYCHQYNSYPFTFGGGTKVEIK |
| | SS (SEQ ID NO: 1453) | (SEQ ID NO: 1688) |
| AB- | QVQLQQWGAGLLKPSETLSLTCAVYGGSFSGYYWSWIRQP | SSELTQDPAVSVASGQTVRITCQGDSLRSYYASWYQQ |
| 009313 | PGKGLEWIGEINHSGSTNYNPSLKSRVTISVDTSKNQFSL | KPGQAPVLVIYGKNNRPSGIPDRFSGSSSGNTASLTIT |
| | KLSSVTAADTAVYYCARGNIVVVPAAMGVFLYYYYSMDVW | GAQAEDEADYYCNSRDSSGNHLVVFGGGTKLTVL |
| | GKGTTVTVSS (SEQ ID NO: 1454) | (SEQ ID NO: 1689) |
| AB- | QVQLQESGPGLVKPSETLSLTCTVSGGSISSHYWSWIRQP | EIVLTQSPGTLSLSPGERATLSCRASQSVSSSYLAWYQ |
| 009314 | PGKGLEWIGYIYYSGSTNYNPSLKSRVTMSVDTSKNQFSL | QKPGQAPRLLIYGASSRATGIPDRFSGSGSGTDFTLTI |
| | KLSSVTAADTAVYYCARGERQWLQLYSYYGMDVWGQGTTV | SRLEPEDFAVYYCQQYGSSPLTFGGGTKVEIK |
| | TVSSA (SEQ ID NO: 1455) | (SEQ ID NO: 1690) |
| AB- | QVQLVQSGAEVKKPGASVKVSCKASGYTFTSYGISWVRQA | SSELTQDPAVSVALGQTVRITCQGDSLRSYYASWYQQ |
| 009315 | PGQGLEWMGWISAYNGNTNYAQKLQGRVTMTTDTSTSTA | KPGQAPVLVIYGKNNRPSGIPDRFSGSSSGNTASLTIT |
| | YMELRSLRSDDTAVYYCARDVRMITFGGVIVYFDYWGQGT | GAQAEDEADYYCNSRDSSGNHRVFGGGTKLTVL |
| | LVTVSS (SEQ ID NO: 1456) | (SEQ ID NO: 1691) |
| AB- | EVQLVESGGGLVQPGGSLRLSCAASGFTFSNYWMHWVRQ | DVVMTQTPLSLSVTPGQPASISCKSSQSLLQSDGKTYL |
| 009316 | VPGKGLVWVSSINNDGSNTRYADSVKGRFTISRDNAKNTV | YWYLQKPGQPPQLLIYEVSKRFSGVPDRFSGSGSGTDF |
| | YMQMNSLRAEDTAVYYCARPWGYWGQGTLVTVSS (SEQ | TLKISRVEADDVGVYYCMQSIQVPITFGPGTKVDIK |
| | ID NO: 1457) | (SEQ ID NO: 1692) |
| AB- | QVQLVQSGAEVKKPGASVKVSCKASGYTFTSYDINWVRQA | SYELTQPPSVSVSPGQTASITCSGDKLGDKYACWYQQ |
| 009317 | TGQGLEWMGWMNPNSGNTGYAQKFQGRVTMTRNTSISTA | KPGQSPVLVIYQDSKRPSGIPERFSGSNSGNTATLTIS |
| | YMELSSLRSEDTAVYYCARGMKVVTIFGRREYYFDYWGQ | GTQAMDEADYYCQAWDSSTVVFGGGTKLTVL |
| | GTLVTVSS (SEQ ID NO: 1458) | (SEQ ID NO: 1693) |
| AB- | QLQLLESGPGRVKPSETLSLTCSVSGAFISTSNSFWGWIR | QSALTQPPSVSGSPGQSVTISCTGTSSDIGGYNYVSWY |
| 009318 | QPPGKGLEWIGSENYGGSSYFNPSLKRRVAISVDTSKNQF | QQHPGKGPKLMIYDVNKRPSGVPDRFSGSKSGNTASL |
| | SLKLRSVTAADTAIYYCARRPSLGFGSSWNFDLWGRGTLV | TISGLQAEDEAHYSCSSYAGSYTYVVFGGGTQLTVL |
| | TVSS (SEQ ID NO: 1459) | (SEQ ID NO: 1694) |
| AB- | EVQLVESGGGLVQTGGSLRLSCRGSGFSFSNDWLSWVRQA | DVVMAQSPLSLSVTLGQPASISCRSSQSLLYSNGNTYL |
| 009319 | PGKGLEWVANINQDGSEKYSVDSVKGRFTISRDNAKISLY | SWFQQRPGQSPRRLIYKVSIRDSGVPDRFSGSGSDTDF |
| | LQMNSLRAEDTAVYYCVRDAGGWGQGTLVTVSS | LKITRVEAEDVGIYYCVQGTHWPFTFGPGTKVDIK |
| | (SEQ ID NO: 1460) | T (SEQ ID NO: 1695) |
| AB- | QVQLVQSGAEVKKPGSSVKVSCKASGGTLSRCVITWVRQA | DIVMTQTPLSLPVTPGEPASISCRSSQSLLDSEDGNTY |
| 009320 | PGQGLEWMGRIIPIFGTVNYAQKFQGRVTIGADESTSTAY | LDWYLQKPGQSPQLLIYTLSYRASGVPDRFIGSGSGTD |
| | MELSRLRSEDTAVYYCANTDIGDVRFYGMDVWGQGTTVTV | FTLEISRVEAEDVGIYYCMQSMEFPWTFGPGTKVEIK |
| | SS (SEQ ID NO: 1461) | (SEQ ID NO: 1696) |
| AB- | QVQLQESGPGLVKPSQTLSLTCTVSGGSISDGDYYWSWIR | EVVLTQSPGTLSLSPGERATLSCRASQSVTSSYLAWYQ |
| 009321 | QPPGKGLEWIAYISYSGSTYYNPSLKSRVALSVDTSKNQF | QKAGQAPRLLIYGASSRATGIPDRFSGSGSGTDFTLTI |
| | SLNLNSVTAADTAVYYCARIRGERITIFAVLVTGAFDVWG | SRLDPEDFAVYYCQQYGSSPLTFGGGTKVEIK |
| | QGTLVTVSA (SEQ ID NO: 1462) | (SEQ ID NO: 1697) |
| AB- | QLQLQESGPGLVRPSETLSLTCSVSGDSISGTKNYWAWIR | HFMLTQPQSVSESPGETVIISCTRSSGSIASNYVQWYQ |
| 009322 | QPPGKGLEWIGSFYYNWGSTSYNASLKSRVSISVDTSRNQ | QRPGSAPTCVIYEDNQRPSGVPDRFSGSIDSSSNTASL |
| | LSLKLTSVSAADTAVYYCARRLEPRHYFDTWGRGSLVTVS | TISGLKTEDEGDYYCQSYDKGWVFGGGTKLTVL |
| | S (SEQ ID NO: 1463) | (SEQ ID NO: 1698) |
| AB- | QVHLVQSGAEVKKPGASVRVSCKVSGYSLSLLTMYWVRQ | EIELTQSPGTLSLSPGERATLSCRASQSVSSNYLAWYQ |
| 009323 | APGKGLEWMGSFDPQDGETMYAQKFQGRVTMTEDTSADT | QKPGQAPRLLIYGASSRATGIPDRLSGSGSGTDFTLTI |
| | AYMELSGLRSEDTAVYYCATDDVSLVVIYALAIWGQGTLV | SSLEPEDFAVYHCHQYGTSPHTFGQGTKLEIK |
| | TVSS (SEQ ID NO: 1464) | (SEQ ID NO: 1699) |
| AB- | EVQLFQSGGGLVQPGGSLRLSCKASGFTFSDYDISWVRQA | EIVLTQSPATLSLSPGERATLSCRASQSVTTFLGWYQQ |
| 009324 | PGKGLEWVSHITLTGRTYYVDSVQGRFTISRDNSKDTVYL | KPGQAPRLLIYDASNRAPGIPARFSGSGSGTDFTLTIS |
| | QMNGLRADDTAVYYCAKHLIRAYDYWGQGALVTVSS | SLEPEDVGVYYCQQRYNWPLTFGPGTKVEIK |
| | (SEQ ID NO: 1465) | (SEQ ID NO: 1700) |
| AB- | QVQLVESGGGVVQPGRSLRLSCAASGFTFNNYGMHWVRQ | QSVLTQPPSVSAAPGQKVTISCSGSSSNIGKNYVSWYR |
| 009325 | APGKGLEWVALISYEGSLKYYGDSVRGRFTVSRDNSRNTL | QFPGTAPKLLIYDNNKRPSGIPDRFSGSKSDTSATLGI |
| | YLQMNSLRVDDTAVYYCAKAGGIFAIHYYGMNVWGQGTT | TGLQTGDEADYYCVTWDSSLSAGVFGGGTKLTVL |
| | VTVSS (SEQ ID NO: 1466) | (SEQ ID NO: 1701) |
| AB- | QLQLQESGPGLVKPSETLSLTCTVSGGSISSSSYYWGWIR | DIQMTQSPSTLSASVGDRVTITCRASQSISSWLAWYQQ |
| 009326 | QPPGKGLEWIGSIYYSGSTYYNPSLKSRVTISVDTSKNQF | KPGKAPKLLIYKASSLESGVPSRFSGSGSGTEFTLTIS |
| | SLKLSSVTAADTAVYYCARDGAPLYDILTHDAFDIWGQGT | SLQPDDFATYYCQQYNSYPYTFGQGTKLEIK |
| | MVTVSS (SEQ ID NO: 1467) | (SEQ ID NO: 1702) |
| AB- | EVQLVESGGGLVRPGGSLRLSCVASGLPFSNPRMSWVRQS | QIVLTQSPDTLSLSPGERATLSCRASQTVSRNYLAWYQ |
| 009327 | PGKGLEWVGRIRSKADGGTSDYAAPVKGRFTISWDDSKNI | HKPGQAPRFLMYSASIRATDTPDRFSGSGSGTDFTLTI |
| | IYLQMDSLETEDTAMYWCATSHSFYYDGPFDYWGQGTLVT | SRLEPEDFAVYYCQHYGSSPPYTFGQGTKLEIK |
| | VSS (SEQ ID NO: 1468) | (SEQ ID NO: 1703) |
| AB- | EVQLVESGGGLVQPGGSLRLSCVVAGTTFSDQFMDWVRQA | AIQMTQSPSSLSASVGDRVTITCRASQVITNDLGWYQQ |
| 009328 | PGKGLEWVGRSRNKARSYTTEYAASVKGRFTISRDDSKKS | RPGKAPKLLIYATSRLQSGVPSRFSGSGSGTDFTLTIS |
| | LYLQMNTLKTEDTAMYYCTRDGGEGGYTAFDIWGQGTMVT | SLQPEDFATYYCLQDSNFPLTFGGGTKVEIK |
| | VSS (SEQ ID NO: 1469) | (SEQ ID NO: 1704) |
| AB- | QLQLQESGPGLVKPSETLSLTCTVSGGSISSSSYYWGWIR | SSELTQDPAVSVALGQTVRITCQGDSLRSYYASWYQQ |
| 009329 | QPPGKGLEWIGSIYYSGSTYYNPSLKSRVTISVDTSKNQF | KPGQAPVLVIYGKNNRPSGIPDRFSGSSSGNTASLTIT |
| | SLKLSSVTAADTAVYYCARHKMGWLQFAGWFDPWGQGTLV | GAQAEDEADYYCNSRDSSGNHWVFGGGTKLTVL |
| | TVSS (SEQ ID NO: 1470) | (SEQ ID NO: 1705) |
| AB- | QLQLQESGPGLVKPSETLSLTCTVSGGSISSSSYYWGWIR | EIVMTQSPATLSVSPGERATLSCRASQSVSSNLAWYQQ |
| 009330 | QPPGKGLEWIGSIYYSGSTYYNPSLKSRVTISVDTSKNQF | KPGQAPRLLIYGASTRATGIPARFSGSGSGTEFTLTIS |
| | SLKLSSVTAADTAVYYCARHAMSGITIFGVVTNNNWFDPW | SLQSEDFAVYYCQQYNNWPPYTFGQGTKLEIK |
| | GQGTLVTVSS (SEQ ID NO: 1471) | (SEQ ID NO: 1706) |
| AB- | QVTLKESGPALIKPTQTLTLTCTFSGFSLNTAGMRVNWIR | QSGLTQPASVSGSPGQSITISCTATSSDIGNYNYVSWY |
| 009331 | QPPGKALEWLAHIDWEDEKFYSTSLKTRLSISKDTSKDQV | QQHPGKAPKLMIYEVSSRPSGVSNRFSGSKSGNTASLT |
| | VLTMTNMDPVDTATYYCARNEWDFDAFDIWGQGILVTVSS | ISGLRAEDEANYYCSSYTTSRTYVFGTGTKVTVL |
| | (SEQ ID NO: 1472) | (SEQ ID NO: 1707) |
| AB- | AVQLVESGGGLVQPGGTLRLSCTASTFIFNNYAMSWVRQV | DIQMTQSPSTLSASVGDRVTITCRASESVSTWLAWYQQ |
| 009332 | PGKGLEWVSVISGSGSATYYADSVKGRFTISRDNSENTVD | KPGKAPQLLIYKASNLESGVPSRFSGSGSGTEFTLTIS |
| | LQMNSLRPEDTATYYCAKVRAGSGSHKAGYYFEKWGQGTL | SLQPDDSATYYCQQYSTYSTFGQGTKVEIK |
| | VIVSS (SEQ ID NO: 1473) | (SEQ ID NO: 1708) |
| AB- | QVQLQQWGTGLLKPSETLSLTCAVYGGSFAGYYWSWVRQ | EIVLTQSPATLSVSPGERATLSCRTSQIVGVNLAWYQQ |
| 009333 | PPGKGLQWIGEINHSGGTNYNPFLKSRVSISVDSSKNQFS | RPGQAPRLLIYGASTRATGIPDRFSGGGSGTQFTLTII |
| | LKLSSVTAADTAVYYCVRDSDMTLAAYYKGFQYGMDVWGQ | SLQSEDLAVYYCQQYDNWPRTFGRGTKVEIK |
| | GTTVTVSS (SEQ ID NO: 1474) | (SEQ ID NO: 1709) |
| AB- | QLQLQESGPGLVKPSETLSLTCTVSGGSITSRFDYWVWIR | QSVLTQPPSVSGTPGQRVSISCSGSSSNIGSNFVHWYQ |
| 009334 | QPPGKGLEWIGSISHGGNTYYTPSLKSRVTISIDASKRQV | QLPGAAPQLLICRNNERPSGVPDRFSGSKSGTSASLAI |
| | FLELSSVTAADTAMYHCARAYCSGGSCYVYYYFYVDVWGE | SGLRSEDEADYYCAAWDDTLSSPNWVFGGGTKLTVL |
| | GTTVTVSS (SEQ ID NO: 1475) | (SEQ ID NO: 1710) |
| AB- | QVQLQESGPGLVKPSETLSLTCTVSGGSISSHYWSWIRQP | DIQMTQSPSSLSASVGDRVTITCRASQGISNYLAWYQQ |
| 009335 | PGKGLEWIGYIYYSGSTNYNPSLKSRVTISVDTSKNQFSL | KPGKVPKLLIYAASTLQSGVPSRFSGSGSGTDFTLTIS |
| | KLSSVTAADTAVYYCARVRREWLAAYFYYGMDVWGQGTTV | SLQPEDVATYYCQKYNSAPLTFGGGTKVEIK |
| | TVSS (SEQ ID NO: 1476) | (SEQ ID NO: 1711) |
| AB- | QVQLVQSGGGVVQPGRSLRLSCAASGFSFSSYAMHWVRQA | EIVLTQSPGTLSLSPGERATLSCRASQSLGSSYLAWYQ |
| 009336 | PGKGLEWVAVISFDQTTKYYADSVKGRFTISKDNSKNMLY | QKPGQTPRLLIYGASTRATGIPDRFSGSGSGTDFTLTI |
| | LQMNTLRAEDTAVYYCARQEDYDYVWGIFRPRGWYIDLW | SRLEPEDFAMYYCQQYDSPPPYTFGQGTKLEIK |
| | GRGTLVTVSS (SEQ ID NO: 1477) | (SEQ ID NO: 1712) |
| AB- | EVQLLESGGGLVQPGGSLRLSCAASGFTFSSYAMSWVRQA | EIVMTQSPATLSVSPGERATLSCRASQSVSSNLAWYQQ |
| 009337 | PGKGLEWVSAISGSGGSTYYADSVKGRFTISRDNSKNTLY | KPGQAPRLLIYGASTRATGIPARFSGSGSGTEFTLTIS |
| | LQMNSLRAEDTAVYYCAKTTGVTMIVVVIEGGYFDYWGQG | SLQSEDFAVYYCQQYNNWLSYTFGQGTKLEIK |
| | TLVTVSS (SEQ ID NO: 1478) | (SEQ ID NO: 1713) |
| AB- | QVQLVESGGGVVQPGRSLRLSCAASGFTFSSYGMHWVRQA | QSALTQPASVSGSPGQSITISCTGTSSDVGGYNYVSWY |
| 009338 | PGKGLEWVAVIWYDGSNKYCADSVKGRFTISRDTSKNTLY | QQHPGKAPKLMIYDVSNRPSGVSNRFSGSKSGNTASLT |
| | LQMNSLRAEDTAVYYCAREAKPAGGRSFTIFGVATYYYYM | ISGLQAEDEADYYCSSYTSSSTLIFGGGTKLTVL |
| | DVWGKGTTVTVSS (SEQ ID NO: 1479) | (SEQ ID NO: 1714) |
| AB- | QVQLQESGPGLVKPSETLSLTCTVSGGSISSHYWSWIRQP | EIVLTQSPGTLSLSPGERATLSCRASQSVSSSYLAWYQ |
| 009339 | PGKGLEWIGYIYYSGSTNYNPSLKSRVTISVDTSKNQFSL | QKPGQAPRLLIYGASSRATGIPDRFSGSGSGTDFSLTI |
| | KLSSVTAADTAVYYCARAPRGWLQLSPYYFDLWGRGTLVT | SRLEPEDFAVYYCHQYGSSPTFGQGTKVEIK |
| | VSS (SEQ ID NO: 1480) | (SEQ ID NO: 1715) |
| AB- | QVQLVQSGAEVKKPGASVKVSCKVSGYTLTELSMHWVRQ | QSALTQPASVSGSPGQSITISCTGTSSDVGGYNYVSWY |
| 009340 | APGKGLEWMGGFDPEDGETIYAQKFQGRVTMTEDTSTDTA | QQHPGKAPKLMIYDVSKRPSGVSNRFSGSKSGNTASLT |
| | YMELSSLRSEDTAVYYCATAFAYCGGDCYYPSYWGQGTL | ISGLQAEDEADYYCCSYAGSSSPWVFGGGTKLTVL |
| | VTVSS (SEQ ID NO: 1481) | (SEQ ID NO: 1716) |
| AB- | QLQLQESGPGLVKPSETLSLTCTVSGGSISSSSYYWGWIR | EIVMTQSPATLSVSPGERATLSCRASQSVSSNLAWYQQ |
| 009341 | QPPGKGLEWIGSIYYSGSTYYNPSLKSRVTISVDTSKNQF | KPGQAPRLLIYGASTRATGIPARFSGSGSGTEFTLTIS |
| | SLKLSSVTAADTAVYYCARQMPNRPMIVVVTIRGWFDPWG | SLQSEDFAVYYCQQYNNWPRTFGQGTKVEIK |
| | QGTLVTVSS (SEQ ID NO: 1482) | (SEQ ID NO: 1717) |
| AB- | EEQLLESGGDLVQPGGSLRLSCAASGFTFSTYAMSWVRQA | ETVMTQSPATLSVSPGEGATLSCRASQSVSSNLAWYQ |
| 009342 | PGKGLEWVSAISGGGHFTYYADSVEGRFTISRDNSKNTLY | QKPGQAPRLLIYGASTRATGFPARFSGSGSGTEFTLTI |
| | LQMDRLRADDTAVYYCAKDPRLVTTGWYYDLWGRGTLVTV | SSMQSEDFAVYFCQQYHNWPPAFGPGTRVEIK |
| | SS (SEQ ID NO: 1483) | (SEQ ID NO: 1718) |
| AB- | QAQLVESGGGVVQPGRSLRLSCAASGFTFNNHGMHWVRQ | SYELTQPPSMSVSPGQTASITCSGYILGDVSASWYQQK |
| 009343 | VPGKGLEWVAVISYEGSKKDYADSVKGRFSISRDNSKQTV | PGQSPVLIIYQDTKRPSGIPDRFSGSDSGNTATLTISG |
| | YLQMNSLRVEDTAVYYCAKDAAIFMIYPSRYFDLWGRGTL | TQATDEADYYCQAWDSRTHVVFGGGTKLTVL |
| | LTVSS (SEQ ID NO: 1484) | (SEQ ID NO: 1719) |
| AB- | EVQLVESGGGLVKPGGSLRLSCAASGFTFSSYSMNWVRQA | EIVLTQSPGTLSLSPGERATLSCRASQSVSSSYLAWYQ |
| 009344 | PGKGLEWVSSISSSSSYIYYADSVKGRFTISRDNAKNSLY | QKPGQAPRLLIYGASSRATGIPDRFSGSGSGTDFTLTI |
| | LQMNSLRAEDTAVYYCARGWYQLLLPYYYMDVWGKGTTVT | SRLEPEDFAVYYCQQYGSSPPKYTFGQGTKLEIK |
| | VSS (SEQ ID NO: 1485) | (SEQ ID NO: 1720) |
| AB- | QVQLVESGGGVVQPGRSLRLSCAASGFSFSTYAMHWVRQA | DIQMTQSPSSLSASVGDRVTITCRASQSISTYLNWYQQ |
| 009345 | PGKGLEWVAFMSYDGRNEYYADSVKGRFTTSRDNSKNTL | KPGKAPKLLIYAASSLQSGVPSRFSARGSGADFTLTIS |
| | YLQMNSLRTEDTAVYYCARVPGGVLGTFDIWGQGTMVTV | LSQPEDFATYFCQQSYSAPPTFGQGTKVEIK |
| | SS (SEQ ID NO: 1486) | (SEQ ID NO: 1721) |
| AB- | EVQLVESGGGVVRPGGSLRLSCAASGFTFDDYGMSWVRQA | QSALTQPRSVSGSPGQSVTISCTGTSSDVGGYNYVSWY |
| 009346 | PGKGLEWVSGINWNGGSTGYADSVKGRFTISRDNAKNSLY | QQHPGKAPKLMIYDVSKRPSGVPDRFSGSKSGNTASLT |
| | LQMNSLRAEDTALYYCARWPLLGISGYYHYYMDVWGKGT | ISGLQAEDEADYYCCSYAGSYTLLFGGGTKLIVL |
| | TVTVSS (SEQ ID NO: 1487) | (SEQ ID NO: 1722) |
| AB- | EVQLVESGGGLVQPGGSLRLSCGASSFIFSRYAMHWVRQA | QPVLTQPPSASASLGASVTLTCTLSSGYYSYKVDWYQ |
| 009347 | PGKGLEYVSTISSNGSNTYYAKSVKDRFIISRDNSKSTLY | QRPGKGPRFVMRVGTGGIVKAKGDGIPDRFSVLGSGL |
| | LQMGSLRVEDMAVYYCARSPGLAIHSSTSLDVWGKGTTVT | NRSLTIKDIQEEDEGDYHCGADHGSGGNFVWVFGGGT |
| | VSS (SEQ ID NO: 1488) | KLTVL (SEQ ID NO: 1723) |
| AB- | QVTLKESGPVLVKPTETLTLTCTVSGFSLSNARMGVSWIR | EIVLTQSPGTLSLSPGERATLSCRASQSVSSSYLAWYQ |
| 009348 | QPPGKALEWLAHIFSNDEKSYSTSLKSRLTISKDTSKSQV | QKPGQAPRLLIYGASSRATGIPDRFSGSGSGTDFTLTI |
| | VLTMTNMDPVDTATYYCARIRAQWLTFYYYMDVWGKGTTV | SRLEPEDFAVYYCQQYGSSPPYTFGQGTKLEIK |
| | TVSS (SEQ ID NO: 1489) | (SEQ ID NO: 1724) |
| AB- | QVQLVESGGGVVQPGRSLRLSCAASGFSFSTHGMHWVRQA | DIQMTQSPSSVSASVGDRVTITCRASQGISSRLAWYQQ |
| 009349 | PGKGLEWVAAIWYDGSNENYADSVRGRFTISRDNSKNILY | KPGKAPKLLIYASSTLQSGVPSRFSGSGSGTDFTLTIS |
| | LQMSSLRAEDTAIYYCARNHIPGSSLGFDPWGQGTLVTVS | SLQPEDFATYYCQQASSLPYTFGQGTKLEIK |
| | S (SEQ ID NO: 1490) | (SEQ ID NO: 1725) |
| AB- | EVQLVESGGGLVQPGRSLRLSCTVSGFIIDDYAMSWVRQA | SSELTQDAAVSVALGQTVKITCQGDSLRRYYASWYQQ |
| 009350 | PGKGLEWVGFIRSKTYGGTTEYAASVKGRFTISRDDSKGI | KPGQAPVLVMYGKNKRPSGIPDRFSVSTSLTTASLTIT |
| | VYLQMNSLQTQDTGVYYCTKDGRYDWNFILDSYYYYYMDV | GAQAEDEADYYCNSRDTSGDHRVFGGGTKLTVL |
| | WGKGTTVTVSS (SEQ ID NO: 1491) | (SEQ ID NO: 1726) |
| AB- | QGQLVQSGAEVRKPGSSVKVSCKASGGTLSTYAISWVRQA | SYVLTQPPSLSVAPGKTATITCGKNNIESESVHWYQSK |
| 009351 | PGQGLEWMGRITPIFGTTNYAQKFQGRVTITADESTSTAY | PGQAPVLVVYDDSDRPSGIPERFSGSNSGNTATLTISR |
| | MEVSSLRFEDTAVYYCARGASGWFGMDVWGQGTTVTVSS | VEAGDEADYYCQVWDSTGVHVVFGGGTKLTVL |
| | (SEQ ID NO: 1492) | (SEQ ID NO: 1727) |
| AB- | EVRLVQSATEMKKPGASVKISCKTSGYMFSKFGISWVRLA | DILLTQSPATLSLSPGARVTLSCRASQNVGENFAWYQR |
| 009352 | PGRGLEWMGWITPYNSDTVYAQNFQGRLTVTLDRAATTVS | RPGQSPRLLIYGKSIRATDVPARFSGSGSGTEFTLAIS |
| | LELHNLRSDDTGRYYCARDPWRDCSSCWGQGTLLTVSS | SLQSEDFAVYYCQQYDLFPWTFGQGTTIEFK |
| | (SEQ ID NO: 1493) | (SEQ ID NO: 1728) |
| AB- | QAQLVQSGAEVKKPGASVKVSCKASGYAFGSSDINWVRQA | EIVLTQSPGTLSLSPGERATLSCRASQSVSNSYLAWYQ |
| 009353 | TGQGLEWMGWMHPDSGNTVYAQNFQDRVTMTRDTSTST | QKPGQAPRLVIYGASSRATGIPDRFSGSGSGTDFTLTI |
| | AYMELRSLRSDDTAVYYCARGPRLVRFLQWSLNYMDVWG | SRLEPEDFAVYYCQQYGSSRGITFGQGTRLEIK |
| | KGTTVTVSS (SEQ ID NO: 1494) | (SEQ ID NO: 1729) |
| AB- | EVQLVESGGGLVQPGGTLRLSCVASGFNFSNYGMSWVRQA | DVVMTQTPLSLSVTPGQPASISCKSSQSLLHSDGKTFL |
| 009354 | PGKGLEWVSAISGGGDGASYADSVKGRFTISRDNSKNTLY | YWYLQKPGQPPQLLIYEVSNRFSGVPDRFTGSGSGTDF |
| | LQMNSLRADDTAVYYCAKIADWGQGTLVTVSS | TLKISRVEAEDVGVYYCMQSIQLPLTFGGGTKVEIK |
| | (SEQ ID NO: 1495) | (SEQ ID NO: 1730) |
| AB- | QLQLQESGPGLVKPSETLSLTCTVSSGSIRSSNYYWGWIR | DIQLTQSPSSLSASVGDRVSITCRASQNIDTYLNWYQQ |
| 009355 | QPPGKGLEWIGSVYDSGRTEYNPSLKSRVTISVDTSKNQF | KPGKAPKLLISAASSLRSGVPSRFSGSGSGTHFTLTIS |
| | SLKLRSVTAADTAVYYCARHAGPHMVRGVMRNNWFDPWGQ | SLQPEDVTTYYCQQSYSIPWTFGQGTKVEIR |
| | GSLVSVSS (SEQ ID NO: 1496) | (SEQ ID NO: 1731) |
| AB- | QVQLVQSGAEVKKPGASVKVSCKASGYTFVDYAIHWVRQ | EIVMTQSPAILSVSPGERATLSCRASQSVANNLAWYQQ |
| 009356 | APGQGLEWMGWIHAGNGNTKDSEKFQGRVTITRDTSANTV | KPGQAPRLVIFTASTRAPGVPDRFSASGSGTEFTLTIN |
| | FLELSSLRSEDTAVYYCAIAAAGTQVVWGQGTLVTVSS | SLQSEDFAVYYCQQYYDWPPVTFGGGTKVEIK |
| | (SEQ ID NO: 1497) | (SEQ ID NO: 1732) |
| AB- | QLQLQESGPGLVKPSETLSLTCTVSGGSISSSSYYWGWIR | DIQMTQSPSSVSASVGDRVTITCRASQGISSWLAWYQQ |
| 009357 | QPPGKGLEWIGSIYYSGSTYYNPSLKSRVTISVDTSKNQF | KPGKAPKLLIYAASSLQSGVPSRFSGSGSGTDFTLTIS |
| | SLKLSSVTAADTAVYYCARHPLSSITIFGVVKREYYFDYW | SLQPEDFATYYCQQANSFPLLFGGGTKVEIK |
| | GQGTLVTVSS (SEQ ID NO: 1498) | (SEQ ID NO: 1733) |
| AB- | QVHLVQSGAEVKKPGASVKISCKASGYTFTSYYVHWVRQA | QSALTQEASVSATVGQKVTLSCTGNTNNIGGYAVGW |
| 009358 | PGQGLEWMGIIKPSGAGTIYAQKFQGRVTMTRDTSTSTVY | HQQISHGAPKTVMFGNSLPSGIPDRFSGSKSGTTASLI |
| | MELSSLISEDTAVYYCARGGGVTDRDYWGQGTLVTVSS | ISGLQPEDEADYYCSTWDSSLGAVLFGGGTKLTVL |
| | (SEQ ID NO: 1499) | (SEQ ID NO: 1734) |
| AB- | GVQLVQSGAEVKKPGDSLKISCKGFGYRYTNYWVAWVRQ | QSVLTQPPSVSGAPGQGVTISCTGTSSNIGAGYGVHWY |
| 009359 | MPGKGLEWLGIIYPADSDTRYSPSFQGQVTISADKSITTA | QQFPGTAPILIIYDNTNRPSGVPDRFSGSRSGASASLA |
| | YLQWKSLKASDTAIYYCARSFEYCTGTTCQSWFDFWGQGT | ITDLQADDEADYYCHSYDTTLSGQGVFGGGTKLTVL |
| | LVTVAS (SEQ ID NO: 1500) | (SEQ ID NO: 1735) |
| AB- | QVQLVQSGAEVKKPGASVKVSCKASGYTFTSYDINWVRQA | SYELTQPPSVSVSPGQTASITCSGDKLGDKYACWYQQ |
| 009360 | TGQGLEWMGWMNPNSGNTGYAQKFQGRVTMTRNTSISTA | KPGQSPVLVIYQDSKRPSGIPERFSGSNSGNTATLTIS |
| | YMELSSLRSEDTAVYYCARGRAYQLLFNYYYYMDVWGKG | GTQAMDEADYYCQAWDSSTVVFGGGTKLTVL |
| | TTVTVSS (SEQ ID NO: 1501) | (SEQ ID NO: 1736) |
| AB- | EVQLVESGGGLAQPGGTLRLSCTGYGFIFNNYGLTWVRQA | QSVLAQPPSASETPGQSVTISCSGSTSNIGSNFVYWYQ |
| 009361 | PGKGLEWLSVLSGSSYIKYEADSVRGRFTISRDNSKNTLY | QLPGTAPKLLIYRNTQRPSGVPDRFSGSKSATSASLAI |
| | LEMNSLTAEDTAVYYCARKGVGSQDYGLDVWGQGTTVIVS | SGLRPEDEADYYCAAWDDSLSGGVFGTGTKVTVL |
| | S (SEQ ID NO: 1502) | (SEQ ID NO: 1737) |
| AB- | QVQLVQSGAEVKKPGASVKVSCKASGYTFTSYYMHWVRQ | SYELTQPPSVSVSPGQTARITCSGDALPKQYAYWYQQ |
| 009362 | APGQGLEWMGIINPSGGSTSYAQKFQGRVTMTRDTSTSTV | KPGQAPVLVIYKDSERPSGIPERFSGSSSGTTVTLTIS |
| | YMELSSLRSEDTAVYYCARDPTTVTTYDYYYGMDVWGQGT | GVQAEDEADYYCQSADSSGTFVVFGGGTKLTVL |
| | TVTVSS (SEQ ID NO: 1503) | (SEQ IDNO: 1738) |
| AB- | EVQLLESGGGLVQPGGSLRLSCAASGFTFSSFAMTWIRQA | DIVMTQSPDSLAVSLGERATLNCKSSQSVLYSSNNKNY |
| 009363 | PGKGLEWISAIGGDGGATYYVDSIKGRFTISRDNSKNTVY | LAWYQQKPGQPPKLLFYWASTRQSGVPDRFSGSGSGT |
| | LHMNGLRVEDTAVYYCATPSPTRYSWGQGTLVTVSS | DFTLTISSLQAEDGAVYYCQQYHKTPHTFGQGTKLEIK |
| | (SEQ ID NO: 1504) | (SEQ ID NO: 1739) |
| AB- | QVQLVESGGGVVQPGRSLRLSCAASGFTFSSYAMHWVRQA | DVVMTQSPLSLPVTLGQPASISCRSSQSLVHSDGNTYL |
| 009364 | PGKGLEWVAVISYDGSNKYYADSVKGRFTISRDNSKNTLY | NWFQQRPGQSPRRLIYKVSNRDSGVPDRFSGSGSGTDF |
| | LQMNSLRAEDTAVYYCARDPDYYDSSSDAFDIWGQGTMV | TLKISRVEAEDVGVYYCMQGTHWPPWTFGQGTKVEIK |
| | TVSS (SEQ ID NO: 1505) | (SEQ ID NO: 1740) |
| AB- | QVQLVQSGAEVKKPGASVKVSCKVSGYTLTELSMHWVRQ | DIMMTQTPLSSPVTLGQPASISCRSSQSLVHSDGNTYL |
| 009609 | APGKGLEWMGGFDPEDAETIYAQKFQGRVTMTEDTSTDTA | SWLQQRPGQPPRLLIYKISNRFSGVPDRFSGSGAGTDF |
| | YMELSSLRSEDTAVYYCATALAVTGTSDSYYYYYGMDVW | TLKISRVEAEDVGVYYCMQATQFPWTFGQGTKVEIK |
| | GQGTTVTVSS (SEQ ID NO: 1506) | (SEQ ID NO: 1741) |
| AB- | QVTLRESGPALVKPTQTPTLTCTFSGFSLSTSGMCVSWIR | DIQMTQSPSSLSASVGDRVTITCRASQSISSYLNWYQQ |
| 009611 | QPPGKALEWLARIDWDDDKYYSTSLKTRLTISKDTSKNQV | KPGKAPKLLIYAASSLQSGVPSRFSGSGSGTDFTLTIS |
| | VLTMTNMDPVDTATYYCAREGVATISLDYWGQGTLVTVSS | SLQPEDFATYYCQQSYSTPRTFGQGTKVEIK |
| | (SEQ ID NO: 1507) | (SEQ ID NO: 1742) |
| AB- | QVTLRESGPALVKPTQTLTLTCTFSGFSLSTSGMCVSWIR | DIQMTQSPSSLSASVGDRVTITCRASQSISSYLNWYQQ |
| 009612 | QPPGKALEWLARIDWDDDKYYSTSLKTRLTISKDTSKNQV | KPGKAPKLLIYAASSLQSGVPSRFSGSGSGTDFTLTIS |
| | VLTMTNMDPVDTATYYCARTQANRVAFDIWGQGTMVTVSS | SLQPEDFATYYCQQSYSTPRTFGQGTKVEIK |
| | (SEQ ID NO: 1508) | (SEQ ID NO: 1743) |
| AB- | QVQLVESGGGVVQPGRSLRLSCAASGFTFSSYGMHWVRQA | EIVMTQSPATLSVSPGERATLSCRASQSVSSNLAWYQQ |
| 009614 | PGKGLEWVAVISYDGSNKYYADSVKGRFTISRDNSKNTLY | KPGQAPRLLIYGASTRATGIPARFSGSGSGTEFTLTIS |
| | LQMNSLRAEDTAVYYCAKDQKYYDILTGYYRSTYYYYYY | SLQSEDFAVYYCQQYNNWPPLTFGGGTKVEIK |
| | GMDVWGQGTTVTVSS (SEQ ID NO: 1509) | (SEQ ID NO: 1744) |
| AB- | QVQLVESGGGVVQPGRSLRLSCAASGFTFSSYGMHWVRQA | DIQMTQSPSSLSASVGDRVTITCQASQDISNYLNWYQQ |
| 009615 | PGKGLEWVAVIWYDGSNKYYADSVKGRFTISRDNSKNTLY | KPGKAPKLLIYDASNLETGVPSRFSGSGSGTDFTFTIS |
| | LQMNSLRAEDTAVYYCAKVGSPYCSGGSCYSGYFDYWGQ | SLQPEDIATYYCQQYDNLPPLTFGGGTKVEIK |
| | GTLVTVSS (SEQ ID NO: 1510) | (SEQ ID NO: 1745) |
| AB- | QVQLVESGGGVVQPGRSLRLSCAASGFTFSSYGMHWVRQA | SYVLTQPPSVSVAPGKTARITCGGNNIGSKSVHWYQQ |
| 009616 | PGKGLEWVAVISYDGSNKYYADSVKGRFTISRDNSKNTLY | KPGQAPVLVVYDDSDRPSGIPERFSGSNSGNTATLTIS |
| | LQMNSLRAEDTAVYYCAKSPEGAAAGTGYFDYWGQGTLV | RVEAGDEADYYCQVWDSSSDPWVFGGGTKLTVL |
| | TVSS (SEQ ID NO: 1511) | (SEQ ID NO: 1746) |
| AB- | QVQLVQSGAEVKKPGASVKVSCKVSGYTLIELSMHWVRQ | QSALTQPASVSGSPGQSITISCTGTSSDVGGYNYVSWY |
| 009619 | APGKGLEWMGGFDPEDGETIYAQKFQGRVTMTEDTSTDTA | QQHPGKAPKLMIYDVSNRPSGVSNRFSGSKSGNTASLT |
| | YMELSSLRSEDTAVYYCATGPAAATSGWFDPWGQGTLVT | ISGLQAEDEADYYCSSYTSSSTRVFGGGTKLTVL |
| | VSS (SEQ ID NO: 1512) | (SEQ ID NO: 1747) |
| AB- | QITLKESGPTLVKPTQTLTLTCTFSGFSLSTSGVGVGWIR | QSALTQPASVSGSPGQSITISCTGTSSDVGAYNYVSWY |
| 009620 | QPPGKALEWLALIYWDDDKRYSPSLKSRLTITKDTSKNQV | QQHPGKAPKLMIYEVSNRPSGVSNRFSGSKSGNTASLT |
| | VLTMTNMDPVDTATYYCAHHEVATVFDYWGQGTLVTVSS | ISGLQAEDEADYYCSSYTSSSTVFGGGTKLTVL |
| | (SEQ ID NO: 1513) | (SEQ ID NO: 1748) |
| AB- | QVQLQQWGAGLLKPSETLSLTCAVYGGSFSGYYWSWIRQP | SSELTQDPAVSVALGQTVRITCQGDSLRSYYASWYQQ |
| 009629 | PGKGLEWIGEINHSGSTNYNPSLKSRVTISVDTSKNQFSL | KPGQAPVLVIYGKNNRPSGIPDRFSGSSSGNTASLTIT |
| | KLSSVTAADTAVYFCARGTGIAVAGVLVNFYYCYYMDVWG | GAQAEDEADYYCNSRDSSGNHLVFGGGTKLTVL |
| | KGTTVTVSS (SEQ ID NO: 1514) | (SEQ ID NO: 1749) |
| AB- | EVQLVESGGGLVQPGGSLRLSCAASGFNFSFYWMSWVRQA | SYELTQPPSVSVSPGQTASITCSGDKLGDKYACWYQQ |
| 009631 | PGKGLEWVANIKQDGSEKYYVDSVKGRFTISRDNAKNSLY | KPGQSPVVVIYQDSKRPSGIPERFSGSNSGNTATLTIS |
| | LQMNSLRAEDTAVYYCARLSGGSWYFDYWGQGTLVTVSS | GTQAMDEADYYCQAWDSSTGVFGGGTKLTVL |
| | (SEQ ID NO: 1515) | (SEQ ID NO: 1750) |
| AB- | QVQLQESGPGLVKPSETLSLTCTVSGGSISSYYWSWIRQP | EIVLTQSPGTLSLSPGERATLSCRASQSVGSIYLAWYQ |
| 009633 | PGKGLEWIGYIYYSGSTNYNPSLKSRVTISVDTSKNQFSL | QKPGQAPRLLIYGASSRATGIPDRFSGSGSGTDFTLTI |
| | KLSSVTAADTAVYYCARGFDYWGQGTLVTVSS | SRLEPEDFAVYYCQQYGSSLTWTFGQGTKVEIK |
| | (SEQ ID NO: 1516) | (SEQ ID NO: 1751) |
| AB- | QVTLKESGPALVKPTQTLTLTCTFSGFSLSTSGMRVSWIR | DIVMTQSPDSLAVSLGERATINCKSSQSVLYSSNNKNY |
| 009634 | QPPGKALEWLARIDWDDDKFYSTSLKTRLTISKDTSKNQV | LAWYQQKPGQPPKLLIYWASTRESGVPDRFSGSGSGT |
| | VLTMTNMDPVDTATYYCARYYYYGMDVWGQGTTVTVSS | DFTLTISSLQAEDVAVYYCQQYYSTLWTFGQGTKVEIK |
| | (SEQ ID NO: 1517) | (SEQ ID NO: 1752) |
| AB- | EVQLVESGGGLVKPGGSLRLSCAASGFTFSNAWMSWVRQA | SYELTQPPSVSVSPGQTARITCSGDALPKKYAYWYQQ |
| 009635 | PGKGLEWVGRIKSKTDGGTTDYAAPVKGRFTISRDDSKNT | KSGQAPVLVIYEDSKRPSGIPERFSGSSSGTMATLTIS |
| | LYLQMNSLKTEDTAVYYCTTGRYYYDSSGYANDAFDIWGQ | GAQVEDEADYYCYSTDSSGNHRVFGGGTKLTVL |
| | GTMVTVSS (SEQ ID NO: 1518) | (SEQ ID NO: 1753) |
| AB- | QVQLVQSGAEVKKPGSSVKVSCKASGGTFSSYAISWVRQA | DIVMTQSPLSLPVTPGEPASISCRSSQSLLHSNGYNYL |
| 009637 | PGQGLEWMGGIIPILGTANYAQKFQGRVTITADKSTSTAY | DWYLQKPGQSPQLLIYLGSNRASGVPDRFSGSGSGTDF |
| | MELSSLRSEDTAMYYCAREGGFYGSGRNFDYWGQGTLVTV | TLKISRVEAEDVGVYYCMQALQTPYTFGQGTKLEIK |
| | SS (SEQ ID NO: 1519) | (SEQ ID NO: 1754) |
| AB- | QVQLVESGGGLVKPGGSLRLSCAASGFTFSDYYMSWIRQA | SYELTQPPSVSVSPGQTARITCSGDALPKQYAYWYQQ |
| 009638 | PGKGLEWVSYISSSGSTIYYADSVKGRFTISRDNAKNSLY | KPGQAPVLVIYKDSERPSGIPERFSGSSSGTTVTLTIS |
| | LQMNSLRAEDTAVYYCARDSTRYCSSTSCSSYYYGMDVWG | GVQAEDEADYYCQSADSSGTWVFGGGTKLTVL |
| | QGTTVTVSS (SEQ ID NO: 1520) | (SEQ ID NO: 1755) |
| AB- | QVQLVESGGGVVQPGRSLRLSCAASGFTFSSYAMHWVRQA | DVVMTQSPLSLPVTLGQPASISCRSSQSLVHSDGNTYL |
| 009640 | PGKGLEWVAVISYDGSNKYYADSVKGRFTISRDNSKNTLY | NWFQQRPGQSPRRLIYKVSNRDSGVPDRFSGSGSGTDF |
| | LQMNSLRAEDTAVYYCARDSDDYGDYYFDYWGQGTLVT | TLKISRVEAEDVGVYYCMQGTHWPPWTFGQGTKVEIK |
| | VSS (SEQ ID NO: 1521) | (SEQ ID NO: 1756) |
| AB- | QVQLQQWGAGLLKPSETLSLTCAVYGGSFSGYSWSWIRQP | SSELTQDPAVSVALGQTVRITCQGDSLRSYYASWYQQ |
| 009642 | PGKGLEWIGEINHSGSTNYNPSLKSRVTISVDTSKNQFSL | KPGQAPVLVIYGKNNRPSGIPDRFSGSNSGNTASLTIT |
| | KLSSVTAADTAVYYCAREGRFLEWLLGVNYYYYMDVWGKG | GAQAEDEADYYCHSRDSSGNHLVFGGGTKLTVL |
| | TTVTVSS (SEQ ID NO: 1522) | (SEQ ID NO: 1757) |
| AB- | EVQLVESGGGLVQPGRSLRLSCTASGFTFGDYAMSWVRQA | DIQMTQSPSTLSASVGDRVTITCRASQSISSWLAWYQQ |
| 009643 | PGKGLEWVGFIRSKAYGGTTEYAASVKGRFTISRDDSKSI | KPGKAPKLLIYKASSLESGVPSRFSGSGSGTEFTLTIS |
| | AYLQMNSLKTEDTAVYYCTREDFWSGYSDYWGQGTLVTVS | SLQPDDFATYYCQQYNSYPWTFGQGTKVEIK |
| | S (SEQ ID NO: 1523) | (SEQ ID NO: 1758) |
| AB- | QVQLVESGGGVVQPGRSLRLSCAASGFTFSSYGMHWVRQA | DIQMTQSPSSLSASVRDRVTITCRASQSISSYLNWYQQ |
| 009644 | PGKGLEWVAVISYDGSNKYYADSVKGRFTISRDNSKNTLY | KPGKAPKLLIYAASSLQSGVPSRFSGSGSGTDFTLTIS |
| | LQMNSLRAEDTAVYYCAKDPGITIFGVVYYYYMDVWGKG | SLQPEDFATYYCQQSYSTPHTFGQGTKLEIK |
| | TTVSISS (SEQ ID NO: 1524) | (SEQ ID NO: 1759) |
| AB- | QVQLVESGGGVVQPGRSLRLSCAASGFTFSSYAMHWVRQA | SYELTQPPSVSVSPGQTARITCSGDALPKQYAYWYQQ |
| 009646 | PGKGLEWVAVISYDGSNKYYADSVKGRFTISRDNSKNTLY | KPGQAPVLVIYKDSERPSGIPERFSGSSSGTTVTLTIS |
| | LQMNSLRAEDTAVYYCARACSSTSCYKGDAFDIWGQGTM | GVQAEDEADYYCQSADSSGTYSWVFGGGTKLTVL |
| | VTVSS (SEQ ID NO: 1525) | (SEQ ID NO: 1760) |
| AB- | QVQLVQSGAEVKKPGASVKVSCKASGYTFAGYDINWVRQ | QAVLTQPASLSASPGASASLTCTLRSGINVDSYRIYWY |
| 009647 | APGQGLEWMGWASTKNDNTGYAQKFQGRVTMTRDIFTST | HQKPGSPPQYLLNYKSAADKEQGSGVPSRFSGSKDAS |
| | VFMELSSLNFEDTAVYYCARGVDQGVDYWGQGTLVTVSS | ANAGILIISGLQSEDEADYYCMIWHNSAYFFGTGTKVT |
| | (SEQ ID NO: 1526) | VL (SEQ ID NO: 1761) |
| AB- | EVQLVESGGGLVQPGGSLRLSCAASGFTFSSYWMSWVRQA | SYELTQPPSVSVSPGQTASITCSGDKLGDKYACWYQQ |
| 009649 | PGKGLEWVANIKQDGSEKYYVDSVKGRFTISRDNAKNSLY | KPGQSPVLVIYQDSKRPSGIPERFSGSNSGNTATLTIS |
| | LQMNSLRAEDTAVYYCARDFNDFWSGYYGPSFDYWGQGT | GTQAMDEADYYCQAWDSSTGVFGGGTKLTVL |
| | LVTVSS (SEQ ID NO: 1527) | (SEQ ID NO: 1762) |
| AB- | QVQLVESGGGVVQPGRSLRLSCAASGFTFSSYAMHWVRQA | DIQMTQSPSSLSASVGDRVTITCQASQDISNYLNWYQQ |
| 009650 | PGKGLEWVAVISYDGSNKYYADSVKGRFTISRDNSKNTLY | KPGKAPKLLIYDASNLETGVPSRFSGSGSGTDFTFTIS |
| | LQMNSLRAEDTAVYYCARVWVPKVYYYGMDVWGQGTTV | SLQPEDIATYYCQQYDNLPITFGQGTRLEIK |
| | TVSS (SEQ ID NO: 1528) | (SEQ ID NO: 1763) |
| AB- | QVQLQQWGAGLLKPSETLSLTCAVYGGSFSGYYWSWIRQP | QSALTQPASVSGSPGQSITISCTGTSSDVGSYNLVSWY |
| 009654 | PGKGLEWIGEINHSGSTNYNPSLKSRVTISVDTSKNQFSL | QQHPGKAPKLMIHEGSKRPSGVSNRFSGSKSGNTASLT |
| | KLSSVTAADTAVYYCARSWSSWSYAFDIWGQGTMVTVSS | ISGLQAEDEADYYCCSYAGSSTWVFGGGTKLTVL |
| | (SEQ ID NO: 1529) | (SEQ ID NO: 1764) |
| AB- | EVQLVESGGGLVQPGGSLRLSCAASGFTFSSYWMSWVRQA | SYELTQPPSVSVSPGQTASITCSGDKLGDKYACWYQQ |
| 009655 | PGKGLEWVANIKQDGSEKYYVDSVKGRFTISRDNAKNSLY | KPGQSPVLVIYQDSKRPSGIPERFSGSNSGNTATLTIS |
| | LQMNSLRAEDTAVYYCARDSSDPIGAFDIWGQGTMVTVSS | GTQAMDEADYYCQAWDSSTVVFGGGTKLTVL |
| | (SEQ ID NO: 1530) | (SEQ ID NO: 1765) |
| AB- | EVQLVESGGGLVKPGGSLRLSCAASGFTFSSYSMNWVRQA | SYELTQPPSVSVSPGQTASITCSGDKLGDKYACWYQQ |
| 009657 | PGKGLEWVSSISSSSSYIYYADSVKGRFTISRDNAKNSLY | KPGQSPVLVIYQDSKRPSGIPERFSGSNSGNTATLTIS |
| | LQMNSLRAEDTAVYYCATIPAAMESAYYYYGMDVWGQGTT | GTQAMDEADYYCQAWDSSTVVFGGGTKLTVL |
| | VTVSS (SEQ ID NO: 1531) | (SEQ ID NO: 1766) |
| AB- | EVQLLESGGGLVQPGGSLRLSCAASGFTFSSYAMSWVRQA | DIVMTQTPLSSPVTLGQPASISCRSSQSLVHSDGNTYL |
| 009659 | PGKGLEWVSAISGSGGSTYYADSVKGRFTISRDNSKNTLY | SWLQQRPGQPPRLLIYKISNRFSGVPDRFSGSGAGTDF |
| | LQMNSLRAEDTAVYYCAKRLDYWGQGTLVTVSS | TLKISRVEAEDVGVYYCTQATQFPWTFGQGTKVEIK |
| | (SEQ ID NO: 1532) | (SEQID NO: 1767) |
| AB- | QVQLVESGGGVVQPGRSLRLSCAASGFTFSSYGMHWVRQA | DIVMTQSPDSLAVSLGERATINCKSSQSVLYSSNNKNY |
| 009661 | PGKGLEWVAVISYDGSNKYYADSVKGRFTISRDNSKNTLY | LAWYQQKPGQPPKLLIYWASTRESGVPDRFSGSGSGT |
| | LQMNSLRAEDTAVYYCAKDYGDYGGMDVWGQGTTVTVS | DFTLTISSLQAEDVAVYYCQQYYSTPRFGPGTKVDIK |
| | S (SEQ ID NO: 1533) | (SEQ ID NO: 1768) |
| AB- | QVQLVESGGGVVQPGRSLRLSCAASGFSFRSFGMHWVRQA | SSKLTQDPAVSVALGQTVTITCQGDSLRNYYASWYQQ |
| 009664 | PGKGLEWVALITYDATEKHFGDSVKGRFTISRDNPKNTVY | KPGQAPVLVIYAQNNRPSGIPDRFSGSISGTTASLTIT |
| | LQMNSLTTEDTAVYYCVKDLGYKDNWGQGTLVIVSS | GAQAEDEADYYCNSRDSSGDRLVFGGGTKLTVV |
| | (SEQ ID NO: 1534) | (SEQ ID NO: 1769) |
| AB- | EVQLLESGGGLVQPGGSLRLSCAASGFTFSSYAMSWVRQA | SYELTQPPSVSVSPGQTASITCSGDKLGDKYACWYQQ |
| 009667 | PGKGLEWVSAISGSGGSTYYADSVKGRFTISRDNSKNTLY | KPGQSPVLVIYQDTKRPSGIPERFSGSNSGNTATLTIS |
| | LQMNSLRAEDTAVYYCAKHARRHDYGDYFDYWGQGTLVT | GTQAMDEADYYCQAWDSSTAWVFGGGTKLTVL |
| | VSS (SEQ ID NO: 1535) | (SEQ ID NO: 1770) |
| AB- | QVQLQESGPGLVKPSETLSLTCTVSGGSISSYYWSWIRQP | EIVLTQSPGTLSLSPGERATLSCRASQSVSSNYLAWYQ |
| 009671 | PGKGLEWIGYIYYSGSTYYNPSLKSRVTISVDTSKNQFSL | QKPGQAPRLLIYGASSRATGIPDRFSGSGSGTDFTLTI |
| | KLSSVTAADTAVYYCARALRGIFGVVNWFDPWGQGTLVTV | SRLEPEDFAVYYCQQYGSSPLTFGGGTKVEIK |
| | SS (SEQ ID NO: 1536) | (SEQ ID NO: 1771) |
| AB- | QVQLQQWGAGLLKPSETLSLTCAVYGGSFSGYYWSWIRQP | DIQMTQSPSTLSASVGDRVTITCRASQSVSSYLNWYQQ |
| 009676 | PGKGLEWIGEINHSGSTNYNPSLKSRVTISVDTSKNQFSL | KPGKAPKLLIYAASSLQSGVPSRFSGSGSGTDFTLTIS |
| | KLSSVTAADTAVYYCARLSRQWLVRPHIYYFDYWGQGTLV | SLQPEDFATYFCQQSYSTPRTFGQGTKLEIK |
| | TVSS (SEQ ID NO: 1537) | (SEQ ID NO: 1772) |
| AB- | QVTLRESGPALVKPTQTLTLTCTFSGFSLSTSGMCVSWIR | DIQMTQSPSSLSASVGDRVTITCRASQSISSYLNWYQQ |
| 009677 | QPPGKALEWLARIDWDDDKYYSTSLKTRLTISKDTSKNQV | KPGKAPKLLIYAASSLQSGVPSRFSGSGSGTDFTLTIS |
| | VLTMTNMDPVDTATYYCARSPIAARAGGFDYWGQGTLVTV | SLQPEDFATYYCQQSYSTPRTFGQGTKLEIK |
| | SS (SEQ ID NO: 1538) | (SEQ ID NO: 1773) |
| AB- | QVQLVESGGGVVQPGGSLRFSCAASGFTFSTYGMHWVRQA | QTVVTQEPSFSVSPGGTVTLTCGLSSGSVSTTYYPSWY |
| 009680 | PGKGLEWVAAIWYDGSNKYYADSVKGRFTISRDNSKNTLY | QQTPGQAPRTLIYNTKTRSSGVPDRFSGSILGNKAALT |
| | LQMDSLRAEDTAVYYCARRGVTISYYYMDVWGKGTTVTV | TGAQADDESDYYCVLYMGSGIWVFGGGTKLTVL |
| | SS (SEQ ID NO: 1539) | I (SEQ ID NO: 1774) |
| AB- | QVQLVQSGAEVKKPGSSVKVSCKASGGTFSSYAISWVRQA | EIVLTQSPATLSLSPGERATLSCRASQSVSSYLAWYQQ |
| 009681 | PGQGLEWMGGIIPIFGTANYAQKFQGRVTITADESTSTAY | KPGQAPRLLIYDASNRATGIPARFSGSGSGTDFTLTIS |
| | MELSSLRSEDTAVYYCASVGGDYDLDYWGQGTLVTVSS | SLEPEDFAVYYCQQRSNWPPYTFGQGTKLEIK |
| | (SEQ ID NO: 1540) | (SEQ ID NO: 1775) |
| AB- | QVQLQESGPGLVKPSETLSLTCTVSGGSISSYYWSWIRQP | EIVLTQSPGTLSLSPGERATLSCRASQSVSSSYLAWYQ |
| 009682 | PGKGLEWIGYIYYSGSTNYNPSLKSRVTISVDTSKNQFSL | QKPGQAPRLLIYGASSRATGIPDRFSGSGSGTDFTLTI |
| | KLSSVTAADTAVYYCARGATIFGVVIRPFDYWGQGTLVTV | SRLEPEDFAVYYCQQYGSSPLTFGGGTKVEIK |
| | SS (SEQ ID NO: 1541) | (SEQ ID NO: 1776) |
| AB- | QLQLQESGPGLVKPSETLSLTCTVSGGSISSSSYYWGWIR | DIQMTQSPSSLSASVGDRVTITCRASQSISSYLNWYQQ |
| 009687 | QPPGKGLEWIGSIYYSGSTYYNPSLKSRVTISVDTSKNQF | KPGKAPKLLIYAASSLQSGVPSRFSGSGSGTDFTLTIS |
| | SLKLSSVTAADTAVYYCARHTFGITIFGVVTRTGFDPWGQ | SLQPEDFATYYCQQSYSTPYTFGQGTKLEIK |
| | GTLVTVSS (SEQ ID NO: 1542) | (SEQ ID NO: 1777) |
| AB- | QVQLVESGGGVVQPGRSLRLSCAASGFTFSSYAMHWVRQA | SYELTQPPSVSVSPGQTASITCSGDKLGDKYACWYQQ |
| 009689 | PGKGLEWVAVISYDGSNKYYADSVKGRFTISRDNSKNTLY | KPGQSPVLVIYQDSKRPSGIPERFSGSNSGNTATLTIS |
| | LQMNSLRAEDTAVYYCAREMGSGMDVWGQGTTVTVSS | GTQAMDEADYYCQAWDSSTGVFGGGTKLTVL |
| | (SEQ ID NO: 1543) | (SEQ ID NO: 1778) |
| AB- | DVQLVESGGGLVQPGGSLRLSCAGFRFSTSENYVDWVRRA | DIVMTQSPLSLPVTPGEPASISCRSSQSLLHSNGYNYL |
| 009691 | PGKGLEWVARSKNRASSFATEYAASVKGRFTISRDDSTNS | VWYLQKPGQSPQLLIYFGSNRASGVPDRFSGSGSGTDF |
| | VSLQMNSLKIEDTALYYCAGSIAVAGAIWGQGTMVTVSS | TLRISRVEAEDVGVYYCMQSLQTPVTFGGGTKVEIK |
| | (SEQ ID NO: 1544) | (SEQ ID NO: 1779) |
| AB- | QVQLVESGGGVVQPGRSLRLSCAASGFTFSSYAMHWVRQA | DIQMTQSPSTLSASVGDRVTITCRASQSISSWLAWYQQ |
| 009695 | PGKGLEWVAVISYDGSNKYYADSVKGRFTISRDNSKNTLY | KPGKAPKLLIYKASSLESGVPSRFSGSGSGTEFTLTIS |
| | LQMNSLRAEDTAVYYCARARGGNYYYGMDVWGQGTTVT | SLQPDDFATYYCQQYNSYSWTFGQGTKVEIK |
| | VSS (SEQ ID NO: 1545) | (SEQ ID NO: 1780) |
| AB- | QLQLQESGPGLVKPSETLSLTCTVSGGSISSSSYYWGWIR | QSALTQPASVSGSPGQSITISCTGTSSDVGGYNYVSWY |
| 009697 | QPPGKGLEWIGSIYYSGSTYYNPSLKSRVTISVDTSKNQF | QQHPGKAPKLMIYDVSNRPSGVSNRFSGSKSGNTASLT |
| | SLKLSSVTAADTAVYYCASREVVLRYFDWSSPMGGFDPWG | ISGLQAEDEADYYCSSYTSSSTVVFGGGTKLTVL |
| | QGTLVTVSS (SEQ ID NO: 1546) | (SEQ ID NO: 1781) |
| AB- | ELQLVETGGGLVQPGGTLTISCAASGFTLNTYAMSWVRQA | ENVLTQSPGTLSLFPGERATLSCGASRSLGSNYLAWYQ |
| 009703 | PGKALEWVASSSGTGATTNYADSVNGRFSISRDNRRNTLF | QKPGQAPRLLIYGAFHRANGIPDRFSGSGSGTDFTLTI |
| | LQMDNLRADDTALYYCVRDGYNMIPFDMWGQGTMVTVSS | SRLEPEDFAVYYCQQYNSSPYTFGQGTKLELK |
| | (SEQ ID NO: 1547) | (SEQ ID NO: 1782) |
| AB- | QVQLQQWGAGLLKPSETLSLTCAVSGGSFSGYYWSWIRQV | LPVVTQAPSASALLGTSIKLTCTLSSEHSTYTIDWYQQ |
| 009706 | PDKGLEWIGEINHAGSLSYNPSLKSRVSMTDDTPKKQFFL | RPGRSPRHIMKIKSDGSHIKGGDIPDRFTGSSSGADRY |
| | KLTSLTAADSGVYYCVRWGPVGNTWAFDYWGPGTAVIVSS | LIISNLQPDDEAEYHCGQSHTNNGQAGVVFGGGTKLTV |
| | (SEQ ID NO: 1548) | L (SEQ ID NO: 1783) |
| AB- | QVQLQESGPGLVTPSGTLSLTCGVSGDSIYSDYWWTWVRQ | QTVVTQEPSLSVSPGGTVTLTCGLSSGSVSAHYFPTWY |
| 009707 | SPGKGLEWIGEVYRSGTRQYSPSLESRVTISIDTSGNQFS | FQTPGQPPRTLIYRTNTRSSGVPDRFSGSVLGDKAALT |
| | LNLISVTAADTAVYYCAAMRDGYGSGWLWGQGTLVTVSS | ITGAQADDEADYYCALSMNSGSFWVFGGGTKLTVL |
| | (SEQ ID NO: 1549) | (SEQ ID NO: 1784) |
| AB- | QVQLQQWGAGRLKPSETLSLTCAVYGGSLGGYYWTWIRQ | DIVMTQSPDSLAVSLGERATITCKSSQSVLYNSNNKDY |
| 009708 | PPGGGLEWIGESDESGKTDYNPSLKSRVTVSVDTSKNQFS | LAWYQQKPGQPPKLLIYWASTRTSGVPDRFTGSGSGT |
| | LKLTSVTAADTAVYYCARGHTSIYGVLIMLFHASWLDPWG | DFTLTINSLQAEDVAVYYCQQYYTSPRTFGQGTKVEIK |
| | QGTLVTVSS (SEQ ID NO: 1550) | (SEQ ID NO: 1785) |
| AB- | EVQLVESGGGLVQPGGSLRLSCAASGFVFRRYWMHWIRQA | NIVMTQSPLSLPVTPGEPASISCRSSESLVHPNGKTWL |
| 009709 | PGKGLAWVSRTNEDGTITNYADSVKGRFTISRDNTKNTLY | DWYLQKPGQSPQVLIHSASIRASGVPDRFSGSGSGTDF |
| | LQMNDLRVEDTATYFCARDVGGADGHWGQGTLVTVSS | TLKISRVEAEDVGVYYCMQGLQTPYTFGQGTKLEIK |
| | (SEQ ID NO: 1551) | (SEQ ID NO: 1786) |
| AB- | LVQLIQSGAEVVKPGSSVKVSCTASGGSYSSYTLSWVRQA | EIVLTQSPGTLSLSPGERATLSCRASQSVYGSYLAWYQ |
| 009710 | PGQGVEWLGRISPVLNTTYYAQKLEGRVTMTADESTSTVY | QKPGQAPRLLIYGGSSRATGIPDRFSGSGSGTDFTLTI |
| | MQLSSLRSEDTAVYYCARLASMNGVDIWGQGTSVTVSS | SRLEPEDFAVYYCHQYGTSRYSFGQGTKLDIK |
| | (SEQ ID NO: 1552) | (SEQ ID NO: 1787) |
| AB- | QVQLVQSGAEVKRPGASVKVSCKASGYTFTHYYIHWVRQ | ENVLTQSPDTLSVSPGERAALFCRASQSVAGSSFGGSY |
| 009711 | APGQGLEWVGRINPNSGGTHYAQKFQGRVTMTRDTSISTV | LAWYQQRPGQPPRLLIYGASTRATGVPDRFSGSGFGTD |
| | YLELTSLRSDDTALYYCARDSETDTSLAVAYYYYYMDVW | FTLTINRLEPEDIAVYYCQQYDKSPITFGQGTRLEIK |
| | GKGTAVAVSS (SEQ ID NO: 1553) | (SEQ ID NO: 1788) |
| AB- | QVQLVESGGSVVQPGRSLTLSCAASQFIFSSYAMHWVRQA | DIQMTQSPSTLSASVGDRVTITCRASQSIRNWLAWYQQ |
| 009712 | PGEGLEWVAMIYSDSSEKHYADSVKGRFTISRDNSKNTVS | KPGKVPKLLISDASNLKSGVPSRFSGSGFGTEFTLTIN |
| | LQMNSLGAEDTAVYYCAREGREYSDNSGKRGYFDSWGQGT | NLQPDDSASYYCQQYILYWTFGHGTKVEIK |
| | LVTVSS (SEQ ID NO: 1554) | (SEQ ID NO: 1789) |
| AB- | QVQLVESGGGVVQPGRSLRLSCAASGFTLRTYAMHWVRQ | QPVLTQSPSASASLGASVKLTCTLSSGYSTYAIAWHQQ |
| 009713 | APGKGLAWVAIISYSGDTEYDAGSVRGRFTISRDTSKNML | LPGKGPRYLMKLNSDGSHTKGDLIPDRFSGSSSGAERY |
| | YLQMNSLRAEDTGIYYCAREGEDYSSSYFDHWGQGTLVTV | LTISSLQSEDEGDYYCQTWGSGIQVFGGGTKLTVL |
| | SS (SEQ ID NO: 1555) | (SEQ ID NO: 1790) |
| AB- | QVQLQQWGAGLVKPSETLSLTCAVYGGSFSYYSWSWSWIR | SSELTQDPAVSVALGQTVRITCQGDSLRTYYATWYQQ |
| 009714 | QPPGKGLEWIGEIIHRGRTNSNPSLKSRVTVSRDTSKNQF | KPGQAPLLVIYAKNSRPSGIPDRFSGSSSGNTASLTIT |
| | SLNLSSVTAADTAVYYCARHRIVEATFSRYDYFDSWGQGT | GALAEDEADYYCNSRDSSGNYVVFGGGTKLTVL |
| | LVTVSS (SEQ ID NO: 1556) | (SEQ ID NO: 1791) |
| AB- | QVQLQQWGAELLKPSETLSLTCAVYGGSFNNHAWTWLRR | QTVVTQEPSLTVSPGGTVTLTCASSTGAVTSGYYPIWF |
| 009715 | PPGKGLEWIGEVIPHGSTKCDPSLKSRVTMSVDASKNQFS | QQKPGQAPRALIYNTNYRHSWTPARFSGSLLGGKAAL |
| | MKLISVSAADTAVYYCARRSGESNGWGTYYHYGMDVWGQ | TLSSVQPEDEAEFHCLLYYGGAQPWVFGGGTKLTVL |
| | GTTVIVSS (SEQ ID NO: 1557) | (SEQ ID NO: 1792) |
| AB- | QVQLQQWGAGLLKPSETLSLTCAVYGGSFTNYWWRWTW | SSELTQDPGVSVALGQTVRITCQGDSLRSYYASWLQQ |
| 009716 | VRQPPGRGLEWIGEIDRSGTTNYSPSLKSRVTISVDTSKN | KPGQAPVVVINGKNSRPSGIPDRFSGSTSGNTASLSIT |
| | QFSLTVTSVTAADTAVYYCARGRTPTGTFDYYSYLDVWGK | GAQAEDEADYYCNSRDSSGNSVVFGGGTKLTVL |
| | GTTIAVSS (SEQ ID NO: 1558) | (SEQ ID NO: 1793) |
| AB- | QVQLQQWGAGLLKPSETLSLTCAAHGGSFSGYYWSWIRRP | SYEVTQPPSVSVSPGQTASITCSGDELGRKYVSWYQQR |
| 009717 | PGKGLEWIGEITHSGRTNYNPSLTRRLSVFVDTPKSQFSL | PGQSPVLVIYRDNRRPSGIPERFSGSNFGNTATLTISG |
| | KLSSVTAADTATYYCARWDCTDGTCYAVYWGQGTLVTVSS | PQAMDEADYYCQAWDSTTVIFGGGTKLTVL |
| | (SEQ ID NO: 1559) | (SEQ ID NO: 1794) |
| AB- | QVQLQQWGAGLLKASETLSLTCAVYGGSFSNYYWNWIRQ | DIQMTQSPSSLSASVRDRVTITCRASQSISSYLNWYRQ |
| 009718 | SPGKGLEWIGEINYRGNTKYNPSLRSRVTISVDTSKNQFS | KPGKAPELLISAASTLQSGVPSRFSASGSGTDFTLTIS |
| | LKLSSLSAADTALYYCARGLGPPVGSLAITFGGLRRSFDI | SLQPEDFATFYCQQSYSAPFTFGGGTKVEIK |
| | WGQGTMVIVSS (SEQ ID NO: 1560) | (SEQ ID NO: 1795) |
| AB- | QVQLVQSGAEVKKPGASVKVSCKVSGYTLSKLFMHWVRQ | DIQMTQSPSSLSASVGDRVTITCQASQDISNYLNWYQQ |
| 009719 | APGKELEWMGGSVPEDDNTIYAEKFRGRVTMTEDTSTDTA | TPGKAPRLLIYDASNLETGVPSRFSGSGFGTDFTFTIS |
| | YMELSSLRSEDTALYFCATFRRQRGGYYYYGLDLWGQGTS | SLQPEDIATYYCQQADNLPYTFGQGTKLEIK |
| | VTVSS (SEQ ID NO: 1561) | (SEQ ID NO: 1796) |
| AB- | EVHLVESGGGLVQPGRSLRLSCAASGFKFDDYAMHWVRQ | DIVMTQSPLSLPVTPGEPASISCRSSQSLLHNDGYNYL |
| 009720 | GPSKGLEWVAGISWNSGSIDYADSVKGRFTISRDNAKNSL | DWYLQRPGQTPQLLIYLGFHRASGVPDRFSGSGSGTDF |
| | FLEMNSLTTEDTALYYCAKVKLTVFGVEQSPLDSWGQGTL | TLKISRVEAEDFGVYYCVQALQTPLTFGGGTKVEIK |
| | VTVSS (SEQ ID NO: 1562) | (SEQ ID NO: 1797) |
| AB- | EVQLLESGGGLVQPGGSLRLSCAVSGFTFSRYAMAWVRQA | QSVLTQPPSVSGAPGQRVTISCTGSSSNIGAGYDVHWY |
| 009721 | PGKGLEWVSTISDTDGSTIYYTDSVKGRFSISRDNSKNTL | QQLPGTAPKLIIYANNNRPSGVPDRFSASKSGTSASLA |
| | YLQMNSLRAEDTAVYYCTIQARVPSAMKVYFQNWGQGTLV | ITGLQPEDEADYYCQSYDNSLSGSRVFGGGTKLTVL |
| | TVSS (SEQ ID NO: 1563) | (SEQ ID NO: 1798) |
| AB- | QVQLVQSGAEVRKPGSSVKVSCNVSGGIFNSYKITWVRQA | EVVLTQSPATLSLSPGERATLSCRASQSVSSFLGWYQQ |
| 009722 | PGQGLEWMGGIIAIFGTADYAQRFQGRLSITADESTSTAY | KPGQAPRLLIYDASNRASGIPARFSGSGSGTDFTLTIS |
| | MELSSLRSDDTAVYYCTRERFSNYFDPWGQGTLVTVSS | KLEPEDFAVYYCQQRSRGPSGITFGQGTRLEIK |
| | (SEQ ID NO: 1564) | (SEQ ID NO: 1799) |
| AB- | QVQLVQSGAEVKKPGASVKVSCKASGYIFTNYDLSWVRQA | AIHLTQSPSSLSASVRDRVTITCRASQDISSALAWYHQ |
| 009723 | TGQGLEWMGWMNPYSGNTGFSQKFQGRVTMTRNTSISTV | KPGKPPKLLIYDASSLENGVPSRFSGSGSGTDFTLTIS |
| | YMELNSLRSEDTAIYYCARGRDVGQWLVGEGFDYWGQGS | SLQPEDFATYYCQQFNTYPLTFGGGTKVEIK |
| | LVTVSS (SEQ ID NO: 1565) | (SEQ ID NO: 1800) |
| AB- | QVQLVQSGAEVKKPGSSVKVSCKTSGGTFSNNAISWVRQA | DIVLTQTPLSLSVTPGQPASISCKSSQTLLHSDGKTYL |
| 009724 | PGQGLEWMGRIIPMVGVADYAQKLQGRVTLTADKSTTTAF | YWYLQKPGQPPQLLIYEVSNRFSGVPDRFSGSGSGTDF |
| | MELNSLTSEDTAVYYCARGKPFDFWSGLFDSWGQGTRVTV | TLTISRVEAEDVGVYYCMQSIELPPLTFGGGTKVEIK |
| | SS (SEQ ID NO: 1566) | (SEQ ID NO: 1801) |
| AB- | QMQLQESGPGLVKPSETLSLTCTVSHGSIRSQYWSWIRQS | DIQMTQSPSSLSASVGDRVTISCQASQDISNFLNWYQQ |
| 009725 | PGKGLEWIGYIYDSGSSKYNPSLKSRVTISLDTSKNEVYL | KPGEAPKLLIYDASILEAGVPSRFSGSGSGTDFTFSIS |
| | KLNSVTSADTAVYYCARRKVSFPRPSDDSSFDWFFDLWGR | SLQPEDIGTYYCQQYDNLAPLTFGGGTKVEIK |
| | GTLVTVSS (SEQ ID NO: 1567) | (SEQ ID NO: 1802) |
| AB- | EVQLVQSGAEVKEPGESLKISCKASGYTFINYWIGWVRQM | SYELTQPPSVSVSPGQTGRITCSGDELPKQYASWYQQK |
| 009726 | PGKGLEWMGIIYPGDSDTRYSPSFQGQVTISADKSITTAY | PGQAPVLVMYKDTERPSGIPERFSGSSSGTTVTLTISG |
| | LRWNSLKASDTAVYYCAREDIIENERGGFDIWGQGTMVTV | VQAEDEADYYCQSPDSSDTYRVFGGGTKLTVL |
| | SS (SEQ ID NO: 1568) | (SEQ ID NO: 1803) |
| AB- | QVQLVESGGGVVQPGRSLRLSCAASGFTFDNFGFHWVRQA | EIVMTQSPATLSVSPGERATFSCRASQSVGNNVAWYQ |
| 009727 | PGKGLEWVAVISHDGTNEYYGDSVKGRFTISRDDSKNTLF | QKPGQSPRLLIYGASLRATGIPARFSGSGSGTEFTLTI |
| | LQMNSLRTEDTAVYYCGRDRIALAEIDYWGQGTLVTVSS | SSLQSEDFAVYYCQQYNNWPPSITFGQGTRLEIK |
| | (SEQ ID NO: 1569) | (SEQ ID NO: 1804) |
| AB- | EVQLVESGGGLVKPGGSLRLSCGASGFNFNYYGMNWVRQ | SYDLTQPPSVSVSPGQTARITCSGDALPNQYVYWYQQ |
| 009728 | APGKGLEWVSSISSSSSHIYYADSVKGRFAISRDNAKNSL | KPGQAPLMVIFKDKERPSGIPERFSGSSSGTIVTLTIS |
| | YLQMNSLRVDDTGLYFCVRDRSFEYGDYGGDNWGQGTLVT | GVQAEDEADYYCQSADSSGTYPNWVFGAGTKLIVL |
| | VSS (SEQ ID NO: 1570) | (SEQ ID NO: 1805) |
| AB- | QVQLVQSGAEVKKPGASVKVSCKPSGYTFTDYYIHWVRQA | QSALTQPASVSGSPGQSITISCTGTSSDVGGYNYVSWY |
| 009729 | PGQGLEWMGWINPDTSATNYAQNFQGRLTMARDTSISTAY | QQHPGKAPKLMIYEVTNRPSGVSYRFSGSRSGNTASLT |
| | MELSGLRSDDTAVYFCTRVPHYYGSDTYYKPWYYYYAMD | ISGLQAEDEADYYCSSYTSVSTLMLFGGGTKLTVL |
| | VWGQGTTVTVSS (SEQ ID NO: 1571) | (SEQ ID NO: 1806) |
| AB- | QVQLVQSGAEVKRPGSSVKVSCKASGGTFSSYAISWVRQA | DIVMTQTPLSLSVTPGQPASISCKSSQSLLHSAGKTYL |
| 009730 | PGQGLEWMGEIIPVFGAPKFAQKFRGRVTITADESTNTAY | YWYLQKPGQSPQLLIYEVSNRFSGVPDRFSGSGSGTDF |
| | MELNSLRSDDTAVYYCAVGYCSSSSCEPQDAFDIWGQGTM | TLKISRVEAEDVGVYYCMQTIQLPISFGQGTRLGIK |
| | VTVSS (SEQ ID NO: 1572) | (SEQ ID NO: 1807) |
| AB- | QVQLVQSGAEVKKTGASVKLSCKASGYSLTSYAMHWVRQ | QSVLTQPPSASGTPGQRVTISCSGSTSNIGSNYVFWYQ |
| 009731 | APGQRLEWMGWLNGGNGNTKYSQKFQARVTITSDTSART | QFPGTAPKLLIYRNDQRPSGVPDRFSASKAGTSASLAI |
| | VYMELSSLTSEDTAVYYCARADHSTYWYGWFDPWGQGTL | SGLRSEDEADYYCTSWDDSLNGPLFGGGTKLTVL |
| | VTVSS (SEQ ID NO: 1573) | (SEQ ID NO: 1808) |
| AB- | EEQLVESGGVVVQPGGSLRLSCAASGFTFDDYTMHWVRQP | DIQMTQSPSSLSASVGDKVTITCRASQSISTFVNWYQQ |
| 009732 | PGKGLEWVSLISWDGGSKYYADSVMGRFTISRDNSKNSLY | KPGKAPKFLIYDASSLQSGVPSRFSGSGSGTDFALTIS |
| | LQMSSLRTEDTALYYCVKDISSSPANYGFAIWGLGTLVTV | SLQPEDFATYYCQQSFSTLLTFGGGTKVDIK |
| | SS (SEQ ID NO: 1574) | (SEQ ID NO: 1809) |
| AB- | QLQLQESGPGLVKPSETLSLTCTVSGGSISSTANYWGWIR | EIVLTQSPGTLSLSPGEGATLSCRASQSVSSGYLAWYQ |
| 009733 | QPPGKRLEWIGCISYSGRTYDNPSLKSRVTFSVDTSKNQF | HKPGQAPRLLIYGASSRATGIPDRFRGSGSGTDFTLTI |
| | SLKLSSLTAADTAMYYCARHAGWFGEHLDYWGQGTLVTVS | TRLEPEDFAVYYCQQYGSSLTWTFGQGTKVEIK |
| | S (SEQ ID NO: 1575) | (SEQ ID NO: 1810) |
| AB- | QVHLVQSGAEVKTPGASVRVSCKASGYTFTSHYMHWVRQ | EIVLTQSPGTLSLSPGERATLSCRASQTISSRYLAWYY |
| 009734 | APGQGLEWMGLINPSGSGTAYPQHFQGRVTMTRDTSTTTD | QKPGQAPRLLIQGSSIRATGIPDRFSGSGSGTDFTLTI |
| | YMDLSSLRSEDTAMYFCARMERSGAFDLWGQGTMVTVSS | SRLEPEDFAVYYCQHYGSSPPMYTFGQGTKLEIK |
| | (SEQ ID NO: 1576) | (SEQ ID NO: 1811) |
| AB- | QVQLVQSGAEVKKPGSSVKVSCKPSGGTFNSYAFTWVRQA | NFMLTQPHSVSESPGKTVTVSCTRSSGSIASNYVQWYQ |
| 009735 | PGQGLEWMGTVLPIFDTTHSAQKFQGRVTISADKSANTVY | QRPGSAPTTVIYEDNQRPSGVPDRFSGSIDSSSNSASL |
| | MELSSLTSGDTAVYYCVGRYGSGNYEGYWGQGTLVTVSS | TISGLKTEDEAYYYCQSYDSRNPQWVFGGGTTLTVL |
| | (SEQ ID NO: 1577) | (SEQ ID NO: 1812) |
| AB- | QVQLVQSGAEVQKPGSSVKVSCKASGGTFVNFAICWVRQA | DVVMTQSPLSLPVTLGQPASISCRSSESLVHTDGNTYL |
| 009736 | PGQGLEWIGGIIPVFGATNYAQKFRGRVTITADESTSTAY | NWFHQRPGHSPRRLIYKVSNRDSGVPDRFGGSGSGTD |
| | MELSGLTSEDTALYYCAKDLLSDRQDPSWGQGTLVTVSS | FTLKISWVEAEDVGVYYCMQGTHWPWTFGQGTKVEI |
| | (SEQ ID NO: 1578) | K (SEQ ID NO: 1813) |
| AB- | QVTLKESGPVLVKPTETLTLTCTVSGLALSNGRMGVSWIR | EVVMTQSPATLSVSPGERATLSCRASQTFSSNLAWYQ |
| 009737 | QSPGKALEWLAHIFSNDAKSYSTSLKGRLTISKDTSRSQV | QKPGQPPRLLIYGASTRATGVPARFSGSGSGTEFTLTI |
| | VLTMTNMDPVDTATYYCASIDERLGILYWYFHLWGRGSLV | SSLQSEDFAVYYCQQYNDWPRTFGQGTKVEMK |
| | SVSS (SEQ ID NO: 1579) | (SEQ ID NO: 1814) |
| AB- | QVQLVQSGAEVKKPGSSVKVSCKASGGTFTNYAINWVRQA | EIVLTQSPGTLSLSPGERATLSCRASQSISSTYLAWYQ |
| 009738 | PGQGLEWMGGIIPVFGAPNYAQKFQGRVTITADTSTSTAY | QKPGQAPRLLIYGASSRATGIPDRFSGTGSGTDFTLTI |
| | MELSSLRSEDTAVYYCARVGYCSTTGCDMGGLDYWGQGTL | SRLEPEDFAVYYCQQYGVSPNTFGGGTKVEIK |
| | VTVSS (SEQ ID NO: 1580) | (SEQ ID NO: 1815) |
| AB- | EVQLEQSGAEVKKPGESLKISCKGSGYTFTSYWIVWVRQM | SYVLTQPPSVSVAPGKTARITCGGNNIGRKSVHWYQQ |
| 009739 | PGKGLEWLGIIYPGDSDTRYSLAFQGQVTISADKSISTAY | KPGQTPVLVVYDDTDRPSRIPERFSGSNSGNTATLTIS |
| | LQWSSLKPSDTAFYYCARLSAQRGNSGPAVYWGQGTLVTV | RVEAGDEADYYCQVWDSSSDHVVFGGGTRLTVP |
| | SS (SEQ ID NO: 1581) | (SEQ ID NO: 1816) |
| AB- | EVLLLESGGDLVQPGGSLRLSCAASGFTFSSYGMNWVRQA | QLVLTQSPSASASLGASVRLTCTLSSGHSSYPIAWHQQ |
| 009740 | PGKGLEWVSSISGSGGSPYYADSVKGRFTISRDNSKNTLY | QPDKGPRYLLKLNSDGSHSKGDGIPDRFSASSSGTERY |
| | LQMKSLRAEDTAIYYCAKSEETMTTVTTFFDYWGQGTLVT | LTISSLQSEDEADYYCQTWGTATPWVFGGGTKLTVL |
| | VSS (SEQ ID NO: 1582) | (SEQ ID NO: 1817) |
| AB- | EVQLLESGGGLVQPGGSLRLSCAASGFTFSSYAMSWVRQA | DIQMTQSPSSLSASVGDRVTITCRTSQSIATYLNWYHQ |
| 009741 | PGKGLECVALINGGGGRTYYTDSVKGRFTISRDNSKNTLY | KPGKPPNLLIFAASSLHSGVPSRFSGSGSGTDFTLTIS |
| | LQMNSLRAEDTAVYYCERSVGYWGQGTLVTVSS | SLQPEDFATYYCQQTYSAPYTFGQGTKLAIK |
| | (SEQ ID NO: 1583) | (SEQ ID NO: 1818) |
| AB- | EVQLVESGGGLEQPGRSLRLSCAASGITFEDYALHWVRQA | DIQMTQSPSSVSASLGDRVTITCRASQGISNLLAWYQQ |
| 009742 | PGKGLEWVSGISWNGANIGYADSVKGRFTISRDNAQKSLY | KPGKAPKLLIYAASRLQSGVPSRFSGSGSGTDFTLTIS |
| | LQMSGLRTEDTALYYCAKSTVTTLRRTYFDSWGHGTLVIV | SLQPEDFATYYCQQANTFPPALTFGGGTKVEIK |
| | SS (SEQ ID NO: 1584) | (SEQ ID NO: 1819) |
| AB- | QVYLVQSGAEVKKPGASVKVSCKVSGNTLSELYIYWVRQA | QAVVTQEPSLTVSPGGTVTLTCGSSTGPVTSGRYPYWL |
| 009743 | PGKGLEWMGGFDPEDGETIYAQNFQGRVTITEDTSTDTVY | QQKPGQAPRTLIYDTNTKYSWTPARFSGSLLGGKAAL |
| | MELSSLRSEDTATYYCATHDFGDSEGEYWGQGTQVTVSS | TLSGAQPEDEADYYCLLSYSGARVFGGGTKLTVL |
| | (SEQ ID NO: 1585) | (SEQ ID NO: 1820) |
| AB- | PVQLVQSGAEVKKPGASVKVSCQTSGYTFTNYAVHWVRQ | QSALTQPRSVSGSPGQPVTISCTGSSSDVGGYNYVSWY |
| 009744 | APGQGLEWMGWINAGNGNTKYSQKFQGRVTITRDTSASTA | QQHPGKAPKLIIYDVGKRPSRVPDRFSGSKSGNTASLT |
| | YMELSSLRSEDTAVYYCAKDQWADYHTTGDFEYWGQGTL | ISGLQAEDEADYYCCSYAGTHTYILFGGGTKVTVL |
| | VTVSS (SEQ ID NO: 1586) | (SEQ ID NO: 1821) |
| AB- | EVQLVESGGGLVPPGRSLRLSCRASGFTFGDYAMNWFRQA | DVVMTQSPLSLPVTLGQPASISCRSSQSLVYSDGNTYL |
| 009745 | PGKGLEWVGFIRSKVYGETTEYAASVKGRFTISRDDSRSI | NWFHQRPGQSPRRLIYRVSNWDSGVPDRFSGSGSGTD |
| | AYLQMDSLKTEDTAVYFCTRDGRLLWFEESDWGDAFGIWG | FTLKISRVEAEDVGVYYCMQGTHWPYTFGQGTKLEIK |
| | QGTMVTVSS (SEQ ID NO: 1587) | (SEQ ID NO: 1822) |
| AB- | QLQLQESGPGLKKPSETLSLTCTVSGASLISNSYYWGWIR | QSVLTQPPSVSGAPGQRVTISCTGSSSNIGAGYDVHWY |
| 009746 | QPPGKGLEWIGSIFYSGTTHYNPSLKSRVTISVDTSKNQF | QQLPGTAPKLLIYDNTNRPSGVPDRFSGSKSGTSASLA |
| | SLRLTSVTAADTAVYYCARLTVDRRLQGYYYYYYMDVWGK | ITGLQAEDEADYYCQSYDNSLSGSLFGGGTKLTVL |
| | GTTVTVSS (SEQ ID NO: 1588) | (SEQ ID NO: 1823) |
| AB- | QVQLVESGGGVVQPGGSLRLSCAASGFTFSNYGMHWVRQ | DIVMTQSPDSLAVSLGERATINCKSSQSVLYSSNNKNY |
| 009747 | APGKGLEWVAFIRYDGSNEYYADSVKGRFTISRDNSKNTL | LTWYQQKPGQPPKLLIYWASTRESGVPDRFSGSGSGT |
| | YLHMNSLRPEDTAVYYCAKQGLLSNYYYGMDVWGQGTTV | DFTLTISSLQAEDVAVYHCQQYYSSVTFGQGTRLEIK |
| | TVSS (SEQ ID NO: 1589) | (SEQ ID NO: 1824) |
| AB- | QVQLAESGGGVVQPGGSLRLSCAASGFTFSSHGMHWVRQA | DIQMTQSPSSLSASVGDRVTITCRASQGISNYLAWYQQ |
| 009748 | PGKGLEWVAFIRYDGSNKYYADSVKGRFTIFRDNSKNTLY | KPGKVPKLLIYAASTLQSGVPSRFSGSGSGTDFTLTIS |
| | LQMNSLRAEDTAVYYCAKDNYYDILTGYYRKYYYFGMDV | SLQPEDVATYYCQKYNSAPFTFGPGTKVDIK |
| | WGQGTTVTVSS (SEQ ID NO: 1590) | (SEQ ID NO: 1825) |
| AB- | QVQLQESGPGLVKPSETLSLTCTVSGGSISSYYWSWIRQP | SYELTQPPSVSVSPGQTARITCSGDALPKKYASWYQQK |
| 009749 | PGKGLEWIGYIHYSGSTIYNPSLKSRVTISVDTSKSQFSL | SGQAPVLVIYEDTKRPSGIPERFSGSSSGTMATLTISG |
| | KLSSVTAADTAVYYCARTAGSDDSGYLPFDYWGQGTLVTV | AQVEDEADYYCYSTDSSGNHRVFGGGTKLTVL |
| | SS (SEQ ID NO: 1591) | (SEQ ID NO: 1826) |
| AB- | QLQLQESGPGLVKPSETLSLTCTVSGGSISSSSYYWGWIR | AIQLTQSPSSLSASVGDRVTITCRASQGISSYLAWYQQ |
| 009750 | QPPGKGLEWIGSIYYSGSTYYNPSLKSRVTISVDTSKNQF | KPGKAPKLLIYAASTLQSGVPSRFSGSGSGTDFTLTIS |
| | SLKLSSVTAADTAVYYCSRSPFSISIFGVVIRYGMDVWGQ | SLQPEDFATYYCQQLNSYPRTFGQGTKLEIK |
| | TGTVTVSS (SEQ ID NO: 1592) | (SEQ ID NO: 1827) |
| AB- | QVQLQESGPGLVKPSQTLSLTCTVSGGSISSGDYYWSWIR | EIVLTQSPGTLSLSPGERATLSCRASQSVSSSYLAWYQ |
| 009751 | QPPGKGLEWIGYIYYSGSTYYNPSLKSRVTISVDTSKNQF | QKPGQAPRLLIYGASSRATGIPDRFSGSGSGTDFTLTI |
| | SLKLSSVTAADTAVYYCARAITKITIFGVVIENFDYWGQG | SRLEPEDFAVYYCQQYGSSPLTFGGGTKVEIK |
| | TLVTVSS (SEQ ID NO: 1593) | (SEQ ID NO: 1828) |
| AB- | QVQLVQSGAEVKKPGASVKVSCKASGYTFTSYAMHWVRQ | SSELTQDPAVSVALGQTVRITCQGDSLRSYYASWYQQ |
| 009752 | APGQRLEWMGWINAGNGNTKYSQKFQGRVTITRDTSASTA | KPGQAPVLVIYGKNNRPSGIPDRFSGASSGNTASLTIT |
| | YMELSSLRSEDTAVYYCAREVFEAAAGMLLPYYYYYMDV | GAQAEDEADYYCNSRDSSGNHLVFGGGTKLTVL |
| | WGKGTTVTVSS (SEQ ID NO: 1594) | (SEQ ID NO: 1829) |
| AB- | QVQLQESGPGLVKPSETLSLTCTVSGGSISSHQWNWIRQP | DIQMTQSPSSLSASVGDRVTITCRASQSISSYLNWYQQ |
| 009753 | PGKGLEWIAYIYYSGSTNYNPSLKSRVTISVDTSKNQFSL | KPGKAPKLLIYAASSLQSGVPSRFSGSGSGADFTLTIS |
| | KLSSVTAADTAVYYCARQRREWLGSYYYYGMDVWGQGTTV | SLQPEDFATYYCQQSYSTPLTFGGGTKVEIK |
| | TVSS (SEQ ID NO: 1595) | (SEQ ID NO: 1830) |
| AB- | QLQLQESGPGLVKPSETLSLTCTVSGGSISSSSYYWGWIR | SSELTQDPAVSVALGQTVRITCQGDSLRSYYASWYQQ |
| 009754 | QPPGRGLEWIGIIYYSGSTYYNPSLKSRVTISVDTSKNQF | KPGQAPLLVIYGKNNRPSGIPDRFSASSSGNTASLTIT |
| | SLKLTSVTAADTAVYYCARVYAGFLEWLSYFDYWGQGTLV | GAQAEHEADYYCNSRDSSGNHWVFGGGTKLTVL |
| | TVSS (SEQ ID NO: 1596) | (SEQ ID NO: 1831) |
| AB- | QVQLQESGPGLVKPSQTLSLTCTVSGDSISSGAYYWSWIR | EIVLTQSPGTLSLSPGERATLSCRASQSVSSSYLAWYQ |
| 009755 | QHPGKGLEWIGYIYYSGSTYYNPSLKSRVTISVDTSKNQF | QKPGQAPRLLIYGASSRATGIPDRFSGSGSGTDFTLTI |
| | SLKLSSVTAADTAVYYCARARRITIFGVVGDFGVWGQGTL | SRLEPEDFAVYYCQQYGSSPLTFGGGTKVEIK |
| | VTVSS (SEQ ID NO: 1597) | (SEQ ID NO: 1832) |
| AB- | EVQLLESGGGLVQPGGSLRLSCAASGFTFSSFAMSWVRQA | SYELTQPPSVSVSPGQTARITCSGDALPKQYAYWYQQ |
| 009756 | PGKGLEWVSAISGSGGSTYYADSVKGRFTISRDNSKNTLY | KPGQAPILMIYKDTERPSGIPERFSGSSSGTTVTLTIS |
| | LQMNSLRAEDTAVYYCAKEERVYDILTGYDYWGQGTLVTV | GVQAEDEADYYCQSADSSGSSVVFGGGTKLTVL |
| | SS (SEQ ID NO: 1598) | (SEQ ID NO: 1833) |
| AB- | QLQLQESGPGLVKPSETLSLTCTVSGGSISSSSYYWGWIR | EIVLTQSPATLSLSPGERATLSCRASQSVSSYLAWYQQ |
| 009757 | QPPGKGLEWIGSIYYSGSTYYNPSLKSRVTISVDTSKNQF | KPGQAPRLLIYDASNRATGIPARFSGSGSGTDFTLTIS |
| | SLKLSSVTAADTAVYYCARLKNWNTIFGVVIMAPYYMDVW | SLEPEDFAVYYCQQRSKGITFGQGTRLEIK |
| | GKGTTVTVSS (SEQ ID NO: 1599) | (SEQ ID NO: 1834) |
| AB- | QVQLVQSGAEVKKPGASVKVSCKVSGYTLTELSMHWVRQ | SYVLTQPPSVSVAPGKTARITCGGNNIGSKSVHWYQQ |
| 009758 | APGKGLEWMGGFDPEDGETIYAQKFQARVTMTEDTSTDTA | KPGQAPVLVVYDDSDRPSGIPERFSGSNSGNTATLTIS |
| | YMELSSLRSEDTAVYFCATGYYGSRFDPWGQGTLVTVSS | RVEAGDEADYYCQVWDSSTDHPGVVFGGGTKLTVL |
| | (SEQ ID NO: 1600) | (SEQ ID NO: 1835) |
| AB- | QVQLVQSGAEVKKPGSSVKVSCKASGGTFSSYAISWVRQA | EIVMTQSPATLSVSPGERATLSCRASQSVSSKLAWYQQ |
| 009759 | PGQGLEWMGGIIPIFGTANYAQKFQGRVTITADESTSTAY | KPGQAPRLLIYGASTRATGIPARFSGSGSGTEFTLTIS |
| | MELSSLRSEDTAVYYCARALVQGVYYYYMDVWGKGTTVTV | SLQSEDFAVYYCQQYNNWPPYTVGQGTKLEIK |
| | SS (SEQ ID NO: 1601) | (SEQ ID NO: 1836) |
| AB- | EVQLVQSGAEVKKPGESLKISCKGSGYSFTSYWIGWVRQM | DIQMTQSPSTLSASVGDRVTITCRASQSISSWLAWYQQ |
| 009760 | PGKGLEWMGIIYPGDSDTRYSPSFQGQVTISADKSISTAY | KPGKAPKLLIYDASSLESGVPSRFSGSGSGTEFTLTIS |
| | LQWSSLKASDTAMYYCARSRSSDYFDYWGQGTLVTVSS | SLQPDDFATYYCQQYNSYSRTFGQGTKVEIK |
| | (SEQ ID NO: 1602) | (SEQ ID NO: 1837) |
| AB- | EVQLVESGGGLVQPGGSLRLSCAASGFTFSSYAMSWVRQA | DIQMTQSPSTLSASVGDRVTITCRASQSISSWLAWYQQ |
| 009761 | PGKGLEWVSAISGSGGSTYYADSVKGRFTISRDNSKNTLY | KPGKAPKLLIYDASSLESGVPSRFSGSGSGTEFTLTIS |
| | LQMNSLRAEDTAVYYCAKGPRSAPDYMDVWGKGTTVTVSS | SLQPDDFATYYCQQYNSYSGTFGQGTKVEIK |
| | (SEQ ID NO: 1603) | (SEQ ID NO: 1838) |
| AB- | QVQLVESGGGLVKPGGSLRLSCAASGFTFSDYYMSWIRQA | DIQMTQSPSSLSASVGDRVTITCRASQSISSYLNWYQQ |
| 009762 | PGKGLEWVSYISRSGSTIYYVDSVKGRFTISRDNAKNSLY | KPGKAPKLLIYAASSLQSGVPSRFSGSGSGTDFTLTIS |
| | LQMNSLRAEDTAVYYCARDQRLWSTLGVYYYYYMDVWGKG | SLQPEDFATYYCQQSYSHPLTFGGGTKVEIK |
| | TTVTVSS (SEQ ID NO: 1604) | (SEQ ID NO: 1839) |
| AB- | QVQLQQWGAGLLKPSETLSLTCAVYGGSFSGYYWSWIRQP | DIQMTQSPSTLSASVGDRVTITCRASQSINNWLAWYQQ |
| 009763 | PGKGLEWIGEINHSGSTNYNPSLKSRVTISVDTSKNQFSL | KPGKAPQLLIYKASSLESGVPSRFSGSGSGTKFTLTIS |
| | KLSSVTAADTAVYYCARRAAPAWTWFGELSPFDYWGQGTL | SLQPDDFATYYCQQYNSYSQYTFGQGTKLEIK |
| | VTVSS (SEQ ID NO: 1605) | (SEQ ID NO: 1840) |
| AB- | QVQLQESGPGLVKPSETLSLTCTVSGGSISSHYWSWIRQP | QSALTQPPSASGSPGQSVTISCTGTSSDVGGYNYVSWY |
| 009764 | PGKGLEWIGYIYYSGSTNYNPSLKSRVTISVDTSKNQFSL | QQHPGKAPKLMIFDVSKRPSGVPDRFSGSKSGNTASLT |
| | KLSSVTAADTAVYYCARSARRWLQYGPDWYFDLWGRGTLV | VSGLQAEDEADYYCSSYAGSNNWVFGGGTKLTVL |
| | TVSS (SEQ ID NO: 1606) | (SEQ ID NO: 1841) |
| AB- | QVQLVESGGGLVKPGGSLRLSCAASGFTFSDYYMSWIRQA | EIVMTQSPATLSVSPGERATLSCRASQSVSSNLAWYQQ |
| 009765 | PGKGLEWVSYISSSGNTIYYADSVKGRFTISRDNAKNSLY | KPGQAPRLLIYGASTRATGFPARFSGSGSGTEFTLTIS |
| | LQMNSLRAEDTAVYYCARDPPPRITIFGVGTRLGFDYWGQ | GLQSEDFAVYYCQQYNNWPPWTFGQGTKVEIK |
| | GTLVTVSS (SEQ ID NO: 1607) | (SEQ ID NO: 1842) |
| AB- | QLQLQESGPGLVKPSETLSLTCTVSGGSISRSSYYWGWIR | DIQLTQSPSFLSASVGDRVTITCRASQGISSYLAWYQQ |
| 009766 | QPPGKGLEWIGSIYYSGSTYYNPSLKSRVTISVDTSKNQF | KPGKAPKLLIYAASTLQSGVPSRFSGSGSGTEFTLTIS |
| | SLKLSSVTAADTAVYYCARQLLGIWGSYRYIDYWGQGTLV | SLQPEDFATYYCQQLNSYPITFGQGTRLEIK |
| | TVSS (SEQ ID NO: 1608) | (SEQ ID NO: 1843) |
| AB- | EVQLVQSGAEVKKPGESLKISCKGSGYSFTSYWIGWVRQM | QSALTQPASVSGSPGQSITISCTGTSSDVGGYNYVSWY |
| 009767 | PGKGLEWMGIIYPGDSDTRYSPSFQGQVTISADKSISTAY | QQHPGKAPKLMIYDVSNRPSGVSNRFSGSKSGNTASLT |
| | LQWSSLKASDTAMYYCASGRLHLGELLDYWGQGTLVTVSS | ISGLQAEDEADYYCSSYTSSSTLVVFGGGTKLTVL |
| | (SEQ ID NO: 1609) | (SEQ ID NO: 1844) |
| AB- | QLQLQESGPGLVKPSETLSLTCTVSGGSISSSSYYWGWIR | QSVLTQPPSVSGAPGQRVTISCTGSSSNIGAGYDVHWY |
| 009768 | QPPGKGLEWIGSIYYSGSTYYNPSLKSRVTISVDTSKNQF | QHLPGTAPKLLIYGNSNRPSGVPDRFSGSKSGTSASLA |
| | SLKLSSVTAADTAVYHCARQPRRITIFGVVIMPTNNWFDP | ITGLQAEDEADYYCQSYDSSLSDVVFGGGTKLTVL |
| | WGQGTLVTVSS (SEQ ID NO: 1610) | (SEQ ID NO: 1845) |
| AB- | EVQLVESGGGLVQPGGSLRLSCAASGFTFSSYWMSWVRQA | SYELTQPPSVSVSPGQTARITCSGDALPKQYAYWYQQ |
| 009769 | PGKGLEWVANIKQDGSEKYYVDSVKGRFTISRDNAKNSLY | KPGQAPVLVIYKDSERPSGIPERFSGSSSGTTVTLTIS |
| | LQMNSLRAEDTAVYYCARSPEVLWFGDPVLDNWFDPWGQ | GVQAEDEADYYCQSADSSGTYPVVFGGGTKLTVL |
| | GTLVTVSS (SEQ ID NO: 1611) | (SEQ ID NO: 1846) |
| AB- | EVQLVESGGGLVKPGGSLRLSCAASGFTFSSYSMNWVRQA | DIQMTQSPSSLSASVGDRVTITCRASQSISSYLNWYQQ |
| 009770 | PGKGLEWVSSISSSSSYIYYADSVKGRFTISRDNAKNSLY | KPGKAPKLLIYAASSLQSGVPSRFSGSGSGTDFTLTIS |
| | LQMNSLRAEDTAVYYCARDPYYDILTGYYLYWGQGTLVTV | SLQPEDFATYYCQQSYSTPLITFGQGTRLEIK |
| | SS (SEQ ID NO: 1612) | (SEQ ID NO: 1847) |
| AB- | EVQLVESGGGLVQPGGSLRLSCAASGFTFSSYWMHWVRQA | DIQMTQSPSTLSASVGDRVTITCRASQSISRWLAWYQQ |
| 009771 | PGKGLVWVSRINSDGSSTSYADSVKGRFTISRDNAKNTLY | KPGKAPKLLIYKASSLESGVPSRFSGSGSGTEFTLTIS |
| | LQMNSLRAEDTAVYYCARDGRNIAVAGLDYWGQGTLVTVS | SLQPDDFATYYCQQYNSYQGTFGQGTKVEIK |
| | S (SEQ ID NO: 1613) | (SEQ ID NO: 1848) |
| AB- | QLQLQESGPGLVKPSETLSLTCTVSGGSISSSSYYWGWIR | QSVLTQPPSVSGAPGQRVTISCTGSSSNIGAGYDVHWY |
| 009772 | QPPGKGLEWIGSIYYSGSTYYNPSLKSRVTISVDTSKNQF | QQLPGTAPKLLIYGNSNRPSGVPDRFSGSKSGTSASLA |
| | SLKLSSVTAADTAVYYCARHLQPSYGGNAFDIWGQGTMVT | ITGLQAEDEADYYCQSYDSSLSGYVFGTGTKVTVL |
| | VSS (SEQ ID NO: 1614) | (SEQ ID NO: 1849) |
| AB- | QVQLVQSGAEVKKPGASVKVSCKASGYTFTGYYMHWVRQ | DIQMTQSPSSLSASVGDRVTITCRASQSISSYLNWYQQ |
| 009773 | APGQGLEWMGRINPNSGGTNYAQKFQGRVTMTRDTSISTA | KPGKAPKLLIYAASSLQSGVPSRFSGSGSGTDFTLTIS |
| | YMELSRLRSDDTA VYYCARDGCSGGSCYSWWFDPWGQGT | SLQPEDFATYYCQQSYSTPLTFGGGTKVEIK |
| | LVTVSS (SEQ ID NO: 1615) | (SEQ ID NO: 1850) |
| AB- | QLQLQESGPGLVKPSETLSLTCTVSGGSISSSSYYWGWIR | DIVMTQSPDSLAVSLGERATINCKSSQSVLYSSNNKNY |
| 009774 | QPPGKGLEWIGSIYYSGSTYYNPSLKSRVTISVDTSKNQF | LAWYQQKPGQPPKLLIYWASTRESGVPDRFSGSGSGT |
| | SLKLSSVTAADTAVYYCARQKTDIVVVVATEVAFDYWGQG | DFTLTISSLQAEDVAVYYCQQYYSTPLTFGQGTKLEIK |
| | TLVTVSS (SEQ ID NO: 1616) | (SEQ ID NO: 1851) |
| AB- | QLQLQESGPGLVKPSETLSLTCTVSGGSISSSSYYWGWIR | EIVLTQSPGTLSLSPGERATLSCRASQSVSSSYLAWYQ |
| 009775 | QPPGKGLEWIGSIYYSGSTYYNPSLKSRVTISVDTSKNQF | QKPGQAPRLLIYGASSRATGIPDRFSGSGSGTDFTLTI |
| | SLKLSSVTAADTAVYYCARQVRSGIVVVTPYYFDYWGQGT | SRLEPEDFAVYYCQQYGSSPRTFGQGTKVEIK |
| | LVTVSS (SEQ ID NO: 1617) | (SEQ ID NO: 1852) |
| AB- | QVQLQESGPGLVKPSQTLSLTCTVSGGSISSGGYYWSWIR | SYELTQPPSVSVSPGQTASITCSGDKLGDKYACWYQQ |
| 009776 | QHPGKGLEWIGYIYYSGSTYYNPSLKSRVTISVDTSKNQF | KPGQSPVLVIYQDSKRPSGIPERFSGSNSGNTATLTIS |
| | SLKLSSVTAADTAVYYCARDQYDSSGYAFGAFDYWGQGTL | GTQAMDEADYYCQAWDSSTAWVFGGGTKLTVL |
| | VTVSS (SEQ ID NO: 1618) | (SEQ ID NO: 1853) |
| AB- | EVQLVESGGGLVKPGGSLRLSCAASGFTFSSYSMNWVRQA | SSELTQDPAVSVALGQTVRITCQGDSLRSYYASWYQQ |
| 009777 | PGKGLEWVSSISSSSSYIYYADSVKGRFTISRDNAKNSLY | KPGQAPVLVIYGKNNRPSGIPDRFSGSSSGNTASLTIT |
| | LQMNSLRAEDTAVYYCARDESVAHYYGSGSVDVWGQGTTV | GAQAEDEADYYCNSRDSSGNRWVFGGGTKLTVL |
| | TVSS (SEQ ID NO: 1619) | (SEQ ID NO: 1854) |
| AB- | QLQLQESGPGLVKPSETLSLTCTVSGGSISSSSYYWGWIR | DIQMTQSPSTLSASVGDRVTITCRASQSISSWLAWYQQ |
| 009778 | QPPGKGLEWIGSIYYSGSTYYNPSLKSRVTISVDTSKNQF | KPGKAPKLLIYKASSLESGVPSRFSGSGSGTEFTLTIS |
| | SLKLSSVTAADTAVYYCARQFRRWLQLGPSYFDYWGQGTL | SLQPDDFATYYCQQYNSYSRAWTFGQGTKVEIK |
| | VTVSS (SEQ ID NO: 1620) | (SEQ ID NO: 1855) |
| AB- | EVQLVESGGGLVKPGGSLRLSCAASGFTFSNAWMSWVRQA | DIQMTQSPSSLSASVGDRVTITCRASQGIKYYLAWFQQ |
| 009779 | PGKGLEWVGRIKTKSDGGTTDYAAPVKGRFIISRDDSKNT | KPGKAPKSLIYAASSLLSGVPSKFSGSGSGTDFTLTIS |
| | LYLQINSLKVEDTAIYYCSRNPFLDYWGQGTLVTVSS | SLQPEDFATYYCQQYNSYPYTFGQGTKLEIK |
| | (SEQ ID NO: 1621) | (SEQ ID NO: 1856) |
| AB- | QVQLQESGPGLVKPSQTLSLTCTVSGGSISSGDYYWSWIR | DIQMTQSPSSLSASVGDRVTITCRASQSISSYLNWYQQ |
| 009780 | QPPGKGLEWIGYIYYSGSTYYNPSLKSRVTISVDTSKNQF | KPGKAPKLLIYAASSLQSGVPSRFSGSGSGTDFTLTIS |
| | SLKLSSVTAADTAVYYCARDRWGITIFGVVNRDGMDVWGQ | SLQPEDFATFYCQQSYSFPYTFGQGTKLEIK |
| | GTTVTVSS (SEQ ID NO: 1622) | (SEQ ID NO: 1857) |
| AB- | EVQLVESGGGLVKPGGSLRLSCAASGFTFSNAWMSWVRQA | DIQMTQSPSTLSASVGDRVTITCRASQSISSWLAWYQQ |
| 009781 | PGKGLEWVGRIKSKTDGGTTDYAAPVKGRFTISRDDSKNT | KPGKAPKLLIYDASSLESGVPSRFSGSGSGTEFTLTIS |
| | LYLQMNSLKTEDTAVYYCTTLGHQYYDSSGYDYYYGMDV | SLQPDDFATYYCQQYNSYTWTFGQGTKVEIK |
| | WGQGTTVTVS (SEQ ID NO: 1623) | (SEQ ID NO: 1858) |
| AB- | QVQLQESGPGLVKPSETLSLTCTVSGGSISSYYWSWIRQP | SYELTQPPSVSVSPGQTARITCSGDALPKKYAYWYQQ |
| 009782 | AGKGLEWIGRIYTSGSTNYNPSLKSRVTMSVDTSKNQFSL | KSGQAPVLVIYEDSKRPSGIPERFSGSSSGTMATLTIS |
| | KLSSVTAADTAVYYCARGRLFYDSSGYYFDYWGQGTLVTV | GAQVEDEADYYCYSTDSSGNHYVFGTGTKVTVL |
| | SS (SEQ ID NO: 1624) | (SEQ ID NO: 1859) |
| AB- | QVQLQESGPGLVKPSETLSLTCTVSAGSISSHYWSWIRQP | EIVLTQSPGTLSLSPGERATLSCRASQSVSSSYLAWYQ |
| 009783 | PGKGLEWIGYIYYSGSTNYNPSLRSRVTISVDTSKNQFSL | QKPGQAPRLLIYGASSRATGIPDRFSGSGSGTDFTLTI |
| | KLSSVTAADTAVYYCARTLRQWLARGYYYFDYWGQGTLVT | SRLEPEDFAVYYCQQYGSSPYTFGQGTKLEIK |
| | VSS (SEQ ID NO: 1625) | (SEQ ID NO: 1860) |
| AB- | QLQLQESGPGLVKPSETLSLTCTVSGGSISSSSYYWGWIR | QSALTQPASVSGSPGQSITISCTGTSSDVGGYNYVSWY |
| 009784 | QPPGKGLEWIGSIYYSGSTYYNPSLKSRVTISVDTSKNQF | QQHPGKAPQLMIYDVINRPSGVSNRFSGSKSGNTASLT |
| | SLKLSSVTAADTAVYYCVRHRGWIQLWFFDYWGQGTLVTV | ISGLQAEDEADYYCSSYTSSSTVVFGGGTKLTVL |
| | SS (SEQ ID NO: 1626) | (SEQ ID NO: 1861) |
| AB- | QVQLQESGPGLVKPSETLSLTCTVSGGSISSYYWSWIRQP | EIVLTQSPGTLSLSPGERATLSCRASQSVSSSYLAWYQ |
| 009785 | PGKGLEWIGYIYYSGSTNYNPSLKSRVTISVDTSKNQFSL | QKPGQAPRLLIYGASSRATGIPDRFSGSGSGTDFTLTI |
| | KLSSVTAADTAVYYCARLRAHTIFGVPIADAFDIWGQGTM | SRLEPEDFAVYYCQQYGSSPLTFGGGTKVEIK |
| | VTVSS (SEQ ID NO: 1627) | (SEQ ID NO: 1862) |
| AB- | QVQLQQWGAGLLKPSETLSLTCAVYGGSFSGYYWSWIRQP | SSELTQDPAVSVALGQTVRITCQGDSLRSYYASWYQQ |
| 009786 | PGKGLEWIGEINHSGSTNYNPSLKSRVTISVDTSKNQFSL | KPGQAPVVVIYGKNNRPSGIPDRFSGSSSGNTASLTIT |
| | KLSSVTAADTAVYYCARHLSLITVIVVVTYQLDAFDIWGQ | GAQAEDEADYYCNSRDSSGNHRVFGGGTKLTVL |
| | GTMVTVSS (SEQ ID NO: 1628) | (SEQ ID NO: 1863) |
| AB- | QLQLQESGPGLVKPSETLSLTCTVSGGSISSSSYYWGWIR | QSALTQPASVSGSPGQSITISCTGTSSDVGSYNLVSWY |
| 009787 | QPPGKGLEWIGSIYYSGSTYYNPSLKSRVTISVDTSKNQF | QQHPGKAPKLMIYEGSKRPSGVSNRFSGSKSGNTASLT |
| | SLKLSSVTAADTAVYYCARHYSSITIFGVVSGRYMDVWGK | ISGLQAEDEADYYCCSYAGSAVFGGGTKLTVL |
| | GTTVTVSS (SEQ ID NO: 1629) | (SEQ ID NO: 1864) |
| AB- | QVQLQESGPGLVKPSETLSLTCTVSGGSISSHYWSWIRQP | EIVLTQSPGTLSLSPGERATLSCRASQSVSSSYLAWYQ |
| 009788 | PGKGLEWIGYIYYSGSTNYNPSLKSRVTISVDTSKNQFSL | QKPGQAPRLLIYGASSRATGIPDRFSGSGSGTDFTLTI |
| | KLSSVTAADTAVYYCARDLRGWLQSPNWYFDLWGRGTLVT | SRLEPEDFAVYYCQQYGSSPAYTFGQGTKLEIK |
| | VSS (SEQ ID NO: 1630) | (SEQ ID NO: 1865) |
| AB- | EVQLVESGGGLVQPGGSLRLSCAASGFTFSSYAMSWVRQA | SYELTQPPSVSVSPGQTASITCSGDKLGDKYACWYQQ |
| 009789 | PGKGLEWVSAISGSGGSTYYADSVKGRFTISRDNSKNTLY | KPGQSPVLVIYQDSKRPSGIPERFSGSNSGNTATLTIS |
| | LQMNSLRAEDTAVYYCAKESGSGYYNWGQGTLVTVSS | GTQAMDEADYYCQAWDSSSVVFGGGTKLTVL |
| | (SEQ ID NO: 1631) | (SEQ ID NO: 1866) |
| AB- | EVQLLESGGGLVQPGGSLRLSCAASGFTFSSYAMSWVRQA | SYELTQPPSVSVSPGQTASITCSGDKLGDKYACWYQQ |
| 009790 | PGKGLEWVSAISGSGGSTYYADSVKGRFTISRDNSKNTLY | KPGQSPVLVIYQDSKRPSGIPERFSGSNSGNTATLTIS |
| | LQMNSLRAEDTAVYYCAKTSGTGTLAYWGQGTLVTVSS | GTQAMDEADYYCQAWDSSTLVFGGGTKLTVL |
| | (SEQ ID NO: 1632) | (SEQ ID NO: 1867) |
| AB- | QLQLQESGPGLVKPSETLSLTCTVSGGSISSSSYYWGWIR | SYELTQPPSVSVSPGQTASITCSGDKLGDKYACWYQQ |
| 009791 | QPPGKGLEWIGSIYYSGSTYYNPSLKSRVTISVDTSKNQF | KPGQSPVLVIYQDSKRPSGIPERFSGSNSGNTATLTIS |
| | SLKLSSVTAADTAVYYCAREPRRVKQWLVVPYYYMDVWGK | GTQAMDEADYYCQAWDSSIVVFGGGTKLTVL |
| | GTTVTVSS (SEQ ID NO: 1633) | (SEQ ID NO: 1868) |
| AB- | QVQLQESGPGLVKPSETLSLTCTVSGGSISSYYWSWIRQP | DIQMTQSPSTLSASVGDRVTITCRASQSISSWLAWYQQ |
| 009792 | PGKGLEWIGYIYYSGSTNYNPSLKSRVTISVDTSKNQFSL | KPGKAPKLLIYKASSLESGVPSRFSGSGSGTEFTLTIS |
| | KLSSVTAADTAVYYCARTLHSTIFGVVIMPSSFDYWGQGT | SLQPDDFATYYCQQYNSYATFGQGTKLEIK |
| | LVTVSS (SEQ ID NO: 1634) | (SEQ ID NO: 1869) |
| AB- | QVQLVQSGAEVKKPGASVKVSCKASGYTFTSYGISWVRQA | EIVLTQSPGTLSLSPGERATLSCRASQSVSSSYLAWYQ |
| 009793 | PGQGLEWMGWISAYNGNTNYAQKLQGRVTMTTDTSTSTA | QKPGQAPRLLIYGASSRATGIPDRFSGSGSGTDFTLTI |
| | YMELRSLRSDDTA VYYCARLHRMITFGGVIAQLDVWGKG | SRLEPEDFAVYYCQQYGSSPWTFGQGTKVEIK |
| | TTVTVSS (SEQ ID NO: 1635) | (SEQ ID NO: 1870) |
| AB- | QVQLVQSGAEVKKPGASVKVSCKVSGYTLTELSMHWVRQ | QSVLTQPPSVSEAPGQRVTISCTGSSSNIGAGYDVHWY |
| 009794 | APGKGLEWMGGFDPEDGETIYAQKFQGRVTMTEDTSTDTA | QQLPGTAPKLLIYGNSNRPSGVPDRFSGSKSGTSASLA |
| | YMELSSLRSEDTAVYYCATAGEQWLDRYYFDYWGQGTLV | ITGLQAEDEADYYCQSYDSSLSARYVFGTGTKVTVL |
| | TVSS (SEQ ID NO: 1636) | (SEQ ID NO: 1871) |
| AB- | QVQLVQSGAEVKKPGASVKVSCKASGYTFTSYDINWVRQA | SYELTQPPSVSVSPGQTASITCSGDKLGDKYACWYQQ |
| 009795 | TGQGLEWMGWMNPNSGNTGYAQKFQGRVTMTRNTSISTA | KPGQSPVLVIYQDSKRPSGIPERFSGSNSGNTATLTIS |
| | YMELSSLRSEDTAVYYCARARGYQLLFNYYYYMDVWGKG | GTQAMDEADYYCQAWDSSTAVFGGGTKLTVL |
| | TTVTVSS (SEQ ID NO: 1637) | (SEQ ID NO: 1872) |
| AB- | QLQLQESGPGLVKPSETLSLTCTVSGGSISSSSYYWGWIR | SSELTQDPAVSVALGQTVRITCQGDSLRSYYASWYQQ |
| 009796 | QPPGKGLEWIGSIYYSGSTYYNPSLKSRVTISVDTSKNQF | KPGQAPVLVIYGKNNRPSGIPDRFSGSSSGNTASLTIT |
| | SLKLSSVTAADTAVYYCARHKGQWLVRANWFDPWGQGTLV | GAQAEDEADYYCNSRDSSGNHLFGGGTKLTVL |
| | TVSS (SEQ ID NO: 1638) | (SEQ ID NO: 1873) |
| AB- | QLQLQESGPGLVKPSETLSLTCTVSGGSISSSSYYWGWIR | EIVLTQSPGTLSLSPGERATLSCRASQSVSSSYLAWYQ |
| 009797 | QPPGKGLEWIGSIYYSGSTYYNPSLKSRVTISVDTSKNQF | QKPGQAPRLLIYGASSRATGIPDRFSGSGSGTDFTLTI |
| | SLKLSSVTAADTAVYYCARHLRGGVIVATIYGVDYWGQGT | SRLEPEDFAVYYCQQYGSSQTFGQGTKVEIK |
| | LVTVSS (SEQ ID NO: 1639) | (SEQ ID NO: 1874) |
| AB- | QVQLVQSGAEVKKPGASVKVSCKASGYTFTSYYMHWVRQ | QSALTQPASVSGSPGQSITISCTGTSSDVGSYNLVSWY |
| 009798 | APGQGLEWMGIINPSGGSTSYAQKFQGRVTMTRDTSTSTV | QQHPGKAPKLMIYEGSKRPSGVSNRFSGSKSGNTASLT |
| | YMELSSLRSEDTAVYYCAREVPTFTIFWPLGRGGGWFDPW | ISGLQAEDEADYYCCSYAGSVVFGGGTKLTVL |
| | GQGTLVTVSS (SEQ ID NO: 1640) | (SEQ ID NO: 1875) |
| AB- | QVQLVQSGAEVKKPGASVKVSCKASGYTFTSYDINWVRQA | SYELTQPPSVSVSPGQTASITCSGDKLGDKYACWYQQ |
| 009799 | TGQGLEWMGWMNPNSGNTGYAQKFQGRVTMTRNTSISTA | KPGQSPVLVIYQDSKRPSGIPERFSGSNSGNTATLTIS |
| | YMELSSLRSEDTAVYYCARGRLRITIFNRVYYFDYWGQGT | GTQAMDEADYYCQAWDSSTAWVFGGGTKLTVL |
| | VLTVSS (SEQ ID NO: 1641) | (SEQ ID NO: 1876) |
| AB- | EVQLVESGGGLVKPGGSLRLSCAASGFTFSSYSMNWVRQA | QSALTQPPSASGSPGQSVTISCTGTSSDVGGYNYVSWY |
| 009800 | PGKGLEWVSSISSSSSYIYYADSVKGRFTISRDNAKNSLY | QQHPGKAPKLMIYEVSKRPSGVPDRFSGSKSGNTASLT |
| | LQMNSLRAEDTAVYYCARGHFDWLLYETYYFDYWGQGTLV | VSGLQAEDEADYYCSSYAGSNNVVFGGGTKLTVL |
| | TVSS (SEQ ID NO: 1642) | (SEQ ID NO: 1877) |
| AB- | EVQLVESGGGLVQPGGSLRLSCAASGFTFSSYWMSWVRQA | SYELTQPPSVSVSPGQTASITCSGDKLGDKYACWYQQ |
| 009801 | PGKGLEWVANIKQDGSEKYYVDSVKGRFTISRDNAKNSLY | KPGQSPVLVIYQDSKRPSGIPERFSGSNSGNTATLTIS |
| | LQMNSLRAEDTAVYYCARDTVRREQWLVLDYYYMDVWG | QGTAMDEADYYCQAWDSSIHVVFGGGTKLTVL |
| | KGTTVTVSS (SEQ ID NO: 1643) | (SEQ ID NO: 1878) |
| AB- | QLQLQESGPGLVKPSETLSLTCTVSGGSISSSSYYWGWIR | QSVLTQPPSVSGAPGQRVTISCTGSSSNIGAGYDVHWY |
| 009802 | QPPGKGLEWIGSIYYSGSTYYNPSLKSRVTISVDTSKNQF | QQLPGTAPKLLIYGNSNRPSGVPDRFSGSKSGTSASLA |
| | SLKLSSVTAADTAVYYCARQPRRITIFGVVITPTNNWFDP | ITGLQAEDEADYYCQSYDSSLSDVVFGGGTKLTVL |
| | WGQGTLVTVSS (SEQ ID NO: 1644) | (SEQ ID NO: 1879) |
| AB- | EVQLVESGGGLVQPGGSLRLSCAASGFTVSSNYMSWVRQA | EIVMTQSPATLSVSPGERATLSCRASQSVSSNLAWYQQ |
| 010019 | PGKGLEWVSVIYSGGSTYYADSVKGRFTISRDNSKNTLYL | KPGQAPRLLIYGASTRATGIPARFSGSGSGTEFTLTIS |
| | QMNSLRAEDTAVYYCARDYGDFYFDYWGQGTLVTVSS | SLQSEDFAVYYCQQYNNWPRTFGQGTKVEIK |
| | (SEQ ID NO: 1645) | (SEQ ID NO: 1880) |
| AB- | QVQLVESGGGVVQPGRSLRLSCAASGFTFSSYGMHWVRQA | DIVMTQSPDSLAVSLGERATINCKSSQSVLYSSNNKNY |
| 4497999 | PGKGLEWVAVISYDGSNKYYADSVKGRFTISRDNSKNTLY | LAWYQQKPGQPPKLLIYWASTRESGVPDRFSGSGSGT |
| 20 | LQMNSLRAEDTAVYYCAKDYGDYGAFDIWGQGTMVTVSS | DFTLTISSLQAEDVAVYYCQQYYSTPHFGPGTKVDIK |
| | (SEQ ID NO: 1929) | (SEQ ID NO: 1937) |
| AB- | QVQLVQSGAEVKKPGSSVKVSCKASGGTFSSYAISWVRQA | EIVLTQSPATLSLSPGERATLSCRASQSVSSYLAWYQQ |
| 4496014 | PGQGLEWMGGIIPIFGTANYAQKFQGRVTITADESTSTAY | KPGQAPRLLIYDASNRATGIPARFSGSGSGTDFTLTIS |
| 61 | MELSSLRSEDTAVYYCASVGGDYDLDYWGQGTLVTVSS | SLEPEDFAVYYCQQRSNWPPYTFGQGTKLEIK |
| | (SEQ ID NO: 1930) | (SEQ ID NO: 1938) |
| AB- | QVQLVQSGAEVKKPGSSVKVSCKASGGTFSSYAISWVRQA | EIVLTQSPATLSLSPGERATLSCRASQSVSSYLAWYQQ |
| 4496352 | PGQGLEWMGGIIPIFGTANYAQKFQGRVTITADESTSTAY | KPGQAPRLLIYDASNRATGIPARFSGSGSGTDFTLTIS |
| 24 | MELSSLRSEDTAVYYCASVGGDYDLDYWGQGTLVTVSS | SLEPEDFAVYYCQQRSNWPPYTFGQGTKLEIK |
| | (SEQ ID NO: 1931) | (SEQ ID NO: 1939) |
| AB- | QVQLVQSGAEVKKPGSSVKVSCKASGGTFSSYAISWVRQA | EIVLTQSPATLSLSPGERATLSCRASQSVSSYLAWYQQ |
| 4497472 | PGQGLEWMGGIIPIFGTANYAQKFQGRVTITADESTSTAY | KPGQAPRLLIYDASNRATGIPARFSGSGSGTDFTLTIS |
| 65 | MELSSLRSEDTAVYYCASVGGDYDLDYWGQGTLVTVSS | SLEPEDFAVYYCQQRSNWPPYTFGQGTKLEIK |
| | (SEQ ID NO: 1932) | (SEQ ID NO: 1940) |
| AB- | QVQLVQSGAEVKKPGSSVKVSCKASGGTFSSYAISWVRQA | EIVLTQSPATLSLSPGERATLSCRASQSVSSYLAWYQQ |
| 4497813 | PGQGLEWMGGIIPIFGTANYAQKFQGRVTITADESTSTAY | KPGQAPRLLIYDASNRATGIPARFSGSGSGTDFTLTIS |
| 53 | MELSSLRSEDTAVYYCASVGGDYDLDYWGQGTLVTVSS | SLEPEDFAVYYCQQRSNWPPYTFGQGTKLEIK |
| | (SEQ ID NO: 1933) | (SEQ ID NO: 1941) |
| AB- | QVQLVQSGAEVKKPGSSVKVSCKASGGTFSSYAISWVRQA | EIVLTQSPATLSLSPGERATLSCRASQSVSSYLAWYQQ |
| 4498417 | PGQGLEWMGGIIPIFGTANYAQKFQGRVTITADESTSTAY | KPGQAPRLLIYDASNRATGIPARFSGSGSGTDFTLTIS |
| 46 | MELSSLRSEDTAVYYCASVGGDYDLDYWGQGTLVTVSS | SLEPEDFAVYYCQQRSNWPPYTFGQGTKLEIK |
| | (SEQ ID NO: 1934) | (SEQ ID NO: 1942) |
| AB- | QITLKESGPTLVKPTQTLTLTCTFSGFSLSTSGVGVGWIR | QSALTQPASVSGSPGQSITISCTGASSDVGAYNYVSWY |
| 4496458 | QPPGKALEWLALIYWDDDKRYSPSLKSRLTITKDTSKNQV | QQHPGKAPKLMIYDVSNRPSGVSNRFSGSKSGNTASLT |
| 91 | VLTMTNLDPVDTATYYCAHLTTLTIFDYWGQGTLVTVSS | ISGLQAEDEADYFCSSYTSSSVVFGGGTKLTVL |
| | (SEQ ID NO: 1935) | (SEQ ID NO: 1943) |
| AB- | QITLKESGPTLVKPTQTLTLTCTFSGFSPSTSGVGVGWIR | QSALTQPASVSGSPGQSITISCTGTRSDVGGYNYVSWY |
| 4495981 | QPPGKALEWLALIYWDDDKRYSPSLKSRLTITQDTSKNQV | QHHPGKAPKLMIYEVSNRPSGVSNRFSGSKSGNTASLT |
| 46 | VLTMTNMDPVDTATYYCAHHSVATIWDYWGQGTLVTVSS | ISGLQAEDEADYYCSSYTSSSTLVFGGGTKLTVL |
| | (SEQ ID NO: 1936) | (SEQ ID NO: 1944) |

TABLE 8
Neutralization data

| | | Pseudovirus Neturalization (μg/mL) | | | | | |
|---|---|---|---|---|---|---|---|
| | | | | | SARS- | | |
| | Neutralization | | | SARS- | CoV2 | SARS- | |
| | (Infectious | SARS- | SARS- | CoV2 | Wuhan | CoV-2 | SARS-CoV-2 |
| | virus; | CoV- | CoV2 | Wuhan | D614G | “Alpha” | “Beta” |
| Antibody ID | (μg/mL)) | 1 | Wuhan | D614G | Repeat* | UKB.1.1.7 | A_SAv2B.1.351 |
| AB-009614 | 0.075 | >50 | 0.142 | 0.040 | 0.114 | >50 | >50 |
| AB-010019 | N/T | 40.46 | 3.050 | 1.040 | 1.605 | 3.402 | >50 |
| AB-009270 | 1.37 | >50 | 0.065 | 0.058 | 0.040 | 0.117 | 0.169 |
| AB-009346 | 2.14 | >50 | 0.774 | 0.177 | 0.189 | 0.114 | >50 |
| AB-009681 | 3.92 | >50 | >50 | 5.514 | 2.83 | >50 | >50 |
| AB-009761 | 4.44 | >50 | 6.53 | 21.68 | 15.74 | 6.07 | >50 |
| AB-009615 | 12.3 | >50 | 27.70 | >50 | >50 | >50 | >50 |
| AB-009620 | 13.3 | >50 | 1.04 | 1.06 | 0.398 | 0.257 | 0.563 |
| AB-009661 | 20.6 | >50 | >50 | >50 | >50 | >50 | >50 |
| AB-009734 | 21.43 | >50 | 42.38 | >50 | >50 | >50 | >50 |
| AB-009769 | 24.47 | >50 | >50 | >50 | 1.78 | >50 | >50 |
| AB-009760 | 32.76 | >50 | 4.10 | 39.1 | 28.0 | 17.7 | >50 |
| AB-009281 | 33.7 | >50 | 0.215 | 0.105 | 0.086 | >50 | >50 |
| SARS-CoV-1 Ab | >50 | 2.61 | >50 | >50 | >50 | >50 | >50 |
| CR3022 (AB- | | | | | | | |
| 009073) | | | | | | | |
| COVA1-21 (AB- | N/T | >50 | 21.0 | 1.72 | 2.30 | >50 | >50 |
| 010159) | | | | | | | |
| CA1 (AB-009265) | 2.46 | >50 | 0.266 | 0.296 | 0.265 | 0.209 | >50 |
| REGN10933 \| | N/T | >50 | 0.0005 | 0.0008 | 0.0014 | 0.0003 | 1.09 |
| Casirivimab (AB- | | | | | | | |
| 010156) | | | | | | | |
| REGN10987 \| | N/T | >50 | 0.0005 | 0.0003 | 0.0004 | 0.0003 | <0.0002 |
| Imdevimab (AB- | | | | | | | |
| 010157) | | | | | | | |
| LY-CoV016 \| JS- | 0.13 | >50 | 0.012 | 0.014 | 0.009 | 0.417 | >50 |
| 016 \|Etesevimab | | | | | | | |
| (AB-009264) | | | | | | | |
| LY-CoV555 \| | N/T | >50 | <0.002 | <0.002 | 0.0002 | 0.0012 | >50 |
| Bamlanivimab | | | | | | | |
| (AB-010160) | | | | | | | |
| Anti-Nucleocapsid | N/T | >50 | >50 | >50 | >50 | >50 | >50 |
| (Neg Control) | | | | | | | |

| | Pseudovirus Neturalization (μg/mL) | | | | | |
|---|---|---|---|---|---|---|
| | | | SARS- | | | |
| | | SARS- | CoV-2 | SARS- | SARS- | SARS- |
| | SARS-CoV-2 | CoV-2 | “Delta - | CoV-2 | CoV-2 | CoV-2 |
| | “Gamma” | “Delta” | plus” | “Epsilon” | “Mu” | “Omicron” |
| Antibody ID | BrazilP.1.B.1.1.28 | B.1.617.2 | B.1.617.2.1 | B.1.427/9 | B.1.621 | B.1.1.529 |
| AB-009614 | >50 | 31.28 | >50 | >50 | >50 | >50 |
| AB-010019 | >50 | 0.158 | >50 | 1.27 | 1.984 | >50 |
| AB-009270 | 0.158 | 0.030 | 0.032 | 0.125 | >50 | |
| AB-009346 | >50 | >50 | >50 | 33.65 | >50 | >50 |
| AB-009681 | >50 | >50 | >50 | >50 | >50 | >50 |
| AB-009761 | 13.9 | >50 | 39.22 | 17.13 | >50 | >50 |
| AB-009615 | >50 | >50 | >50 | 38.35 | >50 | >50 |
| AB-009620 | 0.327 | 0.519 | 0.809 | 0.974 | 0.115 | 12.92 |
| AB-009661 | >50 | >50 | >50 | >50 | >50 | >50 |
| AB-009734 | 40.6 | >50 | >50 | >50 | >50 | >50 |
| AB-009769 | >50 | >50 | >50 | >50 | >50 | >50 |
| AB-009760 | 19.2 | >50 | 46.2 | 14.1 | >50 | >50 |
| AB-009281 | >50 | 0.032 | 2.29 | 0.041 | >50 | >50 |
| SARS-CoV-1 Ab | >50 | >50 | >50 | >50 | >50 | >50 |
| CR3022 (AB- | | | | | | |
| 009073) | | | | | | |
| COVA1-21 (AB- | >50 | >50 | >50 | >50 | >50 | >50 |
| 010159) | | | | | | |
| CA1 (AB-009265) | >50 | 2.19 | 41.49 | 0.33 | 21.19 | >50 |
| REGN10933 \| | 0.666 | 0.001 | 0.001 | 0.001 | 0.0004 | >50 |
| Casirivimab (AB- | | | | | | |
| 010156) | | | | | | |
| REGN10987 \| | 0.0003 | 0.001 | 0.001 | 0.001 | 0.0002 | >50 |
| Imdevimab (AB- | | | | | | |
| 010157) | | | | | | |
| LY-CoV016 \| JS- | >50 | 0.004 | >50 | 0.009 | 0.18 | >50 |
| 016 \|Etesevimab | | | | | | |
| (AB-009264) | | | | | | |
| LY-CoV555 \| | >50 | 19.3 | 6.93 | 7.82 | >50 | >50 |
| Bamlanivimab | | | | | | |
| (AB-010160) | | | | | | |
| Anti-Nucleocapsid | >50 | >50 | >50 | >50 | >50 | >50 |
| (Neg Control) | | | | | | |

TABLE 9
Characteristics of the SARS-CoV2 variants

| Residue | Wuhan | | | | | | | |
|---|---|---|---|---|---|---|---|---|
| in Spike | D614 | Alpha | Beta | Gamma | Delta | Epsilon | Mu | Omicron |
| | | UK | SA | BR | B.1.617.2 | B.1.427/B.1. | B.1.621 | B.1.1.529 |
| | | variant | B.1.351v2 | variant | | | | |
| | | B.1.1.7 | | P.1 | | | | |
| | | | | B.1.1.28 | | | | |
| 13 | | | | | | S13I | | |
| 18 | | | L18F | L18F | | | | |
| 19 | | | | | T19R | | | |
| 20 | | | | T20N | | | | |
| 26 | | | | P26S | | | | |
| 67 | | | | | | | | A67V |
| 69 | | DEL69-70 | | | | | | DEL69-70 |
| 89 | | | D80A | | | | | |
| 95 | | | | | | | T95I | T95I |
| 138 | | | | D138Y | | | | |
| 142 | | | | | | | | DEL142-144 |
| 144 | | | | | | | Y144S | |
| 145 | | DEL145 | | | | | Y145N | Y145D |
| 152 | | | | | | W152C | | |
| 157 | | | | | DEL157-158 | | | |
| 190 | | | | R190S | | | | |
| 211 | | | | | | | | DEL211 |
| 212 | | | | | | | | L212I |
| 215 | | | D215G | | | | | |
| 242 | | | DEL242-244 | | | | | |
| 246 | | | R246I | | | | | |
| 339 | | | | | | | | G339D |
| 346 | | | | | | | R346K | |
| 371 | | | | | | | | S371L |
| 373 | | | | | | | | S373P |
| 375 | | | | | | | | S375F |
| 417 | | | K417N* | K417T* | K417T* | | | K417N* |
| 440 | | | | | | | | N440K |
| 446 | | | | | | | | G446S |
| 452 | | | | | L452R* | L452R* | | |
| 477 | | | | | | | | S477N |
| 478 | | | | | T478K | | | T478K |
| 484 | | | E484K* | E484K* | | | E484K* | E484A* |
| 493 | | | | | | | | Q493R |
| 496 | | | | | | | | G496S |
| 498 | | | | | | | | Q498R |
| 501 | | N501Y* | N501Y* | N501Y* | | | N501Y* | N501Y* |
| 505 | | | | | | | | Y505H |
| 547 | | | | | | | | T547K |
| 570 | | A570D | | | | | | |
| 614 | D614G | D614G | D614G | D614G | D614G | D614G | D614G | D614G |
| 655 | | | | H655Y | | | | H655Y |
| 679 | | | | | | | | N679K |
| 681 | | P681H | | | P681R | | P681H | P681H |
| 701 | | | A701V | | | | | |
| 716 | | T716I | | | | | | |
| 764 | | | | | | | | N764K |
| 796 | | | | | | | | D796Y |
| 856 | | | | | | | | N856K |
| 950 | | | | | D950N | | D950N | |
| 954 | | | | | | | | Q954H |
| 969 | | | | | | | | N969K |
| 981 | | | | | | | | L981F |
| 982 | | S982A | | | | | | |
| 1027 | | | | T1027I | | | | |
| 1118 | | D1118H | | | | | | |

Note:
modifications of residues indicated by * may impact the binding of an antibody that had binding activity against the original SARS-CoV-2 strain.

### REFERENCES

Wrammert J, et al., Rapid and massive virus-specific plasmablast responses during acute dengue virus infection in humans. J Virol. 86, 2911-2918 (2012).
DeFalco J et al., Non-progressing cancer patients have persistent B cell responses expressing shared antibody paratopes that target public tumor antigens. Clin Immunol. 187, 37-45 (2018).
Zhiqiang Ku, et al., Antibody therapies for the treatment of COVID-19, Antibody Therapeutics, Volume 3, Issue 2, April 2020, Pages 101-108.
Walls et al., Structure, Function, and Antigenicity of the SARS-CoV-2 Spike Glycoprotein, 2020, Cell 180, 1-12, Mar. 19, 2020.
Claudia A. Castro Jaramillo, et al. (2017) Toward in vitro-to-in vivo translation of monoclonal antibody pharmacokinetics: Application of a neonatal Fc receptor-mediated transcytosis assay to understand the interplaying clearance mechanisms, mAbs, 9:5, 781-791, DOI: 10.1080/19420862.2017.1320008.
Chung S, Nguyen V, Lin Y L, et al. An in vitro FcRn-dependent transcytosis assay as a screening tool for predictive assessment of nonspecific clearance of antibody therapeutics in humans. MAbs. 2019; 11(5):942-955. doi:10.1080/19420862.2019.1605270.
Rogers et al., “Rapid isolation of potent SARS-CoV-2 neutralizing antibodies and protection in a small animal model.” doi://doi.org/10.1101/2020.05.11.088674.

It is understood that the examples and embodiments described herein are for illustrative purposes only and that various modifications may be suggested to persons skilled in the art after reviewing this disclosure, which are to be included within the scope of the appended claims. All publications, sequence accession numbers, patents, and patent applications cited herein are hereby incorporated by reference in their entirety for all purposes.

1. An antibody that inhibits binding of a coronavirus to a cell or reduces infection of a cell by a coronavirus, optionally wherein the coronavirus is a member of the betacoronavirus genus, and optionally wherein the wherein the coronavirus is a Severe acute respiratory syndrome coronavirus 2 (SARS-CoV-2).

2-3. (canceled)

4. The antibody of claim 1, wherein the coronavirus is a variant of SARS-CoV-2 selected from the group consisting of the Alpha, Beta, Gamma, Delta, Epsilon, Mu, and Omicron variants.

5. The antibody of claim 1, wherein the antibody comprises all six CDRs of an antibody selected from the group consisting of AB-009614, AB-010019, AB-009270, AB-009346, AB-009681, AB-009761, AB-009620, AB-009734, AB-009760, and AB-009281, or wherein the antibody comprises both the VH and VL of an antibody selected from the group consisting of AB-009614, AB-010019, AB-009270, AB-009346, AB-009681, AB-009761, AB-009620, AB-009734, AB-009760, and AB-009281.

6. The antibody of The antibody of claim 1, wherein the antibody inhibits binding of Alpha, Beta, Gamma, Delta, Epsilon, Mu, and Omicron variants of SARS-CoV-2 and/or reduces infection of a cell by Alpha, Beta, Gamma, Delta, Epsilon, Mu, and Omicron variants of SARS-CoV-2.

7. (canceled)

8. The antibody of claim 1, wherein the antibody comprises the LCDRs and HCDRs of an antibody listed in Table 4.

9. The antibody of claim 1, wherein the antibody comprises
i) a VH amino acid sequence and a VL amino acid sequence listed in Table 5, or ii) a VH amino acid sequence with at least 70% identity to the VH amino acid sequence in Table 5 and a VL amino acid sequence with at least 70% identity to the VL amino acid sequence in Table 5, wherein variations as compared to the VH amino acid sequence or the VL amino acid sequence in Table 5 are in the framework regions only.

10. The antibody of claim 1, wherein the antibody comprises all six CDRs of an antibody in Table 4 and wherein the antibody comprises heavy chain framework regions having at least 70% sequence identity to the heavy chain framework regions in the same antibody in Table 4, and/or light chain framework regions having at least 70% sequence identity to the light chain framework regions of the same antibody in Table 4.

11-19. (canceled)

20. The antibody of claim 1, wherein the antibody binds to the S trimer encoded by the coronavirus, and optionally wherein the antibody binds to RBD, S1 monomer and S trimer.

21. (canceled)

22. The antibody of claim 20, wherein the antibody does not bind to the S2 protein.

23. The antibody of claim 1, wherein the antibody inhibits binding of the coronavirus to a receptor on the surface of the cell, optionally wherein the cell surface receptor is ACE2.

24. (canceled)

25. An isolated antibody or an antibody fragment of claim 1, optionally, wherein the antibody is a chimeric antibody, a multispecific antibody, a bispecific antibody, an scFv, or a Fab.

26-27. (canceled)

28. A pharmaceutical composition comprising the antibody of claim 1.

29. A recombinant nucleic acid encoding an antibody or a fragment of an antibody of claim 1.

30. An expression vector comprising a heterologous polynucleotide encoding a heavy chain variable region listed in Table 5, or comprising a heterologous polynucleotide encoding a light chain variable region listed in Table 5, or comprising a heterologous polynucleotide encoding a cognate pair of heavy and light chain variable regions listed in Table 5.

31-32. (canceled)

33. A host cell that comprises an expression vector of claim 30.

34. A method of producing an antibody that inhibits binding of a coronavirus to a cell, the method comprising culturing the host cell of claim 33 under conditions in which the polynucleotide encoding the heavy chain and the polynucleotide encoding the light chain are expressed.

35. (canceled)

36. A method of inducing an immune response, the method comprising administering an antibody of claim 1.

37-38. (canceled)

39. A method of treating a patient infected with a coronavirus, the method comprising administering a therapeutically effective amount of the antibody of claim 1, to the patient, optionally wherein the method further comprises administering a second treatment to the patient, wherein the second treatment is selected from an anti-viral agent or an anti-inflammatory agent, and optionally wherein the second treatment comprises an antibody that binds SARS-CoV-2.

40-44. (canceled)

45. A method of identifying a patient that is infected with a coronavirus, the method comprising detecting binding of the antibody of claim 1 to a sample obtained from the patient, wherein binding greater than a negative control value indicates the patient is infected with the coronavirus.

46-48. (canceled)

49. A method of identifying an antibody having anti-viral activity, the method comprising mutagenizing a polynucleotide encoding a heavy chain variable region or a light chain variable region of an antibody of claim 1, expressing an antibody comprising the mutagenized heavy chain or light chain variable region; and selecting an antibody that inhibits binding of the virus to a cell.

50-56. (canceled)
